SEQUENCE LISTING (1131 sequences)

SEQ ID NO: 1 - protein, 20 aa, synthetic construct
GKPGSGKPGSGKPGSGKPGS

SEQ ID NO: 2 - protein, 5 aa, synthetic construct
GGGGS

SEQ ID NO: 3 - protein, 15 aa, synthetic construct
GGGGSGGGGSGGGGS

SEQ ID NO: 4 - protein, 18 aa, synthetic construct
GSTSGSGKPGSGEGSTKG

SEQ ID NO: 5 - protein, 14 aa, synthetic construct
IRPRAIGGSKPRVA

SEQ ID NO: 6 - protein, 15 aa, synthetic construct
GKGGSGKGGSGKGGS

SEQ ID NO: 7 - protein, 15 aa, synthetic construct
GGKGSGGKGSGGKGS

SEQ ID NO: 8 - protein, 15 aa, synthetic construct
GGGKSGGGKSGGGKS

SEQ ID NO: 9 - protein, 15 aa, synthetic construct
GKGKSGKGKSGKGKS

SEQ ID NO: 10 - protein, 15 aa, synthetic construct
GGGKSGGKGSGKGGS

SEQ ID NO: 11 - protein, 15 aa, synthetic construct
GKPGSGKPGSGKPGS

SEQ ID NO: 12 - protein, 20 aa, synthetic construct
GKGKSGKGKSGKGKSGKGKS

SEQ ID NO: 13 - protein, 20 aa, synthetic construct
GGGGSGGGGSGGGGSGGGGS

SEQ ID NO: 14 - protein, 14 aa, synthetic construct
STAGDTHLGGEDFD

SEQ ID NO: 15 - protein, 15 aa, synthetic construct
GEGGSGEGGSGEGGS

SEQ ID NO: 16 - protein, 15 aa, synthetic construct
GGEGSGGEGSGGEGS

SEQ ID NO: 17 - protein, 15 aa, synthetic construct
GGGESGGGESGGGES

SEQ ID NO: 18 - protein, 15 aa, synthetic construct
GEGESGEGESGEGES

SEQ ID NO: 19 - protein, 15 aa, synthetic construct
GGGESGGEGSGEGGS

SEQ ID NO: 20 - protein, 20 aa, synthetic construct
GEGESGEGESGEGESGEGES

SEQ ID NO: 21 - protein, 19 aa, synthetic construct
PRGASKSGSASQTGSAPGS

SEQ ID NO: 22 - protein, 19 aa, synthetic construct
GTAAAGAGAAGGAAAGAAG

SEQ ID NO: 23 - protein, 19 aa, synthetic construct
GTSGSSGSGSGGSGSGGGG

SEQ ID NO: 24 - protein, 10 aa, synthetic construct
GGGGSGGGGS

SEQ ID NO: 25 - protein, 25 aa, synthetic construct
GGGGSGGGGSGGGGSGGGGSGGGGS

SEQ ID NO: 26 - protein, 30 aa, synthetic construct
GGGGSGGGGSGGGGSGGGGSGGGGSGGGGS

SEQ ID NO: 27 - protein, 35 aa, synthetic construct
GGGGSGGGGSGGGGSGGGGSGGGGSGGGGSGGGGS

SEQ ID NO: 28 - protein, 5 aa, synthetic construct
GGGGA

SEQ ID NO: 29 - protein, 10 aa, synthetic construct
GGGGAGGGGA

SEQ ID NO: 30 - protein, 15 aa, synthetic construct
GGGGAGGGGAGGGGA

SEQ ID NO: 31 - protein, 20 aa, synthetic construct
GGGGAGGGGAGGGGAGGGGA

SEQ ID NO: 32 - protein, 25 aa, synthetic construct
GGGGAGGGGAGGGGAGGGGAGGGGA

SEQ ID NO: 33 - protein, 30 aa, synthetic construct
GGGGAGGGGAGGGGAGGGGAGGGGAGGGGA

SEQ ID NO: 34 - protein, 35 aa, synthetic construct
GGGGAGGGGAGGGGAGGGGAGGGGAGGGGAGGGGA

SEQ ID NO: 35 - protein, 30 aa, synthetic construct
DPALVHQRPAPPGGGGSGGGGSGGGGSGGG

SEQ ID NO: 36 - protein, 5 aa, synthetic construct
GKPGS

SEQ ID NO: 37 - protein, 25 aa, synthetic construct
GKPGSGKPGSGKPGSGKPGSGKPGS

SEQ ID NO: 38 - protein, 30 aa, synthetic construct
GKPGSGKPGSGKPGSGKPGSGKPGSGKPGS

SEQ ID NO: 39 - protein, 5 aa, synthetic construct
GGGES

SEQ ID NO: 40 - protein, 15 aa, synthetic construct
EPKSCDKTHTCPPCP

SEQ ID NO: 41 - protein, 9 aa, synthetic construct
KTHTCPPCP

SEQ ID NO: 42 - protein, 15 aa, synthetic construct
EPKSSDKTHTCPPCP

SEQ ID NO: 43 - protein, 19 aa, synthetic construct
GGGGSGGGGSKTHTCPPCP

SEQ ID NO: 44 - protein, 19 aa, synthetic construct
GKPGSGKPGSKTHTCPPCP

SEQ ID NO: 45 - protein, 14 aa, synthetic construct
GKPGSKTHTCPPCP

SEQ ID NO: 46 - protein, 5 aa, synthetic construct
EPKSC

SEQ ID NO: 47 - protein, 15 aa, synthetic construct
EPKSCGGGGSGGGGS

SEQ ID NO: 48 - protein, 15 aa, synthetic construct
EPKSCGKPGSGKPGS

SEQ ID NO: 49 - protein, 10 aa, synthetic construct
EPKSCGKPGS

SEQ ID NO: 50 - protein, 216 aa, synthetic construct
  source 1..216: note = Heterodimeric Fc Backbone 1 monomer 1 (-)
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 51 - protein, 216 aa, synthetic construct
  source 1..216: note = Heterodimeric Fc Backbone 1 monomer 2 (+)
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 216 aa, synthetic construct; identical to SEQ ID NO: 50
  source 1..216: note = Heterodimeric Fc Backbone 2 monomer 1 (-)

SEQ ID NO: 53 - protein, 216 aa, synthetic construct
  source 1..216: note = Heterodimeric Fc Backbone monomer 2 (+)
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 216 aa, synthetic construct
  source 1..216: note = Heterodimeric Fc Backbone 3 monomer 1 (-)
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCEVSGFYPSDIAVEWESDGQPENNYKTTPPVLDSDGSFFLYSKLTVDKSRWEQGDVFSCSVMHEALHNHYTQKSLSLSPGK

SEQ ID NO: 55 - protein, 216 aa, synthetic construct; identical to SEQ ID NO: 53
  source 1..216: note = Heterodimeric Fc Backbone 3 monomer 2 (+)

SEQ ID NO: 56 - protein, 216 aa, synthetic construct
  source 1..216: note = Heterodimeric Fc Backbone 4 monomer 1 (-)
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTENEVSLTCLVKGFYPSDIAVEWESDGQPENNYKTTPPVLDSDGSFFLYSKLEVDKSRWEQGDVFSCSVMHEALHNHYTQKSLSLSPGK

SEQ ID NO: 57 - protein, 216 aa, synthetic construct
  source 1..216: note = Heterodimeric Fc Backbone 4 monomer 2 (+)
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSKGSFFLYSKLTVDKSRWQQGNVFSCSVMHEALHNHYTQKSLSLSPGK

SEQ ID NO: 58 - protein, 216 aa, synthetic construct
  source 1..216: note = Heterodimeric Fc Backbone 5 monomer 1 (-)
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSRDELTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 59 - protein, 216 aa, synthetic construct
  source 1..216: note = Heterodimeric Fc Backbone 5 monomer 2 (+)
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSRDQLTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 216 aa, synthetic construct
  source 1..216: note = Heterodimeric Fc Backbone 6 monomer 1 (-)
APPVAGPSVFLFPPKPKDTLMISRTPEVTCVVVDVKHEDPEVKFNWYVDGVEVHNAKTKPREEEYASTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 61 - protein, 216 aa, synthetic construct
  source 1..216: note = Heterodimeric Fc Backbone 6 monomer 2 (+)
APPVAGPSVFLFPPKPKDTLMISRTPEVTCVVVDVKHEDPEVKFNWYVDGVEVHNAKTKPREEQYASTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 216 aa, synthetic construct
  source 1..216: note = Heterodimeric Fc Backbone 7 monomer 1 (-)
APPVAGPSVFLFPPKPKDTLMISRTPEVTCVVVDVKHEDPEVKFNWYVDGVEVHNAKTKPREEEYSSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 63 - protein, 216 aa, synthetic construct
  source 1..216: note = Heterodimeric Fc Backbone 7 monomer 2 (+)
APPVAGPSVFLFPPKPKDTLMISRTPEVTCVVVDVKHEDPEVKFNWYVDGVEVHNAKTKPREEQYSSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 217 aa, synthetic construct
  source 1..217: note = Heterodimeric Fc Backbone 8 monomer 1 (-)
APEFLGGPSVFLFPPKPKDTLMISRTPEVTCVVVDVSQEDPEVQFNWYVDGVEVHNAKTKPREEEFNSTYRVVSVLTVLHQDWLNGKEYKCKVSNKGLPSSIEKTISKAKGQPREPQVYTLPPSQEEMTKNQVSLTCDVSGFYPSDIAVEWESDGQPENNYKTTPPVLDSDGSFFLYSRLTVDKSRWEEGDVFSCSVMHEALHNHYTQKSLSLSLGK

SEQ ID NO: 65 - protein, 217 aa, synthetic construct
  source 1..217: note = Heterodimeric Fc Backbone 8 monomer 2 (+)
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEQMTKNQVKLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 66 - protein, 216 aa, synthetic construct
  source 1..216: note = Heterodimeric Fc Backbone 9 monomer 1 (-)
APPVAGPSVFLFPPKPKDTLMISRTPEVTCVVVDVSHEDPEVQFNWYVDGVEVHNAKTKPREEEFNSTFRVVSVLTVVHQDWLNGKEYKCKVSNKGLPAPIEKTISKTKGQPREPQVYTLPPSREEMTKNQVSLTCDVSGFYPSDIAVEWESDGQPENNYKTTPPMLDSDGSFFLYSKLTVDKSRWEQGDVFSCSVMHEALHNHYTQKSLSLSPGK

SEQ ID NO: 67 - protein, 216 aa, synthetic construct
  source 1..216: note = Heterodimeric Fc Backbone 9 monomer 2 (+)
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREQMTKNQVKLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 68 - protein, 216 aa, synthetic construct
  source 1..216: note = Heterodimeric Fc Backbone 10 monomer 1 (-)
APPVAGPSVFLFPPKPKDTLMISRTPEVTCVVVDVKHEDPEVQFNWYVDGVEVHNAKTKPREEEFNSTFRVVSVLTVVHQDWLNGKEYKCKVSNKGLPAPIEKTISKTKGQPREPQVYTLPPSREEMTKNQVSLTCDVSGFYPSDIAVEWESDGQPENNYKTTPPMLDSDGSFFLYSKLTVDKSRWEQGDVFSCSVMHEALHNHYTQKSLSLSPGK

SEQ ID NO: 69 - protein, 216 aa, synthetic construct
  source 1..216: note = Heterodimeric Fc Backbone 10 monomer 2 (+)
APPVAGPSVFLFPPKPKDTLMISRTPEVTCVVVDVKHEDPEVQFNWYVDGVEVHNAKTKPREEQFNSTFRVVSVLTVVHQDWLNGKEYKCKVSNKGLPAPIEKTISKTKGQPREPQVYTLPPSREQMTKNQVKLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 70 - protein, 216 aa, synthetic construct
  source 1..216: note = Heterodimeric Fc Backbone 11 monomer 1 (-)
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 71 - protein, 216 aa, synthetic construct
  source 1..216: note = Heterodimeric Fc Backbone 11 monomer 2 (+)
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 72 - protein, 216 aa, synthetic construct
  source 1..216: note = Heterodimeric Fc Backbone 12 monomer 1 (-)
APPVAGPSVFLFPPKPKDTLMISRTPEVTCVVVDVKHEDPEVKFKWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 216 aa, synthetic construct; identical to SEQ ID NO: 50
  source 1..216: note = Heterodimeric Fc Backbone 12 monomer 2 (+)

SEQ ID NO: 74 - protein, 216 aa, synthetic construct
  source 1..216: note = Heterodimeric Fc Backbone 13 monomer 1
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSRDELTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 75 - protein, 216 aa, synthetic construct
  source 1..216: note = Heterodimeric Fc Backbone 13 monomer 2
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSRDQLTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 76 - protein, 216 aa, synthetic construct
  source 1..216: note = Heterodimeric Fc Backbone 14 monomer 1
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHAHYTQKSLSLSPGK

SEQ ID NO: 77 - protein, 216 aa, synthetic construct
  source 1..216: note = Heterodimeric Fc Backbone 14 monomer 2
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 78 - protein, 216 aa, synthetic construct
  source 1..216: note = Heterodimeric Fc Backbone 15 monomer 1
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSRDELTKNQVSLTCDVSGFYPSDIAVEWESDGQPENNYKTTPPVLDSDGSFFLYSKLTVDKSRWEQGDVFSCSVLHEALHAHYTQKSLSLSPGK

SEQ ID NO: 79 - protein, 216 aa, synthetic construct
  source 1..216: note = Heterodimeric Fc Backbone 15 monomer 2
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSRDQLTKNQVKLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 80 - protein, 216 aa, synthetic construct; identical to SEQ ID NO: 50
  source 1..216: note = 2 + 1 mAb-scFv Heterodimeric Fc Backbone monomer 1

SEQ ID NO: 81 - protein, 215 aa, synthetic construct
  source 1..215: note = 2 + 1 mAb-scFv Heterodimeric Fc Backbone monomer 2
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 82 - protein, 98 aa, synthetic construct
  source 1..98: note = IgG1 CH1(+)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 83 - protein, 98 aa, synthetic construct
  source 1..98: note = IgG1 CH1(-)
ASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKV

SEQ ID NO: 84 - protein, 98 aa, synthetic construct
  source 1..98: note = IgG2 CH1(+)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 85 - protein, 98 aa, synthetic construct
  source 1..98: note = IgG2 CH1(-)
ASTKGPSVFPLAPCSRSTSESTAALGCLVKDYFPEPVTVSWNSGALTSGVHTFPAVLQSSGLYSLSSVVTVPSSNFGTQTYTCNVDHKPSDTKVDKTV

SEQ ID NO: 86 - protein, 98 aa, synthetic construct
  source 1..98: note = IgG4 CH1(+)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 87 - protein, 98 aa, synthetic construct
  source 1..98: note = IgG4 CH1(-)
ASTKGPSVFPLAPCSRSTSESTAALGCLVKDYFPEPVTVSWNSGALTSGVHTFPAVLQSSGLYSLSSVVTVPSSSLGTKTYTCNVDHKPSDTKVDKRV

SEQ ID NO: 88 - protein, 15 aa, synthetic construct
  source 1..15: note = IgG1 hinge
EPKSCDKTHTCPPCP

SEQ ID NO: 89 - protein, 12 aa, synthetic construct
  source 1..12: note = IgG2 hinge
ERKCCVECPPCP

SEQ ID NO: 90 - protein, 12 aa, synthetic construct
  source 1..12: note = IgG4 hinge
ESKYGPPCPSCP

SEQ ID NO: 91 - protein, 107 aa, synthetic construct [sequence omitted]

SEQ ID NO: 92 - protein, 106 aa, synthetic construct
  source 1..106: note = Constant Light Domain - Lambda
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 93 - protein, 254 aa, synthetic construct
  source 1..254: note = CD3 High - [anti-CD3]_H1.30_L1.47_scFv scFv
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 94 - protein, 125 aa, synthetic construct
  source 1..125: note = CD3 High - [anti-CD3]_H1.30_L1.47_scFv Variable Heavy (vh) Domain
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSS

SEQ ID NO: 95 - protein, 5 aa, synthetic construct
  source 1..5: note = CD3 High - [anti-CD3]_H1.30_L1.47_scFv vhCDR1
TYAMN

SEQ ID NO: 96 - protein, 19 aa, synthetic construct
  source 1..19: note = CD3 High - [anti-CD3]_H1.30_L1.47_scFv vhCDR2
RIRSKYNNYATYYADSVKG

SEQ ID NO: 97 - protein, 14 aa, synthetic construct
  source 1..14: note = CD3 High - [anti-CD3]_H1.30_L1.47_scFv vhCDR3
HGNFGDSYVSWFAY

SEQ ID NO: 98 - protein, 109 aa, synthetic construct
  source 1..109: note = CD3 High - [anti-CD3]_H1.30_L1.47_scFv Variable Light (vl) Domain
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 99 - protein, 14 aa, synthetic construct
  source 1..14: note = CD3 High - [anti-CD3]_H1.30_L1.47_scFv vlCDR1
GSSTGAVTTSNYAN

SEQ ID NO: 100 - protein, 7 aa, synthetic construct
  source 1..7: note = CD3 High - [anti-CD3]_H1.30_L1.47_scFv vlCDR2
GTNKRAP

SEQ ID NO: 101 - protein, 9 aa, synthetic construct
  source 1..9: note = CD3 High - [anti-CD3]_H1.30_L1.47_scFv vlCDR3
ALWYSNHWV

SEQ ID NO: 102 - protein, 20 aa, synthetic construct
  source 1..20: note = CD3 High - [anti-CD3]_H1.30_L1.47_scFv Linker
GKPGSGKPGSGKPGSGKPGS

SEQ ID NO: 103 - protein, 254 aa, synthetic construct
  source 1..254: note = CD3 High-Int #1 - [anti-CD3]_H1.32_L1.47_scFv scFv
EVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 104 - protein, 125 aa, synthetic construct
  source 1..125: note = CD3 High-Int #1 - [anti-CD3]_H1.32_L1.47_scFv Variable Heavy (vh) Domain
EVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFAYWGQGTLVTVSS

SEQ ID NO: 105 - protein, 5 aa, synthetic construct
  source 1..5: note = CD3 High-Int #1 - [anti-CD3]_H1.32_L1.47_scFv vhCDR1
TYAMN

SEQ ID NO: 106 - protein, 19 aa, synthetic construct
  source 1..19: note = CD3 High-Int #1 - [anti-CD3]_H1.32_L1.47_scFv vhCDR2
RIRSKANNYATYYADSVKG

SEQ ID NO: 107 - protein, 14 aa, synthetic construct
  source 1..14: note = CD3 High-Int #1 - [anti-CD3]_H1.32_L1.47_scFv vhCDR3
HGNFGDSYVSWFAY

SEQ ID NO: 108 - protein, 109 aa, synthetic construct; identical to SEQ ID NO: 98
  source 1..109: note = CD3 High-Int #1 - [anti-CD3]_H1.32_L1.47_scFv Variable Light (vl) Domain

SEQ ID NO: 109 - protein, 14 aa, synthetic construct
  source 1..14: note = CD3 High-Int #1 - [anti-CD3]_H1.32_L1.47_scFv vlCDR1
GSSTGAVTTSNYAN

SEQ ID NO: 110 - protein, 7 aa, synthetic construct
  source 1..7: note = CD3 High-Int #1 - [anti-CD3]_H1.32_L1.47_scFv vlCDR2
GTNKRAP

SEQ ID NO: 111 - protein, 9 aa, synthetic construct
  source 1..9: note = CD3 High-Int #1 - [anti-CD3]_H1.32_L1.47_scFv vlCDR3
ALWYSNHWV

SEQ ID NO: 112 - protein, 20 aa, synthetic construct
  source 1..20: note = CD3 High-Int #1 - [anti-CD3]_H1.32_L1.47_scFv Linker
GKPGSGKPGSGKPGSGKPGS

SEQ ID NO: 113 - protein, 254 aa, synthetic construct
  source 1..254: note = CD3 High-Int #2 - [anti-CD3]_H1.89_L1.47_scFv scFv
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDEYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 114 - protein, 125 aa, synthetic construct
  source 1..125: note = CD3 High-Int #2 - [anti-CD3]_H1.89_L1.47_scFv Variable Heavy (vh) Domain
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDEYVSWFAYWGQGTLVTVSS

SEQ ID NO: 115 - protein, 5 aa, synthetic construct
  source 1..5: note = CD3 High-Int #2 - [anti-CD3]_H1.89_L1.47_scFv vhCDR1
TYAMN

SEQ ID NO: 116 - protein, 19 aa, synthetic construct
  source 1..19: note = CD3 High-Int #2 - [anti-CD3]_H1.89_L1.47_scFv vhCDR2
RIRSKYNNYATYYADSVKG

SEQ ID NO: 117 - protein, 14 aa, synthetic construct
  source 1..14: note = CD3 High-Int #2 - [anti-CD3]_H1.89_L1.47_scFv vhCDR3
HGNFGDEYVSWFAY

SEQ ID NO: 118 - protein, 109 aa, synthetic construct; identical to SEQ ID NO: 98
  source 1..109: note = CD3 High-Int #2 - [anti-CD3]_H1.89_L1.47_scFv Variable Light (vl) Domain

SEQ ID NO: 119 - protein, 14 aa, synthetic construct
  source 1..14: note = CD3 High-Int #2 - [anti-CD3]_H1.89_L1.47_scFv vlCDR1
GSSTGAVTTSNYAN

SEQ ID NO: 120 - protein, 7 aa, synthetic construct
  source 1..7: note = CD3 High-Int #2 - [anti-CD3]_H1.89_L1.47_scFv vlCDR2
GTNKRAP

SEQ ID NO: 121 - protein, 9 aa, synthetic construct
  source 1..9: note = CD3 High-Int #2 - [anti-CD3]_H1.89_L1.47_scFv vlCDR3
ALWYSNHWV

SEQ ID NO: 122 - protein, 20 aa, synthetic construct
  source 1..20: note = CD3 High-Int #2 - [anti-CD3]_H1.89_L1.47_scFv Linker
GKPGSGKPGSGKPGSGKPGS

SEQ ID NO: 123 - protein, 254 aa, synthetic construct
  source 1..254: note = CD3 High-Int - [anti-CD3]_H1.90_L1.47_scFv scFv
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDPYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 124 - protein, 125 aa, synthetic construct
  source 1..125: note = CD3 High-Int - [anti-CD3]_H1.90_L1.47_scFv Variable Heavy (vh) Domain
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDPYVSWFAYWGQGTLVTVSS

SEQ ID NO: 125 - protein, 5 aa, synthetic construct
  source 1..5: note = CD3 High-Int - [anti-CD3]_H1.90_L1.47_scFv vhCDR1
TYAMN

SEQ ID NO: 126 - protein, 19 aa, synthetic construct
  source 1..19: note = CD3 High-Int - [anti-CD3]_H1.90_L1.47_scFv vhCDR2
RIRSKYNNYATYYADSVKG

SEQ ID NO: 127 - protein, 14 aa, synthetic construct
  source 1..14: note = CD3 High-Int - [anti-CD3]_H1.90_L1.47_scFv vhCDR3
HGNFGDPYVSWFAY

SEQ ID NO: 128 - protein, 109 aa, synthetic construct; identical to SEQ ID NO: 98
  source 1..109: note = CD3 High-Int - [anti-CD3]_H1.90_L1.47_scFv Variable Light (vl) Domain

SEQ ID NO: 129 - protein, 14 aa, synthetic construct
  source 1..14: note = CD3 High-Int - [anti-CD3]_H1.90_L1.47_scFv vlCDR1
GSSTGAVTTSNYAN

SEQ ID NO: 130 - protein, 7 aa, synthetic construct
  source 1..7: note = CD3 High-Int - [anti-CD3]_H1.90_L1.47_scFv vlCDR2
GTNKRAP

SEQ ID NO: 131 - protein, 9 aa, synthetic construct
  source 1..9: note = CD3 High-Int - [anti-CD3]_H1.90_L1.47_scFv vlCDR3
ALWYSNHWV

SEQ ID NO: 132 - protein, 20 aa, synthetic construct
  source 1..20: note = CD3 High-Int - [anti-CD3]_H1.90_L1.47_scFv Linker
GKPGSGKPGSGKPGSGKPGS

SEQ ID NO: 133 - protein, 254 aa, synthetic construct
  source 1..254: note = Anti-CD3-Intermediate - [anti-CD3]_H1.33_L1.47_scFv scFv
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFDYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 134 - protein, 125 aa, synthetic construct
  source 1..125: note = Anti-CD3-Intermediate - [anti-CD3]_H1.33_L1.47_scFv Variable Heavy (vh) Domain
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFDYWGQGTLVTVSS

SEQ ID NO: 135 - protein, 5 aa, synthetic construct
  source 1..5: note = Anti-CD3-Intermediate - [anti-CD3]_H1.33_L1.47_scFv vhCDR1
TYAMN

SEQ ID NO: 136 - protein, 19 aa, synthetic construct
  source 1..19: note = Anti-CD3-Intermediate - [anti-CD3]_H1.33_L1.47_scFv vhCDR2
RIRSKYNNYATYYADSVKG

SEQ ID NO: 137 - protein, 14 aa, synthetic construct
  source 1..14: note = Anti-CD3-Intermediate - [anti-CD3]_H1.33_L1.47_scFv vhCDR3
HGNFGDSYVSWFDY

SEQ ID NO: 138 - protein, 109 aa, synthetic construct; identical to SEQ ID NO: 98
  source 1..109: note = Anti-CD3-Intermediate - [anti-CD3]_H1.33_L1.47_scFv Variable Light (vl) Domain

SEQ ID NO: 139 - protein, 14 aa, synthetic construct
  source 1..14: note = Anti-CD3-Intermediate - [anti-CD3]_H1.33_L1.47_scFv vlCDR1
GSSTGAVTTSNYAN

SEQ ID NO: 140 - protein, 7 aa, synthetic construct
  source 1..7: note = Anti-CD3-Intermediate - [anti-CD3]_H1.33_L1.47_scFv vlCDR2
GTNKRAP

SEQ ID NO: 141 - protein, 9 aa, synthetic construct
  source 1..9: note = Anti-CD3-Intermediate - [anti-CD3]_H1.33_L1.47_scFv vlCDR3
ALWYSNHWV

SEQ ID NO: 142 - protein, 20 aa, synthetic construct
  source 1..20: note = Anti-CD3-Intermediate - [anti-CD3]_H1.33_L1.47_scFv Linker
GKPGSGKPGSGKPGSGKPGS

SEQ ID NO: 143 - protein, 254 aa, synthetic construct
  source 1..254: note = CD3 High-Int - [anti-CD3]_H1.31_L1.47_scFv scFv
EVQLVESGGGLVQPGGSLRLSCAASGFTFSTYAMSWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 144 - protein, 125 aa, synthetic construct
  source 1..125: note = CD3 High-Int - [anti-CD3]_H1.31_L1.47_scFv Variable Heavy (vh) Domain
EVQLVESGGGLVQPGGSLRLSCAASGFTFSTYAMSWVRQAPGKGLEWVGRIRSKYNNYATYYADSVKGRFTISRDDSKNTLYLQMNSLRAEDTAVYYCVRHGNFGDSYVSWFAYWGQGTLVTVSS

SEQ ID NO: 145 - protein, 5 aa, synthetic construct
  source 1..5: note = CD3 High-Int - [anti-CD3]_H1.31_L1.47_scFv vhCDR1
TYAMS

SEQ ID NO: 146 - protein, 19 aa, synthetic construct
  source 1..19: note = CD3 High-Int - [anti-CD3]_H1.31_L1.47_scFv vhCDR2
RIRSKYNNYATYYADSVKG

SEQ ID NO: 147 - protein, 14 aa, synthetic construct
  source 1..14: note = CD3 High-Int - [anti-CD3]_H1.31_L1.47_scFv vhCDR3
HGNFGDSYVSWFAY

SEQ ID NO: 148 - protein, 109 aa, synthetic construct; identical to SEQ ID NO: 98
  source 1..109: note = CD3 High-Int - [anti-CD3]_H1.31_L1.47_scFv Variable Light (vl) Domain

SEQ ID NO: 149 - protein, 14 aa, synthetic construct
  source 1..14: note = CD3 High-Int - [anti-CD3]_H1.31_L1.47_scFv vlCDR1
GSSTGAVTTSNYAN

SEQ ID NO: 150 - protein, 7 aa, synthetic construct
  source 1..7: note = CD3 High-Int - [anti-CD3]_H1.31_L1.47_scFv vlCDR2
GTNKRAP

SEQ ID NO: 151 - protein, 9 aa, synthetic construct
  source 1..9: note = CD3 High-Int - [anti-CD3]_H1.31_L1.47_scFv vlCDR3
ALWYSNHWV

SEQ ID NO: 152 - protein, 20 aa, synthetic construct
  source 1..20: note = CD3 High-Int - [anti-CD3]_H1.31_L1.47_scFv Linker
GKPGSGKPGSGKPGSGKPGS

SEQ ID NO: 153 - protein, 254 aa, synthetic construct
  source 1..254: note = CD3 High[VL-VH] - [anti-CD3]_L1.47_ H1.30_scFv scFv
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGKPGSGKPGSGKPGSGKPGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSS

SEQ ID NO: 154 - protein, 109 aa, synthetic construct; identical to SEQ ID NO: 98
  source 1..109: note = CD3 High[VL-VH] - [anti-CD3]_L1.47_ H1.30_scFv Variable Light (vl) Domain

SEQ ID NO: 155 - protein, 14 aa, synthetic construct
  source 1..14: note = CD3 High[VL-VH] - [anti-CD3]_L1.47_ H1.30_scFv vlCDR1
GSSTGAVTTSNYAN

SEQ ID NO: 156 - protein, 7 aa, synthetic construct
  source 1..7: note = CD3 High[VL-VH] - [anti-CD3]_L1.47_ H1.30_scFv vlCDR2
GTNKRAP

SEQ ID NO: 157 - protein, 9 aa, synthetic construct
  source 1..9: note = CD3 High[VL-VH] - [anti-CD3]_L1.47_ H1.30_scFv vlCDR3
ALWYSNHWV

SEQ ID NO: 158 - protein, 125 aa, synthetic construct; identical to SEQ ID NO: 94
  source 1..125: note = CD3 High[VL-VH] - [anti-CD3]_L1.47_ H1.30_scFv Variable Heavy (vh) Domain

SEQ ID NO: 159 - protein, 5 aa, synthetic construct
  source 1..5: note = CD3 High[VL-VH] - [anti-CD3]_L1.47_ H1.30_scFv vhCDR1
TYAMN

SEQ ID NO: 160 - protein, 19 aa, synthetic construct
  source 1..19: note = CD3 High[VL-VH] - [anti-CD3]_L1.47_ H1.30_scFv vhCDR2
RIRSKYNNYATYYADSVKG

SEQ ID NO: 161 - protein, 14 aa, synthetic construct
  source 1..14: note = CD3 High[VL-VH] - [anti-CD3]_L1.47_ H1.30_scFv vhCDR3
HGNFGDSYVSWFAY

SEQ ID NO: 162 - protein, 20 aa, synthetic construct
  source 1..20: note = CD3 High[VL-VH] - [anti-CD3]_L1.47_ H1.30_scFv Linker
GKPGSGKPGSGKPGSGKPGS

SEQ ID NO: 163 - protein, 254 aa, synthetic construct
  source 1..254: note = CD3 High-Int #1[VL-VH] - [anti-CD3]_L1.47_ H1.32_scFv scFv
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGKPGSGKPGSGKPGSGKPGSEVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFAYWGQGTLVTVSS

SEQ ID NO: 164 - protein, 109 aa, synthetic construct; identical to SEQ ID NO: 98
  source 1..109: note = CD3 High-Int #1[VL-VH] - [anti-CD3]_L1.47_ H1.32_scFv Variable Light (vl) Domain

SEQ ID NO: 165 - protein, 14 aa, synthetic construct
  source 1..14: note = CD3 High-Int #1[VL-VH] - [anti-CD3]_L1.47_ H1.32_scFv vlCDR1
GSSTGAVTTSNYAN

SEQ ID NO: 166 - protein, 7 aa, synthetic construct
  source 1..7: note = CD3 High-Int #1[VL-VH] - [anti-CD3]_L1.47_ H1.32_scFv vlCDR2
GTNKRAP

SEQ ID NO: 167 - protein, 9 aa, synthetic construct
  source 1..9: note = CD3 High-Int #1[VL-VH] - [anti-CD3]_L1.47_ H1.32_scFv vlCDR3
ALWYSNHWV

SEQ ID NO: 168 - protein, 125 aa, synthetic construct; identical to SEQ ID NO: 104
  source 1..125: note = CD3 High-Int #1[VL-VH] - [anti-CD3]_L1.47_ H1.32_scFv Variable Heavy (vh) Domain

SEQ ID NO: 169 - protein, 5 aa, synthetic construct
  source 1..5: note = CD3 High-Int #1[VL-VH] - [anti-CD3]_L1.47_ H1.32_scFv vhCDR1
TYAMN

SEQ ID NO: 170 - protein, 19 aa, synthetic construct
  source 1..19: note = CD3 High-Int #1[VL-VH] - [anti-CD3]_L1.47_ H1.32_scFv vhCDR2
RIRSKANNYATYYADSVKG

SEQ ID NO: 171 - protein, 14 aa, synthetic construct
  source 1..14: note = CD3 High-Int #1[VL-VH] - [anti-CD3]_L1.47_ H1.32_scFv vhCDR3
HGNFGDSYVSWFAY

SEQ ID NO: 172 - protein, 20 aa, synthetic construct
  source 1..20: note = CD3 High-Int #1[VL-VH] - [anti-CD3]_L1.47_ H1.32_scFv Linker
GKPGSGKPGSGKPGSGKPGS

SEQ ID NO: 173 - protein, 254 aa, synthetic construct
  source 1..254: note = CD3 High-Int #2[VL-VH] - [anti-CD3]_L1.47_ H1.89_scFv scFv
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGKPGSGKPGSGKPGSGKPGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDEYVSWFAYWGQGTLVTVSS

SEQ ID NO: 174 - protein, 109 aa, synthetic construct; identical to SEQ ID NO: 98
  source 1..109: note = CD3 High-Int #2[VL-VH] - [anti-CD3]_L1.47_ H1.89_scFv Variable Light (vl) Domain

SEQ ID NO: 175 - protein, 14 aa, synthetic construct
  source 1..14: note = CD3 High-Int #2[VL-VH] - [anti-CD3]_L1.47_ H1.89_scFv vlCDR1
GSSTGAVTTSNYAN

SEQ ID NO: 176 - protein, 7 aa, synthetic construct
  source 1..7: note = CD3 High-Int #2[VL-VH] - [anti-CD3]_L1.47_ H1.89_scFv vlCDR2
GTNKRAP

SEQ ID NO: 177 - protein, 9 aa, synthetic construct
  source 1..9: note = CD3 High-Int #2[VL-VH] - [anti-CD3]_L1.47_ H1.89_scFv vlCDR3
ALWYSNHWV

SEQ ID NO: 178 - protein, 125 aa, synthetic construct; identical to SEQ ID NO: 114
  source 1..125: note = CD3 High-Int #2[VL-VH] - [anti-CD3]_L1.47_ H1.89_scFv Variable Heavy (vh) Domain

SEQ ID NO: 179 - protein, 5 aa, synthetic construct
  source 1..5: note = CD3 High-Int #2[VL-VH] - [anti-CD3]_L1.47_ H1.89_scFv vhCDR1
TYAMN

SEQ ID NO: 180 - protein, 19 aa, synthetic construct
  source 1..19: note = CD3 High-Int #2[VL-VH] - [anti-CD3]_L1.47_ H1.89_scFv vhCDR2
RIRSKYNNYATYYADSVKG

SEQ ID NO: 181 - protein, 14 aa, synthetic construct
  source 1..14: note = CD3 High-Int #2[VL-VH] - [anti-CD3]_L1.47_ H1.89_scFv vhCDR3
HGNFGDEYVSWFAY

SEQ ID NO: 182 - protein, 20 aa, synthetic construct
  source 1..20: note = CD3 High-Int #2[VL-VH] - [anti-CD3]_L1.47_ H1.89_scFv Linker
GKPGSGKPGSGKPGSGKPGS

SEQ ID NO: 183 - protein, 254 aa, synthetic construct
  source 1..254: note = CD3 High-Int[VL-VH] - [anti-CD3]_L1.47_ H1.90_scFv scFv
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGKPGSGKPGSGKPGSGKPGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDPYVSWFAYWGQGTLVTVSS

SEQ ID NO: 184 - protein, 109 aa, synthetic construct; identical to SEQ ID NO: 98
  source 1..109: note = CD3 High-Int[VL-VH] - [anti-CD3]_L1.47_ H1.90_scFv Variable Light (vl) Domain

SEQ ID NO: 185 - protein, 14 aa, synthetic construct
  source 1..14: note = CD3 High-Int[VL-VH] - [anti-CD3]_L1.47_ H1.90_scFv vlCDR1
GSSTGAVTTSNYAN

SEQ ID NO: 186 - protein, 7 aa, synthetic construct
  source 1..7: note = CD3 High-Int[VL-VH] - [anti-CD3]_L1.47_ H1.90_scFv vlCDR2
GTNKRAP

SEQ ID NO: 187 - protein, 9 aa, synthetic construct
  source 1..9: note = CD3 High-Int[VL-VH] - [anti-CD3]_L1.47_ H1.90_scFv vlCDR3
ALWYSNHWV

SEQ ID NO: 188 - protein, 125 aa, synthetic construct; identical to SEQ ID NO: 124
  source 1..125: note = CD3 High-Int[VL-VH] - [anti-CD3]_L1.47_ H1.90_scFv Variable Heavy (vh) Domain

SEQ ID NO: 189 - protein, 5 aa, synthetic construct
  source 1..5: note = CD3 High-Int[VL-VH] - [anti-CD3]_L1.47_ H1.90_scFv vhCDR1
TYAMN

SEQ ID NO: 190 - protein, 19 aa, synthetic construct
  source 1..19: note = CD3 High-Int[VL-VH] - [anti-CD3]_L1.47_ H1.90_scFv vhCDR2
RIRSKYNNYATYYADSVKG

SEQ ID NO: 191 - protein, 14 aa, synthetic construct
  source 1..14: note = CD3 High-Int[VL-VH] - [anti-CD3]_L1.47_ H1.90_scFv vhCDR3
HGNFGDPYVSWFAY

SEQ ID NO: 192 - protein, 20 aa, synthetic construct
  source 1..20: note = CD3 High-Int[VL-VH] - [anti-CD3]_L1.47_ H1.90_scFv Linker
GKPGSGKPGSGKPGSGKPGS

SEQ ID NO: 193 - protein, 254 aa, synthetic construct
  source 1..254: note = Anti-CD3-Intermediate[VL-VH] - [anti-CD3]_L1.47_ H1.33_scFv scFv
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGKPGSGKPGSGKPGSGKPGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFDYWGQGTLVTVSS

SEQ ID NO: 194 - protein, 109 aa, synthetic construct; identical to SEQ ID NO: 98
  source 1..109: note = Anti-CD3-Intermediate[VL-VH] - [anti-CD3]_L1.47_ H1.33_scFv Variable Light (vl) Domain

SEQ ID NO: 195 - protein, 14 aa, synthetic construct
  source 1..14: note = Anti-CD3-Intermediate[VL-VH] - [anti-CD3]_L1.47_ H1.33_scFv vlCDR1
GSSTGAVTTSNYAN

SEQ ID NO: 196 - protein, 7 aa, synthetic construct
  source 1..7: note = Anti-CD3-Intermediate[VL-VH] - [anti-CD3]_L1.47_ H1.33_scFv vlCDR2
GTNKRAP

SEQ ID NO: 197 - protein, 9 aa, synthetic construct
  source 1..9: note = Anti-CD3-Intermediate[VL-VH] - [anti-CD3]_L1.47_ H1.33_scFv vlCDR3
ALWYSNHWV

SEQ ID NO: 198 - protein, 125 aa, synthetic construct; identical to SEQ ID NO: 134
  source 1..125: note = Anti-CD3-Intermediate[VL-VH] - [anti-CD3]_L1.47_ H1.33_scFv Variable Heavy (vh) Domain

SEQ ID NO: 199 - protein, 5 aa, synthetic construct
  source 1..5: note = Anti-CD3-Intermediate[VL-VH] - [anti-CD3]_L1.47_ H1.33_scFv vhCDR1
TYAMN

SEQ ID NO: 200 - protein, 19 aa, synthetic construct
  source 1..19: note = Anti-CD3-Intermediate[VL-VH] - [anti-CD3]_L1.47_ H1.33_scFv vhCDR2
RIRSKYNNYATYYADSVKG

SEQ ID NO: 201 - protein, 14 aa, synthetic construct
  source 1..14: note = Anti-CD3-Intermediate[VL-VH] - [anti-CD3]_L1.47_ H1.33_scFv vhCDR3
HGNFGDSYVSWFDY

SEQ ID NO: 202 - protein, 20 aa, synthetic construct
  source 1..20: note = Anti-CD3-Intermediate[VL-VH] - [anti-CD3]_L1.47_ H1.33_scFv Linker
GKPGSGKPGSGKPGSGKPGS

SEQ ID NO: 203 - protein, 254 aa, synthetic construct
  source 1..254: note = CD3 High-Int[VL-VH] - [anti-CD3]_L1.47_ H1.31_scFv scFv
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGKPGSGKPGSGKPGSGKPGSEVQLVESGGGLVQPGGSLRLSCAASGFTFSTYAMSWVRQAPGKGLEWVGRIRSKYNNYATYYADSVKGRFTISRDDSKNTLYLQMNSLRAEDTAVYYCVRHGNFGDSYVSWFAYWGQGTLVTVSS

SEQ ID NO: 204 - protein, 109 aa, synthetic construct; identical to SEQ ID NO: 98
  source 1..109: note = CD3 High-Int[VL-VH] - [anti-CD3]_L1.47_ H1.31_scFv Variable Light (vl) Domain

SEQ ID NO: 205 - protein, 14 aa, synthetic construct
  source 1..14: note = CD3 High-Int[VL-VH] - [anti-CD3]_L1.47_ H1.31_scFv vlCDR1
GSSTGAVTTSNYAN

SEQ ID NO: 206 - protein, 7 aa, synthetic construct
  source 1..7: note = CD3 High-Int[VL-VH] - [anti-CD3]_L1.47_ H1.31_scFv vlCDR2
GTNKRAP

SEQ ID NO: 207 - protein, 9 aa, synthetic construct
  source 1..9: note = CD3 High-Int[VL-VH] - [anti-CD3]_L1.47_ H1.31_scFv vlCDR3
ALWYSNHWV

SEQ ID NO: 208 - protein, 125 aa, synthetic construct; identical to SEQ ID NO: 144
  source 1..125: note = CD3 High-Int[VL-VH] - [anti-CD3]_L1.47_ H1.31_scFv Variable Heavy (vh) Domain

SEQ ID NO: 209 - protein, 5 aa, synthetic construct
  source 1..5: note = CD3 High-Int[VL-VH] - [anti-CD3]_L1.47_ H1.31_scFv vhCDR1
TYAMS

SEQ ID NO: 210 - protein, 19 aa, synthetic construct
  source 1..19: note = CD3 High-Int[VL-VH] - [anti-CD3]_L1.47_ H1.31_scFv vhCDR2
RIRSKYNNYATYYADSVKG

SEQ ID NO: 211 - protein, 14 aa, synthetic construct
  source 1..14: note = CD3 High-Int[VL-VH] - [anti-CD3]_L1.47_ H1.31_scFv vhCDR3
HGNFGDSYVSWFAY

SEQ ID NO: 212 - protein, 20 aa, synthetic construct
  source 1..20: note = CD3 High-Int[VL-VH] - [anti-CD3]_L1.47_ H1.31_scFv Linker
GKPGSGKPGSGKPGSGKPGS

SEQ ID NO: 213 - protein, 875 aa, Homo sapiens [sequence omitted]

SEQ ID NO: 214 - protein, 845 aa, Homo sapiens
  source 1..845: note = sp|O14638|31-875
LLVIMSLGLGLGLGLRKLEKQGSCRKKCFDASFRGLENCRCDVACKDRGDCCWDFEDTCVESTRIWMCNKFRCGETRLEASLCSCSDDCLQRKDCCADYKSVCQGETSWLEENCDTAQQSQCPEGFDLPPVILFSMDGFRAEYLYTWDTLMPNINKLKTCGIHSKYMRAMYPTKTFPNHYTIVTGLYPESHGIIDNNMYDVNLNKNFSLSSKEQNNPAWWHGQPMWLTAMYQGLKAATYFWPGSEVAINGSFPSIYMPYNGSVPFEERISTLLKWLDLPKAERPRFYTMYFEEPDSSGHAGGPVSARVIKALQVVDHAFGMLMEGLKQRNLHNCVNIILLADHGMDQTYCNKMEYMTDYFPRINFFYMYEGPAPRIRAHNIPHDFFSFNSEEIVRNLSCRKPDQHFKPYLTPDLPKRLHYAKNVRIDKVHLFVDQQWLAVRSKSNTNCGGGNHGYNNEFRSMEAIFLAHGPSFKEKTEVEPFENIEVYNLMCDLLRIQPAPNNGTHGSLNHLLKVPFYEPSHAEEVSKFSVCGFANPLPTESLDCFCPHLQNSTQLEQVNQMLNLTQEEITATVKVNLPFGRPRVLQKNVDHCLLYHREYVSGFGKAMRMPMWSSYTVPQLGDTSPLPPTVPDCLRADVRVPPSESQKCSFYLADKNITHGFLYPPASNRTSDSQYDALITSNLVPMYEEFRKMWDYFHSVLLIKHATERNGVNVVSGPIFDYNYDGHFDAPDEITKHLANTDVPIPTHYFVVLTSCKNKSHTPENCPGWLDVLPFIIPHRPTNVESCPEGKPEALWVEERFTAHIARVRDVELLTGLDFYQDKVQPVSEILQLKTYLPTFETTI

SEQ ID NO: 215 - protein, 874 aa, Mus musculus
  source 1..874: note = sp|Q6DYE8
MDSRLALATEEPIKKDSLKKYKILCVVLLALLVIVSLGLGLGLGLRKPEEQGSCRKKCFDSSHRGLEGCRCDSGCTGRGDCCWDFEDTCVKSTQIWTCNLFRCGENRLETALCSCADDCLQRKDCCADYKTVCQGESPWVTEACASSQEPQCPPGFDLPPVILFSMDGFRAEYLQTWSTLLPNINKLKTCGIHSKYMRAMYPTKTFPNHYTIVTGLYPESHGIIDNNMYDVHLNKNFSLSSVEKSNPAWWSGQPIWLTAMYQGLKAACYYWPGSDVAVNGSFPTIYRNYSNSVPYERRITTLLQWLDLPKADRPSFYTIYVEEPDSAGHSSGPVSAGVIKALQSVDNAFGMLMEGLKQRNLHNCVNIIVLADHGMDQTSCDRVEYMTDYFPKINFYMYQGPAPRIRTRNIPQDFFTFNSEEIVRNLSCRKPDQHFKPYLTPDLPKRLHYAKNVRIDKAHLMVDRQWLAFRSKGSSNCGGGTHGYNNEFKSMEAIFLAHGPSFIEKTVIEPFENIEVYNLLCDLLHIEPAPNNGTHGSLNHLLKTPFYKPSHAGELSTPADCGFTTPLPTDPLDCSCPALQNTPGLEEQANQRLNLSEGEVAATVKANLPFGRPRVMQKNGDHCLLYHRDYISGYGKAMKMPMWSSYTVLKPGDTSSLPPTVPDCLRADVRVAPSESQKCSFYLADKNITHGFLYPAIKGTNESRYDALITSNLVPMYKEFKKMWDYFHEVLLIKYAIERNGLNVVSGPIFDYNYDGHFDAPDEITQYVAGTDVPIPTHYFVVLTSCKDQTHTPDSCPGWLDVLPFIVPHRPTNIESCSENKTEDLWVEERFQAHAARVRDVELLTGLDFYQEKAQPVSQILQLKTYLPTFETII

SEQ ID NO: 216 - protein, 844 aa, Mus musculus
  source 1..844: note = sp|Q6DYE8|31-874
LLVIVSLGLGLGLGLRKPEEQGSCRKKCFDSSHRGLEGCRCDSGCTGRGDCCWDFEDTCVKSTQIWTCNLFRCGENRLETALCSCADDCLQRKDCCADYKTVCQGESPWVTEACASSQEPQCPPGFDLPPVILFSMDGFRAEYLQTWSTLLPNINKLKTCGIHSKYMRAMYPTKTFPNHYTIVTGLYPESHGIIDNNMYDVHLNKNFSLSSVEKSNPAWWSGQPIWLTAMYQGLKAACYYWPGSDVAVNGSFPTIYRNYSNSVPYERRITTLLQWLDLPKADRPSFYTIYVEEPDSAGHSSGPVSAGVIKALQSVDNAFGMLMEGLKQRNLHNCVNIIVLADHGMDQTSCDRVEYMTDYFPKINFYMYQGPAPRIRTRNIPQDFFTFNSEEIVRNLSCRKPDQHFKPYLTPDLPKRLHYAKNVRIDKAHLMVDRQWLAFRSKGSSNCGGGTHGYNNEFKSMEAIFLAHGPSFIEKTVIEPFENIEVYNLLCDLLHIEPAPNNGTHGSLNHLLKTPFYKPSHAGELSTPADCGFTTPLPTDPLDCSCPALQNTPGLEEQANQRLNLSEGEVAATVKANLPFGRPRVMQKNGDHCLLYHRDYISGYGKAMKMPMWSSYTVLKPGDTSSLPPTVPDCLRADVRVAPSESQKCSFYLADKNITHGFLYPAIKGTNESRYDALITSNLVPMYKEFKKMWDYFHEVLLIKYAIERNGLNVVSGPIFDYNYDGHFDAPDEITQYVAGTDVPIPTHYFVVLTSCKDQTHTPDSCPGWLDVLPFIVPHRPTNIESCSENKTEDLWVEERFQAHAARVRDVELLTGLDFYQEKAQPVSQILQLKTYLPTFETII

SEQ ID NO: 217 - protein, 874 aa, Macaca fascicularis
  source 1..874: note = tr|A0A2K5TKP4
MESMLTLATEQPVKKNTLKKYKIACIVLLALLVIMSLGLGLGLGLRKLEKQGSCRKKCFDASFRGLENCRCDVACEDRGDCCWDFEDTCVESTRIWTCNKFRCGETRLEASLCSCSDDCLQRKDCCADYKSVCQGETSWLEENCDTAQQSQCPEGFDLPPVILFSMDGFRAEYLYTWDTLMPNINKLKTCGIHSKYMRAMYPTKTFPNHYTIVTGLYPESHGIIDNNMYDVNLNKNFSLSSEEQNNPAWWHGQPMWLTAMYQGLKAATYFWPGSEVAINGSFPSIYMPYNRSVPYEERISTLLKWLDLPKAERPSFYTMYFEEPDSSGHAGGPVSARVIKALQVVDHAFGMLMEGLKQRNLHNCVNIILLADHGMDQTYCNKMEYMTDYFPRINFYMYEGPAPRIRALNVPHDFFSCKYEDTYMKKRCRKPDQHFKPYLTPDLPKRLHYAKNVRIDKVHLFVDRQWLAVGSKSNTNCGGGNHGYNNEFRSMEAIFLAHGPSFKEKTEVEPFENIEVYNLMCDLLRIQPAPNNGTRGSLNHLLKVPFYEPSHAEEVSKFSVCGFANPLPTNNLSCLCPHLQNSIQLEQVNQMLNLTQEEITATVKVNLPFGRPRVLQKNVDNCLLYHREYVSGFGKAMRMPMWSSYTVPQLGDTSPLPPTVPDCLRADVRVPPSESQKCSFYLADENITHGFLYPPAINRTSDSQYDALIMSNLVPMYEEFRKMWDYFHSVLLIKHATERNGVNVVSGPIFDYNYDGHFDAPEEITKHIANTDIPIPTHYFVVLTSCKNKSHTPENCPGWLDVLPFIIPHRPTNVESCPEGKPEALWVEERFTAHIARVRDVELLTGLDFYQDKAQPVSEILQLKTYLPTFETTI

SEQ ID NO: 218 - protein, 220 aa, Homo sapiens
  source 1..220: note = sp|P10747|CD28_HUMAN T-cell-specific surface glycoprotein CD28 OS=Homo sapiens OX=9606 GN=CD28 PE=1; SV = 1
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 219 - protein, 134 aa, Homo sapiens
  source 1..134: note = sp|P10747|19-152
NKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKP

SEQ ID NO: 220 - protein, 218 aa, Mus musculus
  source 1..218: note = sp|P31041|CD28_MOUSE T-cell-specific surface glycoprotein CD28 OS=Mus musculus OX=10090 GN=Cd28 PE=1; SV = 2
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 221 - protein, 131 aa, Mus musculus
  source 1..131: note = sp|P31041|20-150
NKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKL

SEQ ID NO: 222 - protein, 220 aa, Macaca fascicularis
  source 1..220: note = tr|Q0PDN3|Q0PDN3_MACFA CD28 OS=Macaca fascicularis; OX = 9541 GN=CD28 PE=2 SV=1
MLRLLLALNLLPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWALVVVGGVLACYSLLVTVAFCIFWMRSKRSRLLHSDYMNMTPRRPGPTRKHYQPYAPPRDFAAYRS

SEQ ID NO: 223 - protein, 134 aa, Macaca fascicularis; identical to SEQ ID NO: 219
  source 1..134: note = tr|Q0PDN3|19-152

SEQ ID NO: 224 - protein, 584 aa, Macaca fascicularis
  source 1..584: note = XP_015308533.1 PREDICTED: CD276 antigen isoform X1 [Macaca fascicularis]
MKLSSDHVFPLFRKLQWLPAAFRIQFTPVSPSAGAAFHHGEPSCQLPHSKMLHRRGSPGMGVHVGAALGALWFCLTGALEVQVPEDPVVALVGTDATLRCSFSPEPGFSLAQLNLIWQLTDTKQLVHSFTEGRDQGSAYANRTALFLDLLAQGNASLRLQRVRVADEGSFTCFVSIRDFGSAAVSLQVAAPYSKPSMTLEPNKDLRPGDTVTITCSSYRGYPEAEVFWQDGQGAPLTGNVTTSQMANEQGLFDVHSVLRVVLGANGTYSCLVRNPVLQQDAHGSITITPQRSPTGAVEVQVPEDPVVALVGTDATLRCSFSPEPGFSLAQLNLIWQLTDTKQLVHSFTEGRDQGSAYANRTALFLDLLAQGNASLRLQRVRVADEGSFTCFVSIRDFGSAAVSLQVAAPYSKPSMTLEPNKDLRPGDTVTITCSSYRGYPEAEVFWQDGQGAPLTGNVTTSQMANEQGLFDVHSVLRVVLGANGTYSCLVRNPVLQQDAHGSVTITGQPMTFPPEALWVTVGLSVCLVALLVALAFVCWRKIKQSCEEENAGAEDQDGEGEGSKTALQPLKHSDSKEDDGQELA

SEQ ID NO: 225 - protein, 438 aa, Macaca fascicularis
  source 1..438: note = XP_015308533.1|79-516
LEVQVPEDPVVALVGTDATLRCSFSPEPGFSLAQLNLIWQLTDTKQLVHSFTEGRDQGSAYANRTALFLDLLAQGNASLRLQRVRVADEGSFTCFVSIRDFGSAAVSLQVAAPYSKPSMTLEPNKDLRPGDTVTITCSSYRGYPEAEVFWQDGQGAPLTGNVTTSQMANEQGLFDVHSVLRVVLGANGTYSCLVRNPVLQQDAHGSITITPQRSPTGAVEVQVPEDPVVALVGTDATLRCSFSPEPGFSLAQLNLIWQLTDTKQLVHSFTEGRDQGSAYANRTALFLDLLAQGNASLRLQRVRVADEGSFTCFVSIRDFGSAAVSLQVAAPYSKPSMTLEPNKDLRPGDTVTITCSSYRGYPEAEVFWQDGQGAPLTGNVTTSQMANEQGLFDVHSVLRVVLGANGTYSCLVRNPVLQQDAHGSVTITGQPMTFPPEA

SEQ ID NO: 226 - protein, 124 aa, synthetic construct
  source 1..124: note = MESO-A[MSLN]_H1.1_L1 Variable heavy (vh) domain
EVQLVESGGGLVQPGGSLKLSCAASGFDFSRYWMSWVRQAPGKGLEWVSEINPDSSTIVYTPSVKGRFTISRDNAKNSLYLQMNSLRAEDTAVYYCARRGSHYYGYRTGYFDVWGAGTTVTVSS

SEQ ID NO: 227 - protein, 5 aa, synthetic construct
  source 1..5: note = MESO-A[MSLN]_H1.1_L1 vhCDR1
RYWMS

SEQ ID NO: 228 - protein, 17 aa, synthetic construct
  source 1..17: note = MESO-A[MSLN]_H1.1_L1 vhCDR2
EINPDSSTIVYTPSVKG

SEQ ID NO: 229 - protein, 15 aa, synthetic construct
  source 1..15: note = MESO-A[MSLN]_H1.1_L1 vhCDR3
RGSHYYGYRTGYFDV

SEQ ID NO: 230 - protein, 106 aa, synthetic construct
  source 1..106: note = MESO-A[MSLN]_H1.1_L1 Variable light (vl) domain
DVVLTQSPATLSLSPGERATLSCRASSSVSYMYWHQQKPDQSPKLLIYDTSNLASGVPVRFSGSGSGTDYTLTISSLEPEDFAVYYCQQWSSYPPTFGGGTKLEIK

SEQ ID NO: 231 - protein, 10 aa, synthetic construct
  source 1..10: note = MESO-A[MSLN]_H1.1_L1 vlCDR1
RASSSVSYMY

SEQ ID NO: 232 - protein, 7 aa, synthetic construct
  source 1..7: note = MESO-A[MSLN]_H1.1_L1 vlCDR2
DTSNLAS

SEQ ID NO: 233 - protein, 9 aa, synthetic construct
  source 1..9: note = MESO-A[MSLN]_H1.1_L1 vlCDR3
QQWSSYPPT

SEQ ID NO: 234 - protein, 454 aa, synthetic construct
  source 1..454: note = XENP16249 Chain 1 - MESO-A[MSLN]_H1.1_IgG1 Heavy Chain
EVQLVESGGGLVQPGGSLKLSCAASGFDFSRYWMSWVRQAPGKGLEWVSEINPDSSTIVYTPSVKGRFTISRDNAKNSLYLQMNSLRAEDTAVYYCARRGSHYYGYRTGYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 235 - protein, 213 aa, synthetic construct
  source 1..213: note = XENP16249 Chain 2 - MESO-A[MSLN]_L1 Light Chain
DVVLTQSPATLSLSPGERATLSCRASSSVSYMYWHQQKPDQSPKLLIYDTSNLASGVPVRFSGSGSGTDYTLTISSLEPEDFAVYYCQQWSSYPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 236 - protein, 453 aa, synthetic construct
  source 1..453: note = XENP16250 Chain 1 - MESO-A[MSLN]_H1.1_IgG1_PVA_/S267K Heavy Chain
EVQLVESGGGLVQPGGSLKLSCAASGFDFSRYWMSWVRQAPGKGLEWVSEINPDSSTIVYTPSVKGRFTISRDNAKNSLYLQMNSLRAEDTAVYYCARRGSHYYGYRTGYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 237 - protein, 213 aa, synthetic construct; identical to SEQ ID NO: 235
  source 1..213: note = XENP16250 Chain 2 - MESO-A[MSLN]_L1 Light Chain

SEQ ID NO: 238 - protein, 453 aa, synthetic construct
  source 1..453: note = XENP16253 Chain 1 - MESO-A[MSLN]_H1.1_IgG1_pI(-)_Is osteric_A_/PVA_/S267K/L368D/K370S Heavy Chain
EVQLVESGGGLVQPGGSLKLSCAASGFDFSRYWMSWVRQAPGKGLEWVSEINPDSSTIVYTPSVKGRFTISRDNAKNSLYLQMNSLRAEDTAVYYCARRGSHYYGYRTGYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 239 - protein, 213 aa, synthetic construct; identical to SEQ ID NO: 235
  source 1..213: note = XENP16253 Chain 2 - MESO-A[MSLN]_L1 Light Chain

SEQ ID NO: 240 - protein, 123 aa, synthetic construct
  source 1..123: note = MESO-B[MSLN]_H0L0 Variable heavy (vh) domain
QVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSS

SEQ ID NO: 241 - protein, 7 aa, synthetic construct
  source 1..7: note = MESO-B[MSLN]_H0L0 vhCDR1
SNSATWN

SEQ ID NO: 242 - protein, 18 aa, synthetic construct
  source 1..18: note = MESO-B[MSLN]_H0L0 vhCDR2
RTYYRSKWYNDYAVSVKS

SEQ ID NO: 243 - protein, 11 aa, synthetic construct
  source 1..11: note = MESO-B[MSLN]_H0L0 vhCDR3
GMMTYYYGMDV

SEQ ID NO: 244 - protein, 115 aa, synthetic construct
  source 1..115: note = MESO-B[MSLN]_H0L0 Variable light (vl) domain
QPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVL

SEQ ID NO: 245 - protein, 14 aa, synthetic construct
  source 1..14: note = MESO-B[MSLN]_H0L0 vlCDR1
TLRSGINVGPYRIY

SEQ ID NO: 246 - protein, 7 aa, synthetic construct
  source 1..7: note = MESO-B[MSLN]_H0L0 vlCDR2
SDKQQGS

SEQ ID NO: 247 - protein, 9 aa, synthetic construct
  source 1..9: note = MESO-B[MSLN]_H0L0 vlCDR3
MIWHSSAAV

SEQ ID NO: 248 - protein, 453 aa, synthetic construct
  source 1..453: note = XENP16827 Chain 1 - MESO-B[MSLN]_H0_IgG1 Heavy Chain
QVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 249 - protein, 221 aa, synthetic construct
  source 1..221: note = XENP16827 Chain 2 - MESO-B[MSLN]_L0 Light Chain
QPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 250 - protein, 120 aa, synthetic construct
  source 1..120: note = MESO-C[MSLN]_H1L1 Variable heavy (vh) domain
QVQLVQSGAEVKKPGASVKVSCKASGYTFTSYWMHWVRQAPGQGLEWMGMIHPNSDNTIYYEKFQGRVTMTVDKSISTAYMELSRLRSDDTAVYYCAIIITPVVPKFDYWGQGTLVTVSS

SEQ ID NO: 251 - protein, 5 aa, synthetic construct
  source 1..5: note = MESO-C[MSLN]_H1L1 vhCDR1
SYWMH

SEQ ID NO: 252 - protein, 17 aa, synthetic construct
  source 1..17: note = MESO-C[MSLN]_H1L1 vhCDR2
MIHPNSDNTIYYEKFQG

SEQ ID NO: 253 - protein, 11 aa, synthetic construct
  source 1..11: note = MESO-C[MSLN]_H1L1 vhCDR3
IITPVVPKFDY

SEQ ID NO: 254 - protein, 107 aa, synthetic construct
  source 1..107: note = MESO-C[MSLN]_H1L1 Variable light (vl) domain
DIVMTQSPDSLAVSLGERATINCKASHDVGTSVAWYQQKPGQSPKLLIYWASTRHTGVPDRFTGSGSGTDFTLTISSLQAEDVAVYFCQQYSSYPLTFGAGTKLEIK

SEQ ID NO: 255 - protein, 11 aa, synthetic construct
  source 1..11: note = MESO-C[MSLN]_H1L1 vlCDR1
KASHDVGTSVA

SEQ ID NO: 256 - protein, 7 aa, synthetic construct
  source 1..7: note = MESO-C[MSLN]_H1L1 vlCDR2
WASTRHT

SEQ ID NO: 257 - protein, 9 aa, synthetic construct
  source 1..9: note = MESO-C[MSLN]_H1L1 vlCDR3
QQYSSYPLT

SEQ ID NO: 258 - protein, 449 aa, synthetic construct
  source 1..449: note = XENP31693 Chain 1 - MESO-C[MSLN]_H1_IgG1_PVA_/S267K Heavy Chain
QVQLVQSGAEVKKPGASVKVSCKASGYTFTSYWMHWVRQAPGQGLEWMGMIHPNSDNTIYYEKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 259 - protein, 214 aa, synthetic construct
  source 1..214: note = XENP31693 Chain 2 - MESO-C[MSLN]_L1 Light Chain
DIVMTQSPDSLAVSLGERATINCKASHDVGTSVAWYQQKPGQSPKLLIYWASTRHTGVPDRFTGSGSGTDFTLTISSLQAEDVAVYFCQQYSSYPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 260 - protein, 120 aa, synthetic construct
  source 1..120: note = MESO-C[MSLN]_H1.1 Variable Heavy
QVQLVQSGAEVKKPGASVKVSCKASGYTFTGYWMHWVRQAPGQGLEWMGMIHPNSDNTIYYEKFQGRVTMTVDKSISTAYMELSRLRSDDTAVYYCAIIITPVVPKFDYWGQGTLVTVSS

SEQ ID NO: 261 - protein, 120 aa, synthetic construct
  source 1..120: note = MESO-C[MSLN]_H1.2 Variable Heavy
QVQLVQSGAEVKKPGASVKVSCKASGYTFTSYYMHWVRQAPGQGLEWMGMIHPNSDNTIYYEKFQGRVTMTVDKSISTAYMELSRLRSDDTAVYYCAIIITPVVPKFDYWGQGTLVTVSS

SEQ ID NO: 262 - protein, 120 aa, synthetic construct
  source 1..120: note = MESO-C[MSLN]_H1.3 Variable Heavy
QVQLVQSGAEVKKPGASVKVSCKASGYTFTSYWMHWVRQAPGQGLEWMGWIHPNSDNTIYYEKFQGRVTMTVDKSISTAYMELSRLRSDDTAVYYCAIIITPVVPKFDYWGQGTLVTVSS

SEQ ID NO: 263 - protein, 120 aa, synthetic construct
  source 1..120: note = MESO-C[MSLN]_H1.4 Variable Heavy
QVQLVQSGAEVKKPGASVKVSCKASGYTFTSYWMHWVRQAPGQGLEWMGMINPNSDNTIYYEKFQGRVTMTVDKSISTAYMELSRLRSDDTAVYYCAIIITPVVPKFDYWGQGTLVTVSS

SEQ ID NO: 264 - protein, 120 aa, synthetic construct
  source 1..120: note = MESO-C[MSLN]_H1.5 Variable Heavy
QVQLVQSGAEVKKPGASVKVSCKASGYTFTSYWMHWVRQAPGQGLEWMGMIHPQSDNTIYYEKFQGRVTMTVDKSISTAYMELSRLRSDDTAVYYCAIIITPVVPKFDYWGQGTLVTVSS

SEQ ID NO: 265 - protein, 120 aa, synthetic construct
  source 1..120: note = MESO-C[MSLN]_H1.6 Variable Heavy
QVQLVQSGAEVKKPGASVKVSCKASGYTFTSYWMHWVRQAPGQGLEWMGMIHPNSGNTIYYEKFQGRVTMTVDKSISTAYMELSRLRSDDTAVYYCAIIITPVVPKFDYWGQGTLVTVSS

SEQ ID NO: 266 - protein, 120 aa, synthetic construct
  source 1..120: note = MESO-C[MSLN]_H1.7 Variable Heavy
QVQLVQSGAEVKKPGASVKVSCKASGYTFTSYWMHWVRQAPGQGLEWMGMIHPNSDNTIYYEKFQGRVTMTVDKSISTAYMELSRLRSDDTAVYYCARIITPVVPKFDYWGQGTLVTVSS

SEQ ID NO: 267 - protein, 120 aa, synthetic construct
  source 1..120: note = MESO-C[MSLN]_H1.8 Variable Heavy
QVQLVQSGAEVKKPGASVKVSCKASGYTFTSYWMHWVRQAPGQGLEWMGMIHPNSDNTIYYEKFQGRVTMTVDKSISTAYMELSRLRSDDTAVYYCAIIISPVVPKFDYWGQGTLVTVSS

SEQ ID NO: 268 - protein, 120 aa, synthetic construct
  source 1..120: note = MESO-C[MSLN]_H1.9 Variable Heavy
QVQLVQSGAEVKKPGASVKVSCKASGYTFTSYWMHWVRQAPGQGLEWMGMIHPNSDNTIYYEKFQGRVTMTVDKSISTAYMELSRLRSDDTAVYYCAIIITPVVPRFDYWGQGTLVTVSS

SEQ ID NO: 269 - protein, 120 aa, synthetic construct
  source 1..120: note = MESO-C[MSLN]_H1.12 Variable Heavy
QVQLVQSGAEVKKPGASVKVSCKASGYTFTSYWMHWVRQAPGQGLEWMGMIHPNSDNTIYYQKFQGRVTMTVDKSISTAYMELSRLRSDDTAVYYCAIIITPVVPKFDYWGQGTLVTVSS

SEQ ID NO: 270 - protein, 120 aa, synthetic construct
  source 1..120: note = MESO-C[MSLN]_H1.13 Variable Heavy
QVQLVQSGAEVKKPGASVKVSCKASGYTFTSYWMHWVRQAPGQGLEWMGMIHPNSDNTIYYYKFQGRVTMTVDKSISTAYMELSRLRSDDTAVYYCAIIITPVVPKFDYWGQGTLVTVSS

SEQ ID NO: 271 - protein, 107 aa, synthetic construct
  source 1..107: note = MESO-C[MSLN]_L1.1 Variable Light
DIVMTQSPDSLAVSLGERATINCKSSHDVGTSVAWYQQKPGQSPKLLIYWASTRHTGVPDRFTGSGSGTDFTLTISSLQAEDVAVYFCQQYSSYPLTFGAGTKLEIK

SEQ ID NO: 272 - protein, 107 aa, synthetic construct
  source 1..107: note = MESO-C[MSLN]_L1.2 Variable Light
DIVMTQSPDSLAVSLGERATINCKASQDVGTSVAWYQQKPGQSPKLLIYWASTRHTGVPDRFTGSGSGTDFTLTISSLQAEDVAVYFCQQYSSYPLTFGAGTKLEIK

SEQ ID NO: 273 - protein, 107 aa, synthetic construct
  source 1..107: note = MESO-C[MSLN]_L1.3 Variable Light
DIVMTQSPDSLAVSLGERATINCKASHSVGTSVAWYQQKPGQSPKLLIYWASTRHTGVPDRFTGSGSGTDFTLTISSLQAEDVAVYFCQQYSSYPLTFGAGTKLEIK

SEQ ID NO: 274 - protein, 107 aa, synthetic construct
  source 1..107: note = MESO-C[MSLN]_L1.4 Variable Light
DIVMTQSPDSLAVSLGERATINCKASHDVLTSVAWYQQKPGQSPKLLIYWASTRHTGVPDRFTGSGSGTDFTLTISSLQAEDVAVYFCQQYSSYPLTFGAGTKLEIK

SEQ ID NO: 275 - protein, 107 aa, synthetic construct
  source 1..107: note = MESO-C[MSLN]_L1.5 Variable Light
DIVMTQSPDSLAVSLGERATINCKASHDVGTYVAWYQQKPGQSPKLLIYWASTRHTGVPDRFTGSGSGTDFTLTISSLQAEDVAVYFCQQYSSYPLTFGAGTKLEIK

SEQ ID NO: 276 - protein, 107 aa, synthetic construct
  source 1..107: note = MESO-C[MSLN]_L1.6 Variable Light
DIVMTQSPDSLAVSLGERATINCKASHDVGTSLAWYQQKPGQSPKLLIYWASTRHTGVPDRFTGSGSGTDFTLTISSLQAEDVAVYFCQQYSSYPLTFGAGTKLEIK

SEQ ID NO: 277 - protein, 107 aa, synthetic construct
  source 1..107: note = MESO-C[MSLN]_L1.7 Variable Light
DIVMTQSPDSLAVSLGERATINCKASHDVGTSVAWYQQKPGQSPKLLIYWASTRETGVPDRFTGSGSGTDFTLTISSLQAEDVAVYFCQQYSSYPLTFGAGTKLEIK

SEQ ID NO: 278 - protein, 107 aa, synthetic construct
  source 1..107: note = MESO-C[MSLN]_L1.8 Variable Light
DIVMTQSPDSLAVSLGERATINCKASHDVGTSVAWYQQKPGQSPKLLIYWASTRHSGVPDRFTGSGSGTDFTLTISSLQAEDVAVYFCQQYSSYPLTFGAGTKLEIK

SEQ ID NO: 279 - protein, 107 aa, synthetic construct
  source 1..107: note = MESO-C[MSLN]_L1.9 Variable Light
DIVMTQSPDSLAVSLGERATINCKASHDVGTSVAWYQQKPGQSPKLLIYWASTRHTGVPDRFTGSGSGTDFTLTISSLQAEDVAVYFCQQYYSYPLTFGAGTKLEIK

SEQ ID NO: 280 - protein, 107 aa, synthetic construct
  source 1..107: note = MESO-C[MSLN]_L1.10 Variable Light
DIVMTQSPDSLAVSLGERATINCKASHDVGTSVAWYQQKPGQSPKLLIYWASTRHTGVPDRFTGSGSGTDFTLTISSLQAEDVAVYFCQQYSSTPLTFGAGTKLEIK

SEQ ID NO: 281 - protein, 107 aa, synthetic construct
  source 1..107: note = MESO-C[MSLN]_L1.13 Variable Light
DIVMTQSPDSLAVSLGERATINCKASHDVGQSVAWYQQKPGQSPKLLIYWASTRHTGVPDRFTGSGSGTDFTLTISSLQAEDVAVYFCQQYSSYPLTFGAGTKLEIK

SEQ ID NO: 282 - protein, 107 aa, synthetic construct
  source 1..107: note = MESO-C[MSLN]_L1.14 Variable Light
DIVMTQSPDSLAVSLGERATINCKASHDVGTSVAWYQQKPGQSPKLLIYWASTRHTGVPDRFTGSGSGTDFTLTISSLQAEDVAVYFCQQYSSFPLTFGAGTKLEIK

SEQ ID NO: 283 - protein, 107 aa, synthetic construct
  source 1..107: note = MESO-C[MSLN]_L1.15 Variable Light
DIVMTQSPDSLAVSLGERATINCKASHDVGTSVAWYQQKPGQSPKLLIYWASTRHTGVPDRFTGSGSGTDFTLTISSLQAEDVAVYFCQQYSSLPLTFGAGTKLEIK

SEQ ID NO: 284 - protein, 107 aa, synthetic construct
  source 1..107: note = MESO-C[MSLN]_L1.16 Variable Light
DIVMTQSPDSLAVSLGERATINCKASHDVGTSVAWYQQKPGQSPKLLIYWASTRHTGVPDRFTGSGSGTDFTLTISSLQAEDVAVYFCQQYSSVPLTFGAGTKLEIK

SEQ ID NO: 285 - protein, 107 aa, synthetic construct
  source 1..107: note = MESO-C[MSLN]_L1.17 Variable Light
DIVMTQSPDSLAVSLGERATINCKASHDVGTSVAWYQQKPGQSPKLLIYWASTRHTGVPDRFTGSGSGTDFTLTISSLQAEDVAVYFCQQYSSIPLTFGAGTKLEIK

SEQ ID NO: 286 - protein, 107 aa, synthetic construct
  source 1..107: note = MESO-C[MSLN]_L1.18 Variable Light
DIVMTQSPDSLAVSLGERATINCKASHDVGTSVAWYQQKPGQSPKLLIYWASTRHTGVPDRFTGSGSGTDFTLTISSLQAEDVAVYFCQQYSSHPLTFGAGTKLEIK

SEQ ID NO: 287 - protein, 107 aa, synthetic construct
  source 1..107: note = MESO-C[MSLN]_L1.19 Variable Light
DIVMTQSPDSLAVSLGERATINCKASHDVGTYVAWYQQKPGQSPKLLIYWASTRHTGVPDRFTGSGSGTDFTLTISSLQAEDVAVYFCQQYYSYPLTFGAGTKLEIK

SEQ ID NO: 288 - protein, 107 aa, synthetic construct
  source 1..107: note = MESO-C[MSLN]_L1.20 Variable Light
DIVMTQSPDSLAVSLGERATINCKASHDVGTSVAWYQQKPGQSPKLLIYWASTRHTGVPDRFTGSGSGTDFTLTISSLQAEDVAVYFCQQYYSFPLTFGAGTKLEIK

SEQ ID NO: 289 - protein, 107 aa, synthetic construct
  source 1..107: note = MESO-C[MSLN]_L1.21 Variable Light
DIVMTQSPDSLAVSLGERATINCKASHDVGTSVAWYQQKPGQSPKLLIYWASTRHTGVPDRFTGSGSGTDFTLTISSLQAEDVAVYFCQQYYSVPLTFGAGTKLEIK

SEQ ID NO: 290 - protein, 107 aa, synthetic construct
  source 1..107: note = MESO-C[MSLN]_L1.22 Variable Light
DIVMTQSPDSLAVSLGERATINCKASHDVGTSVAWYQQKPGQSPKLLIYWASTRHTGVPDRFTGSGSGTDFTLTISSLQAEDVAVYFCQQYYSIPLTFGAGTKLEIK

SEQ ID NO: 291 - protein, 131 aa, synthetic construct
  source 1..131: note = US2013/0028906 SEQ 21 and 24 BMS-986148 VH
EVHLVESGGGLVQPGGSLRLSCAASGFTFSRYWMSWVRQAQGKGLEWVASIKQAGSEKTYVDSVKGRFTISRDNAKNSLSLQMNSLRAEDTAVYYCAREGAYYYDSASYYPYYYYYSMDVWGQGTTVTVSS

SEQ ID NO: 292 - protein, 108 aa, synthetic construct
  source 1..108: note = US2013/0028906 SEQ 21 and 24 BMS-986148 VL
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSQYTFGQGTKLEIK

SEQ ID NO: 293 - protein, 113 aa, synthetic construct
  source 1..113: note = Roche RG7882 VH
DIQMTQSPSSLSASVGDRVTITCKSSQSVLYSSNQKNYLAWFQQKPGKAPKLLIYWASTRESGVPSRFSGSGSGTDFTLTISSLQPEDFATYFCHQYLSSYTFGQGTKVEIKR

SEQ ID NO: 294 - protein, 115 aa, synthetic construct
  source 1..115: note = Roche RG7882 VL
EVQLVESGGGLVQPGGSLRLSCAASGYTFTTYWMHWVRQAPGKGLEWVGYIRPSTGYTEYNQKFKDRFTISADTSKNTAYLQMNSLRAEDTAVYYCARSRWLLDYWGQGTLVTVS

SEQ ID NO: 295 - protein, 124 aa, synthetic construct
  source 1..124: note = New1 VH
QVKLKQSGGGLVQPGGSLKVSCAASGFDFSRYWMSWVRQAPGKGLEWIGEINPDSSTIVYTPSLKDKFIMSRDNAKNTLYLQTSKVRSADTALYYCARRGSHYYGYRTGYFDVWGAGTTVTVSS

SEQ ID NO: 296 - protein, 106 aa, synthetic construct
  source 1..106: note = New1 VL
DVVLTQSPAIMSASPGEKVTMTCSASSSVSYMYWGQQKPGSSPRLLIYDTSNLASGVPVRFSGSGSGTSYSLTISRMEAEDAATYYCQQWSSYPPTFGGGTKLEIK

SEQ ID NO: 297 - protein, 120 aa, synthetic construct
  source 1..120: note = New2: NIH patent WO 2006/124641 clone "MN" VH
QVQLQQPGAELVKPGASMKLSCKASGYTFTSYWMHWVKQRPGQGLEWIGMIHPNSDNTIYYEKFKSKATLTVDKSSSTAYMQLSSLTSEDSAVYYCAIIITPVVPKFDYWGQGTTLTVSS

SEQ ID NO: 298 - protein, 107 aa, synthetic construct
  source 1..107: note = New2: NIH patent WO 2006/124641 clone "MN" VL
DIVMTQSHQFMSTSVGDRVSVTCKASHDVGTSVAWYQQKPGQSPKLLIYWASTRHTGVPDRFTGSGSGTDFTLTISNVQSEDLADYFCQQYSSYPLTFGAGTKLELK

SEQ ID NO: 299 - protein, 123 aa, synthetic construct
  source 1..123: note = New3: same patent, clone MB VH
EVKLVESGGGLVQPGGSLSLSCAASGFTFTDYYMSWVRQPPGRALEWLGFIRGKANGYTTEYSASVKGRFTISRDYSQSILYLHMNALRAEDSATYSCARYYYSNYVWYFDVWGTGTTVTVSS

SEQ ID NO: 300 - protein, 107 aa, synthetic construct
  source 1..107: note = New3: same patent, clone MB VL
DIVMTQSQKFMSTSVGDRVSVTCKASQNVGTNVAWYQQKPGLSPKVLIHSASYRFSGVPDRFTGSGSGTDFTLSISNVQSEDLAKYFCQQYNSYPLTFGAGTKLELK

SEQ ID NO: 301 - protein, 128 aa, synthetic construct
  source 1..128: note = New4: Fred Hutch US 7795411 VH
QVQLVQSGAEVKKPGASVKVSCQASGYTFTSNYMHWVRQAPGQGLEWMGIINPSDGSTTYAQRFQGRVTMTRDTSTSRVYMELSSLRSEDTAVYYCAREGAGFGPASYVSPYYAMDVWGQGTTVTVSS

SEQ ID NO: 302 - protein, 109 aa, synthetic construct
  source 1..109: note = New4: Fred Hutch US 7795411 VL
ETTLTQSPGTLSLSPGERATLSCRASQSVSSYFLAWYQQKPGQAPRLLIYGASSRASGIPDRFSGSGSGTDFTLTISRLEPEDFAVYYCQEYGGSTLATFGPGTKVDIK

SEQ ID NO: 303 - protein, 123 aa, synthetic construct; identical to SEQ ID NO: 240
  source 1..123: note = New5: US 7943133 from Boston BioCom VH

SEQ ID NO: 304 - protein, 114 aa, synthetic construct
  source 1..114: note = New5: US 7943133 from Boston BioCom VL
QPVLTQSSSLSASPGASASLTCTLRSGINVGPRIYWYQQKPGSPPQYLLNYKSDSDKQQGSGVPSRFSGSKDASANAGVLLISGLRSEDEADYYCMIWHSSAAVFGGGTQLTVL

SEQ ID NO: 305 - protein, 117 aa, synthetic construct
  source 1..117: note = New6 US 8460660 USA HHS VH
EVQLLESGGGLVQPGGSLRPSCAASGFTFSSYAMSWVRQAPGKGLEWVSAISGSGGSTHYADSVKGRFTISRDNSKNTLYLQMNSLRAEDTAVYYCVRGGALGFDNWGRGTMVTVSS

SEQ ID NO: 306 - protein, 107 aa, synthetic construct
  source 1..107: note = New6 US 8460660 USA HHS VL
VLTQPPSVSEAPRQRVTISCSGSTSNIGSNVVNWYQQFPGKPPKVVIYYDDLVASGVSDRFSGSRSGTSASLAISGLQSEDEADYYCSAWDDSLNAWVFGGGTKVTV

SEQ ID NO: 307 - protein, 119 aa, synthetic construct
  source 1..119: note = New7 USAHHS US 2005/214304 VH
QVQLQQSGPELEKPGASVKLSCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSS

SEQ ID NO: 308 - protein, 106 aa, synthetic construct
  source 1..106: note = New7 USAHHS US 2005/214304 VL
DIELTQSPAIMSASPGEKVTMTCSASSSVSYMHWYQQKSGTSPKRWIYDTSKLASGVPGRFSGSGSGNSYSLTISSVEAEDDATYYCQQWSGYPLTFGAGTKLEIK

SEQ ID NO: 309 - protein, 119 aa, synthetic construct
  source 1..119: note = Same reference new8 VH
QVQLQQSGPELEKPGASVKLSCKASGYSFTGYTMNWVKQSHGKCLEWIGLITPYNGASSYNQKFRGKATLTVDKSSSTAYMDLLSLTSEDSAVYFCARGGYDGRGFDYWGQGTTVTVSS

SEQ ID NO: 310 - protein, 106 aa, synthetic construct
  source 1..106: note = Same reference new8 VL
DIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGCGTKLEIK

SEQ ID NO: 311 - protein, 119 aa, synthetic construct
  source 1..119: note = New9 USA HHS US 7521054 VH
EVQLQQSGPELEQPGASVKLSCKASGYSFTGYTMNWVKQSHGKCLEWIGLITPYNGASSYNQKFRGKATLTVDKSQSTAYMDLLNLTNEDSAVYFCARGGYDGRGFDYWGQGTTVTVSS

SEQ ID NO: 312 - protein, 106 aa, synthetic construct
  source 1..106: note = New9 USA HHS US 7521054 VL
DIELTQDPAIMSASPGEQVTMTCSASSSVSYMHWYQQKSGTSPKRWIYDTSKLASGVPDRFSGSGSGNSYSLTISSVEEEDDATYYCQQWSKHPLTFGCGTKLEIE

SEQ ID NO: 313 - protein, 119 aa, synthetic construct
  source 1..119: note = Next 3 are from US 7592426 Morphotek MSAb-1 (amatuximab) VH
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSS

SEQ ID NO: 314 - protein, 106 aa, synthetic construct
  source 1..106: note = Next 3 are from US 7592426 Morphotek MSAb-1 (amatuximab) VL
DIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIK

SEQ ID NO: 315 - protein, 119 aa, synthetic construct
  source 1..119: note = MsAb-2 VH
QVQLVQSGAEVKKPGASVKVSCKASGYSFTGYTMNWVRQAPGQGLEWMGLITPYNGASSYNQKFRGRVTMTRDTSTSTVYMELSSLRSEDTAVYYCARGGYDGRGFDYWGSGTPVTVSS

SEQ ID NO: 316 - protein, 106 aa, synthetic construct
  source 1..106: note = MsAb-2 VL
EIVLTQSPATLSLSPGERATLSCSASSSVSYMHWYQQKPGQAPRLLIYDTSKLASGVPARFSGSGSGTDFTLTISSLEPEDFAVYYCQQWSKHPLTFGSGTKVEIK

SEQ ID NO: 317 - protein, 119 aa, synthetic construct
  source 1..119: note = MsAb-3 VH
QVQLVQSGAEVKKPGASVKVSCKASGYSFTGYTMNWVKQAPGQGLEWIGLITPYNGASSYNQKFRGKATMTRDTSTSTVYMELSSLRSEDTAVYFCARGGYDGRGFDYWGSGTPVTVSS

SEQ ID NO: 318 - protein, 106 aa, synthetic construct
  source 1..106: note = MsAb-3 VL
EIVLTQSPATLSLSPGERATMTCSASSSVSYMHWYQQKPGQAPRLLIYDTSKLASGVPARFSGSGSGTDFTLTISSLEPEDFAVYYCQQWSKHPLTFGSGTKVEIK

SEQ ID NO: 319 - protein, 121 aa, synthetic construct
  source 1..121: note = MF-J VH
QVELVQSGAEVKKPGESLKISCKGSGYSFTNYWIGWVRQMPGKGLEWMGVIMPSDSYTRYSPSFQGQVTISADKSISTAYLQWSSLKASDTAMYYCARYGHGMYGGALDVWGQGTLVTVSS

SEQ ID NO: 320 - protein, 108 aa, synthetic construct
  source 1..108: note = MF-J VL
DIVLTQSPATLSLSPGERATLSCRASQSVRSSRLAWYQQKPGQAPRLLIYGASKRATGVPARFSGSGSGTDFTLTISSLEPEDFATYYCQQYYDFPPTFGQGTKVEIK

SEQ ID NO: 321 - protein, 121 aa, synthetic construct
  source 1..121: note = "Moro6640" VH
QVQLVQSGAEVKKPGESLKISCKGSGYSFTNYWIGWVRQMPGKGLEWMGVIMPSDSYTRYSPSFQGQVTISADKSISTAYLQWSSLKASDTAMYYCARYGHGMYGGALDVWGQGTLVTVSS

SEQ ID NO: 322 - protein, 109 aa, synthetic construct
  source 1..109: note = "Moro6640" VL
DIVLTQSPATLSLSPGERATLSCRASQSVRSSRLAWYQQKPGQAPRLLIYGASKRATGVPARFSGSGSGTDFTLTISSLEPEDFAVYYCQQYSHDPSGTFGQGTKVEIK

SEQ ID NO: 323 - protein, 119 aa, synthetic construct
  source 1..119: note = MF-226 VH
QVELVQSGAEVKKPGASVKVSCKASGYTFTGNYINWVRQAPGQGLEWMGIINPHGGDTKYAQKFQGRVTMTRDTSISTAYMELSSLRSEDTAVYYCARWHHGTWIFDYWGQGTLVTVSS

SEQ ID NO: 324 - protein, 109 aa, synthetic construct
  source 1..109: note = MF-226 VL
DIVLTQPPSVSGAPGQRVTISCSGSSSNIGSNYVSWYQQLPGTAPKLLIYNDNQRPSGVPDRFSGSKSGTSASLAITGLQSEDEADYYCSTYDRRTFSVFGGGTKLTVL

SEQ ID NO: 325 - protein, 121 aa, synthetic construct
  source 1..121: note = "Moro7265" VH
QVQLVQSGAEVKKPGESLKISCKGSGYSFTNYWIGWVRQMPGKGLEWMGFIWPVDSWTQYSPSFQGQVTISADKSISTAYLQWSSLKASDTAMYYCARYGHGMYGGALDVWGQGTLVTVSS

SEQ ID NO: 326 - protein, 109 aa, synthetic construct; identical to SEQ ID NO: 322
  source 1..109: note = "Moro7265" VL

SEQ ID NO: 327 - protein, 121 aa, synthetic construct; identical to SEQ ID NO: 325
  source 1..121: note = MOR06631 VH

SEQ ID NO: 328 - protein, 108 aa, synthetic construct; identical to SEQ ID NO: 320
  source 1..108: note = MOR06631 VL

SEQ ID NO: 329 - protein, 121 aa, synthetic construct
  source 1..121: note = MOR06669 VH
QVQLVQSGAEVKKPGESLKISCKGSGYSFTNYWIGWVRQMPGKGLEWMGIIWPIDSFTQYSPSFQGQVTISADKSISTAYLQWSSLKASDTAMYYCARYGHGMYGGALDVWGQGTLVTVSS

SEQ ID NO: 330 - protein, 108 aa, synthetic construct; identical to SEQ ID NO: 320
  source 1..108: note = MOR06669 VL

SEQ ID NO: 331 - protein, 121 aa, synthetic construct; identical to SEQ ID NO: 321
  source 1..121: note = "MOR07111 VH

SEQ ID NO: 332 - protein, 108 aa, synthetic construct
  source 1..108: note = "MOR07111 VL
DIVLTQSPATLSLSPGERATLSCRASQSVRSSRLAWYQQKPGQAPRLLIYGASKRATGVPARFSGSGSGTDFTLTISSLEPEDFAVYYCQQFYSKPITFGQGTKVEIK

SEQ ID NO: 333 - protein, 121 aa, synthetic construct; identical to SEQ ID NO: 321
  source 1..121: note = MORO6642 VH

SEQ ID NO: 334 - protein, 109 aa, synthetic construct
  source 1..109: note = MORO6642 VL
DIVLTQSPATLSLSPGERATLSCRASQSVRSSRLAWYQQKPGQAPRLLIYGASKRATGVPARFSGSGSGTDFTLTISSLEPEDFAVYYCQQYSQDPSSTFGQGTKVEIK

SEQ ID NO: 335 - protein, 121 aa, synthetic construct
  source 1..121: note = MOR06643 VH
QVQLVESGGGLVQPGGSLRLSCAASGFTFSDYAMSWVRQAPGKGLEWVSAIMYDSSSTFYADSVKGRFTISRDNSKNTLYLQMNSLRAEDTAVYYCARINYIYKGVHFDYWGQGTLVTVSS

SEQ ID NO: 336 - protein, 108 aa, synthetic construct
  source 1..108: note = MOR06643 VL
DIVLTQSPATLSLSPGERATLSCRASQIVSGYLAWYQQKPGQAPRLLIYGASSRATGVPARFSGSGSGTDFTLTISSLEPEDFAVYYCQQWSLRSPFTFGQGTKVEIK

SEQ ID NO: 337 - protein, 119 aa, synthetic construct
  source 1..119: note = MOR06626 VH
QVQLVQSGAEVKKPGASVKVSCKASGYTFTGNYINWVRQAPGQGLEWMGIINPTKGWTLYAQKFQGRVTMTRDTSISTAYMELSSLRSEDTAVYYCARWHHGTWIFDYWGQGTLVTVSS

SEQ ID NO: 338 - protein, 109 aa, synthetic construct; identical to SEQ ID NO: 324
  source 1..109: note = MOR06626 VL

SEQ ID NO: 339 - protein, 121 aa, synthetic construct
  source 1..121: note = MOR06635 VH
QVQLVQSGAEVKKPGESLKISCKGSGYSFTNYWIGWVRQMPGKGLEWMGFIWPSDSWTSYSPSFQGQVTISADKSISTAYLQWSSLKASDTAMYYCARYGHGMYGGALDVWGQGTLVTVSS

SEQ ID NO: 340 - protein, 108 aa, synthetic construct; identical to SEQ ID NO: 320
  source 1..108: note = MOR06635 VL

SEQ ID NO: 341 - protein, 119 aa, synthetic construct
  source 1..119: note = MOR06638 VH
QVQLVQSGAEVKKPGASVKVSCKASGYTFTGNYINWVRQAPGQGLEWMGIINPHGGDTKYAQKFQGRVTMTRDTSISTAYMELSSLRSEDTAVYYCARWHHGTWIFDYWGQGTLVTVSS

SEQ ID NO: 342 - protein, 110 aa, synthetic construct
  source 1..110: note = MOR06638 VL
DIVLTQPPSVSGAPGQRVTISCSGSSSNIGSNYVSWYQQLPGTAPKLLIYNDNQRPSGVPDRFSGSKSGTSASLAITGLQSEDEADYYCSSWDRADGSYVFGGGTKLTVL

SEQ ID NO: 343 - protein, 115 aa, synthetic construct
  source 1..115: note = MF-A VH
QVQLVQSGAEVKKPGSSVKVSCKASGGTFSSYYFSWVRQAPGQGLEWMGGIIPKFGSANYAQKFQGRVTITADESTSTAYMELSSLRSEDTAVYYCARRTSMDYWGQGTLVTVSS

SEQ ID NO: 344 - protein, 111 aa, synthetic construct
  source 1..111: note = MF-A VL
DIALTQPASVSGSPGQSITISCTGTSSDVGGYNYVSWYQQHPGKAPKLMIYSVSKRPSGVSNRFSGSKSGNTASLTISGLQAEDEADYYCGAWAHMSLGKVFGGGTKLTVL

SEQ ID NO: 345 - protein, 115 aa, synthetic construct; identical to SEQ ID NO: 343
  source 1..115: note = MORo6657 VH

SEQ ID NO: 346 - protein, 111 aa, synthetic construct
  source 1..111: note = MORo6657 VL
DIALTQPASVSGSPGQSITISCTGTSSDVGGYNYVSWYQQHPGKAPKLMIYSVSKRPSGVSNRFSGSKSGNTASLTISGLQAEDEADYYCATWDHSQMGKVFGGGTKLTVL

SEQ ID NO: 347 - protein, 121 aa, synthetic construct; identical to SEQ ID NO: 335
  source 1..121: note = MF-L VH

SEQ ID NO: 348 - protein, 107 aa, synthetic construct
  source 1..107: note = MF-L VL
DIVLTQSPATLSLSPGERATLSCRASQIVSGYLAWYQQKPGQAPRLLIYGASSRATGVPARFSGSGSGTDFTLTISSLEPEDFATYYCQQYYNFSFTFGQGTKVEIK

SEQ ID NO: 349 - protein, 116 aa, synthetic construct
  source 1..116: note = MF-1 VH
QVQLQESGPGLVKPGETLSLTCTVSGGSISNYYWSWIRQAPGKGLEWIGEIYHSGGTYYNPSLKGRVTISVDTSKNQFSLKLSSVTAEDTAVYYCARPMDNLPDIWGQGTLVTVSS

SEQ ID NO: 350 - protein, 107 aa, synthetic construct
  source 1..107: note = MF-1 VL
DIQMTQSPSSLSASVGDRVTITCRASQNIGSYLNWYQQKPGKAPKLLIYNSSTLQSGVPSRFSGSGSGTDFTLTISSLQPEDFATYYCQQRSNMPITFGQGTKVEIK

SEQ ID NO: 351 - protein, 116 aa, synthetic construct
  source 1..116: note = MF-5 VH
QVQLVESGGGLVQPGGSLRLSCAASGFTFSSYGMSWVRQAPGKGLEWVSGISYSSSATYYADSVKGRFTISRDNSKNTLYLQMNSLRAEDTAVYYCARYLYYFDVWGQGTLVTVSS

SEQ ID NO: 352 - protein, 109 aa, synthetic construct
  source 1..109: note = MF-5 VL
DIALTQPASVSGAPGQRVTISCTGTSSDVGASDTVTWYQQHPGKAPKLMIYAVNKRPSGVSNRFSGSKSGNTASLTISGLQAEDEADYYCASRDSSSMVFGGGTKLTVL

SEQ ID NO: 353 - protein, 115 aa, synthetic construct
  source 1..115: note = MF-8 VH
QVQLVESGGGLVQPGGSLRLSCAASGFTFSNSWMSWVRQAPGKGLEWVSAITYWGSNTYYADSVKGRFTISRDNSKNTLYLQMNSLRAEDTAVYYCARTKFFANWGQGTLVTVSS

SEQ ID NO: 354 - protein, 112 aa, synthetic construct
  source 1..112: note = MF-8 VL
DIVMTQSPLSLPVTPGEPASISCRSSQSLLHSNGYTYLSWYLQKPGQSPQLLIYLGSKRASGVPDRFSGSGSGTDFTLKISRVEAEDVGVYYCQQYYDSSSTFGQGTKVEIK

SEQ ID NO: 355 - protein, 115 aa, synthetic construct
  source 1..115: note = MF-24 VH
QVQLVQSGAEVKKPGSSVKVSCKASGGTFSNYSISWVRQAPGQGLEWMGRIIPNFGTANYAQKFQGRVTITADESTSTAYMELSSLRSEDTAVYYCARGIYFAFWGQGTLVTVSS

SEQ ID NO: 356 - protein, 110 aa, synthetic construct
  source 1..110: note = MF-24 VL
DIVLTQPPSVSGAPGQRVTISCSGSSSNIGPNYVSWYQQLPGTAPKLLIHGNANRPSGVPDRFSGSKSGTSASLAITGLQSEDEADYYCQSYDFFTNSSVFGGGTKLTVL

SEQ ID NO: 357 - protein, 120 aa, synthetic construct
  source 1..120: note = MF-25 VH
QVQLVQSGAEVKKPGASVKVSCKASGYTFTSYALHWVRQAPGQGLEWMGIINPQNGGTNYAQKFQGRVTMTRDTSISTAYMELSSLRSEDTAVYYCARKHKYRIGSMDVWGQGTLVTVSS

SEQ ID NO: 358 - protein, 108 aa, synthetic construct
  source 1..108: note = MF-25 VL
DIVLTQSPATLSLSPGERATLSCRASQSLTSNQLAWYQQKPGQAPRLLIYDSSNRATGVPARFSGSGSGTDFTLTISSLEPEDFAVYYCQQYGSFPATFGQGTKVEIK

SEQ ID NO: 359 - protein, 117 aa, synthetic construct
  source 1..117: note = MF-27 VH
QVQLVQSGAEVKKPGESLKISCKGSGYSFTNYWIGWVRQMPGKGLEWMGIIDPRESFTRYSPSFQGQVTISADKSISTAYLQWSSLKASDTAMYYCARYMKGGYDYWGQGTLVTVSS

SEQ ID NO: 360 - protein, 111 aa, synthetic construct
  source 1..111: note = MF-27 VL
DIALTQPASVSGSPGQSITISCTGTSSDVGGNNFVSWYQQHPGKAPKLMIYYGDSRPSGVSNRFSGSKSGNTASLTISGLQAEDEADYYCQSWDAPMGMWVFGGGTKLTVL

SEQ ID NO: 361 - protein, 120 aa, synthetic construct
  source 1..120: note = MF-73 VH
QVQLQQSGPGLVKPSQTLSLTCAISGDSVSSRSAAWGWIRQSPGRGLEWLGRIGYRSKWMNDYAVSVKSRITINPDTSKNQFSLQLNSVTPEDTAVYYCARMQGFQLDYWGQGTLVTVSS

SEQ ID NO: 362 - protein, 107 aa, synthetic construct
  source 1..107: note = MF-73 VL
DIQMTQSPSSLSASVGDRVTITCRASQTISSALAWYQQKPGKAPKLLIYGASTLQSGVPSRFSGSGSGTDFTLTISSLQPEDFATYYCQQYYSYSVTFGQGTKVEIK

SEQ ID NO: 363 - protein, 115 aa, synthetic construct
  source 1..115: note = MF-78 VH
QVQLVQSGAEVKKPGSSVKVSCKASGGTFSNYSLHWVRQAPGQGLEWMGGIVPIFGTANYAQKFQGRVTITADESTSTAYMELSSLRSEDTAVYYCARTYTFAVWGQGTLVTVSS

SEQ ID NO: 364 - protein, 110 aa, synthetic construct
  source 1..110: note = MF-78 VL
DIALTQPASVSGSPGQSITISCTGTSSDVGNFNYVNWYQQHPGKAPKLMIYSVSSRPSGVSNRFSGSKSGNTASLTISGLQAEDEADYYCGAYTTDTLSVFGGGTKLTVL

SEQ ID NO: 365 - protein, 116 aa, synthetic construct
  source 1..116: note = MF-84 VH
QVQLVESGGGLVQPGGSLRLSCAASGFTFSSYAMNWVRQAPGKGLEWVSGISGNGSNTYYADSVKGRFTISRDNSKNTLYLQMNSLRAEDTAVYYCARKWLFYDYWGQGTLVTVSS

SEQ ID NO: 366 - protein, 108 aa, synthetic construct
  source 1..108: note = MF-84 VL
DIVLTQSPATLSLSPGERATLSCRASQSVTSNYLAWYQQKPGQAPRLLIYGSSSRATGVPARFSGSGSGTDFTLTISSLEPEDFAVYYCQQYSDIPATFGQGTKVEIK

SEQ ID NO: 367 - protein, 114 aa, synthetic construct
  source 1..114: note = MF-101 VH
QVQLVQSGAEVKKPGASVKVSCKASGYTFTIYDMHWVRQAPGQGLEWMGYISPYSGDTNYAQKFQGRVTMTRDTSISTAYMELSSLRSEDTAVYYCARGWQDFWGQGTLVTVSS

SEQ ID NO: 368 - protein, 107 aa, synthetic construct
  source 1..107: note = MF-101 VL
DIQMTQSPSSLSASVGDRVTITCRASQSINRSLTWYQQKPGKAPKLLIYAASNLQSGVPSRFSGSGSGTDFTLTISSLQPEDFAVYYCLQVYNLPLTFGQGTKVEIK

SEQ ID NO: 369 - protein, 126 aa, synthetic construct
  source 1..126: note = MF-230 VH
QVQLQQSGPGLVKPSQTLSLTCAISGDSVSSNSAAWGWIRQSPGRGLEWLGRISYRSRWYNNYAVSVKSRITINPDTSKNQFSLQLNSVTPEDTAVYYCARYYSDHFGLYPYFDYWGQGTLVTVSS

SEQ ID NO: 370 - protein, 107 aa, synthetic construct; identical to SEQ ID NO: 368
  source 1..107: note = MF-230 VL

SEQ ID NO: 371 - protein, 126 aa, synthetic construct
  source 1..126: note = MF-236 VH
QVQLVQSGAEVKKPGESLKISCKGSGYSFNTSWIWVRQMPGKGLEWMGIIHPGHSYTRYSPSFQGQVTISADKSISTAYLQWSSLKASDTAMYYCARGDGGPSSQGNYFGWVYDVWGQGTLVTVSS

SEQ ID NO: 372 - protein, 108 aa, synthetic construct
  source 1..108: note = MF-236 VL
DIELTQPPSVSVAPGQTARISCSGDNIPNFYVHWYQQKPGQAPVLVIYEDSDRPSGIPERFSGSNSGNTATLTISGTQAEDEADYYCQSYDKPTFSGVFGGGTKLTVL

SEQ ID NO: 373 - protein, 125 aa, synthetic construct
  source 1..125: note = MF252 VH
QVQLQQSGPGLVKPSQTLSLTCAISGDSVSSNSAAWSWIRQSPGRGLEWLGRIYYRSKKWYNDYAVSVKSRITINPDTSKNQFSLQLNSVTPEDTAVYYCARNYSGPMYYYGDVWGQGTLVTVSS

SEQ ID NO: 374 - protein, 111 aa, synthetic construct
  source 1..111: note = MF252 VL
DIALTQPASVSGSPGQSITISCTGTSSDIGRYHYVSWYQQHPGKAPKVMIYSVSKRPSGVSNRFSGSKSGNTASLTISGLQAEDEADYYCSAYDTNNYLSVFGGGTKLTVL

SEQ ID NO: 375 - protein, 123 aa, synthetic construct
  source 1..123: note = MF-275 VH
QVQLQQSGPGLVKPSQTLSLTCAISGDSVSSNSAAWSWIRQSPGRGLEWLGRIQYRSKWYNAYAVSVKSRITINPDTSKNQFSLQLNSVTPEDTAVYYCARGFHGSTMYFDVWGQGTLVTVSS

SEQ ID NO: 376 - protein, 106 aa, synthetic construct
  source 1..106: note = MF-275 VL
DIVLTQPPSVSGAPGQRVTISCSGSSSNIGNNSVNWYQQLPGTAPKLLIYNNQRPSGVPDRFSGSKSGTSASLAITGLQSEDEADYYCQAYASNIVFGGGTKLTVL

SEQ ID NO: 377 - protein, 117 aa, synthetic construct
  source 1..117: note = MF-423 VH
QVQLVQSGAEVKKPGESLKISCKGSGYSFTTYWIGWVRQAPGKGLEWMGFIYPDKSYTNYSPSFQGQVTISADKSISTAYLQWSSLKASDTAMYYCARGLGGSFDVWGQGTLVTVSS

SEQ ID NO: 378 - protein, 109 aa, synthetic construct
  source 1..109: note = MF-423 VL
DIALTQPASVSGSPGQSITISCTGTSSDVGDYNYVSWYQQHPGKAPKLMIYRVDNRPSGVSNRFSGSKSGNTASLTISGLQAEDEADYYCQSWVGPSTVFGGGTKLTVL

SEQ ID NO: 379 - protein, 120 aa, synthetic construct
  source 1..120: note = MF-428 VH
QVQLVESGGGLVQPGGSLRLSCAASGFTFSTYAMSWVRQAPGKGLEWVSFISGYGSSTYYADSVKGRFTISRDNSKNTLYLQMNSLRAEDTAVYYCARKMYWWSDGFDYWGQGTLVTVSS

SEQ ID NO: 380 - protein, 106 aa, synthetic construct
  source 1..106: note = MF-428 VL
DIELTQPPSVSVAPGQTARISCSGDNLRSKYAHWYQQKPGQAPVLVIYSKDNRPSGIPERFSGSNSGNTATLTISGTQAEDEADYYCSSWAHDHKVFGGGTKLTVL

SEQ ID NO: 381 - protein, 121 aa, synthetic construct
  source 1..121: note = MF-C VH
QVQLVESGGGLVQPGGSLRLSCAASGFTFSRYAISWVRQAPGKGLEWVSSISGGGSKTFYADSVKGRFTISRDNSKNTLYLQMNSLRAEDTAVYYCARYPGPTGHVFFDIWGQGTLVTVSS

SEQ ID NO: 382 - protein, 108 aa, synthetic construct
  source 1..108: note = MF-C VL
DIELTQPPSVSVAPGQTARISCSGDNIGSKVATWYQQKPGQAPVLVIYYDNDRPSGIPERFSGSNSGNTATLTISGTQAEDEADYYCQSYDGQMSTSVFGGGTKLTVL

SEQ ID NO: 383 - protein, 116 aa, synthetic construct
  source 1..116: note = MF-I VH
QVQLVQSGAEVKKPGSSVKVSCKASGGTFSSYYISWVRQAPGQGLEWMGGIIPKFGTANYAQKFQGRVTITADESTSTAYMELSSLRSEDTAVYYCARTIGIYDSWGQGTLVTVSS

SEQ ID NO: 384 - protein, 111 aa, synthetic construct
  source 1..111: note = MF-I VL
DIALTQPASVSGSPGQSITISCTGTSSDVGGYNYVSWYQQHPGKAPKLMIYSVNNRPSGVSNRFSGSKSGNTASLTISGLQAEDEADYYCSTYDHTSSGFVFGGGTKLTVL

SEQ ID NO: 385 - protein, 115 aa, synthetic construct
  source 1..115: note = MF-M VH
QVQLVQSGAEVKKPGSSVKVSCKASGGTFSSHAISWVRQAPGQGLEWMGNIMPIFGVANYAQKFQGRVTITADESTSTAYMELSSLRSEDTAVYYCAREMRLAYWGQGTLVTVSS

SEQ ID NO: 386 - protein, 109 aa, synthetic construct
  source 1..109: note = MF-M VL
DIALTQPASVSGSPGQSITISCTGTSSDIGHFNYVSWYQQHPGKAPKLMIYSVISRPSGVSNRFSGSKSGNTASLTISGLQAEDEADYYCASFTFPSLVFGGGTKLTVL

SEQ ID NO: 387 - protein, 126 aa, synthetic construct
  source 1..126: note = MF-P VH
QVQLQQSGPGLVKPSQTLSLTCAISGDSVSSNTAAWSWIRQSPGRGLEWLGRIRYRSKWYNDYAVSVKSRITINPDTSKNQFSLQLNSVTPEDTAVYYCARGYHQGLYGNHMFDVWGQGTLVTVSS

SEQ ID NO: 388 - protein, 110 aa, synthetic construct
  source 1..110: note = MF-P VL
DIVLTQPPSVSGAPGQRVTISCSGSSSNIGSHTVNWYQQLPGTAPKLLIYDVNKRPSGVPDRFSGSKSGTSASLAITGLQSEDEADYYCASWDSVQVSPVFGGGTKLTVL

SEQ ID NO: 389 - protein, 118 aa, synthetic construct
  source 1..118: note = MF-Q VH
QVQLKESGPALVKPTQTLTLTCTFSGFSLSSSGVGVSWIRQPPGKALEWLALIDWDDDKSYSTSLKTRLTISKDTSKNQVVLTMTNMDPVDTATYYCARIQGWNYDVWGQGTLVTVSS

SEQ ID NO: 390 - protein, 109 aa, synthetic construct
  source 1..109: note = MF-Q VL
DIVLTQPPSVSGAPGQRVTISCSGSSSNIGNNRVSWYQQLPGTAPKLLIYGNNKRPSGVPDRFSGSKSGTSASLAITGLQSEDEADYYCSAWDLLEVYVFGGGTKLTVL

SEQ ID NO: 391 - protein, 120 aa, synthetic construct
  source 1..120: note = MF-S VH
QVQLQQSGPGLVKPSQTLSLTCAISGDSVSSSSAAWSWIRQSPGRGLEWLGRIGQRSKWYNDYAVSVKSRITINPDTSKNQFSLQLNSVTPEDTAVYYCARSRFGYFDVWGQGTLVTVSS

SEQ ID NO: 392 - protein, 109 aa, synthetic construct
  source 1..109: note = MF-S VL
DIALTQPASVSGSPGQSITISCTGTSSDIGTYNHVSWYQQHPGKAPKLMIYNVNKRPSGVSNRFSGSKSGNTASLTISGLQAEDEADYYCSAYAPSAVVFGGGTKLTVL

SEQ ID NO: 393 - protein, 118 aa, synthetic construct
  source 1..118: note = MF-U VH
QVQLVESGGGLVQPGGSLRLSCAASGFTFSSHYSWVRQAPGKGLEWVSTISSNGSYTYYADSVKGRFTISRDNSKNTLYLQMNSLRAEDTAVYYCARFVARLNVFDYWGQGTLVTVSS

SEQ ID NO: 394 - protein, 111 aa, synthetic construct
  source 1..111: note = MF-U VL
DIVLTQPPSVSGAPGQRVTISCTGSSSNIGIGYDVNWYQQLPGTAPKLLIYKNTNRPSGVPDRFSGSKSGTSASLAITGLQSEDEADYYCSSYDSFHHGIVFGGGTKLTVL

SEQ ID NO: 395 - protein, 115 aa, synthetic construct
  source 1..115: note = MF-V VH
QVQLVQSGAEVKKPGSSVKVSCKASGGTFSNYTISWVRQAPGQGLEWMGNIIPAFGYANYAQKFQGRVTITADESTSTAYMELSSLRSEDTAVYYCARKFTFDVWGQGTLVTVSS

SEQ ID NO: 396 - protein, 111 aa, synthetic construct
  source 1..111: note = MF-V VL
DIALTQPASVSGSPGQSITISCTGTSSDLGGYSYVSWYQQHPGKAPKLMIYNVNNRPSGVSNRFSGSKSGNTASLTISGLQAEDEADYYCSSYDYVSSDTVFGGGTKLTVL

SEQ ID NO: 397 - protein, 118 aa, synthetic construct
  source 1..118: note = MF-W VH
QVQLVESGGGLVQPGGSLRLSCAASGFTFSSYYSWVRQAPGKGLEWVSNISGNGSSTYYADSVKGRFTISRDNSKNTLYLQMNSLRAEDTAVYYCARLIATLGTFDYWGQGTLVTVSS

SEQ ID NO: 398 - protein, 111 aa, synthetic construct
  source 1..111: note = MF-W VL
DIALTQPASVSGSPGQSITISCTGTSSDVGGYNYVSWYQQHPGKAPKVMIYGVTKRPSGVSNRFSGSKSGNTASLTISGLQAEDEADYYCQTYDEHGFHIVFGGGTKLTVL

SEQ ID NO: 399 - protein, 120 aa, synthetic construct
  source 1..120: note = MF-Y VH
QVQLVESGGGLVQPGGSLRLSCAASGFTFSNYGIHWVRQAPGKGLEWVSYIRSGSSDTYYADSVKGRFTISRDNSKNTLYLQMNSLRAEDTAVYYCARTAPAGHGVFANWGQGTLVTVSS

SEQ ID NO: 400 - protein, 110 aa, synthetic construct
  source 1..110: note = MF-Y VL
DIVLTQPPSVSGAPGQRVTISCSGSSSNIGNYRVSWYQQLPGTAPKLLIYGNNKRPSGVPDRFSGSKSGTSASLAITGLQSEDEADYYCSTYTGLPFTTVFGGGTKLTVL

SEQ ID NO: 401 - protein, 122 aa, synthetic construct
  source 1..122: note = 3C10 VH
QVYLVESGGGVVQPGRSLRLSCAASGITFSIYGMHWVRQAPGKGLEWVAVIWYDGSHEYYADSVKGRFTISRDNSKNTLYLLMNSLRAEDTAVYYCARDGDYYDSGSPLDYWGQGTLVTVSS

SEQ ID NO: 402 - protein, 107 aa, synthetic construct
  source 1..107: note = 3C10 VL
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIK

SEQ ID NO: 403 - protein, 122 aa, synthetic construct
  source 1..122: note = 6A4 VH
QVHLVESGGGVVQPGRSLRLSCVASGITFRIYGMHWVRQAPGKGLEWVAVLWYDGSHEYYADSVKGRFTISRDNSKNTLYLQMNSLRAEDTAIYYCARDGDYYDSGSPLDYWGQGTLVTVSS

SEQ ID NO: 404 - protein, 107 aa, synthetic construct; identical to SEQ ID NO: 402
  source 1..107: note = 6A4 VL

SEQ ID NO: 405 - protein, 131 aa, synthetic construct; identical to SEQ ID NO: 291
  source 1..131: note = 7B1 VH

SEQ ID NO: 406 - protein, 108 aa, synthetic construct; identical to SEQ ID NO: 292
  source 1..108: note = 7B1 VL

SEQ ID NO: 407 - protein, 117 aa, synthetic construct
  source 1..117: note = 1C14-30 VH
EVQLQQSGADLVKPGASVKLSCIVSAFNIEDTYMHWVKQRPEQGLEWIGSIDPAHDNAKYDPKFQVKATITADTSSNTAYLQLSSLTSEDTAVYYCAKSDGFYFDSWGQGTTLTVSS

SEQ ID NO: 408 - protein, 107 aa, synthetic construct
  source 1..107: note = 1C14-30 VL
DIVMTQSQKFMSTSVGDRVSITCKASQNVRSAVAWYQQKPGQSPKALIYLASNRHTGVPDRFTGSGSGTDFTLTISNVQSEDLADYFCLQHWNYPLSVGGGTKLEIK

SEQ ID NO: 409 - protein, 122 aa, synthetic construct
  source 1..122: note = 11-25 VH
QVQLKESGPGLVAPSQSLSITCTVSGFSLTSYGVQWVRQPPGKGLEWLGVIWAGGSTKYNSALMSRLSISKDNSKSQVFLKMNSLQTDDTAMYYCARDDYYRRTSLYAMDYWGQGTSVTVSS

SEQ ID NO: 410 - protein, 112 aa, synthetic construct
  source 1..112: note = 11-25 VL
DVLMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPYTFGGGTKLEIK

SEQ ID NO: 411 - protein, 119 aa, synthetic construct
  source 1..119: note = m912 VH
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSS

SEQ ID NO: 412 - protein, 107 aa, synthetic construct
  source 1..107: note = m912 VL
DIQMTQSPSSLSASVGDRVTITCRASQSISSYLNWYQQKPGKAPKLLIYAASSLQSGVPSGFSGSGSGTDFTLTISSLQPEDFATYYCQQSYSTPLTFGGGTKVEIK

SEQ ID NO: 413 - protein, 119 aa, synthetic construct
  source 1..119: note = HN1 VH
QVQLVQSGAEVKRPGASVQVSCRASGYSINTYYMQWVRQAPGAGLEWMGVINPSGVTSYAQKFQGRVTLTNDTSTNTVYMQLNSLTSADTAVYYCARWALWGDFGMDVWGKGTLVTVSS

SEQ ID NO: 414 - protein, 107 aa, synthetic construct
  source 1..107: note = HN1 VL
DIQMTQSPSTLSASIGDRVTITCRASEGIYHWLAWYQQKPGKAPKLLIYKASSLASGAPSRFSGSGSGTDFTLTISSLQPDDFATYYCQQYSNYPLTFGGGTKLEIK

SEQ ID NO: 415 - protein, 120 aa, synthetic construct
  source 1..120: note = FF-TA (anetumab) VH
QVELVQSGAEVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSS

SEQ ID NO: 416 - protein, 111 aa, synthetic construct
  source 1..111: note = FF-TA (anetumab) VL
DIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTKLTVL

SEQ ID NO: 417 - protein, 119 aa, synthetic construct
  source 1..119: note = ADCP-XFT VH
QVELVQSGAVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSS

SEQ ID NO: 418 - protein, 111 aa, synthetic construct
  source 1..111: note = ADCP-XFT VL
DIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTKLEIK

SEQ ID NO: 419 - protein, 116 aa, synthetic construct
  source 1..116: note = Genentech 7D9 VH
EVQLVESGGGLVQPGGSLRLSCAASGYTFTTYWMHWVRQAPGKGLEWVGYIRPSTGYTEYNQKFKDRFTISADTSKNTAYLQMNSLRAEDTAVYYCARSRWLLDYWGQGTLVTVSS

SEQ ID NO: 420 - protein, 112 aa, synthetic construct
  source 1..112: note = Genentech 7D9 VL
DIQMTQSPSSLSASVGDRVTITCKSSQSVLYSSNQKNYLAWFQQKPGKAPKLLIYWASTRESGVPSRFSGSGSGTDFTLTISSLQPEDFATYFCHQYLSSYTFGQGTKVEIK

SEQ ID NO: 421 - protein, 117 aa, synthetic construct
  source 1..117: note = 22A10 VH
EVQLVESGGGLVQPGGSLRLSCAASGFTFSDYFMSWVRQAPGKGLEWVATISNGGTYTYYPDSVKGRFTISRDNSKNTLYLQMNSLRAEDTAVYYCARFDGYIFDYWGQGTLVTVSS

SEQ ID NO: 422 - protein, 107 aa, synthetic construct
  source 1..107: note = 22A10 VL
DIQMTQSPSSLSASVGDRVTITCRASQDISNYLNWYQQKPGKAPKLLIYYTSRLHSGVPSRFSGSGSGTDFTLTISSLQPEDFATYYCQQGNTLPYTFGQGTKVEIK

SEQ ID NO: 423 - protein, 119 aa, synthetic construct
  source 1..119: note = From WO 2015/051199 humanized SS1 VH
QVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSS

SEQ ID NO: 424 - protein, 106 aa, synthetic construct
  source 1..106: note = From WO 2015/051199 humanized SS1 VL
DIQMTQSPSSLSASVGDRVTITCSASSSVSYMHWYQQKSGKAPKLLIYDTSKLASGVPSRFSGSGSGTDFTLTISSLQPEDFATYYCQQWSKHPLTFGQGTKLEIK

SEQ ID NO: 425 - protein, 119 aa, synthetic construct
  source 1..119: note = Amgen US 10100121 unknown1 VH
QVQLQESGPGLVKPSQTLSLTCTVSGGSINNNNYYWTWIRQHPGKGLEWIGYIYYSGSTFYNPSLKSRVTISVDTSKTQFSLKLSSVTAADTAVYYCAREDTMTGLDVWGQGTLVTVSS

SEQ ID NO: 426 - protein, 106 aa, synthetic construct
  source 1..106: note = Amgen US 10100121 unknown1 VL
DIQMTQSPSSLSASVGDRVTITCRASQSINNYLNWYQQKPGKAPTLLIYAASSLQSGVPSRFSGSRSGTDFTLTISSLQPEDFAAYFCQQTYSNPTFGQGTKVEVK

SEQ ID NO: 427 - protein, 119 aa, synthetic construct
  source 1..119: note = AB237 VH
QVQLQESGPGLVKPSQTLSLTCTVSGGSINNNNYYWTWIRQHPGKGLEWIGYIYYSGSTFYNPSLKSRVTISVDTSKTQFSLKLSSVTAADTAVYYCAREDTMTGLDVWGQGTTVTVSS

SEQ ID NO: 428 - protein, 106 aa, synthetic construct; identical to SEQ ID NO: 426
  source 1..106: note = AB237 VL

SEQ ID NO: 429 - protein, 123 aa, synthetic construct
  source 1..123: note = Eisai US 2016/369011 xi33o11 VH
QSVEESGGRLVTPGTPLTLTCTVSGISLSSDAISWVRQAPGKGLEYIGIINGGGNTYYASWAKGRFTISKTSTTVDLKITSPTTEDTATYFCARGIQHGGGNSDYYYYGMDLWGPGTLVTVSS

SEQ ID NO: 430 - protein, 112 aa, synthetic construct
  source 1..112: note = Eisai US 2016/369011 xi33o11 VL
EVLMTQTPSSVSAAVGDTVTIKCQASQSISSVLSWYQQKPGQPPKLLIYLASTLASGVPSRFSGSRSGTEFTLTISDLECDDAATYYCQTNYGTSSSNYGFAFGGGTEVVVK

SEQ ID NO: 431 - protein, 126 aa, synthetic construct
  source 1..126: note = Zu33011 VH
EVQLVESGGGLVQPGGSLRLSCAASGISLSSDAISWVRQAPGKGLEYIGIINGGGNTYYASWAKGRFTISRHNSKNTLYLQMNSLRAEDTAVYYCARGIQHGGGNSDYYYYGMDLWGQGTLVTVSS

SEQ ID NO: 432 - protein, 112 aa, synthetic construct
  source 1..112: note = Zu33011 VL
DIQMTQSPSSLSASVGDRVTITCQASQSISSVLSWYQQKPGKAPKLLIYLASTLASGVPSRFSGSGSGTDFTLTISSLQCEDFATYYCQTNYGTSSSNYGFAFGGGTKVEIK

SEQ ID NO: 433 - protein, 118 aa, synthetic construct
  source 1..118: note = xi324O5 VH
QSLEESGGRLVTPGTPLTLTCTASGFSLSNYAMTWVRQAPGKGLEYIGIISTGGITYYMDSAKGRFTISRTSTTVDLKMTSLTTEDTATYFCGRNAGGSYIFYYFDLWGQGTLVTVSS

SEQ ID NO: 434 - protein, 108 aa, synthetic construct
  source 1..108: note = xi324O5 VL
AFELTQTPSSVEAAVGGTVTIKCQASQSIYSYLAWYQQKPGQPPKLLIYSASTLASGVSSRFRGSGSGTEYTLTISDLECADAATYYCQTYYDIVTSTFGGGTEVVVK

SEQ ID NO: 435 - protein, 118 aa, synthetic construct; identical to SEQ ID NO: 433
  source 1..118: note = xi178F16 VH

SEQ ID NO: 436 - protein, 108 aa, synthetic construct
  source 1..108: note = xi178F16 VL
AFELTQTPSSVEAAVGGTVTIKCQASQSIYSYLAWYQQKPGQPPKLLIYSASTLASGVSSRFRGSGSGTEYILTISDLECADAATYYCQTYYDIVTSTFGGGTEVVVK

SEQ ID NO: 437 - protein, 113 aa, synthetic construct
  source 1..113: note = xi237N18 VH
QSLEESGGRLVAPGTPLTLTCTVSGFSLSSYHMSWVRQAPGEGLEWIGGITAMSRTYYASWAKGRFTISKTSTTVHLKITSPTTEDTATYFCAREPGFVSNIWGPGTLVTVSL

SEQ ID NO: 438 - protein, 108 aa, synthetic construct
  source 1..108: note = xi237N18 VL
AVVLTQTASPVSGVVGGTVTIKCQASQNIYSNLAWYQQKPGQRPKLLMYDASTLASGVPSRFKGSGSGTQFTLTISDLECADAATYYCQSISSVDNNVFGGGTEVVVK

SEQ ID NO: 439 - protein, 118 aa, synthetic construct
  source 1..118: note = xi383118 VH
QSVEESGGRLVTPGTPLTLTCTVSGFSLSSYAMGWVRQAPGEGLEWIGTISTGGITYYASWAKGRFTISKTSTTVDLKITSPTTEDTATYFCARVGSSGYLFYFFNLWGQGTLVTVSS

SEQ ID NO: 440 - protein, 108 aa, synthetic construct
  source 1..108: note = xi383118 VL
VFELTQTPSPVSAAVGGTVTIRCQASQNIKSYLAWYQQKPGQPPKLLIYEASILASGVSSRFKGSGSGTEFTLTISDLECADAATYYCQSYYAASSNAFGGGTEVVVK

SEQ ID NO: 441 - protein, 117 aa, synthetic construct
  source 1..117: note = Amgen US 2017/349668 MSLN_5 VH
QVQLVESGGGLVKPGGSLRLSCAASGFTFSDYYMTWIRQAPGKGLEWLSYISSSGSTIYYADSVKGRFTISRDNAKNSLFLQMNSLRAEDTAVYYCARDRNSHFDYWGQGTLVTVSS

SEQ ID NO: 442 - protein, 107 aa, synthetic construct
  source 1..107: note = Amgen US 2017/349668 MSLN_5 VL
DIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGQGTKVEIK

SEQ ID NO: 443 - protein, 115 aa, synthetic construct
  source 1..115: note = U Penn and Novartis US 2016/311917 MSLN5 (different from above) VH
QVQLVQSGAEVEKPGASVKVSCKASGYTFTDYYMHWVRQAPGQGLEWMGWINPNSGGTNYAQKFQGRVTMTRDTSISTAYMELSRLRSDDTAVYYCASGWDFDYWGQGTLVTVSS

SEQ ID NO: 444 - protein, 106 aa, synthetic construct
  source 1..106: note = U Penn and Novartis US 2016/311917 MSLN5 (different from above) VL
DIVMTQSPSSLSASVGDRVTITCRASQSIRYYLSWYQQKPGKAPKLLIYTASILQNGVPSRFSGSGSGTDFTLTISSLQPEDFATYYCLQTYTTPDFGPGTKVEIK

SEQ ID NO: 445 - protein, 119 aa, synthetic construct
  source 1..119: note = City of Hope WO 2016/187158 MORab-009(I83) VH
EVQLQQSGGELEKPGASVKISCKASGYSFTGYTMNWVKQSHGKSLEWIGLITPYNGASSYNQKFRGKATLTVDKSSSTAYMDLLSLTSEDSAIYFCARGGYDGRGFDYWGSGTPVTVSS

SEQ ID NO: 446 - protein, 106 aa, synthetic construct
  source 1..106: note = City of Hope WO 2016/187158 MORab-009(I83) VL
DIELTQSPIIMSASPGEKVTMTCSASSSVSYMHWYQQRTNGSPRRWIYDTSKLASGVPGRFSGSGSGNSYSLTISSVEAEDIADYYCQQWSKHPLTFGSGTKVEIK

SEQ ID NO: 447 - protein, 119 aa, synthetic construct; identical to SEQ ID NO: 445
  source 1..119: note = MORab-009(E83) VH

SEQ ID NO: 448 - protein, 106 aa, synthetic construct
  source 1..106: note = MORab-009(E83) VL
DIELTQSPIIMSASPGEKVTMTCSASSSVSYMHWYQQRTNGSPRRWIYDTSKLASGVPGRFSGSGSGNSYSLTISSVEAEDEADYYCQQWSKHPLTFGSGTKVEIK

SEQ ID NO: 449 - protein, 122 aa, synthetic construct
  source 1..122: note = USA HHS US 9409992 YP223 VH
QEQLEESGGDLVQPEGSLTLTCKASGLDFSSSYWICWVRQAPGKGLEWIGCRHTFTANTWSASWVNGRFTISRSTSLGTVDLKMTSLTAADTATYFCARDESNNDGWDFKLWGPGTLVTVSS

SEQ ID NO: 450 - protein, 109 aa, synthetic construct
  source 1..109: note = USA HHS US 9409992 YP223 VL
AYDMTQTPASVSAAVGGTVTIKCQASQSISNYLAWYQQKPGQPPKLLIYQASTLAPGVSSRFKGSGSGTEFTLTISGVECADAATYYCQQGYTSSNVENVFGGGTGVVV

SEQ ID NO: 451 - protein, 123 aa, synthetic construct
  source 1..123: note = YP218 VH
QQQLEESGGGLVKPEGSLTLTCKASGFDLGFYFYACWVRQAPGKGLEWIACIYTAGSGSTYYASWAKGRFTISKASSTTVTLQMTSLAAADTATYFCARSTANTRSTYYLNLWGPGTLVTVSS

SEQ ID NO: 452 - protein, 110 aa, synthetic construct
  source 1..110: note = YP218 VL
DVVMTQTPASVSEPVGGTVTIKCQASQRISSYLSWYQQKPGQRPKLLIFGASTLASGVPSRFKGSGSGTEYTLTISDLECADAATYYCQSYAYFDSNNWHAFGGGTEVVV

SEQ ID NO: 453 - protein, 119 aa, synthetic construct
  source 1..119: note = YP3 VH
QEQLVESGGGLVQPGASLTLTCTASGIDFSRYYMCWVRQAPGKGLEGIACIYIGGSGSTYYASWAKGRFTISKASSTTVTLQMTSLTAADTATYFCARGTNLNYIFRLWGPGTLVTVSS

SEQ ID NO: 454 - protein, 109 aa, synthetic construct
  source 1..109: note = YP3 VL
DVVMTQTPSPVSAAVGGTVTIKCQASQSINNGLAWYQQKPGQPPRLLIYSASNLESGVPSRFKGSGSGTEFTLTISDLECDDAATYYCQCIWDGNSYVNAFGGGTEVVV

SEQ ID NO: 455 - protein, 115 aa, synthetic construct
  source 1..115: note = YP187 VH
QSLEESGGDLVKPGASLTLTCKASGFDFSSNAMCWVRQAPGKGLEWIACIYVGDGNTYYASWAKGRFTISKTSSTTVTLQMTSLTAADTATYFCARGYASYGSDYYWDYFKLWGP

SEQ ID NO: 456 - protein, 109 aa, synthetic construct
  source 1..109: note = YP187 VL
AYDMTQTPASVSAAVGGTVTIKCQASQSISTALAWYQQKPGQPPKVLIYAASNLASGVSSRFKGSGSGTEFTLTISDLECADAATYYCQQAATIINVDNVFGGGTEVVV

SEQ ID NO: 457 - protein, 120 aa, synthetic construct
  source 1..120: note = YP158 VH
QSLEESGGDLVKPGASLTLTCTASGFSFSGDYYMCWVRQAPGKGLEWIACIGGGSNTATYYATWAKGRFTISKTSSTTVTLQMTSLTAADTATYFCARDLGFVDYALELWGPGTLVTVSS

SEQ ID NO: 458 - protein, 111 aa, synthetic construct
  source 1..111: note = YP158 VL
DIVMTQTPASVEVAVGGTVTIKCQASENMYNSLAWYQQKPGQPPKLLIYRASTLESGVPSRFKGSGSGTEYTLTISDLECADAATYYCQCTFYSHNNNYGGAFGGGTEVVV

SEQ ID NO: 459 - protein, 120 aa, synthetic construct
  source 1..120: note = Lentigen Tech WO 2018/045325 M1-4S VH
EVQLVQSGGGLVQPGGSLRLSCAASGFTFDDYAMHWVRQAPGKGLEWVSGISWNSGSIGYADSVKGRFTISRDNAKNSLYLQMNSLRAEDTALYYCAKDLSSVAGPFNYWGQGTLVTVSS

SEQ ID NO: 460 - protein, 108 aa, synthetic construct
  source 1..108: note = Lentigen Tech WO 2018/045325 M1-4S VL
SSELTQDPAVSVALGQTVRITCQGDSLRSYYASWYQQKPGQAPVLVIYGKNNRPSGIPDRFSGSSSGNTASLTITGAQAEDEADYYCNSRDSSGNHLVFGGGTQLTVL

SEQ ID NO: 461 - protein, 118 aa, synthetic construct
  source 1..118: note = Mab Designs WO 2018/060480 1H07 VH
EVQLQQSGTVLARPGASVKMSCKASGYSFTNYRMNWVKQRPGQGLEWIGGIYPGNRDTTYNQKFKDKAKLTAVTSANTAYMELSSLTNEDSAVYYCTRGVIGIYFDYWGQGTTLTVSS

SEQ ID NO: 462 - protein, 112 aa, synthetic construct
  source 1..112: note = Mab Designs WO 2018/060480 1H07 VL
LDIVMTQSPASLAVSLGQRATISCKASQSVDYDGDSYMNWYQQKPGQPPKLLIYAASNLESGIPARFSGSGSGTDFTLNIHPVEEEDAATYYCQQNNEAPLTFGAGTKLELK

SEQ ID NO: 463 - protein, 118 aa, synthetic construct
  source 1..118: note = 3C02 VH
QVQLQQSGTVLARPGASVKMSCKASGYSFTNYRMYWVKQRPGQGLEWIGAIYPGNSDTTYKQKFKGKAKLTAVTSASTAYMELSSLTNEDSAVYYCTRGIRGSYFDVWGAGTTVTVSS

SEQ ID NO: 464 - protein, 112 aa, synthetic construct
  source 1..112: note = 3C02 VL
LDIVMTQSPASLAVSLGQRATISCKASQSVDYDGDSYMNWYQQKPGQPPKLLIYAASNLESGIPARFSGSGSGTDFTLNIHPVEEEDAATYYCQQSNEDPYTFGGGTKLEIK

SEQ ID NO: 465 - protein, 122 aa, synthetic construct
  source 1..122: note = Abbvie US 2017/342169 HuAb17 VH
EVQLVQSGAEVKEPGASVKVSCKASGDTFNRYYVHWARQAPGQGLEWMGIINPSGVSTTYAQKFQGRVTMTRDTPTNTVYMELGSLRPEDTAVYFCAESRGSGYNYFAMDVWGQGTLVTVSS

SEQ ID NO: 466 - protein, 106 aa, synthetic construct
  source 1..106: note = Abbvie US 2017/342169 HuAb17 VL
SYELTQPPSVSVSPGQTADITCSGDKLGDKYASWYQQKPGQSPVLVIYQDNRRPSGIPERFSGSNSGNTATLTISGTQAMDEADYYCQAWDSDTYVFGPGTKVTVL

SEQ ID NO: 467 - protein, 122 aa, synthetic construct; identical to SEQ ID NO: 465
  source 1..122: note = HuAM1 VH

SEQ ID NO: 468 - protein, 106 aa, synthetic construct; identical to SEQ ID NO: 466
  source 1..106: note = HuAM1 VL

SEQ ID NO: 469 - protein, 122 aa, synthetic construct
  source 1..122: note = HuAM2 VH
EVQLVQSGAEVKKPGASVKVSCKASGDTFKRYYVQWVRQAPGQGLEWMGIINPSGVSTTYAQKFQGRVTMTRDTSTSTVYMELSSLRSEDTAVYYCAESRGSGYNYFAMDVWGQGTLVTVSS

SEQ ID NO: 470 - protein, 106 aa, synthetic construct
  source 1..106: note = HuAM2 VL
SYELTQPPSVSVSPGQTASITCSGDKLGDKYASWYQQKPGQSPVLVIYQDNRRPSGIPERFSGSNSGNTATLTISGTQAMDEADYYCQAWDSDTYVFGTGTKVTVL

SEQ ID NO: 471 - protein, 122 aa, synthetic construct
  source 1..122: note = HuAM3 VH
EVQLVQSGAEVKKPGASVKVSCKASGYTFKRYYVHWVRQAPGQGLEWMGIINPSGVSTTYAQKFQGRVTMTRDTSTSTVYMELSSLRSEDTAVYYCAESRGSGYNYFAMDVWGQGTLVTVSS

SEQ ID NO: 472 - protein, 106 aa, synthetic construct; identical to SEQ ID NO: 470
  source 1..106: note = HuAM3 VL

SEQ ID NO: 473 - protein, 122 aa, synthetic construct
  source 1..122: note = HuAM4 VH
EVQLVQSGAEVKKPGASVKVSCKASGDTFKRYYVHWVRQAPGQGLEWMGIINPSGVSTTYAQKFQGRVTMTRDTSTSTVYMELSSLRSEDTAVYYCAESRGSGYNYFAMDVWGQGTLVTVSS

SEQ ID NO: 474 - protein, 106 aa, synthetic construct; identical to SEQ ID NO: 470
  source 1..106: note = HuAM4 VL

SEQ ID NO: 475 - protein, 122 aa, synthetic construct
  source 1..122: note = HuAM5 VH
EVQLVQSGAEVKKPGASVKVSCKASGDTFNRYYVHWVRQAPGQGLEWMGIINPSGVSTTYAQKFQGRVTMTRDTSTSTVYMELSSLRSEDTAVYYCAESRIPGYNNFAMDVWGQGTLVTVSS

SEQ ID NO: 476 - protein, 106 aa, synthetic construct; identical to SEQ ID NO: 470
  source 1..106: note = HuAM5 VL

SEQ ID NO: 477 - protein, 122 aa, synthetic construct
  source 1..122: note = HuAM6 VH
EVQLVQSGAEVKKPGASVKVSCKASGDTFNRYYVHWVRQAPGQGLEWMGIINPSGVSTTYAQKFQGRVTMTRDTSTSTVYMELSSLRSEDTAVYYCAEVRGSGYNYFAMDVWGQGTLVTVSS

SEQ ID NO: 478 - protein, 106 aa, synthetic construct; identical to SEQ ID NO: 470
  source 1..106: note = HuAM6 VL

SEQ ID NO: 479 - protein, 122 aa, synthetic construct
  source 1..122: note = HuAM7 VH
EVQLVQSGAEVKKPGASVKVSCKASGDTFNRYYVHWVRQAPGQGLEWMGIINPSGVSTTYAQKFQGRVTMTRDTSTSTVYMELSSLRSEDTAVYYCAESRVPGYNNFAMDVWGQGTLVTVSS

SEQ ID NO: 480 - protein, 106 aa, synthetic construct; identical to SEQ ID NO: 470
  source 1..106: note = HuAM7 VL

SEQ ID NO: 481 - protein, 122 aa, synthetic construct
  source 1..122: note = HuAM8 VH
EVQLVQSGAEVKKPGASVKVSCKASGDTFNRYYVHWVRQAPGQGLEWMGIINPSGVSTTYAQKFQGRVTMTRDTSTSTVYMELSSLRSEDTAVYYCAEVRGSGYNYFGMDVWGQGTLVTVSS

SEQ ID NO: 482 - protein, 106 aa, synthetic construct; identical to SEQ ID NO: 470
  source 1..106: note = HuAM8 VL

SEQ ID NO: 483 - protein, 122 aa, synthetic construct
  source 1..122: note = HuAM9 VH
EVQLVQSGAEVKKPGASVKVSCKASGDTFNRYYVHWVRQAPGQGLEWMGIINPSGVSTTYAQKFQGRVTMTRDTSTSTVYMELSSLRSEDTAVYYCAESRGSGYNYFAMDVWGQGTLVTVSS

SEQ ID NO: 484 - protein, 106 aa, synthetic construct
  source 1..106: note = HuAM9 VL
SYELTQPPSVSVSPGQTASITCSGDNLGYKYVSWYQQKPGQSPVLVIYQDHRRPSGIPERFSGSNSGNTATLTISGTQAMDEADYYCQAWDTDTYVFGTGTKVTVL

SEQ ID NO: 485 - protein, 122 aa, synthetic construct
  source 1..122: note = HuAM10 VH
EVQLVQSGAEVKKPGASVKVSCKASGDTFNRYYVQWVRQAPGQGLEWMGIINPSGVSTTYAQKFQGRVTMTRDTSTSTVYMELSSLRSEDTAVYYCAETRGSGYNYFGMDVWGQGTLVTVSS

SEQ ID NO: 486 - protein, 106 aa, synthetic construct; identical to SEQ ID NO: 470
  source 1..106: note = HuAM10 VL

SEQ ID NO: 487 - protein, 122 aa, synthetic construct
  source 1..122: note = HuAM11 VH
EVQLVQSGAEVKKPGASVKVSCKASGDTFKRYYMHWVRQAPGQGLEWMGIINPSGVSTTYAQKFQGRVTMTRDTSTSTVYMELSSLRSEDTAVYYCAEVRGSGFNYFGMDVWGQGTLVTVSS

SEQ ID NO: 488 - protein, 106 aa, synthetic construct
  source 1..106: note = HuAM11 VL
SYELTQPPSVSVSPGQTASITCSGDKLGDKYASWYQQKPGQSPVLVIYQDNRRPSGIPERFSGSNSGNTATLTISGTQAMDEADYYCQVWDSDTYVFGTGTKVTVL

SEQ ID NO: 489 - protein, 122 aa, synthetic construct
  source 1..122: note = HuAM12 VH
EVQLVQSGAEVKKPGASVKVSCKASGDTFHRYYVHWVRQAPGQGLEWMGIINPSGVSTTYAQKFQGRVTMTRDTSTSTVYMELSSLRSEDTAVYYCAEVRGSGFNYFGMDVWGQGTLVTVSS

SEQ ID NO: 490 - protein, 106 aa, synthetic construct; identical to SEQ ID NO: 470
  source 1..106: note = HuAM12 VL

SEQ ID NO: 491 - protein, 122 aa, synthetic construct
  source 1..122: note = HuAM13 VH
EVQLVQSGAEVKKPGASVKVSCKASGDTFNRYYVQWVRQAPGQGLEWMGIINPSGVSTTYAQKFQGRVTMTRDTSTSTVYMELSSLRSEDTAVYYCAETRGSGFNYFGMDVWGQGTLVTVSS

SEQ ID NO: 492 - protein, 106 aa, synthetic construct; identical to SEQ ID NO: 488
  source 1..106: note = HuAM13 VL

SEQ ID NO: 493 - protein, 122 aa, synthetic construct; identical to SEQ ID NO: 487
  source 1..122: note = HuAM14 VH

SEQ ID NO: 494 - protein, 106 aa, synthetic construct; identical to SEQ ID NO: 470
  source 1..106: note = HuAM14 VL

SEQ ID NO: 495 - protein, 122 aa, synthetic construct
  source 1..122: note = HuAM15 VH
EVQLVQSGAEVKKPGASVKVSCKASGDTFKRYYVHWVRQAPGQGLEWMGIINPSGVSTTYAQKFQGRVTMTRDTSTSTVYMELSSLRSEDTAVYYCAEVRGSGFNYFGMDVWGQGTLVTVSS

SEQ ID NO: 496 - protein, 106 aa, synthetic construct; identical to SEQ ID NO: 470
  source 1..106: note = HuAM15 VL

SEQ ID NO: 497 - protein, 122 aa, synthetic construct; identical to SEQ ID NO: 491
  source 1..122: note = HuAM16 VH

SEQ ID NO: 498 - protein, 106 aa, synthetic construct; identical to SEQ ID NO: 470
  source 1..106: note = HuAM16 VL

SEQ ID NO: 499 - protein, 122 aa, synthetic construct
  source 1..122: note = HuAM17 VH
EVQLVQSGAEVKKPGASVKVSCKASGDTFNRYYVQWVRQAPGQGLEWMGIINPSGVSTTYAQKFQGRVTMTRDTSTSTVYMELSSLRSEDTAVYYCAESRVPGYNIFAMDVWGQGTLVTVSS

SEQ ID NO: 500 - protein, 106 aa, synthetic construct; identical to SEQ ID NO: 470
  source 1..106: note = HuAM17 VL

SEQ ID NO: 501 - protein, 122 aa, synthetic construct
  source 1..122: note = HuAM18 VH
EVQLVQSGAEVKKPGASVKVSCKASGDTFNRYYVHWVRQAPGQGLEWMGIINPSGVSTTYAQKFQGRVTMTRDTSTSTVYMELSSLRSEDTAVYYCAETRGSGYNYFGMDVWGQGTLVTVSS

SEQ ID NO: 502 - protein, 106 aa, synthetic construct; identical to SEQ ID NO: 470
  source 1..106: note = HuAM18 VL

SEQ ID NO: 503 - protein, 122 aa, synthetic construct
  source 1..122: note = HuAM19 VH
EVQLVQSGAEVKKPGASVKVSCKASGDTFKRYYAHWVRQAPGQGLEWMGIINPSGVSTTYAQKFQGRVTMTRDTSTSTVYMELSSLRSEDTAVYYCAESRLPGYNAFAMDVWGQGTLVTVSS

SEQ ID NO: 504 - protein, 106 aa, synthetic construct
  source 1..106: note = HuAM19 VL
SYELTQPPSVSVSPGQTASITCSGDMLGYQYGSWYQQKPGQSPVLVIYQDNRRPSGIPERFSGSNSGNTATLTISGTQAMDEADYYCQAWDGDAFVFGTGTKVTVL

SEQ ID NO: 505 - protein, 122 aa, synthetic construct
  source 1..122: note = HuAM21 VH
EVQLVQSGAEVKKPGASVKVSCKASGDTFNRYYVQWVRQAPGQGLEWMGIINPSGVSTTYAQKFQGRVTMTRDTSTSTVYMELSSLRSEDTAVYYCAETRGPGFNYFGMDVWGQGTLVTVSS

SEQ ID NO: 506 - protein, 106 aa, synthetic construct; identical to SEQ ID NO: 470
  source 1..106: note = HuAM21 VL

SEQ ID NO: 507 - protein, 122 aa, synthetic construct; identical to SEQ ID NO: 505
  source 1..122: note = MSLN76923 VH

SEQ ID NO: 508 - protein, 106 aa, synthetic construct; identical to SEQ ID NO: 470
  source 1..106: note = MSLN76923 VL

SEQ ID NO: 509 - protein, 123 aa, synthetic construct
  source 1..123: note = US 2018/208645 USA HHS and Lentigen MH1P VH
QVQLQSGPGLVKPSRTLSLTCAISGDSVSSNSAAWNWIRQSPSRGLEWLGRTYYRSKWYNDYAVSVKSRITINPDTSKNQFSLQLNSVTPEDTAVYYCARGKGGKKGGAFDIWGQGTMVTVSS

SEQ ID NO: 510 - protein, 106 aa, synthetic construct
  source 1..106: note = US 2018/208645 USA HHS and Lentigen MH1P VL
SSELTQDPAVSVALGQTVRITCQGDSLRSYYASWYQQKPGQAPVLVIYGKNNRPSGIPDRFSGSSSGNTASLTITGAQAEDEAEYYCSSSTRNHVFFGRGTKVTVL

SEQ ID NO: 511 - protein, 121 aa, synthetic construct
  source 1..121: note = MH2P VH
EVQLVQSGGGLVQPGRSQRLSCAASGFTFDDYAMHWVRQAPGKGLEWVSGISWNSGSIGYADSVKGRFTISRDNAKNSLYLQMNSLRAEDTALYYCAKDISSSAGNAFDIWGQGTMVTVSS

SEQ ID NO: 512 - protein, 108 aa, synthetic construct
  source 1..108: note = MH2P VL
SSELTQDPAVSVALGQTVRITCQGDRLRSYYASWYQQKPGQAPVLVIYGKNNRPSGIPDRFSGSDSGDTASLTITGAQAEDEADYYCHSRDSGGNHVVFGGGTQLTVL

SEQ ID NO: 513 - protein, 118 aa, synthetic construct
  source 1..118: note = MH6P VH
QVQLVQSGAEVKKPGASVKVSCKASGYTFTGYYMHWVRQAPGQGLEWMGRINPNSGGTNYAQKFQGRVTMTRNTSISTAYMELSRLRSDDTAVYYCARSGYYYGLDVWGQGTTVTVSS

SEQ ID NO: 514 - protein, 110 aa, synthetic construct
  source 1..110: note = MH6P VL
QSVLTQPPSASGTPGQRVTISCSGSRSNIGRNTVNWYQQLPGLAPKLIIQRSDQRPSGVPDRFSGSKSVTSASLAISGLRSEDEADYYCGTWDNSLSAYVFGTGTKLTVL

SEQ ID NO: 515 - protein, 123 aa, synthetic construct
  source 1..123: note = CAFA therapeutics US 2018/244796 P1A6E VH
QVQLEQSGLGLVKPSQTLSLTCAISGDTVSSDSAAWNWIRQSPSRGLEWLGRTYYRSKWFNDYAVSVKGRITINSDTSKNQFSLQLNSVTPEDTAVYYCARSNSYYYYAMDVWGQGTLVTVSS

SEQ ID NO: 516 - protein, 115 aa, synthetic construct
  source 1..115: note = CAFA therapeutics US 2018/244796 P1A6E VL
QAVLTQPSSLSASPGASASLTCTLRSGINVGIYRIYWYQQRPGSPPQILLTYKSDSDKYQGSGVPSRFSGSKDASANAGILLISGLQSEDEADYYCMIWHSGGWVFGGGTKVTVL

SEQ ID NO: 517 - protein, 124 aa, synthetic construct
  source 1..124: note = P3F2 VH
QMQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCASSRSGTTVVNHDAFDIWGKGTTVTVSS

SEQ ID NO: 518 - protein, 107 aa, synthetic construct
  source 1..107: note = P3F2 VL
DIQLTQSPSSLSASVGDRVTITCRASQVISRALAWYQQTPGKPPKLLIYDASNLQSGVPSRFSGSGSGTDFTLTISRLQPEDFATYYCQQFNSYPLTFGGGTKLEIK

SEQ ID NO: 519 - protein, 120 aa, synthetic construct
  source 1..120: note = WO 2017/072366 NBE therapeutics MN clone 3-1 VH
QVQLQQSGAELVKPGASMKLSCKASGYTFTSYWMHWVRQAPGQGLEWIGAIHPNSDNTIYYQKFKSRVTLTVDKSISTAYMQLSSLTSEDTAVYYCAIIITPVVPKFDYWGQGTTVTVSS

SEQ ID NO: 520 - protein, 107 aa, synthetic construct
  source 1..107: note = WO 2017/072366 NBE therapeutics MN clone 3-1 VL
DIQMTQSPSFLSASVGDRVTITCRASHDVGTSLAWYQQKPGQAPKLLIYWASTLQTGVPSRFSGSGSGTEFTLTISSLQPEDFATYYCQQYSSYPLTFGPGTTVDMK

SEQ ID NO: 521 - protein, 120 aa, synthetic construct
  source 1..120: note = MN clone 5-2 VH
QVQLQQSGAELVKPGASLKLSCKASGYTFTSYWMHWVRQAPGQGLEWIGAIHPNSDNTIYYQKFKSRFTITVDKSTSTAYMQLSSLTSEDTAVYYCAIIITPVVPKFDYWGQGTTVTVSS

SEQ ID NO: 522 - protein, 107 aa, synthetic construct
  source 1..107: note = MN clone 5-2 VL
DIVMTQSSSFMSASVGDRVSITCKASHDVGTSLAWYQQKPGQSPKLLIYWASTRQTGVPDRFSGSGSGTDFTLTISSVQSEDVATYFCQQYSSYPLTFGQGTKLEIK

SEQ ID NO: 523 - protein, 120 aa, synthetic construct; identical to SEQ ID NO: 521
  source 1..120: note = MN-clone 5-3 VH

SEQ ID NO: 524 - protein, 107 aa, synthetic construct
  source 1..107: note = MN-clone 5-3 VL
DIVMTQSPDSLAVSLGERATITCKSSHDVGTSLAWYQQKPGQPPKLLIYWASTRESGVPDRFSGSGSGTDFTLTISSLQAEDVAVYYCQQYSSYPLTFGQGTKLEIK

SEQ ID NO: 525 - protein, 122 aa, synthetic construct
  source 1..122: note = Amgen US 10100121 AB1.1.2 VH
QVQLVESGGGVVQPGRSLRLSCAASGFTFSSYGMHWVRQAPGKGLEWVAAIWYDGSNKYYADSVKGRFTISRDNSKNMLYLQMNSLRAEDTAVYYCARDLSIFGVVILSDYWGQGTLVTVSS

SEQ ID NO: 526 - protein, 107 aa, synthetic construct
  source 1..107: note = Amgen US 10100121 AB1.1.2 VL
EIVLTQSPDFQSVTPKEKVTITCRASQYIGSNLHWYQQTPDQSPKLLIKYASQSFSGVPSRFSGSGSGTDFTLTINSLEAEDAATYYCHQSSSLPWTFGQGTKVEIK

SEQ ID NO: 527 - protein, 119 aa, synthetic construct
  source 1..119: note = AB1.33.1 VH
QVQLQESGPGLVKPSQTLSLTCTVSGGSISGDGHFWSWIRQHPGKGLEWIGYIYYSGSTYYNPSLKSRVTISVDTSKNQFSLKLSSVTAADTAVYYCARLRGGYKFDYWGQGTLVTVSS

SEQ ID NO: 528 - protein, 112 aa, synthetic construct
  source 1..112: note = AB1.33.1 VL
DIVMTQTPLSLSVAPGQPASISCQSSLSLQHSNGKTYLYWYLQKPGQPPQLLIYEVSNRFSGVPDRFSGSGSGTDFTLKISRVEAEDVGIYYCMQSKQLPCSFGQGTKLEIK

SEQ ID NO: 529 - protein, 119 aa, synthetic construct; identical to SEQ ID NO: 527
  source 1..119: note = AB1.11.1-y VH

SEQ ID NO: 530 - protein, 112 aa, synthetic construct
  source 1..112: note = AB1.11.1-y VL
DIVMTQTPLSLSVAPGQPASISCQSSLSLQHSNGKTYLYWYLQKPGQPPQLLIYEVSNRFSGVPDRFSGSGSGTDFTLKISRVEAEDVGIYYCMQSKQLPYSFGQGTKLEIK

SEQ ID NO: 531 - protein, 128 aa, synthetic construct
  source 1..128: note = AB1.68.1 VH
QVQLQESGPGLVKPSETLSLTCTVSGDSINNYYWSWIRQPAGKGLEWIGRIFTSGSTNYNPSLKSRVTMSIDTSKNQFSLKLSSVTAADTAVYYCAREGGHYGSSGYLYYYYFGMDVWGQGTTVTVSS

SEQ ID NO: 532 - protein, 107 aa, synthetic construct
  source 1..107: note = AB1.68.1 VL
EIVLTQSPGTLSLSPGERATLSCRASQTVSSSYLAWYQQKPGQAPRLLIYGASIRATGIPDRFSGSGSGTDFTLTISRLEPEDFAVYYCQQYGSSLTFGGGTKVEIK

SEQ ID NO: 533 - protein, 122 aa, synthetic construct; identical to SEQ ID NO: 525
  source 1..122: note = AB1.78.1 VH

SEQ ID NO: 534 - protein, 107 aa, synthetic construct
  source 1..107: note = AB1.78.1 VL
EIVLTQSPDFQSVTPKEKVTITCRASQSIGSSLHWYRQKPDQSPKLLIKYASQSFSGVPSRFSGGGSGTDFTLTINSLEAEDAATYYCHQSSSLPWTFGQGTKVEIK

SEQ ID NO: 535 - protein, 121 aa, synthetic construct
  source 1..121: note = AB1.119.1 VH
QVQLQESGPGLVKPSQTLSLTCTVSGGSISSGGYYWNWIRQHPGKGLEWIGYIYYSGSTYYNPSLRGRLIISVDTSKNQFSLRLSSVTAADTAVYYCARDGGDSYGRMDVWGQGTTVTVSA

SEQ ID NO: 536 - protein, 112 aa, synthetic construct
  source 1..112: note = AB1.119.1 VL
DIVMTQTPLSSPVTLGQPASISCRSSQSLVHSDRNTYLSWLQQRPGQPPRLLIYKISNRFSGVPDRFSGSGAGTDFTLKISRVEAEDVGVYYCMQATQFPLTFGGGTKVEIK

SEQ ID NO: 537 - protein, 125 aa, synthetic construct
  source 1..125: note = AB.2.69.2 VH
QVQLQQSGPGLVKPSQTLSLTCAISGDSVSSNSVAWNWIRQSPSRGLEWLGRTYYRSKWYNDYAVSVKSRITINPDTSKNQFSLQLNSVTPEDTAVYYCARGIFVVPAVPRFDYWGQGTLVTVSS

SEQ ID NO: 538 - protein, 112 aa, synthetic construct
  source 1..112: note = AB.2.69.2 VL
DIVMTQSPLSLSVTPGEPASISCRSSLSLLHSNGYNYLDWFLQKPGQSPQLLIYLGSNRASGVPDRFSGSGSDTDFTLKISRVEAEDVGVYYCMQGLHTPPSFGQGTKLEIK

SEQ ID NO: 539 - protein, 125 aa, synthetic construct; identical to SEQ ID NO: 537
  source 1..125: note = AB.2.69.2-s VH

SEQ ID NO: 540 - protein, 105 aa, synthetic construct
  source 1..105: note = AB.2.69.2-s VL
DIQMTQSPSSLSASVGDRVTITCQASQDISNYLNWYQQKPGKAPNLLIYAASSLETGVPSRFSGGGSGTDFAFTISSLQPEDIAPYYCQQYDNLPFGPGTKVDIK

SEQ ID NO: 541 - protein, 125 aa, synthetic construct; identical to SEQ ID NO: 537
  source 1..125: note = AB.2.69.2-cv VH

SEQ ID NO: 542 - protein, 105 aa, synthetic construct
  source 1..105: note = AB.2.69.2-cv VL
DIQMTQSPSSLSASVGDRVTITCQASQDISNYLNWYQQKPGKAPKLLIYAASSLETGVPSRFSGSGSGTDFTLTISSLQPEDFATYYCQQYDNLPFGQGTKVDIK

SEQ ID NO: 543 - protein, 116 aa, synthetic construct
  source 1..116: note = AB1.58 VH
QVQLQESGPGLVKPSETLSLTCTVSGGSIISHYWSWIRQPPGKGLEWIGYIYYSGSTNYNPSLKSRVTISVDTSKNQFSLKLTSVTAADTAVYYCARDGWSAFDYWGQGTLVTVSS

SEQ ID NO: 544 - protein, 107 aa, synthetic construct
  source 1..107: note = AB1.58 VL
DIQMTQSPSSLSASVGDRVTITCRASQGIRNALGWYQQKPGKAPKRLIYAASSLQSGVPSRFSGSGSGTEFTLTISSLQPEDFATYYCLQHNSYPRTFGQGTKVEIR

SEQ ID NO: 545 - protein, 116 aa, synthetic construct
  source 1..116: note = AB1.51 VH
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCARVDYKAFDIWGQGTMVTVSS

SEQ ID NO: 546 - protein, 107 aa, synthetic construct
  source 1..107: note = AB1.51 VL
DIQMTQSPSSLSASVGDRVTITCRASQGIRNDLHWYQQKPGKAPKRLIYAASSLQSGVPSRFSGSGSGTEFTLTISSLQPEDFATYYCLQHYSYPWTFGQGTKVEIK

SEQ ID NO: 547 - protein, 116 aa, synthetic construct
  source 1..116: note = O25 VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGAFDYWGQGTLVTVSS

SEQ ID NO: 548 - protein, 118 aa, synthetic construct
  source 1..118: note = O25 VL
QPVLTQPASLSASPGASASLTCTLHSGISVKDYRIYWYQQKPGRPPQYLLRYKSNSDMQQGSGVPSRFSGSKDASANAGILLISGLQSEDEADYYCMIWHHGHGTSLVFGGGTKLTVL

SEQ ID NO: 549 - protein, 116 aa, synthetic construct; identical to SEQ ID NO: 547
  source 1..116: note = O30 VH

SEQ ID NO: 550 - protein, 123 aa, synthetic construct
  source 1..123: note = O30 VL
QSVLTQPPSASGTPGQRVTISCSGSSSNIAHGPVNWYQQLPGTAPKLLIYATNHRPSGVPDRFSGSKSGTTASLTISGLQSEDEADYYCAAYDLTGWFAYAVFGGGTKLTVLGQPKAAPSVTL

SEQ ID NO: 551 - protein, 116 aa, synthetic construct; identical to SEQ ID NO: 547
  source 1..116: note = O32 VH

SEQ ID NO: 552 - protein, 121 aa, synthetic construct
  source 1..121: note = O32 VL
SYVLTQPPSVSVAPGKTARITCGGNKIGHRAVHWYQQKPGQAPVLVIYYTYDRPSGIPERFSGSNSGNTATLTISRVEAGDEADYYCQVWDASRRDANVVFGGGTKLTVLGQPKAAPSVTL

SEQ ID NO: 553 - protein, 116 aa, synthetic construct; identical to SEQ ID NO: 547
  source 1..116: note = O35 VH

SEQ ID NO: 554 - protein, 118 aa, synthetic construct
  source 1..118: note = O35 VL
QPVLTQPVSLSASPGASVSLTCTLRSDIRVRDYRIFWYQQKPGSPPQYLLRYKTDSDKQQGSGVPSRFSGSKDASANAGILLISGLQSEDEADYYCMIWHRTTGTSLVFGGGTKLTVL

SEQ ID NO: 555 - protein, 116 aa, synthetic construct; identical to SEQ ID NO: 547
  source 1..116: note = O37 VH

SEQ ID NO: 556 - protein, 119 aa, synthetic construct
  source 1..119: note = O37 VL
QPVLTQPASLSASPGASASLTCTLRSGINVKDYRIFWYQQKPGSPPQYLLRYKSESDKQQGSGVPSRFSGSKDASANAGILLISGLQSEDEADYYCMIWHKDREGHAFVFGGGTKLTVL

SEQ ID NO: 557 - protein, 116 aa, synthetic construct; identical to SEQ ID NO: 547
  source 1..116: note = O38 VH

SEQ ID NO: 558 - protein, 119 aa, synthetic construct
  source 1..119: note = O38 VL
QPVLTQPASLSASPGASASLTCTLRSGINVRDYRIFWYQQKPGSPPQYLLRYKSASDKQQGSGVPSRFSGSKDASANAGILLISGLQSEDEADYYCMIWHHDSEGHAFVFGGGTKLTVL

SEQ ID NO: 559 - protein, 116 aa, synthetic construct; identical to SEQ ID NO: 547
  source 1..116: note = O41 VH

SEQ ID NO: 560 - protein, 120 aa, synthetic construct
  source 1..120: note = O41 VL
SYVLTQPPSVSVAPGKTARITCGGNKIGHRAVHWYQQKPGQAPVLVIYYTYERPSGIPERFSGSNSGNTATLTISRVEAGDEADYYCQVWDWYSEGGVVFGGGTKLTVLGQPKAAPSVTL

SEQ ID NO: 561 - protein, 115 aa, synthetic construct
  source 1..115: note = WO 2018/213612 USA HHS 2B4 VH
EVQLQQSGPELVKPGASVKISCKASGYPFTGHYMHWVKQGHVKSLEWIGRINPYTGAINYNQNFKDKASLSVEKSSSTAYMGLHSLTSEDSAVYYCVRDLGGGYWGQGTTLTVSS

SEQ ID NO: 562 - protein, 109 aa, synthetic construct
  source 1..109: note = WO 2018/213612 USA HHS 2B4 VL
QAVVTQESALTTSPGETVTLTCRSSTGAVTTSNYANWVQEKPDHLFTGLIAGTNNRAPGVPARFSGSLIGDKAALTITGAQPEDEAIFFCALWYSSHWVFGGGTKLTVL

SEQ ID NO: 563 - protein, 115 aa, synthetic construct
  source 1..115: note = 7B5 VH
EVQLQQSGPELVKPGTSVKISCKASGYPFTGHYMHWVKQGHVKSLEWIGRINPYTGAINYNQNFKDKASLTVEKSSSTAYMGLHSLTSEDSAVYYCVRDLGGGYWGQGTTLTVSS

SEQ ID NO: 564 - protein, 109 aa, synthetic construct
  source 1..109: note = 7B5 VL
QAVVTQESALTTSPGETVTLTCRSSTGAVTTSNYANWVQEKPDHLFTGLIAGTNNRAPGVPARFSGSLIGDKAALTITGAQPEDEAMFFCALWFGSHWVFGGGTKLTVL

SEQ ID NO: 565 - protein, 114 aa, synthetic construct
  source 1..114: note = 15B6 VH
EVQLQQSGPVLVKPGASVKISCKASGYSFTGYYMHWVRQSLVKRLEWIGRINPYTGVPSYKHNFKDKASLTVDKSSSTAYMELHSLTSEDSAVYYCARELGGYWGQGTTLTVSS

SEQ ID NO: 566 - protein, 109 aa, synthetic construct
  source 1..109: note = 15B6 VL
QAVVTQESALTTSPGETVTLTCRSSTGAVTTGNYPNWVQEKPDHLFTGLIAGTNNRAPGVPARFSGSLIGDKAALTITGAQTEDEAIYFCALWFSSHWVFGGGTKLTVL

SEQ ID NO: 567 - protein, 119 aa, synthetic construct
  source 1..119: note = Mogam Institute US 2018/346588 MI323 VH
EVQLQQSGPELVKPGTSVKISCKASGYSFTSYFIQWVKQRPGQGLEWIGWIFPGSGNTKYNEMFKGKATLAADTSSSTAYMQLSSLTSEDSAVYFCARSGGYQYYFDYWGQGTSVTVSS

SEQ ID NO: 568 - protein, 107 aa, synthetic construct
  source 1..107: note = Mogam Institute US 2018/346588 MI323 VL
DIVMTQSHKFMSTSVGDRVSITCKASQDVSTAVAWYQQKPGQSPKLLIYSASYRYPGVPDRFTGSGSGTDFTFTISSVQAEDLALYYCQQHYSTPWTFGGGTKLEIK

SEQ ID NO: 569 - protein, 118 aa, synthetic construct
  source 1..118: note = MI329 VH
EVMLVESGGDLVKPGGSLKLSCAASGFTFSSYAMSWVRRTPEKRLEWVATINSDGSYTFYPDSVKGRFTISRDNAKNTLYLQMNSLRSEDTAMYYCARWGENWYFDVWGAGTTVTVSS

SEQ ID NO: 570 - protein, 112 aa, synthetic construct
  source 1..112: note = MI329 VL
DVVMTQTPLSLPVSLGDQASISCRSSQSLVHSNGNTYLHWYLQKPGQSPKLLIYKVSNRFSGVPDRFSGSGSGTDFTLKISRVEAEDLGIYFCSQSTHFPRTFGGATKLELK

SEQ ID NO: 571 - protein, 122 aa, synthetic construct
  source 1..122: note = MI403 VH
EVQVVESGGGLVKPGGSLKLSCAASGFAFSSYDMSWVRQTPEKRLEWVAYISSGGGSTYYPDTVKGRFTISRDNAKNTLYLQMNSLKSEDTAMYYCARQGTAVKNYWYFDVWGAGTSVTVSS

SEQ ID NO: 572 - protein, 111 aa, synthetic construct
  source 1..111: note = MI403 VL
DIVMTQSPASLAVSLGQRATISCRASQSVSTSSSSYVHWYQQRPGQPPKLLIKYASNLESGVPARFSGSGSGTDFTLNIHPVEEEDTGTYYCQHSWEIPFTFGSGTKLEIK

SEQ ID NO: 573 - protein, 122 aa, synthetic construct
  source 1..122: note = MI407 VH
EVKLVESGGGLVKPGGSLKLSCAASGFPFSNYDMSWVRQTPEKRLEWVAYISSGGGNTYYPDTVKGRFTISRDNAKNTLYLQMSSLKSEDTALYFCVRQGTSVESYWYFDVWGAGTTVTVSS

SEQ ID NO: 574 - protein, 111 aa, synthetic construct
  source 1..111: note = MI407 VL
DIVLTQSPASLAVSLGQRATISCRASQSVSTSSSSYIHWYQQKPGQPPKLLIKYASNLESGVPARFSGSGSGTDFTLNIHPVEEDDTATYYCQHSWEIPFTFGSGTELEIK

SEQ ID NO: 575 - protein, 116 aa, synthetic construct
  source 1..116: note = MS501 VH
EVQLLESGGGLVQPGGSLRLSCAASGFTFSNYAMSWVRQAPGKGLEWVSGIYPDSGSTYYADSVKGRFTISRDNSKNTLYLQMNSLRAEDTAVYYCARNIYTFDYWGQGTLVTVSS

SEQ ID NO: 576 - protein, 110 aa, synthetic construct
  source 1..110: note = MS501 VL
QSVLTQPPSASGTPGQRVTISCSGSSSNIGSNAVSWYQQLPGTAPKLLIYYNNQRPSGVPDRFSGSKSGTSASLAISGLRSEDEADYYCGSWDSSLSGYVFGGGTKLTVL

SEQ ID NO: 577 - protein, 116 aa, synthetic construct
  source 1..116: note = MS502 VH
EVQLLESGGGLVQPGGSLRLSCAASGFTFSNYAMSWVRQAPGKGLEWVSGIPPDSGSKYYADSVRGRFTVSRDNSKNTLYLQMNSLRAEDTAVYYCAKNMLSFDYWGQGTLVTVSS

SEQ ID NO: 578 - protein, 110 aa, synthetic construct
  source 1..110: note = MS502 VL
QSVLTQPPSASGTPGQRVTISCTGSSSNIGSNAVSWYQQLPGTAPKLLIYYNSKRPSGVPDRFSGSKSGTSASLAISGLRSEDEADYYCGSWDSSLNGYVFGGGTKVTVL

SEQ ID NO: 579 - protein, 116 aa, synthetic construct; identical to SEQ ID NO: 577
  source 1..116: note = MS502-1 VH

SEQ ID NO: 580 - protein, 110 aa, synthetic construct
  source 1..110: note = MS502-1 VL
QSVLTQPPSASGTPGQRVTISCICSSSNIGSNAVSWYQQLPGTAPKLLIYYNSKRPSGVPDRFSGSKSGTSASLAISGLRSEDEADYYCGSWDSSLNGYVFGGGTKLTVL

SEQ ID NO: 581 - protein, 116 aa, synthetic construct
  source 1..116: note = MS503 VH
EVQLLESGGGLVQPGGSLRLSCAASGFTFSNYAMSWVRQAPGKGLEWVSSIYPGDGSTYYADSVKGRFTISRDNSKNTLYLQMNSLRAEDTAVYYCAKNAFTFDYWGQGTLVTVSS

SEQ ID NO: 582 - protein, 110 aa, synthetic construct
  source 1..110: note = MS503 VL
QSVLTQPPSASGTPGQRVTISCSGSSSNIGSNAVSWYQQLPGTAPKLLIYYNSHRPSGVPDRFSGSKSGTSASLAISGLRSEDEADYYCGTWDSSLSGYVFGGGTKLTVL

SEQ ID NO: 583 - protein, 116 aa, synthetic construct
  source 1..116: note = MS504 VH
EVQLLESGGGLVQPGGSLRLSCAASGFTFSNYAMSWVRQAPGKGLEWVSSIYPNGSSKYYADSVKGRFTISRDNSKNTLYLQMNSLRAEDMAVYYCAKNLLTFDYWGQGTLVTVSS

SEQ ID NO: 584 - protein, 110 aa, synthetic construct
  source 1..110: note = MS504 VL
QSVLTQPPSASGPPGQRVTISCTGSSSNIGNNSVSWYQQLPGTAPKLLIYYDSHRPSGVPDRFSGSKSGTSASLAIGGLRSEDEADYYCGAWDDSLNAYVFGGGTKLTVL

SEQ ID NO: 585 - protein, 116 aa, synthetic construct
  source 1..116: note = MS505 VH
EVQLLESGGGLVQPGGSLRLSCAASGFTFSNYAMSWVRQAPGKGLEWVSAIYPDGSNKYYADSVKGRFTVSRDNSKNTLYLQMNSLRAEDTAVYYCARNAYTFDYWGQGTLVTVSS

SEQ ID NO: 586 - protein, 110 aa, synthetic construct
  source 1..110: note = MS505 VL
QSVLTQPPSASGTPGRRVTISCSGSSSNIGSNAVSWYQQLPGTAPKLLIYYNSQRPSGVPDRFSGSKSGTSASLAISGLRSEDEADYYCGSWDSSLNGYVFGGGTKLTVL

SEQ ID NO: 587 - protein, 116 aa, synthetic construct
  source 1..116: note = MS506 VH
EVQLLESGGGLVQPGGSLRLSCAASGFTFSNYAMSWVRQAPGKGLEWVSSIYPGSGSTYYADSVKGRFTISRDNSKNTLYLQMNSLRAEDTAVYYCARNLYTFDYWGQGTLVTVSS

SEQ ID NO: 588 - protein, 110 aa, synthetic construct
  source 1..110: note = MS506 VL
QSVLTQPPSASGTPGQRVTISCTGSSSNIGSNAVTWYQQLPGTAPKLLIYYDSHRPSGVPDRFSGSKSGTSASLAISGPRSEDEADYYCGAWDSSLSAYVFGGGTKLTVL

SEQ ID NO: 589 - protein, 116 aa, synthetic construct; identical to SEQ ID NO: 577
  source 1..116: note = C2G1 VH

SEQ ID NO: 590 - protein, 110 aa, synthetic construct
  source 1..110: note = C2G1 VL
QSVLTQPPSASGTPGQRVTISCTGSSSNIGPNAVSWYQQLPGTAPKLLIYYNSKRPSGVPDRFSGSKSGTSASLAISGLRSEDEADYYCGSWDSSLSGYVFGGGTKVTVL

SEQ ID NO: 591 - protein, 116 aa, synthetic construct; identical to SEQ ID NO: 577
  source 1..116: note = C2G4 VH

SEQ ID NO: 592 - protein, 110 aa, synthetic construct
  source 1..110: note = C2G4 VL
QSVLTQPPSASGTPGQRVTISCTGSSSNIGSNAVSWYQQLPGTAPKLLIYYNSKRPSGVPDRFSGSKSGTSASLAISGLRSEDEADYYCGSWDPSLNGYVFGGGTKVTVL

SEQ ID NO: 593 - protein, 116 aa, synthetic construct; identical to SEQ ID NO: 577
  source 1..116: note = C3C8 VH

SEQ ID NO: 594 - protein, 110 aa, synthetic construct
  source 1..110: note = C3C8 VL
QSVLTQPPSASGTPGQRVTISCTGSSSNIGPNAVSWYQQLPGTAPKLLIYYNSKRPSGVPDRFSGSKSGTSASLAISGLRSEDEADYYCGSWDSDLRGYVFGGGTKVTVL

SEQ ID NO: 595 - protein, 116 aa, synthetic construct
  source 1..116: note = 54 VH
EVQLLESGGGLVQPGGSLRLSCAASGFTFSNYAMSWVRQAPGKGLEWVSGIYPDSSSTYYADSVKGRFTISRDNSKNTLYLQMNSLRAEDTAVYYCARNIYTFDYWGQGTLVTVSS

SEQ ID NO: 596 - protein, 110 aa, synthetic construct; identical to SEQ ID NO: 578
  source 1..110: note = 54 VL

SEQ ID NO: 597 - protein, 116 aa, synthetic construct
  source 1..116: note = 56 VH
EVQLLESGGGLVQPGGSLRLSCAASGFTFSNYAMSWVRQAPGKGLEWVSGIPPDSASKYYADSVRGRFTVSRDNSKNTLYLQMNSLRAEDTAVYYCAKNMLSFDYWGQGTLVTVSS

SEQ ID NO: 598 - protein, 110 aa, synthetic construct; identical to SEQ ID NO: 578
  source 1..110: note = 56 VL

SEQ ID NO: 599 - protein, 116 aa, synthetic construct
  source 1..116: note = 2-30 VH
EVQLLESGGGLVQPGGSLRLSCAASGFTFSNYAMSWVRQAPGKGLEWVSGIPPDSNSKYYADSVRGRFTVSRDNSKNTLYLQMNSLRAEDTAVYYCAKNMRTFDYWGQGTLVTVSS

SEQ ID NO: 600 - protein, 110 aa, synthetic construct; identical to SEQ ID NO: 578
  source 1..110: note = 2-30 VL

SEQ ID NO: 601 - protein, 116 aa, synthetic construct
  source 1..116: note = 2-73 VH
EVQLLESGGGLVQPGGSLRLSCAASGFTFSNYAMSWVRQAPGKGLEWVSGIPPNSDSKYYADSVRGRFTVSRDNSKNTLYLQMNSLRAEDTAVYYCAKNMLSFDYWGQGTLVTVSS

SEQ ID NO: 602 - protein, 110 aa, synthetic construct; identical to SEQ ID NO: 578
  source 1..110: note = 2-73 VL

SEQ ID NO: 603 - protein, 116 aa, synthetic construct
  source 1..116: note = 2-78 VH
EVQLLESGGGLVQPGGSLRLSCAASGFTFSNYAMSWVRQAPGKGLEWVSGIPPDSGSKYYADSVRGRFTVSRDNSKNTLYLQMNSLRAEDTAVYYCAKNMFSFDYWGQGTLVTVSS

SEQ ID NO: 604 - protein, 110 aa, synthetic construct; identical to SEQ ID NO: 578
  source 1..110: note = 2-78 VL

SEQ ID NO: 605 - protein, 116 aa, synthetic construct; identical to SEQ ID NO: 597
  source 1..116: note = 56-C2G4 VH

SEQ ID NO: 606 - protein, 110 aa, synthetic construct; identical to SEQ ID NO: 592
  source 1..110: note = 56-C2G4 VL

SEQ ID NO: 607 - protein, 116 aa, synthetic construct; identical to SEQ ID NO: 599
  source 1..116: note = 2-30-C2G4 VH

SEQ ID NO: 608 - protein, 110 aa, synthetic construct; identical to SEQ ID NO: 592
  source 1..110: note = 2-30-C2G4 VL

SEQ ID NO: 609 - protein, 116 aa, synthetic construct; identical to SEQ ID NO: 601
  source 1..116: note = 2-73-C2G4 VH

SEQ ID NO: 610 - protein, 110 aa, synthetic construct; identical to SEQ ID NO: 592
  source 1..110: note = 2-73-C2G4 VL

SEQ ID NO: 611 - protein, 116 aa, synthetic construct; identical to SEQ ID NO: 603
  source 1..116: note = 2-78-C2G4 VH

SEQ ID NO: 612 - protein, 110 aa, synthetic construct; identical to SEQ ID NO: 592
  source 1..110: note = 2-78-C2G4 VL

SEQ ID NO: 613 - protein, 124 aa, synthetic construct
  source 1..124: note = M10MS-1 VH
QVQLQQSGPGLVKPSRTLSLTCAISGDSVSSNSAAWNWIRQSPSRGLEWLGRTYYRSKWYNDYAVSVKSRITINPDTSKNQFSLQLNSVTPEDTAVYYCARGKGGKKGGAFDIWGQGTMVTVSS

SEQ ID NO: 614 - protein, 106 aa, synthetic construct; identical to SEQ ID NO: 510
  source 1..106: note = M10MS-1 VL

SEQ ID NO: 615 - protein, 115 aa, synthetic construct; identical to SEQ ID NO: 294
  source 1..115: note = RG7882 VH

SEQ ID NO: 616 - protein, 113 aa, synthetic construct; identical to SEQ ID NO: 293
  source 1..113: note = RG7882 VL

SEQ ID NO: 617 - protein, 449 aa, synthetic construct
  source 1..449: note = XENP39532 Chain 1-MESO-C[mesothelin]_H1_Fab-IgG1_pI (-)_Isosteric_A_PVA_/S267K/L368D/K370S/M428L/N434S
QVQLVQSGAEVKKPGASVKVSCKASGYTFTSYWMHWVRQAPGQGLEWMGMIHPNSDNTIYYEKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 618 - protein, 711 aa, synthetic construct
  source 1..711: note = XENP39532 Chain 2- MESO-C[mesothelin]_H1_Fab-IgG1_P VA_/S267K/S364K/E357Q/M428L/N434S/K447del_(G4S)3_1A7[CD28] _H1.14_L1_scFv(GKPGS)4
QVQLVQSGAEVKKPGASVKVSCKASGYTFTSYWMHWVRQAPGQGLEWMGMIHPNSDNTIYYEKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGGGGGSGGGGSGGGGSEVQLLESGGGLVQPGGSLRLSCAASGFTFSSYYMSWVRQAPGKGLEWVSTISESGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGQGTKLEIK

SEQ ID NO: 619 - protein, 214 aa, synthetic construct; identical to SEQ ID NO: 259
  source 1..214: note = XENP39532 Chain 3-MESO-C[mesothelin]_L1_Light Chain

SEQ ID NO: 620 - protein, 449 aa, synthetic construct; identical to SEQ ID NO: 617
  source 1..449: note = XENP39533 Chain 1-MESO-C[mesothelin]_H1_Fab-IgG1_pI (-)_Isosteric_A_PVA_/S267K/L368D/K370S/M428L/N434S

SEQ ID NO: 621 - protein, 711 aa, synthetic construct
  source 1..711: note = XENP39533 Chain 2-MESO-C[mesothelin]_H1_Fab-IgG1_PV A_/S267K/S364K/E357Q/M428L/N434S/K447del_(G4S)3_1A7[CD28]_ H1.14_L1.71_scFv(GKPGS)4
QVQLVQSGAEVKKPGASVKVSCKASGYTFTSYWMHWVRQAPGQGLEWMGMIHPNSDNTIYYEKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGGGGGSGGGGSGGGGSEVQLLESGGGLVQPGGSLRLSCAASGFTFSSYYMSWVRQAPGKGLEWVSTISESGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIK

SEQ ID NO: 622 - protein, 214 aa, synthetic construct; identical to SEQ ID NO: 259
  source 1..214: note = XENP39533 Chain 3-MESO-C[mesothelin]_L1_Light Chain

SEQ ID NO: 623 - protein, 449 aa, synthetic construct; identical to SEQ ID NO: 617
  source 1..449: note = XENP39534 Chain 1-MESO-C[mesothelin]_H1_Fab-IgG1_pI (-)_Isosteric_A_PVA_/S267K/L368D/K370S/M428L/N434S

SEQ ID NO: 624 - protein, 711 aa, synthetic construct
  source 1..711: note = XENP39534 Chain 2-MESO-C[mesothelin]_H1_Fab-IgG1_PV A_/S267K/S364K/E357Q/M428L/N434S/K447del_(G4S)3_1A7[CD28]_ H1_L1.71_scFv(GKPGS)4
QVQLVQSGAEVKKPGASVKVSCKASGYTFTSYWMHWVRQAPGQGLEWMGMIHPNSDNTIYYEKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGGGGGSGGGGSGGGGSEVQLLESGGGLVQPGGSLRLSCAASGFTFSSYAMSWVRQAPGKGLEWVSTISGSGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIK

SEQ ID NO: 625 - protein, 214 aa, synthetic construct; identical to SEQ ID NO: 259
  source 1..214: note = XENP39534 Chain 3-MESO-C[mesothelin]_L1_Light Chain

SEQ ID NO: 626 - protein, 449 aa, synthetic construct; identical to SEQ ID NO: 617
  source 1..449: note = XENP39535 Chain 1-MESO-C[mesothelin]_H1_Fab-IgG1_pI (-)_Isosteric_A_PVA_/S267K/L368D/K370S/M428L/N434S

SEQ ID NO: 627 - protein, 711 aa, synthetic construct
  source 1..711: note = XENP39535 Chain 2-MESO-C[mesothelin]_H1_Fab-IgG1_PV A_/S267K/S364K/E357Q/M428L/N434S/K447del_(G4S)3_1A7[CD28]_ H1.1_L1.71_scFv(GKPGS)4
QVQLVQSGAEVKKPGASVKVSCKASGYTFTSYWMHWVRQAPGQGLEWMGMIHPNSDNTIYYEKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGGGGGSGGGGSGGGGSEVQLLESGGGLVQPGGSLRLSCAASGFTFSSYYMSWVRQAPGKGLEWVSTISGSGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIK

SEQ ID NO: 628 - protein, 214 aa, synthetic construct; identical to SEQ ID NO: 259
  source 1..214: note = XENP39535 Chain 3-MESO-C[mesothelin]_L1_Light Chain

SEQ ID NO: 629 - protein, 449 aa, synthetic construct; identical to SEQ ID NO: 617
  source 1..449: note = XENP39536 Chain 1-MESO-C[mesothelin]_H1_Fab-IgG1_pI (-)_Isosteric_A_PVA_/S267K/L368D/K370S/M428L/N434S

SEQ ID NO: 630 - protein, 711 aa, synthetic construct; identical to SEQ ID NO: 618
  source 1..711: note = XENP39536 Chain 2-MESO-C[mesothelin]_H1_Fab-IgG1_PV A_/S267K/S364K/E357Q/M428L/N434S/K447del_(G4S)3_1A7[CD28]_ H1.14_L1_scFv(GKPGS)4

SEQ ID NO: 631 - protein, 214 aa, synthetic construct
  source 1..214: note = XENP39536 Chain 3-MESO-C[mesothelin]_L1.3_Light Chain
DIVMTQSPDSLAVSLGERATINCKASHSVGTSVAWYQQKPGQSPKLLIYWASTRHTGVPDRFTGSGSGTDFTLTISSLQAEDVAVYFCQQYSSYPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 632 - protein, 449 aa, synthetic construct; identical to SEQ ID NO: 617
  source 1..449: note = XENP39537 Chain 1-MESO-C[mesothelin]_H1_L1_Fab-IgG1 _pI(-)_Isosteric_A_PVA_/S267K/L368D/K370S/M428L/N434

SEQ ID NO: 633 - protein, 711 aa, synthetic construct; identical to SEQ ID NO: 621
  source 1..711: note = XENP39537 Chain 2-MESO-C[mesothelin]_H1_Fab-IgG1_PV A_/S267K/S364K/E357Q/M428L/N434S/K447del_(G4S)3_1A7[CD28]_ H1.14_L1.71_scFv(GKPGS)4

SEQ ID NO: 634 - protein, 214 aa, synthetic construct; identical to SEQ ID NO: 631
  source 1..214: note = XENP39537 Chain 3-MESO-C[mesothelin]_L1.3_Light Chain

SEQ ID NO: 635 - protein, 449 aa, synthetic construct; identical to SEQ ID NO: 617
  source 1..449: note = XENP39538 Chain 1-MESO-C[mesothelin]_H1_Fab-IgG1_pI (-)_Isosteric_A_PVA_/S267K/L368D/K370S/M428L/N434S

SEQ ID NO: 636 - protein, 711 aa, synthetic construct; identical to SEQ ID NO: 624
  source 1..711: note = XENP39538 Chain 2-MESO-C[mesothelin]_H1_Fab-IgG1_PV A_/S267K/S364K/E357Q/M428L/N434S/K447del_(G4S)3_1A7[CD28]_ H1_L1.71_scFv(GKPGS)4

SEQ ID NO: 637 - protein, 214 aa, synthetic construct; identical to SEQ ID NO: 631
  source 1..214: note = XENP39538 Chain 3-MESO-C[mesothelin]_L1.3_Light Chain

SEQ ID NO: 638 - protein, 449 aa, synthetic construct; identical to SEQ ID NO: 617
  source 1..449: note = XENP39539 Chain 1-MESO-C[mesothelin]_H1_Fab-IgG1_pI (-)_Isosteric_A_PVA_/S267K/L368D/K370S/M428L/N434S

SEQ ID NO: 639 - protein, 711 aa, synthetic construct; identical to SEQ ID NO: 627
  source 1..711: note = XENP39539 Chain 2-MESO-C[mesothelin]_H1_Fab-IgG1_PV A_/S267K/S364K/E357Q/M428L/N434S/K447del_(G4S)3_1A7[CD28]_ H1.1_L1.71_scFv(GKPGS)4

SEQ ID NO: 640 - protein, 214 aa, synthetic construct; identical to SEQ ID NO: 631
  source 1..214: note = XENP39539 Chain 3-MESO-C[mesothelin]_L1.3_Light Chain

SEQ ID NO: 641 - protein, 449 aa, synthetic construct
  source 1..449: note = XENP39540 Chain 1-MESO-C[mesothelin]_H1.12_Fab-IgG1 _pI(-)_Isosteric_A_PVA_/S267K/L368D/K370S/M428L/N434S
QVQLVQSGAEVKKPGASVKVSCKASGYTFTSYWMHWVRQAPGQGLEWMGMIHPNSDNTIYYQKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 642 - protein, 711 aa, synthetic construct
  source 1..711: note = XENP39540 Chain 2-MESO-C[mesothelin]_H1.12_Fab-IgG1 _PVA_/S267K/S364K/E357Q/M428L/N434S/K447del_(G4S)3_1A7[CD2 8]_H1.14_L1_scFv(GKPGS)4
QVQLVQSGAEVKKPGASVKVSCKASGYTFTSYWMHWVRQAPGQGLEWMGMIHPNSDNTIYYQKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGGGGGSGGGGSGGGGSEVQLLESGGGLVQPGGSLRLSCAASGFTFSSYYMSWVRQAPGKGLEWVSTISESGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGQGTKLEIK

SEQ ID NO: 643 - protein, 214 aa, synthetic construct
  source 1..214: note = XENP39540 Chain 3-MESO-C[mesothelin]_L1.9_Light Chain
DIVMTQSPDSLAVSLGERATINCKASHDVGTSVAWYQQKPGQSPKLLIYWASTRHTGVPDRFTGSGSGTDFTLTISSLQAEDVAVYFCQQYYSYPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 644 - protein, 449 aa, synthetic construct; identical to SEQ ID NO: 641
  source 1..449: note = XENP39541 Chain 1-MESO-C[mesothelin]_H1.12_Fab-IgG1 _pI(-)_Isosteric_A_PVA_/S267K/L368D/K370S/M428L/N434S

SEQ ID NO: 645 - protein, 711 aa, synthetic construct
  source 1..711: note = XENP39541 Chain 2-MESO-C[mesothelin]_H1.12_Fab-IgG1 _PVA_/S267K/S364K/E357Q/M428L/N434S/K447del_(G4S)3_1A7[CD2 8]_H1.14_L1.71_scFv(GKPGS)4
QVQLVQSGAEVKKPGASVKVSCKASGYTFTSYWMHWVRQAPGQGLEWMGMIHPNSDNTIYYQKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGGGGGSGGGGSGGGGSEVQLLESGGGLVQPGGSLRLSCAASGFTFSSYYMSWVRQAPGKGLEWVSTISESGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIK

SEQ ID NO: 646 - protein, 214 aa, synthetic construct; identical to SEQ ID NO: 643
  source 1..214: note = XENP39541 Chain 3-MESO-C[mesothelin]_L1.9_Light Chain

SEQ ID NO: 647 - protein, 449 aa, synthetic construct; identical to SEQ ID NO: 641
  source 1..449: note = XENP39542 Chain 1-MESO-C[mesothelin]_H1.12_Fab-IgG1 _pI(-)_Isosteric_A_PVA_/S267K/L368D/K370S/M428L/N434S

SEQ ID NO: 648 - protein, 711 aa, synthetic construct
  source 1..711: note = XENP39542 Chain 2-MESO-C[mesothelin]_H1.12_Fab-IgG1 _PVA_/S267K/S364K/E357Q/M428L/N434S/K447del_(G4S)3_1A7[CD2 8]_H1_L1.71_scFv(GKPGS)4
QVQLVQSGAEVKKPGASVKVSCKASGYTFTSYWMHWVRQAPGQGLEWMGMIHPNSDNTIYYQKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGGGGGSGGGGSGGGGSEVQLLESGGGLVQPGGSLRLSCAASGFTFSSYAMSWVRQAPGKGLEWVSTISGSGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIK

SEQ ID NO: 649 - protein, 214 aa, synthetic construct; identical to SEQ ID NO: 643
  source 1..214: note = XENP39542 Chain 3-MESO-C[mesothelin]_L1.9_Light Chain

SEQ ID NO: 650 - protein, 449 aa, synthetic construct; identical to SEQ ID NO: 641
  source 1..449: note = XENP39543 Chain 1-MESO-C[mesothelin]_H1.12_Fab-IgG1 _pI(-)_Isosteric_A_PVA_/S267K/L368D/K370S/M428L/N434S

SEQ ID NO: 651 - protein, 711 aa, synthetic construct
  source 1..711: note = XENP39543 Chain 2-MESO-C[mesothelin]_H1.12_Fab-IgG1 _PVA_/S267K/S364K/E357Q/M428L/N434S/K447del_(G4S)3_1A7[CD2 8]_H1.1_L1.71_scFv(GKPGS)4
QVQLVQSGAEVKKPGASVKVSCKASGYTFTSYWMHWVRQAPGQGLEWMGMIHPNSDNTIYYQKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGGGGGSGGGGSGGGGSEVQLLESGGGLVQPGGSLRLSCAASGFTFSSYYMSWVRQAPGKGLEWVSTISGSGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIK

SEQ ID NO: 652 - protein, 214 aa, synthetic construct; identical to SEQ ID NO: 643
  source 1..214: note = XENP39543 Chain 3-MESO-C[mesothelin]_L1.9_Light Chain

SEQ ID NO: 653 - protein, 449 aa, synthetic construct
  source 1..449: note = XENP39544 Chain 1-MESO-C[mesothelin]_H1.3_Fab-IgG1_ pI(-)_Isosteric_A_PVA_/S267K/L368D/K370S/M428L/N434S
QVQLVQSGAEVKKPGASVKVSCKASGYTFTSYWMHWVRQAPGQGLEWMGWIHPNSDNTIYYEKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 654 - protein, 711 aa, synthetic construct
  source 1..711: note = XENP39544 Chain 2-MESO-C[mesothelin]_H1.3_Fab-IgG1_ PVA_/S267K/S364K/E357Q/M428L/N434S/K447del_(G4S)3_1A7[CD28 ]_H1.14_L1_scFv(GKPGS)4
QVQLVQSGAEVKKPGASVKVSCKASGYTFTSYWMHWVRQAPGQGLEWMGWIHPNSDNTIYYEKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGGGGGSGGGGSGGGGSEVQLLESGGGLVQPGGSLRLSCAASGFTFSSYYMSWVRQAPGKGLEWVSTISESGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGQGTKLEIK

SEQ ID NO: 655 - protein, 214 aa, synthetic construct; identical to SEQ ID NO: 259
  source 1..214: note = XENP39544 Chain 3-MESO-C[mesothelin]_L1_Light Chain

SEQ ID NO: 656 - protein, 449 aa, synthetic construct; identical to SEQ ID NO: 653
  source 1..449: note = XENP39545 Chain 1-MESO-C[mesothelin]_H1.3_Fab--IgG1 _pI(-)_Isosteric_A_PVA_/S267K/L368D/K370S/M428L/N434S

SEQ ID NO: 657 - protein, 711 aa, synthetic construct
  source 1..711: note = XENP39545 Chain 2-MESO-C[mesothelin]_H1.3_Fab-IgG1_ PVA_/S267K/S364K/E357Q/M428L/N434S/K447del_(G4S)3_1A7[CD28 ]_H1.14_L1.71_scFv(GKPGS)4
QVQLVQSGAEVKKPGASVKVSCKASGYTFTSYWMHWVRQAPGQGLEWMGWIHPNSDNTIYYEKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGGGGGSGGGGSGGGGSEVQLLESGGGLVQPGGSLRLSCAASGFTFSSYYMSWVRQAPGKGLEWVSTISESGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIK

SEQ ID NO: 658 - protein, 214 aa, synthetic construct; identical to SEQ ID NO: 259
  source 1..214: note = XENP39545 Chain 3-MESO-C[mesothelin]_L1_Light Chain

SEQ ID NO: 659 - protein, 449 aa, synthetic construct; identical to SEQ ID NO: 653
  source 1..449: note = XENP39546 Chain 1-MESO-C[mesothelin]_H1.3_Fab-IgG1_ pI(-)_Isosteric_A_PVA_/S267K/L368D/K370S/M428L/N434S

SEQ ID NO: 660 - protein, 711 aa, synthetic construct
  source 1..711: note = XENP39546 Chain 2-MESO-C[mesothelin]_H1.3_Fab-IgG1_ PVA_/S267K/S364K/E357Q/M428L/N434S/K447del_(G4S)3_1A7[CD28 ]_H1_L1.71_scFv(GKPGS)4
QVQLVQSGAEVKKPGASVKVSCKASGYTFTSYWMHWVRQAPGQGLEWMGWIHPNSDNTIYYEKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGGGGGSGGGGSGGGGSEVQLLESGGGLVQPGGSLRLSCAASGFTFSSYAMSWVRQAPGKGLEWVSTISGSGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIK

SEQ ID NO: 661 - protein, 214 aa, synthetic construct; identical to SEQ ID NO: 259
  source 1..214: note = XENP39546 Chain 3-MESO-C[mesothelin]_L1_Light Chain

SEQ ID NO: 662 - protein, 449 aa, synthetic construct; identical to SEQ ID NO: 653
  source 1..449: note = XENP39547 Chain 1-MESO-C[mesothelin]_H1.3_Fab-IgG1_ pI(-)_Isosteric_A_PVA_/S267K/L368D/K370S/M428L/N434S

SEQ ID NO: 663 - protein, 711 aa, synthetic construct
  source 1..711: note = XENP39547 Chain 2-MESO-C[mesothelin]_H1.3_Fab-IgG1_ PVA_/S267K/S364K/E357Q/M428L/N434S/K447del_(G4S)3_1A7[CD28 ]_H1.1_L1.71_scFv(GKPGS)4
QVQLVQSGAEVKKPGASVKVSCKASGYTFTSYWMHWVRQAPGQGLEWMGWIHPNSDNTIYYEKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGGGGGSGGGGSGGGGSEVQLLESGGGLVQPGGSLRLSCAASGFTFSSYYMSWVRQAPGKGLEWVSTISGSGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIK

SEQ ID NO: 664 - protein, 214 aa, synthetic construct; identical to SEQ ID NO: 259
  source 1..214: note = XENP39547 Chain 3-MESO-C[mesothelin]_L1_Light Chain

SEQ ID NO: 665 - protein, 449 aa, synthetic construct; identical to SEQ ID NO: 653
  source 1..449: note = XENP39548 Chain 1-MESO-C[mesothelin]_H1.3_Fab-IgG1_ pI(-)_Isosteric_A_PVA_/S267K/L368D/K370S/M428L/N434S

SEQ ID NO: 666 - protein, 711 aa, synthetic construct; identical to SEQ ID NO: 654
  source 1..711: note = XENP39548 Chain 2-MESO-C[mesothelin]_H1.3_Fab-IgG1_ PVA_/S267K/S364K/E357Q/M428L/N434S/K447del_(G4S)3_1A7[CD28 ]_H1.14_L1_scFv(GKPGS)4

SEQ ID NO: 667 - protein, 214 aa, synthetic construct
  source 1..214: note = XENP39548 Chain 3-MESO-C[mesothelin]_L1.13_Light Chain
DIVMTQSPDSLAVSLGERATINCKASHDVGQSVAWYQQKPGQSPKLLIYWASTRHTGVPDRFTGSGSGTDFTLTISSLQAEDVAVYFCQQYSSYPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 668 - protein, 449 aa, synthetic construct; identical to SEQ ID NO: 653
  source 1..449: note = XENP39549 Chain 1-MESO-C[mesothelin]_H1.3_Fab-IgG1_ pI(-)_Isosteric_A_PVA_/S267K/L368D/K370S/M428L/N434S

SEQ ID NO: 669 - protein, 711 aa, synthetic construct; identical to SEQ ID NO: 657
  source 1..711: note = XENP39549 Chain 2-MESO-C[mesothelin]_H1.3_Fab-IgG1_ PVA_/S267K/S364K/E357Q/M428L/N434S/K447del_(G4S)3_1A7[CD28 ]_H1.14_L1.71_scFv(GKPGS)4

SEQ ID NO: 670 - protein, 214 aa, synthetic construct; identical to SEQ ID NO: 667
  source 1..214: note = XENP39549 Chain 3-MESO-C[mesothelin]_L1.13_Light Chain

SEQ ID NO: 671 - protein, 449 aa, synthetic construct; identical to SEQ ID NO: 653
  source 1..449: note = XENP39550 Chain 1-MESO-C[mesothelin]_H1.3_L1_Fab-Ig G1_pI(-)_Isosteric_A_PVA_/S267K/L368D/K370S/M428L/N434S

SEQ ID NO: 672 - protein, 711 aa, synthetic construct; identical to SEQ ID NO: 660
  source 1..711: note = XENP39550 Chain 2-MESO-C[mesothelin]_H1.3_Fab-IgG1_ PVA_/S267K/S364K/E357Q/M428L/N434S/K447del_(G4S)3_1A7[CD28 ]_H1_L1.71_scFv(GKPGS)4

SEQ ID NO: 673 - protein, 214 aa, synthetic construct; identical to SEQ ID NO: 667
  source 1..214: note = XENP39550 Chain 3-MESO-C[mesothelin]_L1.13_Light Chain

SEQ ID NO: 674 - protein, 449 aa, synthetic construct; identical to SEQ ID NO: 653
  source 1..449: note = XENP39551 Chain 1-MESO-C[mesothelin]_H1.3_Fab-IgG1_ pI(-)_Isosteric_A_PVA_/S267K/L368D/K370S/M428L/N434S

SEQ ID NO: 675 - protein, 711 aa, synthetic construct; identical to SEQ ID NO: 663
  source 1..711: note = XENP39551 Chain 2-MESO-C[mesothelin]_H1.3_Fab-IgG1_ PVA_/S267K/S364K/E357Q/M428L/N434S/K447del_(G4S)3_1A7[CD28 ]_H1.1_L1.71_scFv(GKPGS)4

SEQ ID NO: 676 - protein, 214 aa, synthetic construct; identical to SEQ ID NO: 667
  source 1..214: note = XENP39551 Chain 3-MESO-C[mesothelin]_L1.13_Light Chain

SEQ ID NO: 677 - protein, 449 aa, synthetic construct; identical to SEQ ID NO: 617
  source 1..449: note = XENP39552 Chain 1-MESO-C[mesothelin]_H1_Fab-IgG1_pI (-)_Isosteric_A_PVA_/S267K/L368D/K370S/M428L/N434S

SEQ ID NO: 678 - protein, 479 aa, synthetic construct
  source 1..479: note = XENP39552 Chain 2-1A7[CD28]_H1.14_L1_scFv(GKPGS)4-F c(216)_IgG1_C220S/PVA_/S267K/S364K/E357Q/M428L/N434S
EVQLLESGGGLVQPGGSLRLSCAASGFTFSSYYMSWVRQAPGKGLEWVSTISESGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGQGTKLEIKEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 679 - protein, 214 aa, synthetic construct; identical to SEQ ID NO: 259
  source 1..214: note = XENP39552 Chain 3-MESO-C[mesothelin]_L1_Light Chain

SEQ ID NO: 680 - protein, 449 aa, synthetic construct; identical to SEQ ID NO: 617
  source 1..449: note = XENP39553 Chain 1-MESO-C[mesothelin]_H1_Fab-IgG1_pI (-)_Isosteric_A_PVA_/S267K/L368D/K370S/M428L/N434S

SEQ ID NO: 681 - protein, 479 aa, synthetic construct
  source 1..479: note = XENP39553 Chain 2-1A7[CD28]_H1.14_L1.71_scFv(GKPGS) 4-Fc(216)_IgG1_C220S/PVA_/S267K/S364K/E357Q/M428L/N434S
EVQLLESGGGLVQPGGSLRLSCAASGFTFSSYYMSWVRQAPGKGLEWVSTISESGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIKEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 682 - protein, 214 aa, synthetic construct; identical to SEQ ID NO: 259
  source 1..214: note = XENP39553 Chain 3-MESO-C[mesothelin]_L1_Light Chain

SEQ ID NO: 683 - protein, 449 aa, synthetic construct; identical to SEQ ID NO: 617
  source 1..449: note = XENP39554 Chain 1-MESO-C[mesothelin]_H1_Fab-IgG1_pI (-)_Isosteric_A_PVA_/S267K/L368D/K370S/M428L/N434S

SEQ ID NO: 684 - protein, 479 aa, synthetic construct
  source 1..479: note = XENP39554 Chain 2-1A7[CD28]_H1_L1.71_scFv(GKPGS)4-F c(216)_IgG1_C220S/PVA_/S267K/S364K/E357Q/M428L/N434S
EVQLLESGGGLVQPGGSLRLSCAASGFTFSSYAMSWVRQAPGKGLEWVSTISGSGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIKEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 685 - protein, 214 aa, synthetic construct; identical to SEQ ID NO: 259
  source 1..214: note = XENP39554 Chain 3-MESO-C[mesothelin]_L1_Light Chain

SEQ ID NO: 686 - protein, 449 aa, synthetic construct; identical to SEQ ID NO: 617
  source 1..449: note = XENP39555 Chain 1-MESO-C[mesothelin]_H1_Fab-IgG1_pI (-)_Isosteric_A_PVA_/S267K/L368D/K370S/M428L/N434S

SEQ ID NO: 687 - protein, 479 aa, synthetic construct
  source 1..479: note = XENP39555 Chain 2-1A7[CD28]_H1.1_L1.71_scFv(GKPGS)4 -Fc(216)_IgG1_C220S/PVA_/S267K/S364K/E357Q/M428L/N434S
EVQLLESGGGLVQPGGSLRLSCAASGFTFSSYYMSWVRQAPGKGLEWVSTISGSGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIKEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 688 - protein, 214 aa, synthetic construct; identical to SEQ ID NO: 259
  source 1..214: note = XENP39555 Chain 3-MESO-C[mesothelin]_L1_Light Chain

SEQ ID NO: 689 - protein, 449 aa, synthetic construct; identical to SEQ ID NO: 617
  source 1..449: note = XENP39556 Chain 1-MESO-C[mesothelin]_H1_Fab-IgG1_pI (-)_Isosteric_A_PVA_/S267K/L368D/K370S/M428L/N434S

SEQ ID NO: 690 - protein, 479 aa, synthetic construct; identical to SEQ ID NO: 678
  source 1..479: note = XENP39556 Chain 2-1A7[CD28]_H1.14_L1_scFv(GKPGS)4-F c(216)_IgG1_C220S/PVA_/S267K/S364K/E357Q/M428L/N434S

SEQ ID NO: 691 - protein, 214 aa, synthetic construct; identical to SEQ ID NO: 631
  source 1..214: note = XENP39556 Chain 3-MESO-C[mesothelin]_L1.3_Light Chain

SEQ ID NO: 692 - protein, 449 aa, synthetic construct; identical to SEQ ID NO: 617
  source 1..449: note = XENP39557 Chain 1-MESO-C[mesothelin]_H1_Fab-IgG1_pI (-)_Isosteric_A_PVA_/S267K/L368D/K370S/M428L/N434S

SEQ ID NO: 693 - protein, 479 aa, synthetic construct; identical to SEQ ID NO: 681
  source 1..479: note = XENP39557 Chain 2-1A7[CD28]_H1.14_L1.71_scFv(GKPGS) 4-Fc(216)_IgG1_C220S/PVA_/S267K/S364K/E357Q/M428L/N434S

SEQ ID NO: 694 - protein, 214 aa, synthetic construct; identical to SEQ ID NO: 631
  source 1..214: note = XENP39557 Chain 3-MESO-C[mesothelin]_L1.3_Light Chain

SEQ ID NO: 695 - protein, 449 aa, synthetic construct; identical to SEQ ID NO: 617
  source 1..449: note = XENP39558 Chain 1-MESO-C[mesothelin]_H1_Fab-IgG1_pI (-)_Isosteric_A_PVA_/S267K/L368D/K370S/M428L/N434S

SEQ ID NO: 696 - protein, 479 aa, synthetic construct; identical to SEQ ID NO: 684
  source 1..479: note = XENP39558 Chain 2-1A7[CD28]_H1_L1.71_scFv(GKPGS)4-F c(216)_IgG1_C220S/PVA_/S267K/S364K/E357Q/M428L/N434S

SEQ ID NO: 697 - protein, 214 aa, synthetic construct; identical to SEQ ID NO: 631
  source 1..214: note = XENP39558 Chain 3-MESO-C[mesothelin]_L1.3_Light Chain

SEQ ID NO: 698 - protein, 449 aa, synthetic construct; identical to SEQ ID NO: 617
  source 1..449: note = XENP39559 Chain 1-MESO-C[mesothelin]_H1_Fab-IgG1_pI (-)_Isosteric_A_PVA_/S267K/L368D/K370S/M428L/N434S

SEQ ID NO: 699 - protein, 479 aa, synthetic construct; identical to SEQ ID NO: 687
  source 1..479: note = XENP39559 Chain 2-1A7[CD28]_H1.1_L1.71_scFv(GKPGS)4 -Fc(216)_IgG1_C220S/PVA_/S267K/S364K/E357Q/M428L/N434S

SEQ ID NO: 700 - protein, 214 aa, synthetic construct; identical to SEQ ID NO: 631
  source 1..214: note = XENP39559 Chain 3-MESO-C[mesothelin]_L1.3_Light Chain

SEQ ID NO: 701 - protein, 449 aa, synthetic construct; identical to SEQ ID NO: 641
  source 1..449: note = XENP39560 Chain 1-MESO-C[mesothelin]_H1.12_Fab-IgG1 _pI(-)_Isosteric_A_PVA_/S267K/L368D/K370S/M428L/N434S

SEQ ID NO: 702 - protein, 479 aa, synthetic construct; identical to SEQ ID NO: 678
  source 1..479: note = XENP39560 Chain 2-1A7[CD28]_H1.14_L1_scFv(GKPGS)4-F c(216)_IgG1_C220S/PVA_/S267K/S364K/E357Q/M428L/N434S

SEQ ID NO: 703 - protein, 214 aa, synthetic construct; identical to SEQ ID NO: 643
  source 1..214: note = XENP39560 Chain 3-MESO-C[mesothelin]_L1.9_Light Chain

SEQ ID NO: 704 - protein, 449 aa, synthetic construct; identical to SEQ ID NO: 641
  source 1..449: note = XENP39561 Chain 1-MESO-C[mesothelin]_H1.12_Fab-IgG1 _pI(-)_Isosteric_A_PVA_/S267K/L368D/K370S/M428L/N434S

SEQ ID NO: 705 - protein, 479 aa, synthetic construct; identical to SEQ ID NO: 681
  source 1..479: note = XENP39561 Chain 2-1A7[CD28]_H1.14_L1.71_scFv(GKPGS) 4-Fc(216)_IgG1_C220S/PVA_/S267K/S364K/E357Q/M428L/N434S

SEQ ID NO: 706 - protein, 214 aa, synthetic construct; identical to SEQ ID NO: 643
  source 1..214: note = XENP39561 Chain 3-MESO-C[mesothelin]_L1.9_Light Chain

SEQ ID NO: 707 - protein, 449 aa, synthetic construct; identical to SEQ ID NO: 641
  source 1..449: note = XENP39562 Chain 1-MESO-C[mesothelin]_H1.12_Fab-IgG1 _pI(-)_Isosteric_A_PVA_/S267K/L368D/K370S/M428L/N434S

SEQ ID NO: 708 - protein, 479 aa, synthetic construct; identical to SEQ ID NO: 684
  source 1..479: note = XENP39562 Chain 2-1A7[CD28]_H1_L1.71_scFv(GKPGS)4-F c(216)_IgG1_C220S/PVA_/S267K/S364K/E357Q/M428L/N434S

SEQ ID NO: 709 - protein, 214 aa, synthetic construct; identical to SEQ ID NO: 643
  source 1..214: note = XENP39562 Chain 3-MESO-C[mesothelin]_L1.9_Light Chain

SEQ ID NO: 710 - protein, 449 aa, synthetic construct; identical to SEQ ID NO: 641
  source 1..449: note = XENP39563 Chain 1-MESO-C[mesothelin]_H1.12_Fab-IgG1 _pI(-)_Isosteric_A_PVA_/S267K/L368D/K370S/M428L/N434S

SEQ ID NO: 711 - protein, 479 aa, synthetic construct; identical to SEQ ID NO: 687
  source 1..479: note = XENP39563 Chain 2-1A7[CD28]_H1.1_L1.71_scFv(GKPGS)4 -Fc(216)_IgG1_C220S/PVA_/S267K/S364K/E357Q/M428L/N434S

SEQ ID NO: 712 - protein, 214 aa, synthetic construct; identical to SEQ ID NO: 643
  source 1..214: note = XENP39563 Chain 3-MESO-C[mesothelin]_L1.9_Light Chain

SEQ ID NO: 713 - protein, 449 aa, synthetic construct; identical to SEQ ID NO: 653
  source 1..449: note = XENP39564 Chain 1-MESO-C[mesothelin]_H1.3_Fab-IgG1_ pI(-)_Isosteric_A_PVA_/S267K/L368D/K370S/M428L/N434S

SEQ ID NO: 714 - protein, 479 aa, synthetic construct; identical to SEQ ID NO: 678
  source 1..479: note = XENP39564 Chain 2-1A7[CD28]_H1.14_L1_scFv(GKPGS)4-F c(216)_IgG1_C220S/PVA_/S267K/S364K/E357Q/M428L/N434S

SEQ ID NO: 715 - protein, 214 aa, synthetic construct; identical to SEQ ID NO: 259
  source 1..214: note = XENP39564 Chain 3-MESO-C[mesothelin]_L1_Light Chain

SEQ ID NO: 716 - protein, 449 aa, synthetic construct; identical to SEQ ID NO: 653
  source 1..449: note = XENP39565 Chain 1-MESO-C[mesothelin]_H1.3_Fab-IgG1_ pI(-)_Isosteric_A_PVA_/S267K/L368D/K370S/M428L/N434S

SEQ ID NO: 717 - protein, 479 aa, synthetic construct; identical to SEQ ID NO: 681
  source 1..479: note = XENP39565 Chain 2-1A7[CD28]_H1.14_L1.71_scFv(GKPGS) 4-Fc(216)_IgG1_C220S/PVA_/S267K/S364K/E357Q/M428L/N434S

SEQ ID NO: 718 - protein, 214 aa, synthetic construct; identical to SEQ ID NO: 259
  source 1..214: note = XENP39565 Chain 3-MESO-C[mesothelin]_L1_Light Chain

SEQ ID NO: 719 - protein, 449 aa, synthetic construct; identical to SEQ ID NO: 653
  source 1..449: note = XENP39566 Chain 1-MESO-C[mesothelin]_H1.3_Fab-IgG1_ pI(-)_Isosteric_A_PVA_/S267K/L368D/K370S/M428L/N434S

SEQ ID NO: 720 - protein, 479 aa, synthetic construct; identical to SEQ ID NO: 684
  source 1..479: note = XENP39566 Chain 2-1A7[CD28]_H1_L1.71_scFv(GKPGS)4-F c(216)_IgG1_C220S/PVA_/S267K/S364K/E357Q/M428L/N434S

SEQ ID NO: 721 - protein, 214 aa, synthetic construct; identical to SEQ ID NO: 259
  source 1..214: note = XENP39566 Chain 3-MESO-C[mesothelin]_L1_Light Chain

SEQ ID NO: 722 - protein, 449 aa, synthetic construct; identical to SEQ ID NO: 653
  source 1..449: note = XENP39567 Chain 1-MESO-C[mesothelin]_H1.3_Fab-IgG1_ pI(-)_Isosteric_A_PVA_/S267K/L368D/K370S/M428L/N434S

SEQ ID NO: 723 - protein, 479 aa, synthetic construct; identical to SEQ ID NO: 687
  source 1..479: note = XENP39567 Chain 2-1A7[CD28]_H1.1_L1.71_scFv(GKPGS)4 -Fc(216)_IgG1_C220S/PVA_/S267K/S364K/E357Q/M428L/N434S

SEQ ID NO: 724 - protein, 214 aa, synthetic construct; identical to SEQ ID NO: 259
  source 1..214: note = XENP39567 Chain 3-MESO-C[mesothelin]_L1_Light Chain

SEQ ID NO: 725 - protein, 449 aa, synthetic construct; identical to SEQ ID NO: 653
  source 1..449: note = XENP39568 Chain 1-MESO-C[mesothelin]_H1.3_Fab-IgG1_ pI(-)_Isosteric_A_PVA_/S267K/L368D/K370S/M428L/N434S

SEQ ID NO: 726 - protein, 479 aa, synthetic construct; identical to SEQ ID NO: 678
  source 1..479: note = XENP39568 Chain 2-1A7[CD28]_H1.14_L1_scFv(GKPGS)4-F c(216)_IgG1_C220S/PVA_/S267K/S364K/E357Q/M428L/N434S

SEQ ID NO: 727 - protein, 214 aa, synthetic construct; identical to SEQ ID NO: 667
  source 1..214: note = XENP39568 Chain 3-MESO-C[mesothelin]_L1.13_Light Chain

SEQ ID NO: 728 - protein, 449 aa, synthetic construct; identical to SEQ ID NO: 653
  source 1..449: note = XENP39569 Chain 1-MESO-C[mesothelin]_H1.3_Fab-IgG1_ pI(-)_Isosteric_A_PVA_/S267K/L368D/K370S/M428L/N434S

SEQ ID NO: 729 - protein, 479 aa, synthetic construct; identical to SEQ ID NO: 681
  source 1..479: note = XENP39569 Chain 2-1A7[CD28]_H1.14_L1.71_scFv(GKPGS) 4-Fc(216)_IgG1_C220S/PVA_/S267K/S364K/E357Q/M428L/N434S

SEQ ID NO: 730 - protein, 214 aa, synthetic construct; identical to SEQ ID NO: 667
  source 1..214: note = XENP39569 Chain 3-MESO-C[mesothelin]_L1.13_Light Chain

SEQ ID NO: 731 - protein, 449 aa, synthetic construct; identical to SEQ ID NO: 653
  source 1..449: note = XENP39570 Chain 1-MESO-C[mesothelin]_H1.3_Fab-IgG1_ pI(-)_Isosteric_A_PVA_/S267K/L368D/K370S/M428L/N434S

SEQ ID NO: 732 - protein, 479 aa, synthetic construct; identical to SEQ ID NO: 684
  source 1..479: note = XENP39570 Chain 2-1A7[CD28]_H1_L1.71_scFv(GKPGS)4-F c(216)_IgG1_C220S/PVA_/S267K/S364K/E357Q/M428L/N434S

SEQ ID NO: 733 - protein, 214 aa, synthetic construct; identical to SEQ ID NO: 667
  source 1..214: note = XENP39570 Chain 3-MESO-C[mesothelin]_L1.13_Light Chain

SEQ ID NO: 734 - protein, 449 aa, synthetic construct; identical to SEQ ID NO: 653
  source 1..449: note = XENP39571 Chain 1-MESO-C[mesothelin]_H1.3_Fab--IgG1 _pI(-)_Isosteric_A_PVA_/S267K/L368D/K370S/M428L/N434S

SEQ ID NO: 735 - protein, 479 aa, synthetic construct; identical to SEQ ID NO: 687
  source 1..479: note = XENP39571 Chain 2-1A7[CD28]_H1.1_L1.71_scFv(GKPGS)4 -Fc(216)_IgG1_C220S/PVA_/S267K/S364K/E357Q/M428L/N434S

SEQ ID NO: 736 - protein, 214 aa, synthetic construct; identical to SEQ ID NO: 667
  source 1..214: note = XENP39571 Chain 3-MESO-C[mesothelin]_L1.13_Light Chain

SEQ ID NO: 737 - protein, 121 aa, synthetic construct
  source 1..121: note = 1A7[CD28]_H1L1 Variable heavy (vh) domain
EVQLLESGGGLVQPGGSLRLSCAASGFTFSSYAMSWVRQAPGKGLEWVSTISGSGDSTYYADSVKGRFTISRDNSKNTLYLQMNSLRAEDTAVYYCAKSGPGLRQVGFDYWGQGTLVTVSS

SEQ ID NO: 738 - protein, 5 aa, synthetic construct
  source 1..5: note = 1A7[CD28]_H1L1 vhCDR1
SYAMS

SEQ ID NO: 739 - protein, 17 aa, synthetic construct
  source 1..17: note = 1A7[CD28]_H1L1 vhCDR2
TISGSGDSTYYADSVKG

SEQ ID NO: 740 - protein, 12 aa, synthetic construct
  source 1..12: note = 1A7[CD28]_H1L1 vhCDR3
SGPGLRQVGFDY

SEQ ID NO: 741 - protein, 107 aa, synthetic construct
  source 1..107: note = 1A7[CD28]_H1L1 Variable light (vl) domain
DIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGQGTKLEIK

SEQ ID NO: 742 - protein, 11 aa, synthetic construct
  source 1..11: note = 1A7[CD28]_H1L1 vlCDR1
RASQSISSYLN

SEQ ID NO: 743 - protein, 7 aa, synthetic construct
  source 1..7: note = 1A7[CD28]_H1L1 vlCDR2
AASSLQS

SEQ ID NO: 744 - protein, 9 aa, synthetic construct
  source 1..9: note = 1A7[CD28]_H1L1 vlCDR3
QQSYSTPFT

SEQ ID NO: 745 - protein, 121 aa, synthetic construct
  source 1..121: note = 1A7[CD28]_H1.1
EVQLLESGGGLVQPGGSLRLSCAASGFTFSSYYMSWVRQAPGKGLEWVSTISGSGDSTYYADSVKGRFTISRDNSKNTLYLQMNSLRAEDTAVYYCAKSGPGLRQVGFDYWGQGTLVTVSS

SEQ ID NO: 746 - protein, 121 aa, synthetic construct
  source 1..121: note = 1A7[CD28]_H1.2
EVQLLESGGGLVQPGGSLRLSCAASGFTFSSYAMSWVRQAPGKGLEWVSTIDGSGDSTYYADSVKGRFTISRDNSKNTLYLQMNSLRAEDTAVYYCAKSGPGLRQVGFDYWGQGTLVTVSS

SEQ ID NO: 747 - protein, 121 aa, synthetic construct
  source 1..121: note = 1A7[CD28]_H1.3
EVQLLESGGGLVQPGGSLRLSCAASGFTFSSYAMSWVRQAPGKGLEWVSTIEGSGDSTYYADSVKGRFTISRDNSKNTLYLQMNSLRAEDTAVYYCAKSGPGLRQVGFDYWGQGTLVTVSS

SEQ ID NO: 748 - protein, 121 aa, synthetic construct
  source 1..121: note = 1A7[CD28]_H1.4
EVQLLESGGGLVQPGGSLRLSCAASGFTFSSYAMSWVRQAPGKGLEWVSTIYGSGDSTYYADSVKGRFTISRDNSKNTLYLQMNSLRAEDTAVYYCAKSGPGLRQVGFDYWGQGTLVTVSS

SEQ ID NO: 749 - protein, 121 aa, synthetic construct
  source 1..121: note = 1A7[CD28]_H1.5
EVQLLESGGGLVQPGGSLRLSCAASGFTFSSYAMSWVRQAPGKGLEWVSTISDSGDSTYYADSVKGRFTISRDNSKNTLYLQMNSLRAEDTAVYYCAKSGPGLRQVGFDYWGQGTLVTVSS

SEQ ID NO: 750 - protein, 121 aa, synthetic construct
  source 1..121: note = 1A7[CD28]_H1.6
EVQLLESGGGLVQPGGSLRLSCAASGFTFSSYAMSWVRQAPGKGLEWVSTISESGDSTYYADSVKGRFTISRDNSKNTLYLQMNSLRAEDTAVYYCAKSGPGLRQVGFDYWGQGTLVTVSS

SEQ ID NO: 751 - protein, 121 aa, synthetic construct
  source 1..121: note = 1A7[CD28]_H1.7
EVQLLESGGGLVQPGGSLRLSCAASGFTFSSYAMSWVRQAPGKGLEWVSTISGSGTSTYYADSVKGRFTISRDNSKNTLYLQMNSLRAEDTAVYYCAKSGPGLRQVGFDYWGQGTLVTVSS

SEQ ID NO: 752 - protein, 121 aa, synthetic construct
  source 1..121: note = 1A7[CD28]_H1.8
EVQLLESGGGLVQPGGSLRLSCAASGFTFSSYAMSWVRQAPGKGLEWVSTISGSGYSTYYADSVKGRFTISRDNSKNTLYLQMNSLRAEDTAVYYCAKSGPGLRQVGFDYWGQGTLVTVSS

SEQ ID NO: 753 - protein, 121 aa, synthetic construct
  source 1..121: note = 1A7[CD28]_H1.9
EVQLLESGGGLVQPGGSLRLSCAASGFTFSSYAMSWVRQAPGKGLEWVSTISGSGDYTYYADSVKGRFTISRDNSKNTLYLQMNSLRAEDTAVYYCAKSGPGLRQVGFDYWGQGTLVTVSS

SEQ ID NO: 754 - protein, 121 aa, synthetic construct
  source 1..121: note = 1A7[CD28]_H1.10
EVQLLESGGGLVQPGGSLRLSCAASGFTFSSYYMSWVRQAPGKGLEWVSTIDGSGDSTYYADSVKGRFTISRDNSKNTLYLQMNSLRAEDTAVYYCAKSGPGLRQVGFDYWGQGTLVTVSS

SEQ ID NO: 755 - protein, 121 aa, synthetic construct
  source 1..121: note = 1A7[CD28]_H1.11
EVQLLESGGGLVQPGGSLRLSCAASGFTFSSYYMSWVRQAPGKGLEWVSTIEGSGDSTYYADSVKGRFTISRDNSKNTLYLQMNSLRAEDTAVYYCAKSGPGLRQVGFDYWGQGTLVTVSS

SEQ ID NO: 756 - protein, 121 aa, synthetic construct
  source 1..121: note = 1A7[CD28]_H1.12
EVQLLESGGGLVQPGGSLRLSCAASGFTFSSYYMSWVRQAPGKGLEWVSTIYGSGDSTYYADSVKGRFTISRDNSKNTLYLQMNSLRAEDTAVYYCAKSGPGLRQVGFDYWGQGTLVTVSS

SEQ ID NO: 757 - protein, 121 aa, synthetic construct
  source 1..121: note = 1A7[CD28]_H1.13
EVQLLESGGGLVQPGGSLRLSCAASGFTFSSYYMSWVRQAPGKGLEWVSTISDSGDSTYYADSVKGRFTISRDNSKNTLYLQMNSLRAEDTAVYYCAKSGPGLRQVGFDYWGQGTLVTVSS

SEQ ID NO: 758 - protein, 121 aa, synthetic construct
  source 1..121: note = 1A7[CD28]_H1.14
EVQLLESGGGLVQPGGSLRLSCAASGFTFSSYYMSWVRQAPGKGLEWVSTISESGDSTYYADSVKGRFTISRDNSKNTLYLQMNSLRAEDTAVYYCAKSGPGLRQVGFDYWGQGTLVTVSS

SEQ ID NO: 759 - protein, 121 aa, synthetic construct
  source 1..121: note = 1A7[CD28]_H1.15
EVQLLESGGGLVQPGGSLRLSCAASGFTFSSYYMSWVRQAPGKGLEWVSTISGSGTSTYYADSVKGRFTISRDNSKNTLYLQMNSLRAEDTAVYYCAKSGPGLRQVGFDYWGQGTLVTVSS

SEQ ID NO: 760 - protein, 121 aa, synthetic construct
  source 1..121: note = 1A7[CD28]_H1.16
EVQLLESGGGLVQPGGSLRLSCAASGFTFSSYYMSWVRQAPGKGLEWVSTISGSGYSTYYADSVKGRFTISRDNSKNTLYLQMNSLRAEDTAVYYCAKSGPGLRQVGFDYWGQGTLVTVSS

SEQ ID NO: 761 - protein, 121 aa, synthetic construct
  source 1..121: note = 1A7[CD28]_H1.17
EVQLLESGGGLVQPGGSLRLSCAASGFTFSSYYMSWVRQAPGKGLEWVSTISGSGDYTYYADSVKGRFTISRDNSKNTLYLQMNSLRAEDTAVYYCAKSGPGLRQVGFDYWGQGTLVTVSS

SEQ ID NO: 762 - protein, 121 aa, synthetic construct
  source 1..121: note = 1A7[CD28]_H1.18
EVQLLESGGGLVQPGGSLRLSCAASGFTFSSYAMSWVRQAPGKGLEWVSTIDDSGDSTYYADSVKGRFTISRDNSKNTLYLQMNSLRAEDTAVYYCAKSGPGLRQVGFDYWGQGTLVTVSS

SEQ ID NO: 763 - protein, 121 aa, synthetic construct
  source 1..121: note = 1A7[CD28]_H1.19
EVQLLESGGGLVQPGGSLRLSCAASGFTFSSYAMSWVRQAPGKGLEWVSTIDESGDSTYYADSVKGRFTISRDNSKNTLYLQMNSLRAEDTAVYYCAKSGPGLRQVGFDYWGQGTLVTVSS

SEQ ID NO: 764 - protein, 121 aa, synthetic construct
  source 1..121: note = 1A7[CD28]_H1.20
EVQLLESGGGLVQPGGSLRLSCAASGFTFSSYAMSWVRQAPGKGLEWVSTIDGSGTSTYYADSVKGRFTISRDNSKNTLYLQMNSLRAEDTAVYYCAKSGPGLRQVGFDYWGQGTLVTVSS

SEQ ID NO: 765 - protein, 121 aa, synthetic construct
  source 1..121: note = 1A7[CD28]_H1.21
EVQLLESGGGLVQPGGSLRLSCAASGFTFSSYAMSWVRQAPGKGLEWVSTIDGSGYSTYYADSVKGRFTISRDNSKNTLYLQMNSLRAEDTAVYYCAKSGPGLRQVGFDYWGQGTLVTVSS

SEQ ID NO: 766 - protein, 121 aa, synthetic construct
  source 1..121: note = 1A7[CD28]_H1.22
EVQLLESGGGLVQPGGSLRLSCAASGFTFSSYAMSWVRQAPGKGLEWVSTIDGSGDYTYYADSVKGRFTISRDNSKNTLYLQMNSLRAEDTAVYYCAKSGPGLRQVGFDYWGQGTLVTVSS

SEQ ID NO: 767 - protein, 121 aa, synthetic construct
  source 1..121: note = 1A7[CD28]_H1.23
EVQLLESGGGLVQPGGSLRLSCAASGFTFSSYAMSWVRQAPGKGLEWVSTIEDSGDSTYYADSVKGRFTISRDNSKNTLYLQMNSLRAEDTAVYYCAKSGPGLRQVGFDYWGQGTLVTVSS

SEQ ID NO: 768 - protein, 121 aa, synthetic construct
  source 1..121: note = 1A7[CD28]_H1.24
EVQLLESGGGLVQPGGSLRLSCAASGFTFSSYAMSWVRQAPGKGLEWVSTIEESGDSTYYADSVKGRFTISRDNSKNTLYLQMNSLRAEDTAVYYCAKSGPGLRQVGFDYWGQGTLVTVSS

SEQ ID NO: 769 - protein, 121 aa, synthetic construct
  source 1..121: note = 1A7[CD28]_H1.25
EVQLLESGGGLVQPGGSLRLSCAASGFTFSSYAMSWVRQAPGKGLEWVSTIEGSGTSTYYADSVKGRFTISRDNSKNTLYLQMNSLRAEDTAVYYCAKSGPGLRQVGFDYWGQGTLVTVSS

SEQ ID NO: 770 - protein, 121 aa, synthetic construct
  source 1..121: note = 1A7[CD28]_H1.26
EVQLLESGGGLVQPGGSLRLSCAASGFTFSSYAMSWVRQAPGKGLEWVSTIEGSGYSTYYADSVKGRFTISRDNSKNTLYLQMNSLRAEDTAVYYCAKSGPGLRQVGFDYWGQGTLVTVSS

SEQ ID NO: 771 - protein, 121 aa, synthetic construct
  source 1..121: note = 1A7[CD28]_H1.27
EVQLLESGGGLVQPGGSLRLSCAASGFTFSSYAMSWVRQAPGKGLEWVSTIEGSGDYTYYADSVKGRFTISRDNSKNTLYLQMNSLRAEDTAVYYCAKSGPGLRQVGFDYWGQGTLVTVSS

SEQ ID NO: 772 - protein, 121 aa, synthetic construct
  source 1..121: note = 1A7[CD28]_H1.28
EVQLLESGGGLVQPGGSLRLSCAASGFTFSSYAMSWVRQAPGKGLEWVSTIYDSGDSTYYADSVKGRFTISRDNSKNTLYLQMNSLRAEDTAVYYCAKSGPGLRQVGFDYWGQGTLVTVSS

SEQ ID NO: 773 - protein, 121 aa, synthetic construct
  source 1..121: note = 1A7[CD28]_H1.29
EVQLLESGGGLVQPGGSLRLSCAASGFTFSSYAMSWVRQAPGKGLEWVSTIYESGDSTYYADSVKGRFTISRDNSKNTLYLQMNSLRAEDTAVYYCAKSGPGLRQVGFDYWGQGTLVTVSS

SEQ ID NO: 774 - protein, 121 aa, synthetic construct
  source 1..121: note = 1A7[CD28]_H1.30
EVQLLESGGGLVQPGGSLRLSCAASGFTFSSYAMSWVRQAPGKGLEWVSTIYGSGTSTYYADSVKGRFTISRDNSKNTLYLQMNSLRAEDTAVYYCAKSGPGLRQVGFDYWGQGTLVTVSS

SEQ ID NO: 775 - protein, 121 aa, synthetic construct
  source 1..121: note = 1A7[CD28]_H1.31
EVQLLESGGGLVQPGGSLRLSCAASGFTFSSYAMSWVRQAPGKGLEWVSTIYGSGYSTYYADSVKGRFTISRDNSKNTLYLQMNSLRAEDTAVYYCAKSGPGLRQVGFDYWGQGTLVTVSS

SEQ ID NO: 776 - protein, 121 aa, synthetic construct
  source 1..121: note = 1A7[CD28]_H1.32
EVQLLESGGGLVQPGGSLRLSCAASGFTFSSYAMSWVRQAPGKGLEWVSTIYGSGDYTYYADSVKGRFTISRDNSKNTLYLQMNSLRAEDTAVYYCAKSGPGLRQVGFDYWGQGTLVTVSS

SEQ ID NO: 777 - protein, 121 aa, synthetic construct
  source 1..121: note = 1A7[CD28]_H1.33
EVQLLESGGGLVQPGGSLRLSCAASGFTFSSYAMSWVRQAPGKGLEWVSTISDSGTSTYYADSVKGRFTISRDNSKNTLYLQMNSLRAEDTAVYYCAKSGPGLRQVGFDYWGQGTLVTVSS

SEQ ID NO: 778 - protein, 121 aa, synthetic construct
  source 1..121: note = 1A7[CD28]_H1.34
EVQLLESGGGLVQPGGSLRLSCAASGFTFSSYAMSWVRQAPGKGLEWVSTISDSGYSTYYADSVKGRFTISRDNSKNTLYLQMNSLRAEDTAVYYCAKSGPGLRQVGFDYWGQGTLVTVSS

SEQ ID NO: 779 - protein, 121 aa, synthetic construct
  source 1..121: note = 1A7[CD28]_H1.35
EVQLLESGGGLVQPGGSLRLSCAASGFTFSSYAMSWVRQAPGKGLEWVSTISDSGDYTYYADSVKGRFTISRDNSKNTLYLQMNSLRAEDTAVYYCAKSGPGLRQVGFDYWGQGTLVTVSS

SEQ ID NO: 780 - protein, 121 aa, synthetic construct
  source 1..121: note = 1A7[CD28]_H1.36
EVQLLESGGGLVQPGGSLRLSCAASGFTFSSYAMSWVRQAPGKGLEWVSTISESGTSTYYADSVKGRFTISRDNSKNTLYLQMNSLRAEDTAVYYCAKSGPGLRQVGFDYWGQGTLVTVSS

SEQ ID NO: 781 - protein, 121 aa, synthetic construct
  source 1..121: note = 1A7[CD28]_H1.37
EVQLLESGGGLVQPGGSLRLSCAASGFTFSSYAMSWVRQAPGKGLEWVSTISESGYSTYYADSVKGRFTISRDNSKNTLYLQMNSLRAEDTAVYYCAKSGPGLRQVGFDYWGQGTLVTVSS

SEQ ID NO: 782 - protein, 121 aa, synthetic construct
  source 1..121: note = 1A7[CD28]_H1.38
EVQLLESGGGLVQPGGSLRLSCAASGFTFSSYAMSWVRQAPGKGLEWVSTISESGDYTYYADSVKGRFTISRDNSKNTLYLQMNSLRAEDTAVYYCAKSGPGLRQVGFDYWGQGTLVTVSS

SEQ ID NO: 783 - protein, 121 aa, synthetic construct
  source 1..121: note = 1A7[CD28]_H1.39
EVQLLESGGGLVQPGGSLRLSCAASGFTFSSYAMSWVRQAPGKGLEWVSTISGSGTYTYYADSVKGRFTISRDNSKNTLYLQMNSLRAEDTAVYYCAKSGPGLRQVGFDYWGQGTLVTVSS

SEQ ID NO: 784 - protein, 121 aa, synthetic construct
  source 1..121: note = 1A7[CD28]_H1.40
EVQLLESGGGLVQPGGSLRLSCAASGFTFSSYAMSWVRQAPGKGLEWVSTISGSGYYTYYADSVKGRFTISRDNSKNTLYLQMNSLRAEDTAVYYCAKSGPGLRQVGFDYWGQGTLVTVSS

SEQ ID NO: 785 - protein, 121 aa, synthetic construct
  source 1..121: note = 1A7[CD28]_H1.41
EVQLLESGGGLVQPGGSLRLSCAASGFTFSSYYMSWVRQAPGKGLEWVSTIEGSGDYTYYADSVKGRFTISRDNSKNTLYLQMNSLRAEDTAVYYCAKSGPGLRQVGFDYWGQGTLVTVSS

SEQ ID NO: 786 - protein, 121 aa, synthetic construct
  source 1..121: note = 1A7[CD28]_H1.42
EVQLLESGGGLVQPGGSLRLSCAASGFTFSSYYMSWVRQAPGKGLEWVSTISESGDYTYYADSVKGRFTISRDNSKNTLYLQMNSLRAEDTAVYYCAKSGPGLRQVGFDYWGQGTLVTVSS

SEQ ID NO: 787 - protein, 121 aa, synthetic construct
  source 1..121: note = 1A7[CD28]_H1.43
EVQLLESGGGLVQPGGSLRLSCAASGFSFSGNYMTWVRQAPGKGLEWVATITADSDATYYADSVKGRFTISRDNSKNTLYLQMNSLRAEDTAVYYCAKSGPGLRQVGFDYWGQGTLVTVSS

SEQ ID NO: 788 - protein, 121 aa, synthetic construct
  source 1..121: note = 1A7[CD28]_H1.44
EVQLLESGGGLVQPGGSLRLSCAASGFTFSGYSMNWVRQAPGKGLEWVATIYANGSYTYYADSVKGRFTISRDNSKNTLYLQMNSLRAEDTAVYYCAKSGPGLRQVGFDYWGQGTLVTVSS

SEQ ID NO: 789 - protein, 121 aa, synthetic construct
  source 1..121: note = 1A7[CD28]_H1.45
EVQLLESGGGLVQPGGSLRLSCAASGFNFEEYSMNWVRQAPGKGLEWVATITYNGDYTYYADSVKGRFTISRDNSKNTLYLQMNSLRAEDTAVYYCAKSGPGLRQVGFDYWGQGTLVTVSS

SEQ ID NO: 790 - protein, 121 aa, synthetic construct
  source 1..121: note = 1A7[CD28]_H1.46
EVQLLESGGGLVQPGGSLRLSCAASGFSFRTYYMTWVRQAPGKGLEWVATITSDGDYTYYADSVKGRFTISRDNSKNTLYLQMNSLRAEDTAVYYCAKSGPGLRQVGFDYWGQGTLVTVSS

SEQ ID NO: 791 - protein, 121 aa, synthetic construct
  source 1..121: note = 1A7[CD28]_H1.47
EVQLLESGGGLVQPGGSLRLSCAASGFSFKGYSMNWVRQAPGKGLEWVATIYASSDSTYYADSVKGRFTISRDNSKNTLYLQMNSLRAEDTAVYYCAKSGPGLRQVGFDYWGQGTLVTVSS

SEQ ID NO: 792 - protein, 121 aa, synthetic construct
  source 1..121: note = 1A7[CD28]_H1.48
EVQLLESGGGLVQPGGSLRLSCAASGFSFGEYSMNWVRQAPGKGLEWVSTIYADGDYTYYADSVKGRFTISRDNSKNTLYLQMNSLRAEDTAVYYCAKSGPGLRQVGFDYWGQGTLVTVSS

SEQ ID NO: 793 - protein, 121 aa, synthetic construct
  source 1..121: note = 1A7[CD28]_H1.49
EVQLLESGGGLVQPGGSLRLSCAASGFTFTDYSMNWVRQAPGKGLEWVATIYADSSSTYYADSVKGRFTISRDNSKNTLYLQMNSLRAEDTAVYYCAKSGPGLRQVGFDYWGQGTLVTVSS

SEQ ID NO: 794 - protein, 121 aa, synthetic construct
  source 1..121: note = 1A7[CD28]_H1.50
EVQLLESGGGLVQPGGSLRLSCAASGFTFGGYSMNWVRQAPGKGLEWVATIYYDSTTTYYADSVKGRFTISRDNSKNTLYLQMNSLRAEDTAVYYCAKSGPGLRQVGFDYWGQGTLVTVSS

SEQ ID NO: 795 - protein, 121 aa, synthetic construct
  source 1..121: note = 1A7[CD28]_H1.51
EVQLLESGGGLVQPGGSLRLSCAASGFTFGAYSMNWVRQAPGKGLEWVSTIYNDGATTYYADSVKGRFTISRDNSKNTLYLQMNSLRAEDTAVYYCAKSGPGLRQVGFDYWGQGTLVTVSS

SEQ ID NO: 796 - protein, 121 aa, synthetic construct
  source 1..121: note = 1A7[CD28]_H1.52
EVQLLESGGGLVQPGGSLRLSCAASGFSFEAYSMNWVRQAPGKGLEWVATIYYDSSYTYYADSVKGRFTISRDNSKNTLYLQMNSLRAEDTAVYYCAKSGPGLRQVGFDYWGQGTLVTVSS

SEQ ID NO: 797 - protein, 121 aa, synthetic construct
  source 1..121: note = 1A7[CD28]_H1.53
EVQLLESGGGLVQPGGSLRLSCAASGFSFGSYYMSWVRQAPGKGLEWVASIYYGGYDTYYADSVKGRFTISRDNSKNTLYLQMNSLRAEDTAVYYCAKSGPGLRQVGFDYWGQGTLVTVSS

SEQ ID NO: 798 - protein, 121 aa, synthetic construct
  source 1..121: note = 1A7[CD28]_H1.54
EVQLLESGGGLVQPGGSLRLSCAASGFNFAEYSMSWVRQAPGKGLEWVATIYAGSDYTYYADSVKGRFTISRDNSKNTLYLQMNSLRAEDTAVYYCAKSGPGLRQVGFDYWGQGTLVTVSS

SEQ ID NO: 799 - protein, 121 aa, synthetic construct
  source 1..121: note = 1A7[CD28]_H1.55
EVQLLESGGGLVQPGGSLRLSCAASGFTFGAYSINWVRQAPGKGLEWVATIYYDGSYTYYADSVKGRFTISRDNSKNTLYLQMNSLRAEDTAVYYCAKSGPGLRQVGFDYWGQGTLVTVSS

SEQ ID NO: 800 - protein, 121 aa, synthetic construct
  source 1..121: note = 1A7[CD28]_H1.56
EVQLLESGGGLVQPGGSLRLSCAASGFTFSRYYINWVRQAPGKGLEWVASIYDGGADTYYADSVKGRFTISRDNSKNTLYLQMNSLRAEDTAVYYCAKSGPGLRQVGFDYWGQGTLVTVSS

SEQ ID NO: 801 - protein, 121 aa, synthetic construct
  source 1..121: note = 1A7[CD28]_H1.57
EVQLLESGGGLVQPGGSLRLSCAASGFSFGTYSINWVRQAPGKGLEWVSTIYYDGASTYYADSVKGRFTISRDNSKNTLYLQMNSLRAEDTAVYYCAKSGPGLRQVGFDYWGQGTLVTVSS

SEQ ID NO: 802 - protein, 121 aa, synthetic construct
  source 1..121: note = 1A7[CD28]_H1.58
EVQLLESGGGLVQPGGSLRLSCAASGFTFGKYSISWVRQAPGKGLEWVATIYNDGYYTYYADSVKGRFTISRDNSKNTLYLQMNSLRAEDTAVYYCAKSGPGLRQVGFDYWGQGTLVTVSS

SEQ ID NO: 803 - protein, 121 aa, synthetic construct
  source 1..121: note = 1A7[CD28]_H1.59
EVQLLESGGGLVQPGGSLRLSCAASGFSFEKYYISWVRQAPGKGLEWVASIYDGSYDTYYADSVKGRFTISRDNSKNTLYLQMNSLRAEDTAVYYCAKSGPGLRQVGFDYWGQGTLVTVSS

SEQ ID NO: 804 - protein, 121 aa, synthetic construct
  source 1..121: note = 1A7[CD28]_H1.60
EVQLLESGGGLVQPGGSLRLSCAASGFSFGTYSMNWVRQAPGKGLEWVATIDYDGSNTYYADSVKGRFTISRDNSKNTLYLQMNSLRAEDTAVYYCAKSGPGLRQVGFDYWGQGTLVTVSS

SEQ ID NO: 805 - protein, 121 aa, synthetic construct
  source 1..121: note = 1A7[CD28]_H1.61
EVQLLESGGGLVQPGGSLRLSCAASGFSFAAYSMSWVRQAPGKGLEWVATIYAGSDYTYYADSVKGRFTISRDNSKNTLYLQMNSLRAEDTAVYYCAKSGPGLRQVGFDYWGQGTLVTVSS

SEQ ID NO: 806 - protein, 121 aa, synthetic construct
  source 1..121: note = 1A7[CD28]_H1.62
EVQLLESGGGLVQPGGSLRLSCAASGFTFTTYSMTWVRQAPGKGLEWVATIYNDGYYTYYADSVKGRFTISRDNSKNTLYLQMNSLRAEDTAVYYCAKSGPGLRQVGFDYWGQGTLVTVSS

SEQ ID NO: 807 - protein, 121 aa, synthetic construct
  source 1..121: note = 1A7[CD28]_H1.63
EVQLLESGGGLVQPGGSLRLSCAASGFSFKDYSMNWVRQAPGKGLEWVATIYYDGTYTYYADSVKGRFTISRDNSKNTLYLQMNSLRAEDTAVYYCAKSGPGLRQVGFDYWGQGTLVTVSS

SEQ ID NO: 808 - protein, 121 aa, synthetic construct
  source 1..121: note = 1A7[CD28]_H1.64
EVQLLESGGGLVQPGGSLRLSCAASGFSFAAYSMNWVRQAPGKGLEWVATIYAGGAYTYYADSVKGRFTISRDNSKNTLYLQMNSLRAEDTAVYYCAKSGPGLRQVGFDYWGQGTLVTVSS

SEQ ID NO: 809 - protein, 121 aa, synthetic construct
  source 1..121: note = 1A7[CD28]_H1.65
EVQLLESGGGLVQPGGSLRLSCAASGFNFAEYYISWVRQAPGKGLEWVASIYNGGSDTYYADSVKGRFTISRDNSKNTLYLQMNSLRAEDTAVYYCAKSGPGLRQVGFDYWGQGTLVTVSS

SEQ ID NO: 810 - protein, 121 aa, synthetic construct
  source 1..121: note = 1A7[CD28]_H1.66
EVQLLESGGGLVQPGGSLRLSCAASGFSLGEYYMNWVRQAPGKGLEWVSTIDYDGTYTYYADSVKGRFTISRDNSKNTLYLQMNSLRAEDTAVYYCAKSGPGLRQVGFDYWGQGTLVTVSS

SEQ ID NO: 811 - protein, 121 aa, synthetic construct
  source 1..121: note = 1A7[CD28]_H1.67
EVQLLESGGGLVQPGGSLRLSCAASGFTFNEYYITWVRQAPGKGLEWVSSIYSSSYDTYYADSVKGRFTISRDNSKNTLYLQMNSLRAEDTAVYYCAKSGPGLRQVGFDYWGQGTLVTVSS

SEQ ID NO: 812 - protein, 121 aa, synthetic construct
  source 1..121: note = 1A7[CD28]_H1.68
EVQLLESGGGLVQPGGSLRLSCAASGFTFKDYYITWVRQAPGKGLEWVSSIYTSGYDTYYADSVKGRFTISRDNSKNTLYLQMNSLRAEDTAVYYCAKSGPGLRQVGFDYWGQGTLVTVSS

SEQ ID NO: 813 - protein, 121 aa, synthetic construct
  source 1..121: note = 1A7[CD28]_H1.69
EVQLLESGGGLVQPGGSLRLSCAASGFSFGKYSINWVRQAPGKGLEWVATIYSDGTDTYYADSVKGRFTISRDNSKNTLYLQMNSLRAEDTAVYYCAKSGPGLRQVGFDYWGQGTLVTVSS

SEQ ID NO: 814 - protein, 107 aa, synthetic construct
  source 1..107: note = 1A7[CD28]_L1.1
DIQMTQSPSSLSASVGDRVTITCRASQSISAYLNWYQQKPGKAPKLLIYAASSLQSGVPSRFSGSGSGTDFTLTISSLQPEDFATYYCQQSYSTPFTFGQGTKLEIK

SEQ ID NO: 815 - protein, 107 aa, synthetic construct
  source 1..107: note = 1A7[CD28]_L1.2
DIQMTQSPSSLSASVGDRVTITCRASQSISDYLNWYQQKPGKAPKLLIYAASSLQSGVPSRFSGSGSGTDFTLTISSLQPEDFATYYCQQSYSTPFTFGQGTKLEIK

SEQ ID NO: 816 - protein, 107 aa, synthetic construct
  source 1..107: note = 1A7[CD28]_L1.3
DIQMTQSPSSLSASVGDRVTITCRASQSISGYLNWYQQKPGKAPKLLIYAASSLQSGVPSRFSGSGSGTDFTLTISSLQPEDFATYYCQQSYSTPFTFGQGTKLEIK

SEQ ID NO: 817 - protein, 107 aa, synthetic construct
  source 1..107: note = 1A7[CD28]_L1.4
DIQMTQSPSSLSASVGDRVTITCRASQSISHYLNWYQQKPGKAPKLLIYAASSLQSGVPSRFSGSGSGTDFTLTISSLQPEDFATYYCQQSYSTPFTFGQGTKLEIK

SEQ ID NO: 818 - protein, 107 aa, synthetic construct
  source 1..107: note = 1A7[CD28]_L1.5
DIQMTQSPSSLSASVGDRVTITCRASQSISKYLNWYQQKPGKAPKLLIYAASSLQSGVPSRFSGSGSGTDFTLTISSLQPEDFATYYCQQSYSTPFTFGQGTKLEIK

SEQ ID NO: 819 - protein, 107 aa, synthetic construct
  source 1..107: note = 1A7[CD28]_L1.6
DIQMTQSPSSLSASVGDRVTITCRASQSISNYLNWYQQKPGKAPKLLIYAASSLQSGVPSRFSGSGSGTDFTLTISSLQPEDFATYYCQQSYSTPFTFGQGTKLEIK

SEQ ID NO: 820 - protein, 107 aa, synthetic construct
  source 1..107: note = 1A7[CD28]_L1.7
DIQMTQSPSSLSASVGDRVTITCRASQSISQYLNWYQQKPGKAPKLLIYAASSLQSGVPSRFSGSGSGTDFTLTISSLQPEDFATYYCQQSYSTPFTFGQGTKLEIK

SEQ ID NO: 821 - protein, 107 aa, synthetic construct
  source 1..107: note = 1A7[CD28]_L1.8
DIQMTQSPSSLSASVGDRVTITCRASQSISTYLNWYQQKPGKAPKLLIYAASSLQSGVPSRFSGSGSGTDFTLTISSLQPEDFATYYCQQSYSTPFTFGQGTKLEIK

SEQ ID NO: 822 - protein, 107 aa, synthetic construct
  source 1..107: note = 1A7[CD28]_L1.9
DIQMTQSPSSLSASVGDRVTITCRASQSISVYLNWYQQKPGKAPKLLIYAASSLQSGVPSRFSGSGSGTDFTLTISSLQPEDFATYYCQQSYSTPFTFGQGTKLEIK

SEQ ID NO: 823 - protein, 107 aa, synthetic construct
  source 1..107: note = 1A7[CD28]_L1.10
DIQMTQSPSSLSASVGDRVTITCRASQSISYYLNWYQQKPGKAPKLLIYAASSLQSGVPSRFSGSGSGTDFTLTISSLQPEDFATYYCQQSYSTPFTFGQGTKLEIK

SEQ ID NO: 824 - protein, 107 aa, synthetic construct
  source 1..107: note = 1A7[CD28]_L1.11
DIQMTQSPSSLSASVGDRVTITCRASQSISSALNWYQQKPGKAPKLLIYAASSLQSGVPSRFSGSGSGTDFTLTISSLQPEDFATYYCQQSYSTPFTFGQGTKLEIK

SEQ ID NO: 825 - protein, 107 aa, synthetic construct
  source 1..107: note = 1A7[CD28]_L1.12
DIQMTQSPSSLSASVGDRVTITCRASQSISSDLNWYQQKPGKAPKLLIYAASSLQSGVPSRFSGSGSGTDFTLTISSLQPEDFATYYCQQSYSTPFTFGQGTKLEIK

SEQ ID NO: 826 - protein, 107 aa, synthetic construct
  source 1..107: note = 1A7[CD28]_L1.13
DIQMTQSPSSLSASVGDRVTITCRASQSISSFLNWYQQKPGKAPKLLIYAASSLQSGVPSRFSGSGSGTDFTLTISSLQPEDFATYYCQQSYSTPFTFGQGTKLEIK

SEQ ID NO: 827 - protein, 107 aa, synthetic construct
  source 1..107: note = 1A7[CD28]_L1.14
DIQMTQSPSSLSASVGDRVTITCRASQSISSHLNWYQQKPGKAPKLLIYAASSLQSGVPSRFSGSGSGTDFTLTISSLQPEDFATYYCQQSYSTPFTFGQGTKLEIK

SEQ ID NO: 828 - protein, 107 aa, synthetic construct
  source 1..107: note = 1A7[CD28]_L1.15
DIQMTQSPSSLSASVGDRVTITCRASQSISSKLNWYQQKPGKAPKLLIYAASSLQSGVPSRFSGSGSGTDFTLTISSLQPEDFATYYCQQSYSTPFTFGQGTKLEIK

SEQ ID NO: 829 - protein, 107 aa, synthetic construct
  source 1..107: note = 1A7[CD28]_L1.16
DIQMTQSPSSLSASVGDRVTITCRASQSISSLLNWYQQKPGKAPKLLIYAASSLQSGVPSRFSGSGSGTDFTLTISSLQPEDFATYYCQQSYSTPFTFGQGTKLEIK

SEQ ID NO: 830 - protein, 107 aa, synthetic construct
  source 1..107: note = 1A7[CD28]_L1.17
DIQMTQSPSSLSASVGDRVTITCRASQSISSNLNWYQQKPGKAPKLLIYAASSLQSGVPSRFSGSGSGTDFTLTISSLQPEDFATYYCQQSYSTPFTFGQGTKLEIK

SEQ ID NO: 831 - protein, 107 aa, synthetic construct
  source 1..107: note = 1A7[CD28]_L1.18
DIQMTQSPSSLSASVGDRVTITCRASQSISSQLNWYQQKPGKAPKLLIYAASSLQSGVPSRFSGSGSGTDFTLTISSLQPEDFATYYCQQSYSTPFTFGQGTKLEIK

SEQ ID NO: 832 - protein, 107 aa, synthetic construct
  source 1..107: note = 1A7[CD28]_L1.19
DIQMTQSPSSLSASVGDRVTITCRASQSISSSLNWYQQKPGKAPKLLIYAASSLQSGVPSRFSGSGSGTDFTLTISSLQPEDFATYYCQQSYSTPFTFGQGTKLEIK

SEQ ID NO: 833 - protein, 107 aa, synthetic construct
  source 1..107: note = 1A7[CD28]_L1.20
DIQMTQSPSSLSASVGDRVTITCRASQSISSWLNWYQQKPGKAPKLLIYAASSLQSGVPSRFSGSGSGTDFTLTISSLQPEDFATYYCQQSYSTPFTFGQGTKLEIK

SEQ ID NO: 834 - protein, 107 aa, synthetic construct
  source 1..107: note = 1A7[CD28]_L1.21
DIQMTQSPSSLSASVGDRVTITCRASQSISSYLAWYQQKPGKAPKLLIYAASSLQSGVPSRFSGSGSGTDFTLTISSLQPEDFATYYCQQSYSTPFTFGQGTKLEIK

SEQ ID NO: 835 - protein, 107 aa, synthetic construct
  source 1..107: note = 1A7[CD28]_L1.22
DIQMTQSPSSLSASVGDRVTITCRASQSISSYLDWYQQKPGKAPKLLIYAASSLQSGVPSRFSGSGSGTDFTLTISSLQPEDFATYYCQQSYSTPFTFGQGTKLEIK

SEQ ID NO: 836 - protein, 107 aa, synthetic construct
  source 1..107: note = 1A7[CD28]_L1.23
DIQMTQSPSSLSASVGDRVTITCRASQSISSYLGWYQQKPGKAPKLLIYAASSLQSGVPSRFSGSGSGTDFTLTISSLQPEDFATYYCQQSYSTPFTFGQGTKLEIK

SEQ ID NO: 837 - protein, 107 aa, synthetic construct
  source 1..107: note = 1A7[CD28]_L1.24
DIQMTQSPSSLSASVGDRVTITCRASQSISSYLHWYQQKPGKAPKLLIYAASSLQSGVPSRFSGSGSGTDFTLTISSLQPEDFATYYCQQSYSTPFTFGQGTKLEIK

SEQ ID NO: 838 - protein, 107 aa, synthetic construct
  source 1..107: note = 1A7[CD28]_L1.25
DIQMTQSPSSLSASVGDRVTITCRASQSISSYLQWYQQKPGKAPKLLIYAASSLQSGVPSRFSGSGSGTDFTLTISSLQPEDFATYYCQQSYSTPFTFGQGTKLEIK

SEQ ID NO: 839 - protein, 107 aa, synthetic construct
  source 1..107: note = 1A7[CD28]_L1.26
DIQMTQSPSSLSASVGDRVTITCRASQSISSYLSWYQQKPGKAPKLLIYAASSLQSGVPSRFSGSGSGTDFTLTISSLQPEDFATYYCQQSYSTPFTFGQGTKLEIK

SEQ ID NO: 840 - protein, 107 aa, synthetic construct
  source 1..107: note = 1A7[CD28]_L1.27
DIQMTQSPSSLSASVGDRVTITCRASQSISSYLTWYQQKPGKAPKLLIYAASSLQSGVPSRFSGSGSGTDFTLTISSLQPEDFATYYCQQSYSTPFTFGQGTKLEIK

SEQ ID NO: 841 - protein, 107 aa, synthetic construct
  source 1..107: note = 1A7[CD28]_L1.28
DIQMTQSPSSLSASVGDRVTITCRASQSISSYLYWYQQKPGKAPKLLIYAASSLQSGVPSRFSGSGSGTDFTLTISSLQPEDFATYYCQQSYSTPFTFGQGTKLEIK

SEQ ID NO: 842 - protein, 107 aa, synthetic construct
  source 1..107: note = 1A7[CD28]_L1.29
DIQMTQSPSSLSASVGDRVTITCRASQSISSYLNWYQQKPGKAPKLLIYDASSLQSGVPSRFSGSGSGTDFTLTISSLQPEDFATYYCQQSYSTPFTFGQGTKLEIK

SEQ ID NO: 843 - protein, 107 aa, synthetic construct
  source 1..107: note = 1A7[CD28]_L1.30
DIQMTQSPSSLSASVGDRVTITCRASQSISSYLNWYQQKPGKAPKLLIYGASSLQSGVPSRFSGSGSGTDFTLTISSLQPEDFATYYCQQSYSTPFTFGQGTKLEIK

SEQ ID NO: 844 - protein, 107 aa, synthetic construct
  source 1..107: note = 1A7[CD28]_L1.31
DIQMTQSPSSLSASVGDRVTITCRASQSISSYLNWYQQKPGKAPKLLIYKASSLQSGVPSRFSGSGSGTDFTLTISSLQPEDFATYYCQQSYSTPFTFGQGTKLEIK

SEQ ID NO: 845 - protein, 107 aa, synthetic construct
  source 1..107: note = 1A7[CD28]_L1.32
DIQMTQSPSSLSASVGDRVTITCRASQSISSYLNWYQQKPGKAPKLLIYLASSLQSGVPSRFSGSGSGTDFTLTISSLQPEDFATYYCQQSYSTPFTFGQGTKLEIK

SEQ ID NO: 846 - protein, 107 aa, synthetic construct
  source 1..107: note = 1A7[CD28]_L1.33
DIQMTQSPSSLSASVGDRVTITCRASQSISSYLNWYQQKPGKAPKLLIYQASSLQSGVPSRFSGSGSGTDFTLTISSLQPEDFATYYCQQSYSTPFTFGQGTKLEIK

SEQ ID NO: 847 - protein, 107 aa, synthetic construct
  source 1..107: note = 1A7[CD28]_L1.34
DIQMTQSPSSLSASVGDRVTITCRASQSISSYLNWYQQKPGKAPKLLIYSASSLQSGVPSRFSGSGSGTDFTLTISSLQPEDFATYYCQQSYSTPFTFGQGTKLEIK

SEQ ID NO: 848 - protein, 107 aa, synthetic construct
  source 1..107: note = 1A7[CD28]_L1.35
DIQMTQSPSSLSASVGDRVTITCRASQSISSYLNWYQQKPGKAPKLLIYTASSLQSGVPSRFSGSGSGTDFTLTISSLQPEDFATYYCQQSYSTPFTFGQGTKLEIK

SEQ ID NO: 849 - protein, 107 aa, synthetic construct
  source 1..107: note = 1A7[CD28]_L1.36
DIQMTQSPSSLSASVGDRVTITCRASQSISSYLNWYQQKPGKAPKLLIYWASSLQSGVPSRFSGSGSGTDFTLTISSLQPEDFATYYCQQSYSTPFTFGQGTKLEIK

SEQ ID NO: 850 - protein, 107 aa, synthetic construct
  source 1..107: note = 1A7[CD28]_L1.37
DIQMTQSPSSLSASVGDRVTITCRASQSISSYLNWYQQKPGKAPKLLIYYASSLQSGVPSRFSGSGSGTDFTLTISSLQPEDFATYYCQQSYSTPFTFGQGTKLEIK

SEQ ID NO: 851 - protein, 107 aa, synthetic construct
  source 1..107: note = 1A7[CD28]_L1.38
DIQMTQSPSSLSASVGDRVTITCRASQSISSYLNWYQQKPGKAPKLLIYAASALQSGVPSRFSGSGSGTDFTLTISSLQPEDFATYYCQQSYSTPFTFGQGTKLEIK

SEQ ID NO: 852 - protein, 107 aa, synthetic construct
  source 1..107: note = 1A7[CD28]_L1.39
DIQMTQSPSSLSASVGDRVTITCRASQSISSYLNWYQQKPGKAPKLLIYAASDLQSGVPSRFSGSGSGTDFTLTISSLQPEDFATYYCQQSYSTPFTFGQGTKLEIK

SEQ ID NO: 853 - protein, 107 aa, synthetic construct
  source 1..107: note = 1A7[CD28]_L1.40
DIQMTQSPSSLSASVGDRVTITCRASQSISSYLNWYQQKPGKAPKLLIYAASKLQSGVPSRFSGSGSGTDFTLTISSLQPEDFATYYCQQSYSTPFTFGQGTKLEIK

SEQ ID NO: 854 - protein, 107 aa, synthetic construct
  source 1..107: note = 1A7[CD28]_L1.41
DIQMTQSPSSLSASVGDRVTITCRASQSISSYLNWYQQKPGKAPKLLIYAASNLQSGVPSRFSGSGSGTDFTLTISSLQPEDFATYYCQQSYSTPFTFGQGTKLEIK

SEQ ID NO: 855 - protein, 107 aa, synthetic construct
  source 1..107: note = 1A7[CD28]_L1.42
DIQMTQSPSSLSASVGDRVTITCRASQSISSYLNWYQQKPGKAPKLLIYAASQLQSGVPSRFSGSGSGTDFTLTISSLQPEDFATYYCQQSYSTPFTFGQGTKLEIK

SEQ ID NO: 856 - protein, 107 aa, synthetic construct
  source 1..107: note = 1A7[CD28]_L1.43
DIQMTQSPSSLSASVGDRVTITCRASQSISSYLNWYQQKPGKAPKLLIYAASTLQSGVPSRFSGSGSGTDFTLTISSLQPEDFATYYCQQSYSTPFTFGQGTKLEIK

SEQ ID NO: 857 - protein, 107 aa, synthetic construct
  source 1..107: note = 1A7[CD28]_L1.44
DIQMTQSPSSLSASVGDRVTITCRASQSISSYLNWYQQKPGKAPKLLIYAASYLQSGVPSRFSGSGSGTDFTLTISSLQPEDFATYYCQQSYSTPFTFGQGTKLEIK

SEQ ID NO: 858 - protein, 107 aa, synthetic construct
  source 1..107: note = 1A7[CD28]_L1.45
DIQMTQSPSSLSASVGDRVTITCRASQSISSYLNWYQQKPGKAPKLLIYAASSLASGVPSRFSGSGSGTDFTLTISSLQPEDFATYYCQQSYSTPFTFGQGTKLEIK

SEQ ID NO: 859 - protein, 107 aa, synthetic construct
  source 1..107: note = 1A7[CD28]_L1.46
DIQMTQSPSSLSASVGDRVTITCRASQSISSYLNWYQQKPGKAPKLLIYAASSLESGVPSRFSGSGSGTDFTLTISSLQPEDFATYYCQQSYSTPFTFGQGTKLEIK

SEQ ID NO: 860 - protein, 107 aa, synthetic construct
  source 1..107: note = 1A7[CD28]_L1.47
DIQMTQSPSSLSASVGDRVTITCRASQSISSYLNWYQQKPGKAPKLLIYAASSLFSGVPSRFSGSGSGTDFTLTISSLQPEDFATYYCQQSYSTPFTFGQGTKLEIK

SEQ ID NO: 861 - protein, 107 aa, synthetic construct
  source 1..107: note = 1A7[CD28]_L1.48
DIQMTQSPSSLSASVGDRVTITCRASQSISSYLNWYQQKPGKAPKLLIYAASSLHSGVPSRFSGSGSGTDFTLTISSLQPEDFATYYCQQSYSTPFTFGQGTKLEIK

SEQ ID NO: 862 - protein, 107 aa, synthetic construct
  source 1..107: note = 1A7[CD28]_L1.49
DIQMTQSPSSLSASVGDRVTITCRASQSISSYLNWYQQKPGKAPKLLIYAASSLISGVPSRFSGSGSGTDFTLTISSLQPEDFATYYCQQSYSTPFTFGQGTKLEIK

SEQ ID NO: 863 - protein, 107 aa, synthetic construct
  source 1..107: note = 1A7[CD28]_L1.50
DIQMTQSPSSLSASVGDRVTITCRASQSISSYLNWYQQKPGKAPKLLIYAASSLKSGVPSRFSGSGSGTDFTLTISSLQPEDFATYYCQQSYSTPFTFGQGTKLEIK

SEQ ID NO: 864 - protein, 107 aa, synthetic construct
  source 1..107: note = 1A7[CD28]_L1.51
DIQMTQSPSSLSASVGDRVTITCRASQSISSYLNWYQQKPGKAPKLLIYAASSLNSGVPSRFSGSGSGTDFTLTISSLQPEDFATYYCQQSYSTPFTFGQGTKLEIK

SEQ ID NO: 865 - protein, 107 aa, synthetic construct
  source 1..107: note = 1A7[CD28]_L1.52
DIQMTQSPSSLSASVGDRVTITCRASQSISSYLNWYQQKPGKAPKLLIYAASSLSSGVPSRFSGSGSGTDFTLTISSLQPEDFATYYCQQSYSTPFTFGQGTKLEIK

SEQ ID NO: 866 - protein, 107 aa, synthetic construct
  source 1..107: note = 1A7[CD28]_L1.53
DIQMTQSPSSLSASVGDRVTITCRASQSISSYLNWYQQKPGKAPKLLIYAASSLVSGVPSRFSGSGSGTDFTLTISSLQPEDFATYYCQQSYSTPFTFGQGTKLEIK

SEQ ID NO: 867 - protein, 107 aa, synthetic construct
  source 1..107: note = 1A7[CD28]_L1.54
DIQMTQSPSSLSASVGDRVTITCRASQSISSYLNWYQQKPGKAPKLLIYAASSLYSGVPSRFSGSGSGTDFTLTISSLQPEDFATYYCQQSYSTPFTFGQGTKLEIK

SEQ ID NO: 868 - protein, 107 aa, synthetic construct
  source 1..107: note = 1A7[CD28]_L1.55
DIQMTQSPSSLSASVGDRVTITCRASQSISSYLNWYQQKPGKAPKLLIYAASSLQAGVPSRFSGSGSGTDFTLTISSLQPEDFATYYCQQSYSTPFTFGQGTKLEIK

SEQ ID NO: 869 - protein, 107 aa, synthetic construct
  source 1..107: note = 1A7[CD28]_L1.56
DIQMTQSPSSLSASVGDRVTITCRASQSISSYLNWYQQKPGKAPKLLIYAASSLQDGVPSRFSGSGSGTDFTLTISSLQPEDFATYYCQQSYSTPFTFGQGTKLEIK

SEQ ID NO: 870 - protein, 107 aa, synthetic construct
  source 1..107: note = 1A7[CD28]_L1.57
DIQMTQSPSSLSASVGDRVTITCRASQSISSYLNWYQQKPGKAPKLLIYAASSLQGGVPSRFSGSGSGTDFTLTISSLQPEDFATYYCQQSYSTPFTFGQGTKLEIK

SEQ ID NO: 871 - protein, 107 aa, synthetic construct
  source 1..107: note = 1A7[CD28]_L1.58
DIQMTQSPSSLSASVGDRVTITCRASQSISSYLNWYQQKPGKAPKLLIYAASSLQHGVPSRFSGSGSGTDFTLTISSLQPEDFATYYCQQSYSTPFTFGQGTKLEIK

SEQ ID NO: 872 - protein, 107 aa, synthetic construct
  source 1..107: note = 1A7[CD28]_L1.59
DIQMTQSPSSLSASVGDRVTITCRASQSISSYLNWYQQKPGKAPKLLIYAASSLQKGVPSRFSGSGSGTDFTLTISSLQPEDFATYYCQQSYSTPFTFGQGTKLEIK

SEQ ID NO: 873 - protein, 107 aa, synthetic construct
  source 1..107: note = 1A7[CD28]_L1.60
DIQMTQSPSSLSASVGDRVTITCRASQSISSYLNWYQQKPGKAPKLLIYAASSLQQGVPSRFSGSGSGTDFTLTISSLQPEDFATYYCQQSYSTPFTFGQGTKLEIK

SEQ ID NO: 874 - protein, 107 aa, synthetic construct
  source 1..107: note = 1A7[CD28]_L1.61
DIQMTQSPSSLSASVGDRVTITCRASQSISSYLNWYQQKPGKAPKLLIYAASSLQTGVPSRFSGSGSGTDFTLTISSLQPEDFATYYCQQSYSTPFTFGQGTKLEIK

SEQ ID NO: 875 - protein, 107 aa, synthetic construct
  source 1..107: note = 1A7[CD28]_L1.62
DIQMTQSPSSLSASVGDRVTITCRASQSISSYLNWYQQKPGKAPKLLIYAASSLQVGVPSRFSGSGSGTDFTLTISSLQPEDFATYYCQQSYSTPFTFGQGTKLEIK

SEQ ID NO: 876 - protein, 107 aa, synthetic construct
  source 1..107: note = 1A7[CD28]_L1.63
DIQMTQSPSSLSASVGDRVTITCRASQSISSYLNWYQQKPGKAPKLLIYAASSLQYGVPSRFSGSGSGTDFTLTISSLQPEDFATYYCQQSYSTPFTFGQGTKLEIK

SEQ ID NO: 877 - protein, 107 aa, synthetic construct
  source 1..107: note = 1A7[CD28]_L1.64
DIQMTQSPSSLSASVGDRVTITCRASQSISSYLNWYQQKPGKAPKLLIYAASSLQSGVPSRFSGSGSGTDFTLTISSLQPEDFATYYCQQAYSTPFTFGQGTKLEIK

SEQ ID NO: 878 - protein, 107 aa, synthetic construct
  source 1..107: note = 1A7[CD28]_L1.65
DIQMTQSPSSLSASVGDRVTITCRASQSISSYLNWYQQKPGKAPKLLIYAASSLQSGVPSRFSGSGSGTDFTLTISSLQPEDFATYYCQQDYSTPFTFGQGTKLEIK

SEQ ID NO: 879 - protein, 107 aa, synthetic construct
  source 1..107: note = 1A7[CD28]_L1.66
DIQMTQSPSSLSASVGDRVTITCRASQSISSYLNWYQQKPGKAPKLLIYAASSLQSGVPSRFSGSGSGTDFTLTISSLQPEDFATYYCQQFYSTPFTFGQGTKLEIK

SEQ ID NO: 880 - protein, 107 aa, synthetic construct
  source 1..107: note = 1A7[CD28]_L1.67
DIQMTQSPSSLSASVGDRVTITCRASQSISSYLNWYQQKPGKAPKLLIYAASSLQSGVPSRFSGSGSGTDFTLTISSLQPEDFATYYCQQHYSTPFTFGQGTKLEIK

SEQ ID NO: 881 - protein, 107 aa, synthetic construct
  source 1..107: note = 1A7[CD28]_L1.68
DIQMTQSPSSLSASVGDRVTITCRASQSISSYLNWYQQKPGKAPKLLIYAASSLQSGVPSRFSGSGSGTDFTLTISSLQPEDFATYYCQQKYSTPFTFGQGTKLEIK

SEQ ID NO: 882 - protein, 107 aa, synthetic construct
  source 1..107: note = 1A7[CD28]_L1.69
DIQMTQSPSSLSASVGDRVTITCRASQSISSYLNWYQQKPGKAPKLLIYAASSLQSGVPSRFSGSGSGTDFTLTISSLQPEDFATYYCQQLYSTPFTFGQGTKLEIK

SEQ ID NO: 883 - protein, 107 aa, synthetic construct
  source 1..107: note = 1A7[CD28]_L1.70
DIQMTQSPSSLSASVGDRVTITCRASQSISSYLNWYQQKPGKAPKLLIYAASSLQSGVPSRFSGSGSGTDFTLTISSLQPEDFATYYCQQTYSTPFTFGQGTKLEIK

SEQ ID NO: 884 - protein, 107 aa, synthetic construct
  source 1..107: note = 1A7[CD28]_L1.71
DIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIK

SEQ ID NO: 885 - protein, 107 aa, synthetic construct
  source 1..107: note = 1A7[CD28]_L1.72
DIQMTQSPSSLSASVGDRVTITCRASQSISSYLNWYQQKPGKAPKLLIYAASSLQSGVPSRFSGSGSGTDFTLTISSLQPEDFATYYCQQYYSTPFTFGQGTKLEIK

SEQ ID NO: 886 - protein, 107 aa, synthetic construct
  source 1..107: note = 1A7[CD28]_L1.73
DIQMTQSPSSLSASVGDRVTITCRASQSISSYLNWYQQKPGKAPKLLIYAASSLQSGVPSRFSGSGSGTDFTLTISSLQPEDFATYYCQQSASTPFTFGQGTKLEIK

SEQ ID NO: 887 - protein, 107 aa, synthetic construct
  source 1..107: note = 1A7[CD28]_L1.74
DIQMTQSPSSLSASVGDRVTITCRASQSISSYLNWYQQKPGKAPKLLIYAASSLQSGVPSRFSGSGSGTDFTLTISSLQPEDFATYYCQQSDSTPFTFGQGTKLEIK

SEQ ID NO: 888 - protein, 107 aa, synthetic construct
  source 1..107: note = 1A7[CD28]_L1.75
DIQMTQSPSSLSASVGDRVTITCRASQSISSYLNWYQQKPGKAPKLLIYAASSLQSGVPSRFSGSGSGTDFTLTISSLQPEDFATYYCQQSFSTPFTFGQGTKLEIK

SEQ ID NO: 889 - protein, 107 aa, synthetic construct
  source 1..107: note = 1A7[CD28]_L1.76
DIQMTQSPSSLSASVGDRVTITCRASQSISSYLNWYQQKPGKAPKLLIYAASSLQSGVPSRFSGSGSGTDFTLTISSLQPEDFATYYCQQSHSTPFTFGQGTKLEIK

SEQ ID NO: 890 - protein, 107 aa, synthetic construct
  source 1..107: note = 1A7[CD28]_L1.77
DIQMTQSPSSLSASVGDRVTITCRASQSISSYLNWYQQKPGKAPKLLIYAASSLQSGVPSRFSGSGSGTDFTLTISSLQPEDFATYYCQQSKSTPFTFGQGTKLEIK

SEQ ID NO: 891 - protein, 107 aa, synthetic construct
  source 1..107: note = 1A7[CD28]_L1.78
DIQMTQSPSSLSASVGDRVTITCRASQSISSYLNWYQQKPGKAPKLLIYAASSLQSGVPSRFSGSGSGTDFTLTISSLQPEDFATYYCQQSLSTPFTFGQGTKLEIK

SEQ ID NO: 892 - protein, 107 aa, synthetic construct
  source 1..107: note = 1A7[CD28]_L1.79
DIQMTQSPSSLSASVGDRVTITCRASQSISSYLNWYQQKPGKAPKLLIYAASSLQSGVPSRFSGSGSGTDFTLTISSLQPEDFATYYCQQSQSTPFTFGQGTKLEIK

SEQ ID NO: 893 - protein, 107 aa, synthetic construct
  source 1..107: note = 1A7[CD28]_L1.80
DIQMTQSPSSLSASVGDRVTITCRASQSISSYLNWYQQKPGKAPKLLIYAASSLQSGVPSRFSGSGSGTDFTLTISSLQPEDFATYYCQQSVSTPFTFGQGTKLEIK

SEQ ID NO: 894 - protein, 107 aa, synthetic construct
  source 1..107: note = 1A7[CD28]_L1.81
DIQMTQSPSSLSASVGDRVTITCRASQSISSYLNWYQQKPGKAPKLLIYAASSLQSGVPSRFSGSGSGTDFTLTISSLQPEDFATYYCQQSWSTPFTFGQGTKLEIK

SEQ ID NO: 895 - protein, 107 aa, synthetic construct
  source 1..107: note = 1A7[CD28]_L1.82
DIQMTQSPSSLSASVGDRVTITCRASQSISSYLNWYQQKPGKAPKLLIYAASSLQSGVPSRFSGSGSGTDFTLTISSLQPEDFATYYCQQSYATPFTFGQGTKLEIK

SEQ ID NO: 896 - protein, 107 aa, synthetic construct
  source 1..107: note = 1A7[CD28]_L1.83
DIQMTQSPSSLSASVGDRVTITCRASQSISSYLNWYQQKPGKAPKLLIYAASSLQSGVPSRFSGSGSGTDFTLTISSLQPEDFATYYCQQSYDTPFTFGQGTKLEIK

SEQ ID NO: 897 - protein, 107 aa, synthetic construct
  source 1..107: note = 1A7[CD28]_L1.84
DIQMTQSPSSLSASVGDRVTITCRASQSISSYLNWYQQKPGKAPKLLIYAASSLQSGVPSRFSGSGSGTDFTLTISSLQPEDFATYYCQQSYGTPFTFGQGTKLEIK

SEQ ID NO: 898 - protein, 107 aa, synthetic construct
  source 1..107: note = 1A7[CD28]_L1.85
DIQMTQSPSSLSASVGDRVTITCRASQSISSYLNWYQQKPGKAPKLLIYAASSLQSGVPSRFSGSGSGTDFTLTISSLQPEDFATYYCQQSYHTPFTFGQGTKLEIK

SEQ ID NO: 899 - protein, 107 aa, synthetic construct
  source 1..107: note = 1A7[CD28]_L1.86
DIQMTQSPSSLSASVGDRVTITCRASQSISSYLNWYQQKPGKAPKLLIYAASSLQSGVPSRFSGSGSGTDFTLTISSLQPEDFATYYCQQSYKTPFTFGQGTKLEIK

SEQ ID NO: 900 - protein, 107 aa, synthetic construct
  source 1..107: note = 1A7[CD28]_L1.87
DIQMTQSPSSLSASVGDRVTITCRASQSISSYLNWYQQKPGKAPKLLIYAASSLQSGVPSRFSGSGSGTDFTLTISSLQPEDFATYYCQQSYNTPFTFGQGTKLEIK

SEQ ID NO: 901 - protein, 107 aa, synthetic construct
  source 1..107: note = 1A7[CD28]_L1.88
DIQMTQSPSSLSASVGDRVTITCRASQSISSYLNWYQQKPGKAPKLLIYAASSLQSGVPSRFSGSGSGTDFTLTISSLQPEDFATYYCQQSYQTPFTFGQGTKLEIK

SEQ ID NO: 902 - protein, 107 aa, synthetic construct
  source 1..107: note = 1A7[CD28]_L1.89
DIQMTQSPSSLSASVGDRVTITCRASQSISSYLNWYQQKPGKAPKLLIYAASSLQSGVPSRFSGSGSGTDFTLTISSLQPEDFATYYCQQSYTTPFTFGQGTKLEIK

SEQ ID NO: 903 - protein, 107 aa, synthetic construct
  source 1..107: note = 1A7[CD28]_L1.90
DIQMTQSPSSLSASVGDRVTITCRASQSISSYLNWYQQKPGKAPKLLIYAASSLQSGVPSRFSGSGSGTDFTLTISSLQPEDFATYYCQQSYVTPFTFGQGTKLEIK

SEQ ID NO: 904 - protein, 107 aa, synthetic construct
  source 1..107: note = 1A7[CD28]_L1.91
DIQMTQSPSSLSASVGDRVTITCRASQSISSYLNWYQQKPGKAPKLLIYAASSLQSGVPSRFSGSGSGTDFTLTISSLQPEDFATYYCQQSYYTPFTFGQGTKLEIK

SEQ ID NO: 905 - protein, 107 aa, synthetic construct
  source 1..107: note = 1A7[CD28]_L1.92
DIQMTQSPSSLSASVGDRVTITCRASQSISSYLNWYQQKPGKAPKLLIYAASSLQSGVPSRFSGSGSGTDFTLTISSLQPEDFATYYCQQSYSAPFTFGQGTKLEIK

SEQ ID NO: 906 - protein, 107 aa, synthetic construct
  source 1..107: note = 1A7[CD28]_L1.93
DIQMTQSPSSLSASVGDRVTITCRASQSISSYLNWYQQKPGKAPKLLIYAASSLQSGVPSRFSGSGSGTDFTLTISSLQPEDFATYYCQQSYSDPFTFGQGTKLEIK

SEQ ID NO: 907 - protein, 107 aa, synthetic construct
  source 1..107: note = 1A7[CD28]_L1.94
DIQMTQSPSSLSASVGDRVTITCRASQSISSYLNWYQQKPGKAPKLLIYAASSLQSGVPSRFSGSGSGTDFTLTISSLQPEDFATYYCQQSYSFPFTFGQGTKLEIK

SEQ ID NO: 908 - protein, 107 aa, synthetic construct
  source 1..107: note = 1A7[CD28]_L1.95
DIQMTQSPSSLSASVGDRVTITCRASQSISSYLNWYQQKPGKAPKLLIYAASSLQSGVPSRFSGSGSGTDFTLTISSLQPEDFATYYCQQSYSIPFTFGQGTKLEIK

SEQ ID NO: 909 - protein, 107 aa, synthetic construct
  source 1..107: note = 1A7[CD28]_L1.96
DIQMTQSPSSLSASVGDRVTITCRASQSISSYLNWYQQKPGKAPKLLIYAASSLQSGVPSRFSGSGSGTDFTLTISSLQPEDFATYYCQQSYSKPFTFGQGTKLEIK

SEQ ID NO: 910 - protein, 107 aa, synthetic construct
  source 1..107: note = 1A7[CD28]_L1.97
DIQMTQSPSSLSASVGDRVTITCRASQSISSYLNWYQQKPGKAPKLLIYAASSLQSGVPSRFSGSGSGTDFTLTISSLQPEDFATYYCQQSYSLPFTFGQGTKLEIK

SEQ ID NO: 911 - protein, 107 aa, synthetic construct
  source 1..107: note = 1A7[CD28]_L1.98
DIQMTQSPSSLSASVGDRVTITCRASQSISSYLNWYQQKPGKAPKLLIYAASSLQSGVPSRFSGSGSGTDFTLTISSLQPEDFATYYCQQSYSQPFTFGQGTKLEIK

SEQ ID NO: 912 - protein, 107 aa, synthetic construct
  source 1..107: note = 1A7[CD28]_L1.99
DIQMTQSPSSLSASVGDRVTITCRASQSISSYLNWYQQKPGKAPKLLIYAASSLQSGVPSRFSGSGSGTDFTLTISSLQPEDFATYYCQQSYSSPFTFGQGTKLEIK

SEQ ID NO: 913 - protein, 107 aa, synthetic construct
  source 1..107: note = 1A7[CD28]_L1.100
DIQMTQSPSSLSASVGDRVTITCRASQSISSYLNWYQQKPGKAPKLLIYAASSLQSGVPSRFSGSGSGTDFTLTISSLQPEDFATYYCQQSYSVPFTFGQGTKLEIK

SEQ ID NO: 914 - protein, 107 aa, synthetic construct
  source 1..107: note = 1A7[CD28]_L1.101
DIQMTQSPSSLSASVGDRVTITCRASQSISSYLNWYQQKPGKAPKLLIYAASSLQSGVPSRFSGSGSGTDFTLTISSLQPEDFATYYCQQSYSYPFTFGQGTKLEIK

SEQ ID NO: 915 - protein, 107 aa, synthetic construct
  source 1..107: note = 1A7[CD28]_L1.102
DIQMTQSPSSLSASVGDRVTITCRASQSISSYLNWYQQKPGKAPKLLIYAASSLQSGVPSRFSGSGSGTDFTLTISSLQPEDFATYYCQQSYSTPITFGQGTKLEIK

SEQ ID NO: 916 - protein, 107 aa, synthetic construct
  source 1..107: note = 1A7[CD28]_L1.103
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKLEIK

SEQ ID NO: 917 - protein, 107 aa, synthetic construct
  source 1..107: note = 1A7[CD28]_L1.104
DIQMTQSPSSLSASVGDRVTITCRASQSISSYLNWYQQKPGKAPKLLIYAASSLQSGVPSRFSGSGSGTDFTLTISSLQPEDFATYYCQQSYSTPWTFGQGTKLEIK

SEQ ID NO: 918 - protein, 107 aa, synthetic construct
  source 1..107: note = 1A7[CD28]_L1.105
DIQMTQSPSSLSASVGDRVTITCRASQSISSYLTWYQQKPGKAPKLLIYAASSLQSGVPSRFSGSGSGTDFTLTISSLQPEDFATYYCQQVYSTPFTFGQGTKLEIK

SEQ ID NO: 919 - protein, 107 aa, synthetic construct
  source 1..107: note = 1A7[CD28]_L1.106
DIQMTQSPSSLSASVGDRVTITCRASQSISSYLTWYQQKPGKAPKLLIYAASSLQSGVPSRFSGSGSGTDFTLTISSLQPEDFATYYCQQSYNTPFTFGQGTKLEIK

SEQ ID NO: 920 - protein, 121 aa, synthetic construct; identical to SEQ ID NO: 745
  source 1..121: note = 1A7[CD28]_H1.1_L1 Variable Heavy (vh) Domain

SEQ ID NO: 921 - protein, 5 aa, synthetic construct
  source 1..5: note = 1A7[CD28]_H1.1_L1 vhCDR1
SYYMS

SEQ ID NO: 922 - protein, 17 aa, synthetic construct
  source 1..17: note = 1A7[CD28]_H1.1_L1 vhCDR2
TISGSGDSTYYADSVKG

SEQ ID NO: 923 - protein, 12 aa, synthetic construct
  source 1..12: note = 1A7[CD28]_H1.1_L1 vhCDR3
SGPGLRQVGFDY

SEQ ID NO: 924 - protein, 107 aa, synthetic construct; identical to SEQ ID NO: 741
  source 1..107: note = 1A7[CD28]_H1.1_L1 Variable Light (vl) Domain

SEQ ID NO: 925 - protein, 11 aa, synthetic construct
  source 1..11: note = 1A7[CD28]_H1.1_L1 vlCDR1
RASQSISSYLN

SEQ ID NO: 926 - protein, 7 aa, synthetic construct
  source 1..7: note = 1A7[CD28]_H1.1_L1 vlCDR2
AASSLQS

SEQ ID NO: 927 - protein, 9 aa, synthetic construct
  source 1..9: note = 1A7[CD28]_H1.1_L1 vlCDR3
QQSYSTPFT

SEQ ID NO: 928 - protein, 121 aa, synthetic construct; identical to SEQ ID NO: 737
  source 1..121: note = 1A7[CD28]_H1_L1.71 Variable Heavy (vh) Domain

SEQ ID NO: 929 - protein, 5 aa, synthetic construct
  source 1..5: note = 1A7[CD28]_H1_L1.71 vhCDR1
SYAMS

SEQ ID NO: 930 - protein, 17 aa, synthetic construct
  source 1..17: note = 1A7[CD28]_H1_L1.71 vhCDR2
TISGSGDSTYYADSVKG

SEQ ID NO: 931 - protein, 12 aa, synthetic construct
  source 1..12: note = 1A7[CD28]_H1_L1.71 vhCDR3
SGPGLRQVGFDY

SEQ ID NO: 932 - protein, 107 aa, synthetic construct; identical to SEQ ID NO: 884
  source 1..107: note = 1A7[CD28]_H1_L1.71 Variable Light (vl) Domain

SEQ ID NO: 933 - protein, 11 aa, synthetic construct
  source 1..11: note = 1A7[CD28]_H1_L1.71 vlCDR1
RASQSISSYLN

SEQ ID NO: 934 - protein, 7 aa, synthetic construct
  source 1..7: note = 1A7[CD28]_H1_L1.71 vlCDR2
AASSLQS

SEQ ID NO: 935 - protein, 9 aa, synthetic construct
  source 1..9: note = 1A7[CD28]_H1_L1.71 vlCDR3
QQVYSTPFT

SEQ ID NO: 936 - protein, 121 aa, synthetic construct; identical to SEQ ID NO: 745
  source 1..121: note = 1A7[CD28]_H1.1_L1.71 Variable Heavy (vh) Domain

SEQ ID NO: 937 - protein, 5 aa, synthetic construct
  source 1..5: note = 1A7[CD28]_H1.1_L1.71 vhCDR1
SYYMS

SEQ ID NO: 938 - protein, 17 aa, synthetic construct
  source 1..17: note = 1A7[CD28]_H1.1_L1.71 vhCDR2
TISGSGDSTYYADSVKG

SEQ ID NO: 939 - protein, 12 aa, synthetic construct
  source 1..12: note = 1A7[CD28]_H1.1_L1.71 vhCDR3
SGPGLRQVGFDY

SEQ ID NO: 940 - protein, 107 aa, synthetic construct; identical to SEQ ID NO: 884
  source 1..107: note = 1A7[CD28]_H1.1_L1.71 Variable Light (vl) Domain

SEQ ID NO: 941 - protein, 11 aa, synthetic construct
  source 1..11: note = 1A7[CD28]_H1.1_L1.71 vlCDR1
RASQSISSYLN

SEQ ID NO: 942 - protein, 7 aa, synthetic construct
  source 1..7: note = 1A7[CD28]_H1.1_L1.71 vlCDR2
AASSLQS

SEQ ID NO: 943 - protein, 9 aa, synthetic construct
  source 1..9: note = 1A7[CD28]_H1.1_L1.71 vlCDR3
QQVYSTPFT

SEQ ID NO: 944 - protein, 121 aa, synthetic construct; identical to SEQ ID NO: 758
  source 1..121: note = 1A7[CD28]_H1.14_L1 Variable Heavy (vh) Domain

SEQ ID NO: 945 - protein, 5 aa, synthetic construct
  source 1..5: note = 1A7[CD28]_H1.14_L1 vhCDR1
SYYMS

SEQ ID NO: 946 - protein, 17 aa, synthetic construct
  source 1..17: note = 1A7[CD28]_H1.14_L1 vhCDR2
TISESGDSTYYADSVKG

SEQ ID NO: 947 - protein, 12 aa, synthetic construct
  source 1..12: note = 1A7[CD28]_H1.14_L1 vhCDR3
SGPGLRQVGFDY

SEQ ID NO: 948 - protein, 107 aa, synthetic construct; identical to SEQ ID NO: 741
  source 1..107: note = 1A7[CD28]_H1.14_L1 Variable Light (vl) Domain

SEQ ID NO: 949 - protein, 11 aa, synthetic construct
  source 1..11: note = 1A7[CD28]_H1.14_L1 vlCDR1
RASQSISSYLN

SEQ ID NO: 950 - protein, 7 aa, synthetic construct
  source 1..7: note = 1A7[CD28]_H1.14_L1 vlCDR2
AASSLQS

SEQ ID NO: 951 - protein, 9 aa, synthetic construct
  source 1..9: note = 1A7[CD28]_H1.14_L1 vlCDR3
QQSYSTPFT

SEQ ID NO: 952 - protein, 121 aa, synthetic construct; identical to SEQ ID NO: 758
  source 1..121: note = 1A7[CD28]_H1.14_L1.71 Variable Heavy (vh) Domain

SEQ ID NO: 953 - protein, 5 aa, synthetic construct
  source 1..5: note = 1A7[CD28]_H1.14_L1.71 vhCDR1
SYYMS

SEQ ID NO: 954 - protein, 17 aa, synthetic construct
  source 1..17: note = 1A7[CD28]_H1.14_L1.71 vhCDR2
TISESGDSTYYADSVKG

SEQ ID NO: 955 - protein, 12 aa, synthetic construct
  source 1..12: note = 1A7[CD28]_H1.14_L1.71 vhCDR3
SGPGLRQVGFDY

SEQ ID NO: 956 - protein, 107 aa, synthetic construct; identical to SEQ ID NO: 884
  source 1..107: note = 1A7[CD28]_H1.14_L1.71 Variable Light (vl) Domain

SEQ ID NO: 957 - protein, 11 aa, synthetic construct
  source 1..11: note = 1A7[CD28]_H1.14_L1.71 vlCDR1
RASQSISSYLN

SEQ ID NO: 958 - protein, 7 aa, synthetic construct
  source 1..7: note = 1A7[CD28]_H1.14_L1.71 vlCDR2
AASSLQS

SEQ ID NO: 959 - protein, 9 aa, synthetic construct
  source 1..9: note = 1A7[CD28]_H1.14_L1.71 vlCDR3
QQVYSTPFT

SEQ ID NO: 960 - protein, 30 aa, synthetic construct
  source 1..30: note = 1A7 VH HFR1
  VARIANT 28: note = T, S, or N
  VARIANT 29: note = F or L
  VARIANT 30: note = S, E, R, K, G, T, A, or N
EVQLLESGGGLVQPGGSLRLSCAASGFXXX

SEQ ID NO: 961 - protein, 14 aa, synthetic construct
  source 1..14: note = 1A7 VH HFR2
  VARIANT 14: note = S or A
WVRQAPGKGLEWVX

SEQ ID NO: 962 - protein, 17 aa, synthetic construct
  source 1..17: note = 1A7 VH HCDR2
  VARIANT 1: note = T or S
  VARIANT 3: note = S, D, E, Y, or T
  VARIANT 4: note = G, D, E, A, Y, S, N, or T
  VARIANT 5: note = S, D, N, or G
  VARIANT 6: note = D, T, Y, S, or A
  VARIANT 7: note = D, T, Y, S, or A
  VARIANT 8: note = S, Y, A, T, D, or N
XIXXXXXXTYYADSVKG

SEQ ID NO: 963 - protein, 32 aa, synthetic construct
  source 1..32: note = 1A7 VH HFR3
RFTISRDNSKNTLYLQMNSLRAEDTAVYYCAK

SEQ ID NO: 964 - protein, 12 aa, synthetic construct
  source 1..12: note = 1A7 VH HCDR3
SGPGLRQVGFDY

SEQ ID NO: 965 - protein, 11 aa, synthetic construct
  source 1..11: note = 1A7 VH HFR4
WGQGTLVTVSS

SEQ ID NO: 966 - protein, 23 aa, synthetic construct
  source 1..23: note = 1A7 VL LFR1
DIQMTQSPSSLSASVGDRVTITC

SEQ ID NO: 967 - protein, 11 aa, synthetic construct
  source 1..11: note = 1A7 VL LCDR1
  VARIANT 8: note = S, A, D, G, H, K, N, Q, T, V, or Y
  VARIANT 9: note = Y, A, D, F, H, K, L, N, Q, S, or W
  VARIANT 11: note = N, A, D, G, H, Q, S, T, or Y
RASQSISXXLX

SEQ ID NO: 968 - protein, 15 aa, synthetic construct
  source 1..15: note = 1A7 VL LFR2
WYQQKPGKAPKLLIY

SEQ ID NO: 969 - protein, 32 aa, synthetic construct
  source 1..32: note = 1A7 VL LFR3
GVPSRFSGSGSGTDFTLTISSLQPEDFATYYC

SEQ ID NO: 970 - protein, 9 aa, synthetic construct
  source 1..9: note = 1A7 VL LCDR3
  VARIANT 3: note = S, A, D, F, H, K, L, T, V, or Y
  VARIANT 4: note = Y, A, D, F, H, K, L, Q, V, or W
  VARIANT 5: note = S, A, D, G, H, K, N, Q, T, V, or Y
  VARIANT 6: note = T, A, D, F, I, K, L, Q, S, V, or Y
  VARIANT 8: note = F, I, L, or W
QQXXXXPXT

SEQ ID NO: 971 - protein, 10 aa, synthetic construct
  source 1..10: note = 1A7 VL LFR4
FGQGTKLEIK

SEQ ID NO: 972 - protein, 121 aa, synthetic construct
  source 1..121: note = CD28.3[CD28]_H0L0 Variable heavy (vh) domain
EVKLQQSGAELVKPGASVRLSCKASGYTFTEYIIHWIKLRSGQGLEWIGWFYPGSNDIQYNAKFKGKATLTADKSSSTVYMELTGLTSEDSAVYFCARRDDFSGYDALPYWGQGTMVTVSS

SEQ ID NO: 973 - protein, 5 aa, synthetic construct
  source 1..5: note = CD28.3[CD28]_H0L0 vhCDR1
EYIIH

SEQ ID NO: 974 - protein, 17 aa, synthetic construct
  source 1..17: note = CD28.3[CD28]_H0L0 vhCDR2
WFYPGSNDIQYNAKFKG

SEQ ID NO: 975 - protein, 12 aa, synthetic construct
  source 1..12: note = CD28.3[CD28]_H0L0 vhCDR3
RDDFSGYDALPY

SEQ ID NO: 976 - protein, 107 aa, synthetic construct
  source 1..107: note = CD28.3[CD28]_H0L0 Variable light (vl) domain
DIQMTQSPASLSVSVGETVTITCRTNENIYSNLAWYQQKQGKSPQLLIYAATHLVEGVPSRFSGSGSGTQYSLKITSLQSEDFGNYYCQHFWGTPCTFGGGTKLEIK

SEQ ID NO: 977 - protein, 11 aa, synthetic construct
  source 1..11: note = CD28.3[CD28]_H0L0 vlCDR1
RTNENIYSNLA

SEQ ID NO: 978 - protein, 7 aa, synthetic construct
  source 1..7: note = CD28.3[CD28]_H0L0 vlCDR2
AATHLVE

SEQ ID NO: 979 - protein, 9 aa, synthetic construct
  source 1..9: note = CD28.3[CD28]_H0L0 vlCDR3
QHFWGTPCT

SEQ ID NO: 980 - protein, 121 aa, synthetic construct
  source 1..121: note = hCD28.3[CD28]_H1L1 Variable heavy (vh) domain
QVQLQQSGAELKKPGASVKVSCKASGYTFTEYIIHWIKLRSGQGLEWIGWFYPGSNDIQYNAQFKGKATLTADKSSSTVYMELTGLTPEDSAVYFCARRDDFSGYDALPYWGQGTLVTVSA

SEQ ID NO: 981 - protein, 5 aa, synthetic construct
  source 1..5: note = hCD28.3[CD28]_H1L1 vhCDR1
EYIIH

SEQ ID NO: 982 - protein, 17 aa, synthetic construct
  source 1..17: note = hCD28.3[CD28]_H1L1 vhCDR2
WFYPGSNDIQYNAQFKG

SEQ ID NO: 983 - protein, 12 aa, synthetic construct
  source 1..12: note = hCD28.3[CD28]_H1L1 vhCDR3
RDDFSGYDALPY

SEQ ID NO: 984 - protein, 107 aa, synthetic construct
  source 1..107: note = hCD28.3[CD28]_H1L1 Variable light (vl) domain
DIQMTQSPSSLSASVGDRVTITCKTNENIYSNLAWYQQKDGKSPQLLIYAATHLVEGVPSRFSGSGSGTQYSLTISSLQPEDFGNYYCQHFWGTPCTFGGGTKLEIK

SEQ ID NO: 985 - protein, 11 aa, synthetic construct
  source 1..11: note = hCD28.3[CD28]_H1L1 vlCDR1
KTNENIYSNLA

SEQ ID NO: 986 - protein, 7 aa, synthetic construct
  source 1..7: note = hCD28.3[CD28]_H1L1 vlCDR2
AATHLVE

SEQ ID NO: 987 - protein, 9 aa, synthetic construct
  source 1..9: note = hCD28.3[CD28]_H1L1 vlCDR3
QHFWGTPCT

SEQ ID NO: 988 - protein, 120 aa, synthetic construct
  source 1..120: note = 5.11A1[CD28]_H0L0 Variable heavy (vh) domain
QVQLQQSGPELVKPGTSVRISCEASGYTFTSYYIHWVKQRPGQGLEWIGCIYPGNVNTNYNEKFKDKATLIVDTSSNTAYMQLSRMTSEDSAVYFCTRSHYGLDWNFDVWGAGTTVTVSS

SEQ ID NO: 989 - protein, 5 aa, synthetic construct
  source 1..5: note = 5.11A1[CD28]_H0L0 vhCDR1
SYYIH

SEQ ID NO: 990 - protein, 17 aa, synthetic construct
  source 1..17: note = 5.11A1[CD28]_H0L0 vhCDR2
CIYPGNVNTNYNEKFKD

SEQ ID NO: 991 - protein, 11 aa, synthetic construct
  source 1..11: note = 5.11A1[CD28]_H0L0 vhCDR3
SHYGLDWNFDV

SEQ ID NO: 992 - protein, 107 aa, synthetic construct
  source 1..107: note = 5.11A1[CD28]_H0L0 Variable light (vl) domain
DIQMNQSPSSLSASLGDTITITCHASQNIYVWLNWYQQKPGNIPKLLIYKASNLHTGVPSRFSGSGSGTGFTLTISSLQPEDIATYYCQQGQTYPYTFGGGTKLEIK

SEQ ID NO: 993 - protein, 11 aa, synthetic construct
  source 1..11: note = 5.11A1[CD28]_H0L0 vlCDR1
HASQNIYVWLN

SEQ ID NO: 994 - protein, 7 aa, synthetic construct
  source 1..7: note = 5.11A1[CD28]_H0L0 vlCDR2
KASNLHT

SEQ ID NO: 995 - protein, 9 aa, synthetic construct
  source 1..9: note = 5.11A1[CD28]_H0L0 vlCDR3
QQGQTYPYT

SEQ ID NO: 996 - protein, 120 aa, synthetic construct
  source 1..120: note = TGN1412_H1L1 Variable heavy (vh) domain
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSS

SEQ ID NO: 997 - protein, 5 aa, synthetic construct
  source 1..5: note = TGN1412_H1L1 vhCDR1
SYYIH

SEQ ID NO: 998 - protein, 17 aa, synthetic construct
  source 1..17: note = TGN1412_H1L1 vhCDR2
CIYPGNVNTNYNEKFKD

SEQ ID NO: 999 - protein, 11 aa, synthetic construct
  source 1..11: note = TGN1412_H1L1 vhCDR3
SHYGLDWNFDV

SEQ ID NO: 1000 - protein, 107 aa, synthetic construct
  source 1..107: note = TGN1412_H1L1 Variable light (vl) domain
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIK

SEQ ID NO: 1001 - protein, 11 aa, synthetic construct
  source 1..11: note = TGN1412_H1L1 vlCDR1
HASQNIYVWLN

SEQ ID NO: 1002 - protein, 7 aa, synthetic construct
  source 1..7: note = TGN1412_H1L1 vlCDR2
KASNLHT

SEQ ID NO: 1003 - protein, 9 aa, synthetic construct
  source 1..9: note = TGN1412_H1L1 vlCDR3
QQGQTYPYT

SEQ ID NO: 1004 - protein, 127 aa, synthetic construct
  source 1..127: note = 341VL34[CD28]_H1L1 Variable heavy (vh) domain
EVQLVESGGGLVKPGGSLRLSCGGSGFTFNNAWMNWVRQAPGKGLEWVGRIKGKTDGGTADYAAPVKGRFTISRDYSKNTLYLQMNSLTTEDTAVYYCNTDLPYYYGSGRYSGMDVWGQGTTVTVSS

SEQ ID NO: 1005 - protein, 5 aa, synthetic construct
  source 1..5: note = 341VL34[CD28]_H1L1 vhCDR1
NAWMN

SEQ ID NO: 1006 - protein, 19 aa, synthetic construct
  source 1..19: note = 341VL34[CD28]_H1L1 vhCDR2
RIKGKTDGGTADYAAPVKG

SEQ ID NO: 1007 - protein, 16 aa, synthetic construct
  source 1..16: note = 341VL34[CD28]_H1L1 vhCDR3
DLPYYYGSGRYSGMDV

SEQ ID NO: 1008 - protein, 104 aa, synthetic construct
  source 1..104: note = 341VL34[CD28]_H1L1 Variable light (vl) domain
EIVLTQSPATLSLSPGERATLSCRASQSVSSYLAWYQQKPGQAPRLLIYDASNRATGIPARFSGSGSGTDFTLTISSLEPEDFAVYYCQQRSNTFGPGTKVDIK

SEQ ID NO: 1009 - protein, 11 aa, synthetic construct
  source 1..11: note = 341VL34[CD28]_H1L1 vlCDR1
RASQSVSSYLA

SEQ ID NO: 1010 - protein, 7 aa, synthetic construct
  source 1..7: note = 341VL34[CD28]_H1L1 vlCDR2
DASNRAT

SEQ ID NO: 1011 - protein, 6 aa, synthetic construct
  source 1..6: note = 341VL34[CD28]_H1L1 vlCDR3
QQRSNT

SEQ ID NO: 1012 - protein, 127 aa, synthetic construct
  source 1..127: note = 341VL36[CD28]_H1L1 Variable heavy (vh) domain
EVQLVESGGGLVKPGGSLRLSCGGSGFTFNNAWMNWVRQAPGKGLEWVGRIKGKTDGGTADYAAPVKGRFTISRDYSKNTLYLQMNSLKTEDTGVYYCTTYLPYYYGSERWSGMDVWGQGTTVTVSS

SEQ ID NO: 1013 - protein, 5 aa, synthetic construct
  source 1..5: note = 341VL36[CD28]_H1L1 vhCDR1
NAWMN

SEQ ID NO: 1014 - protein, 19 aa, synthetic construct
  source 1..19: note = 341VL36[CD28]_H1L1 vhCDR2
RIKGKTDGGTADYAAPVKG

SEQ ID NO: 1015 - protein, 16 aa, synthetic construct
  source 1..16: note = 341VL36[CD28]_H1L1 vhCDR3
YLPYYYGSERWSGMDV

SEQ ID NO: 1016 - protein, 104 aa, synthetic construct; identical to SEQ ID NO: 1008
  source 1..104: note = 341VL36[CD28]_H1L1 Variable light (vl) domain

SEQ ID NO: 1017 - protein, 11 aa, synthetic construct
  source 1..11: note = 341VL36[CD28]_H1L1 vlCDR1
RASQSVSSYLA

SEQ ID NO: 1018 - protein, 7 aa, synthetic construct
  source 1..7: note = 341VL36[CD28]_H1L1 vlCDR2
DASNRAT

SEQ ID NO: 1019 - protein, 6 aa, synthetic construct
  source 1..6: note = 341VL36[CD28]_H1L1 vlCDR3
QQRSNT

SEQ ID NO: 1020 - protein, 126 aa, synthetic construct
  source 1..126: note = 281VL4[CD28]_H1L1 Variable heavy (vh) domain
EVQLVQSGSELKKPGSSVKVSCKASGGTSRSFAISWVRQAPGQGLEWMGGIIPIFGPANYAQKFQGRVTITADESTSTAYMELSSLRSEDTAVYYCARHAIAMGWGVITTNYFDSWGQGTMVTVSS

SEQ ID NO: 1021 - protein, 5 aa, synthetic construct
  source 1..5: note = 281VL4[CD28]_H1L1 vhCDR1
SFAIS

SEQ ID NO: 1022 - protein, 17 aa, synthetic construct
  source 1..17: note = 281VL4[CD28]_H1L1 vhCDR2
GIIPIFGPANYAQKFQG

SEQ ID NO: 1023 - protein, 17 aa, synthetic construct
  source 1..17: note = 281VL4[CD28]_H1L1 vhCDR3
HAIAMGWGVITTNYFDS

SEQ ID NO: 1024 - protein, 108 aa, synthetic construct
  source 1..108: note = 281VL4[CD28]_H1L1 Variable light (vl) domain
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPITFGQGTRLEIK

SEQ ID NO: 1025 - protein, 12 aa, synthetic construct
  source 1..12: note = 281VL4[CD28]_H1L1 vlCDR1
RASQSVSSSYLA

SEQ ID NO: 1026 - protein, 7 aa, synthetic construct
  source 1..7: note = 281VL4[CD28]_H1L1 vlCDR2
GASSRAT

SEQ ID NO: 1027 - protein, 9 aa, synthetic construct
  source 1..9: note = 281VL4[CD28]_H1L1 vlCDR3
QQYGSSPIT

SEQ ID NO: 1028 - protein, 121 aa, synthetic construct
  source 1..121: note = HuTN228[CD28]_H1L1 Variable heavy (vh) domain
QVQLQESGPGLVKPSETLSLTCAVSGFSLTSYGVHWIRQPPGKGLEWLGVIWPGGGTNFNSALMSRLTISEDTSKNQVSLKLSSVTAADTAVYYCARDRAYGNYLYAMDYWGQGTLVTVSS

SEQ ID NO: 1029 - protein, 5 aa, synthetic construct
  source 1..5: note = HuTN228[CD28]_H1L1 vhCDR1
SYGVH

SEQ ID NO: 1030 - protein, 16 aa, synthetic construct
  source 1..16: note = HuTN228[CD28]_H1L1 vhCDR2
VIWPGGGTNFNSALMS

SEQ ID NO: 1031 - protein, 13 aa, synthetic construct
  source 1..13: note = HuTN228[CD28]_H1L1 vhCDR3
DRAYGNYLYAMDY

SEQ ID NO: 1032 - protein, 111 aa, synthetic construct
  source 1..111: note = HuTN228[CD28]_H1L1 Variable light (vl) domain
DIQMTQSPSSLSASVGDRVTITCRASESVEYYVTSLMQWYQQKPGKAPKLLIYAASNVDSGVPSRFSGSGSGTDFTLTISSLQPEDIATYYCQQSRKVPFTFGGGTKVEIK

SEQ ID NO: 1033 - protein, 15 aa, synthetic construct
  source 1..15: note = HuTN228[CD28]_H1L1 vlCDR1
RASESVEYYVTSLMQ

SEQ ID NO: 1034 - protein, 7 aa, synthetic construct
  source 1..7: note = HuTN228[CD28]_H1L1 vlCDR2
AASNVDS

SEQ ID NO: 1035 - protein, 9 aa, synthetic construct
  source 1..9: note = HuTN228[CD28]_H1L1 vlCDR3
QQSRKVPFT

SEQ ID NO: 1036 - protein, 124 aa, synthetic construct
  source 1..124: note = PV1[CD28]_H0L0 Variable heavy (vh) domain
QVQLKQSGAELVKPGASVKISCKTSGYTFTDGYMNWVEQKPGQGLEWIGRIDPDSGNTRYNQKFQGKATLTRDKSSSTVYMDLRSLTSEDSAVYYCARDGTFYGTYGYWYFDFWGQGTQVTVSS

SEQ ID NO: 1037 - protein, 5 aa, synthetic construct
  source 1..5: note = PV1[CD28]_H0L0 vhCDR1
DGYMN

SEQ ID NO: 1038 - protein, 17 aa, synthetic construct
  source 1..17: note = PV1[CD28]_H0L0 vhCDR2
RIDPDSGNTRYNQKFQG

SEQ ID NO: 1039 - protein, 15 aa, synthetic construct
  source 1..15: note = PV1[CD28]_H0L0 vhCDR3
DGTFYGTYGYWYFDF

SEQ ID NO: 1040 - protein, 113 aa, synthetic construct
  source 1..113: note = PV1[CD28]_H0L0 Variable light (vl) domain
DIVMTQSPYSLAVSAGEKVTMSCRSSQSLYYSGIKKNLLAWYQQKPGQSPKLLIYFTSTRLPGVPDRFTGSGSGTDYTLTITSVQAEDMGHYFCQQGISTPLTFGDGTKLEIR

SEQ ID NO: 1041 - protein, 17 aa, synthetic construct
  source 1..17: note = PV1[CD28]_H0L0 vlCDR1
RSSQSLYYSGIKKNLLA

SEQ ID NO: 1042 - protein, 7 aa, synthetic construct
  source 1..7: note = PV1[CD28]_H0L0 vlCDR2
FTSTRLP

SEQ ID NO: 1043 - protein, 9 aa, synthetic construct
  source 1..9: note = PV1[CD28]_H0L0 vlCDR3
QQGISTPLT

SEQ ID NO: 1044 - protein, 120 aa, synthetic construct
  source 1..120: note = m9.3[CD28]_H0L0 Variable heavy (vh) domain
EVKLQQSGPGLVTPSQSLSITCTVSGFSLSDYGVHWVRQSPGQGLEWLGVIWAGGGTNYNSALMSRKSISKDNSKSQVFLKMNSLQADDTAVYYCARDKGYSYYYSMDYWGQGTSVTVSS

SEQ ID NO: 1045 - protein, 5 aa, synthetic construct
  source 1..5: note = m9.3[CD28]_H0L0 vhCDR1
DYGVH

SEQ ID NO: 1046 - protein, 16 aa, synthetic construct
  source 1..16: note = m9.3[CD28]_H0L0 vhCDR2
VIWAGGGTNYNSALMS

SEQ ID NO: 1047 - protein, 12 aa, synthetic construct
  source 1..12: note = m9.3[CD28]_H0L0 vhCDR3
DKGYSYYYSMDY

SEQ ID NO: 1048 - protein, 111 aa, synthetic construct
  source 1..111: note = m9.3[CD28]_H0L0 Variable light (vl) domain
DIELTQSPASLAVSLGQRATISCRASESVEYYVTSLMQWYQQKPGQPPKLLIFAASNVESGVPARFSGSGSGTNFSLNIHPVDEDDVAMYFCQQSRKVPYTFGGGTKLEIK

SEQ ID NO: 1049 - protein, 15 aa, synthetic construct
  source 1..15: note = m9.3[CD28]_H0L0 vlCDR1
RASESVEYYVTSLMQ

SEQ ID NO: 1050 - protein, 7 aa, synthetic construct
  source 1..7: note = m9.3[CD28]_H0L0 vlCDR2
AASNVES

SEQ ID NO: 1051 - protein, 9 aa, synthetic construct
  source 1..9: note = m9.3[CD28]_H0L0 vlCDR3
QQSRKVPYT

SEQ ID NO: 1052 - protein, 120 aa, synthetic construct
  source 1..120: note = hu9.3[CD28]_H1L1 Variable heavy (vh) domain
EVQLVQSGGGLVQPGGSLRLSCAGSGFTFSDYGVHWVRQAPGKGLEWVSAIWAGGGTNYASSVMGRFTISRDNAKNSLYLQMNSLRAEDMAVYYCARDKGYSYYYSMDYWGQGTLVTVSS

SEQ ID NO: 1053 - protein, 5 aa, synthetic construct
  source 1..5: note = hu9.3[CD28]_H1L1 vhCDR1
DYGVH

SEQ ID NO: 1054 - protein, 16 aa, synthetic construct
  source 1..16: note = hu9.3[CD28]_H1L1 vhCDR2
AIWAGGGTNYASSVMG

SEQ ID NO: 1055 - protein, 12 aa, synthetic construct
  source 1..12: note = hu9.3[CD28]_H1L1 vhCDR3
DKGYSYYYSMDY

SEQ ID NO: 1056 - protein, 111 aa, synthetic construct
  source 1..111: note = hu9.3[CD28]_H1L1 Variable light (vl) domain
DIVMTQSPDSLAVSLGERATINCRASESVEYYVTSLMAWYQQKPGQPPKLLIYAASNVESGVPDRFSGSGSGTNFSLTISSLQAEDVAVYYCQQSRKVPYTFGQGTKLEIK

SEQ ID NO: 1057 - protein, 15 aa, synthetic construct
  source 1..15: note = hu9.3[CD28]_H1L1 vlCDR1
RASESVEYYVTSLMA

SEQ ID NO: 1058 - protein, 7 aa, synthetic construct
  source 1..7: note = hu9.3[CD28]_H1L1 vlCDR2
AASNVES

SEQ ID NO: 1059 - protein, 9 aa, synthetic construct
  source 1..9: note = hu9.3[CD28]_H1L1 vlCDR3
QQSRKVPYT

SEQ ID NO: 1060 - protein, 127 aa, synthetic construct
  source 1..127: note = 9G2[CD28]_H0L0 Variable heavy (vh) domain
EVQLVESGGGLVQPGRSLKLSCAASGFTFSNYYMAWVRQAPTKGLEWVASITNTGGSTYYRDSVKGRFTISRDNAKSTLYLQMDSLRSEDTATYYCTRGLIYYYDGRNYYDYVMDAWGQGASVTVSS

SEQ ID NO: 1061 - protein, 5 aa, synthetic construct
  source 1..5: note = 9G2[CD28]_H0L0 vhCDR1
NYYMA

SEQ ID NO: 1062 - protein, 17 aa, synthetic construct
  source 1..17: note = 9G2[CD28]_H0L0 vhCDR2
SITNTGGSTYYRDSVKG

SEQ ID NO: 1063 - protein, 18 aa, synthetic construct
  source 1..18: note = 9G2[CD28]_H0L0 vhCDR3
GLIYYYDGRNYYDYVMDA

SEQ ID NO: 1064 - protein, 107 aa, synthetic construct
  source 1..107: note = 9G2[CD28]_H0L0 Variable light (vl) domain
DIQMTQSPASLSASLGETVSIECLASEGISNSLAWYQQKPGKSPQLLIYGASSLQDGVPSRFSGSGSGTQYSLKISGMQPEDEGVYYCQQGYKYPLTFGSGTKLEIK

SEQ ID NO: 1065 - protein, 11 aa, synthetic construct
  source 1..11: note = 9G2[CD28]_H0L0 vlCDR1
LASEGISNSLA

SEQ ID NO: 1066 - protein, 7 aa, synthetic construct
  source 1..7: note = 9G2[CD28]_H0L0 vlCDR2
GASSLQD

SEQ ID NO: 1067 - protein, 9 aa, synthetic construct
  source 1..9: note = 9G2[CD28]_H0L0 vlCDR3
QQGYKYPLT

SEQ ID NO: 1068 - protein, 127 aa, synthetic construct
  source 1..127: note = 9G2[CD28]_H1L1 Variable heavy (vh) domain
EVQLLESGGGLVQPGGSLRLSCAASGFTFSNYYMAWVRQAPGKGLEWVSSITNTGGSTYYRDSVKGRFTISRDNSKSTLYLQMNSLRAEDTAVYYCTRGLIYYYDGRNYYDYVMDAWGQGTTVTVSS

SEQ ID NO: 1069 - protein, 5 aa, synthetic construct
  source 1..5: note = 9G2[CD28]_H1L1 vhCDR1
NYYMA

SEQ ID NO: 1070 - protein, 17 aa, synthetic construct
  source 1..17: note = 9G2[CD28]_H1L1 vhCDR2
SITNTGGSTYYRDSVKG

SEQ ID NO: 1071 - protein, 18 aa, synthetic construct
  source 1..18: note = 9G2[CD28]_H1L1 vhCDR3
GLIYYYDGRNYYDYVMDA

SEQ ID NO: 1072 - protein, 107 aa, synthetic construct
  source 1..107: note = 9G2[CD28]_H1L1 Variable light (vl) domain
DIQMTQSPSSLSASVGDRVTITCRASEGISNSLAWYQQKPGKSPKLLIYGASSLQDGVPSRFSGSGSGTDYTLTISSLQPEDFATYYCQQGYKYPLTFGSGTKVEIK

SEQ ID NO: 1073 - protein, 11 aa, synthetic construct
  source 1..11: note = 9G2[CD28]_H1L1 vlCDR1
RASEGISNSLA

SEQ ID NO: 1074 - protein, 7 aa, synthetic construct
  source 1..7: note = 9G2[CD28]_H1L1 vlCDR2
GASSLQD

SEQ ID NO: 1075 - protein, 9 aa, synthetic construct
  source 1..9: note = 9G2[CD28]_H1L1 vlCDR3
QQGYKYPLT

SEQ ID NO: 1076 - protein, 121 aa, synthetic construct
  source 1..121: note = 2F10A3.140[CD28]_H1L1 Variable heavy (vh) domain
EVQLLESGGGLVQPGGSLRLSCAASGFSFGGNSMSWVRQAPGKGLEWVATISDNSYSTYYADSVKGRFTISRDNSKNTLYLQMNSLRAEDTAVYYCAKSGPGLRQVGFDYWGQGTLVTVSS

SEQ ID NO: 1077 - protein, 5 aa, synthetic construct
  source 1..5: note = 2F10A3.140[CD28]_H1L1 vhCDR1
GNSMS

SEQ ID NO: 1078 - protein, 17 aa, synthetic construct
  source 1..17: note = 2F10A3.140[CD28]_H1L1 vhCDR2
TISDNSYSTYYADSVKG

SEQ ID NO: 1079 - protein, 12 aa, synthetic construct
  source 1..12: note = 2F10A3.140[CD28]_H1L1 vhCDR3
SGPGLRQVGFDY

SEQ ID NO: 1080 - protein, 107 aa, synthetic construct; identical to SEQ ID NO: 741
  source 1..107: note = 2F10A3.140[CD28]_H1L1 Variable light (vl) domain

SEQ ID NO: 1081 - protein, 11 aa, synthetic construct
  source 1..11: note = 2F10A3.140[CD28]_H1L1 vlCDR1
RASQSISSYLN

SEQ ID NO: 1082 - protein, 7 aa, synthetic construct
  source 1..7: note = 2F10A3.140[CD28]_H1L1 vlCDR2
AASSLQS

SEQ ID NO: 1083 - protein, 9 aa, synthetic construct
  source 1..9: note = 2F10A3.140[CD28]_H1L1 vlCDR3
QQSYSTPFT

SEQ ID NO: 1084 - protein, 121 aa, synthetic construct
  source 1..121: note = TN228[CD28]_H4L2 Variable heavy (vh) domain
QVQLQESGPGLVKPSETLSLTCTVSGFSLTSYGVHWVRQPPGKGLEWIGVIWPGGGTNYNSALKSRVTISEDTSKSQVSLKLSSVTAADTAVYYCARDRAYGNYLYAMDYWGQGTLVTVSS

SEQ ID NO: 1085 - protein, 5 aa, synthetic construct
  source 1..5: note = TN228[CD28]_H4L2 vhCDR1
SYGVH

SEQ ID NO: 1086 - protein, 16 aa, synthetic construct
  source 1..16: note = TN228[CD28]_H4L2 vhCDR2
VIWPGGGTNYNSALKS

SEQ ID NO: 1087 - protein, 13 aa, synthetic construct
  source 1..13: note = TN228[CD28]_H4L2 vhCDR3
DRAYGNYLYAMDY

SEQ ID NO: 1088 - protein, 111 aa, synthetic construct
  source 1..111: note = TN228[CD28]_H4L2 Variable light (vl) domain
EIVLTQSPATLSLSPGERATLSCRASESVEYYVTSLMQWYQQKPGQAPRLLIYAASNVDSGVPARFSGSGSGTDFTLTISSLEPEDFAVYYCQQSRKVPFTFGGGTKVEIK

SEQ ID NO: 1089 - protein, 15 aa, synthetic construct
  source 1..15: note = TN228[CD28]_H4L2 vlCDR1
RASESVEYYVTSLMQ

SEQ ID NO: 1090 - protein, 7 aa, synthetic construct
  source 1..7: note = TN228[CD28]_H4L2 vlCDR2
AASNVDS

SEQ ID NO: 1091 - protein, 9 aa, synthetic construct
  source 1..9: note = TN228[CD28]_H4L2 vlCDR3
QQSRKVPFT

SEQ ID NO: 1092 - protein, 449 aa, synthetic construct; identical to SEQ ID NO: 617
  source 1..449: note = XENP44468 Chain 1- MESO-C[mesothelin]_H1_Fab-IgG1_p I(-)_Isosteric_A_PVA_/S267K/L368D/K370S/M428L/N434S

SEQ ID NO: 1093 - protein, 716 aa, synthetic construct
  source 1..716: note = XENP44468 Chain 2- MESO-C[mesothelin]_H1_Fab_(G4S)2 _1A7[CD28]_H1.14_L1.71_scFv(GKPGS)4_(G4S)2-Fc(222)_IgG1_PV A_/S267K/S364K/E357Q/M428L/N434S
QVQLVQSGAEVKKPGASVKVSCKASGYTFTSYWMHWVRQAPGQGLEWMGMIHPNSDNTIYYEKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLLESGGGLVQPGGSLRLSCAASGFTFSSYYMSWVRQAPGKGLEWVSTISESGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIKGGGGSGGGGSKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 1094 - protein, 214 aa, synthetic construct; identical to SEQ ID NO: 259
  source 1..214: note = XENP44468 Chain 3- MESO-C[mesothelin]_L1_Light Chain

SEQ ID NO: 1095 - protein, 449 aa, synthetic construct; identical to SEQ ID NO: 617
  source 1..449: note = XENP44469 Chain 1- MESO-C[mesothelin]_H1_Fab-IgG1_p I(-)_Isosteric_A_PVA_/S267K/L368D/K370S/M428L/N434S

SEQ ID NO: 1096 - protein, 716 aa, synthetic construct
  source 1..716: note = XENP44469 Chain 2- MESO-C[mesothelin]_H1_Fab_(G4S)2 _1A7[CD28]_H1_L1.71_scFv(GKPGS)4_(G4S)2-Fc(222)_IgG1_PVA_/ S267K/S364K/E357Q/M428L/N434S
QVQLVQSGAEVKKPGASVKVSCKASGYTFTSYWMHWVRQAPGQGLEWMGMIHPNSDNTIYYEKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLLESGGGLVQPGGSLRLSCAASGFTFSSYAMSWVRQAPGKGLEWVSTISGSGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIKGGGGSGGGGSKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 1097 - protein, 214 aa, synthetic construct; identical to SEQ ID NO: 259
  source 1..214: note = XENP44469 Chain 3- MESO-C[mesothelin]_L1_Light Chain

SEQ ID NO: 1098 - protein, 449 aa, synthetic construct; identical to SEQ ID NO: 653
  source 1..449: note = XENP44470 Chain 1- MESO-C[mesothelin]_H1.3_IgG1_pI( -)_Isosteric_A_PVA_/S267K/L368D/K370S/M428L/N434S

SEQ ID NO: 1099 - protein, 716 aa, synthetic construct
  source 1..716: note = XENP44470 Chain 2- MESO-C[mesothelin]_H1.3_Fab_(G4S )2_1A7[CD28]_H1.14_L1.71_scFv(GKPGS)4_(G4S)2-Fc(222)_IgG1_ PVA_/S267K/S364K/E357Q/M428L/N434S
QVQLVQSGAEVKKPGASVKVSCKASGYTFTSYWMHWVRQAPGQGLEWMGWIHPNSDNTIYYEKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLLESGGGLVQPGGSLRLSCAASGFTFSSYYMSWVRQAPGKGLEWVSTISESGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIKGGGGSGGGGSKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 1100 - protein, 214 aa, synthetic construct; identical to SEQ ID NO: 259
  source 1..214: note = XENP44470 Chain 3- MESO-C[mesothelin]_L1_Light Chain

SEQ ID NO: 1101 - protein, 449 aa, synthetic construct; identical to SEQ ID NO: 653
  source 1..449: note = XENP44471 Chain 1- MESO-C[mesothelin]_H1.3_IgG1_pI( -)_Isosteric_A_PVA_/S267K/L368D/K370S/M428L/N434S

SEQ ID NO: 1102 - protein, 716 aa, synthetic construct
  source 1..716: note = XENP44471 Chain 2- MESO-C[mesothelin]_H1.3_Fab_(G4S )2_1A7[CD28]_H1_L1.71_scFv(GKPGS)4_(G4S)2-Fc(222)_IgG1_PVA _/S267K/S364K/E357Q/M428L/N434S
QVQLVQSGAEVKKPGASVKVSCKASGYTFTSYWMHWVRQAPGQGLEWMGWIHPNSDNTIYYEKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLLESGGGLVQPGGSLRLSCAASGFTFSSYAMSWVRQAPGKGLEWVSTISGSGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIKGGGGSGGGGSKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 1103 - protein, 214 aa, synthetic construct; identical to SEQ ID NO: 259
  source 1..214: note = XENP44471 Chain 3- MESO-C[mesothelin]_L1_Light Chain

SEQ ID NO: 1104 - protein, 449 aa, synthetic construct; identical to SEQ ID NO: 653
  source 1..449: note = XENP44472 Chain 1- MESO-C[mesothelin]_H1.3_IgG1_pI( -)_Isosteric_A_PVA_/S267K/L368D/K370S/M428L/N434S

SEQ ID NO: 1105 - protein, 716 aa, synthetic construct; identical to SEQ ID NO: 1099
  source 1..716: note = XENP44472 Chain 2- MESO-C[mesothelin]_H1.3_Fab_(G4S )2_1A7[CD28]_H1.14_L1.71_scFv(GKPGS)4_(G4S)2-Fc(222)_IgG1_ PVA_/S267K/S364K/E357Q/M428L/N434S

SEQ ID NO: 1106 - protein, 214 aa, synthetic construct; identical to SEQ ID NO: 667
  source 1..214: note = XENP44472 Chain 3- MESO-C[mesothelin]_L1.13_Light Chain

SEQ ID NO: 1107 - protein, 449 aa, synthetic construct; identical to SEQ ID NO: 653
  source 1..449: note = XENP44473 Chain 1- MESO-C[mesothelin]_H1.3_IgG1_pI( -)_Isosteric_A_PVA_/S267K/L368D/K370S/M428L/N434S

SEQ ID NO: 1108 - protein, 716 aa, synthetic construct; identical to SEQ ID NO: 1102
  source 1..716: note = XENP44473 Chain 2- MESO-C[mesothelin]_H1.3_Fab_(G4S )2_1A7[CD28]_H1_L1.71_scFv(GKPGS)4_(G4S)2-Fc(222)_IgG1_PVA _/S267K/S364K/E357Q/M428L/N434S

SEQ ID NO: 1109 - protein, 214 aa, synthetic construct; identical to SEQ ID NO: 667
  source 1..214: note = XENP44473 Chain 3- MESO-C[mesothelin]_L1.13_Light Chain

SEQ ID NO: 1110 - protein, 682 aa, synthetic construct
  source 1..682: note = XENP44474 Chain 1- MESO-C[mesothelin]_H1_Fab_(G4S)2 _MESO-C[mesothelin]_H1_Fab-IgG1_pI(-)_Isosteric_A_PVA_/S26 7K/L368D/K370S/M428L/N434S
QVQLVQSGAEVKKPGASVKVSCKASGYTFTSYWMHWVRQAPGQGLEWMGMIHPNSDNTIYYEKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCGGGGSGGGGSQVQLVQSGAEVKKPGASVKVSCKASGYTFTSYWMHWVRQAPGQGLEWMGMIHPNSDNTIYYEKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 1111 - protein, 479 aa, synthetic construct; identical to SEQ ID NO: 681
  source 1..479: note = XENP44474 Chain 2- 1A7[CD28]_H1.14_L1.71_scFv(GKPGS )4-Fc(216)_IgG1_C220S/PVA_/S267K/S364K/E357Q/M428L/N434S

SEQ ID NO: 1112 - protein, 214 aa, synthetic construct; identical to SEQ ID NO: 259
  source 1..214: note = XENP44474 Chain 3- MESO-C[mesothelin]_L1_Light Chain

SEQ ID NO: 1113 - protein, 682 aa, synthetic construct; identical to SEQ ID NO: 1110
  source 1..682: note = XENP44475 Chain 1- MESO-C[mesothelin]_H1_Fab_(G4S)2 _MESO-C[mesothelin]_H1_Fab-IgG1_pI(-)_Isosteric_A_PVA_/S26 7K/L368D/K370S/M428L/N434S

SEQ ID NO: 1114 - protein, 479 aa, synthetic construct; identical to SEQ ID NO: 684
  source 1..479: note = XENP44475 Chain 2- 1A7[CD28]_H1_L1.71_scFv(GKPGS)4- Fc(216)_IgG1_C220S/PVA_/S267K/S364K/E357Q/M428L/N434S

SEQ ID NO: 1115 - protein, 214 aa, synthetic construct; identical to SEQ ID NO: 259
  source 1..214: note = XENP44475 Chain 3- MESO-C[mesothelin]_L1_Light Chain

SEQ ID NO: 1116 - protein, 682 aa, synthetic construct
  source 1..682: note = XENP44476 Chain 1- MESO-C[mesothelin]_H1.3_Fab_(G4S )2_MESO-C[mesothelin]_H1.3_Fab-IgG1_pI(-)_Isosteric_A_PVA_ /S267K/L368D/K370S/M428L/N434S
QVQLVQSGAEVKKPGASVKVSCKASGYTFTSYWMHWVRQAPGQGLEWMGWIHPNSDNTIYYEKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCGGGGSGGGGSQVQLVQSGAEVKKPGASVKVSCKASGYTFTSYWMHWVRQAPGQGLEWMGWIHPNSDNTIYYEKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 1117 - protein, 479 aa, synthetic construct; identical to SEQ ID NO: 681
  source 1..479: note = XENP44476 Chain 2- 1A7[CD28]_H1.14_L1.71_scFv(GKPGS )4-Fc(216)_IgG1_C220S/PVA_/S267K/S364K/E357Q/M428L/N434S

SEQ ID NO: 1118 - protein, 214 aa, synthetic construct; identical to SEQ ID NO: 259
  source 1..214: note = XENP44476 Chain 3- MESO-C[mesothelin]_L1_Light Chain

SEQ ID NO: 1119 - protein, 682 aa, synthetic construct; identical to SEQ ID NO: 1116
  source 1..682: note = XENP44477 Chain 1- MESO-C[mesothelin]_H1.3_Fab_(G4S )2_MESO-C[mesothelin]_H1.3_Fab-IgG1_pI(-)_Isosteric_A_PVA_ /S267K/L368D/K370S/M428L/N434S

SEQ ID NO: 1120 - protein, 479 aa, synthetic construct; identical to SEQ ID NO: 684
  source 1..479: note = XENP44477 Chain 2- 1A7[CD28]_H1_L1.71_scFv(GKPGS)4- Fc(216)_IgG1_C220S/PVA_/S267K/S364K/E357Q/M428L/N434S

SEQ ID NO: 1121 - protein, 214 aa, synthetic construct; identical to SEQ ID NO: 259
  source 1..214: note = XENP44477 Chain 3- MESO-C[mesothelin]_L1_Light Chain

SEQ ID NO: 1122 - protein, 682 aa, synthetic construct; identical to SEQ ID NO: 1116
  source 1..682: note = XENP44478 Chain 1- MESO-C[mesothelin]_H1.3_Fab_(G4S )2_MESO-C[mesothelin]_H1.3_Fab-IgG1_pI(-)_Isosteric_A_PVA_ /S267K/L368D/K370S/M428L/N434S

SEQ ID NO: 1123 - protein, 479 aa, synthetic construct; identical to SEQ ID NO: 681
  source 1..479: note = XENP44478 Chain 2- 1A7[CD28]_H1.14_L1.71_scFv(GKPGS )4-Fc(216)_IgG1_C220S/PVA_/S267K/S364K/E357Q/M428L/N434S

SEQ ID NO: 1124 - protein, 214 aa, synthetic construct; identical to SEQ ID NO: 667
  source 1..214: note = XENP44478 Chain 3- MESO-C[mesothelin]_L1.13_Light Chain

SEQ ID NO: 1125 - protein, 682 aa, synthetic construct; identical to SEQ ID NO: 1116
  source 1..682: note = XENP44479 Chain 1- MESO-C[mesothelin]_H1.3_Fab_(G4S )2_MESO-C[mesothelin]_H1.3_Fab-IgG1_pI(-)_Isosteric_A_PVA_ /S267K/L368D/K370S/M428L/N434S

SEQ ID NO: 1126 - protein, 479 aa, synthetic construct; identical to SEQ ID NO: 684
  source 1..479: note = XENP44479 Chain 2- 1A7[CD28]_H1_L1.71_scFv(GKPGS)4- Fc(216)_IgG1_C220S/PVA_/S267K/S364K/E357Q/M428L/N434S

SEQ ID NO: 1127 - protein, 214 aa, synthetic construct; identical to SEQ ID NO: 667
  source 1..214: note = XENP44479 Chain 3- MESO-C[mesothelin]_L1.13_Light Chain

SEQ ID NO: 1128 - protein, 4 aa, synthetic construct
YMNM

SEQ ID NO: 1129 - protein, 5 aa, synthetic construct
GSGGS

SEQ ID NO: 1130 - protein, 5 aa, synthetic construct
GGGGS

SEQ ID NO: 1131 - protein, 4 aa, synthetic construct
GGGS

(19) United States
(12) Patent Application Publication
(10) Pub. No.: US 2026/0193354 A1
(43) Pub. Date: Jul. 9, 2026

(54) ANTI-CD28 X ANTI-MSLN ANTIBODIES

(71) Applicant: Xencor, Inc., Pasadena, CA (US)

(72) Inventors: John R. Desjarlais, Pasadena, CA (US); Gregory Moore, Azusa, CA (US); Michael Hedvat, Encino, CA (US); Juan Diaz, Anaheim Hills, CA (US)

(21) Appl. No.: 19/571,197

(22) Filed: Mar. 18, 2026

Related U.S. Application Data
(63) Continuation of application No. 18/174,353, filed on Feb. 24, 2023, now abandoned.
(60) Provisional application No. 63/313,707, filed on Feb. 24, 2022.

(51) Int. Cl. C07K 16/28; C07K 16/18

(52) U.S. Cl. CPC C07K 16/2818; C07K 16/18; C07K 16/2809; C07K 2317/31; C07K 2317/35; C07K 2317/52; C07K 2317/522; C07K 2317/524; C07K 2317/526; C07K 2317/53; C07K 2317/55; C07K 2317/622; C07K 2317/92

(57) ABSTRACT

Provided herein are novel anti-CD28×anti-MSLN antibodies and methods of using such antibodies for the treatment of MSLN-associated cancers. Subject anti-CD28×anti-MSLN antibodies are capable of agonistically binding to CD28 costimulatory molecules on T cells and MSLN on tumor cells. Thus, such antibodies selectively enhance anti-tumor activity at tumor sites while minimizing peripheral toxicity. The subject antibodies provided herein are particularly useful in combination with other anti-cancer therapies, including, for example, bispecific antibodies for the treatment of MSLN-associated cancers (e.g., ovarian cancers).

### PRIORITY CLAIM

This application is a continuation of U.S. patent application Ser. No. 18/174,353, filed Feb. 24, 2023, which claims priority to and benefit of U.S. Provisional Application No. 63/313,707, filed on Feb. 24, 2022, the contents of which are hereby incorporated by reference in their entirety.

### SEQUENCE LISTING

The instant application contains a Sequence Listing which has been submitted electronically in XML file format and is hereby incorporated by reference in its entirety. Said XML copy, created on Feb. 22, 2023, is named 067461-5303-WO_SL.xml and is 1,354,117 bytes in size.

### BACKGROUND

Mesothelin (MSLN) has previously been reported to be highly expressed in several cancers, including mesothelioma, ovarian cancer, pancreatic adenocarcinoma, lung adenocarcinoma, and cholangiocarcinoma. Current treatment options for such cancers, however, have only modest efficacy and there remains a large unmet need for new therapies.

Antibody-based therapeutics have been used successfully to treat a variety of diseases, including cancer. An increasingly prevalent avenue being explored is the engineering of single immunoglobulin molecules that co-engage two different antigens. Such alternate antibody formats that engage two different antigens are often referred to as bispecific antibodies. One particular approach for bispecific antibodies is to engineer a first binding domain which engages CD3 and a second binding domain which engages an antigen associated with or upregulated on cancer cells (e.g., MSLN) so that the bispecific antibody redirects CD3⁺ T cells to destroy the cancer cells.

TILs, however, lose their cytotoxic ability over time due to upregulation of inhibitory immune checkpoints. While checkpoint blockade has demonstrated increased clinical response rates relative to other treatment options, many patients still fail to achieve a response to checkpoint blockade. Engagement of costimulatory receptors on TILs could provide a positive signal capable of overcoming negative signals of immune checkpoints. Preclinical and clinical studies of agonistic costimulatory receptor antibodies have indeed demonstrated that agonism of costimulatory receptors can result in impressive anti-tumor responses, activating T cells to attack tumor cells.

It is also important for cancer therapy to enhance anti-tumor activity by specifically destroying tumor cells while minimizing peripheral toxicity. In this context, it is crucial that only T cells in the presence of the target tumor cells are provided a costimulatory signal. However, agonism of costimulatory receptors with monospecific full-length antibodies is likely nondiscriminatory with regards to TILs vs. peripheral T cells vs. autoantigen-reactive T cells that contribute to autoimmune toxicities. Thus, there remains a need for novel immune response enhancing compositions for the treatment of cancers, including MSLN-associated cancers.

### SUMMARY

Provided herein are novel anti-CD28×anti-MSLN antibodies and methods of using such antibodies for the treatment of MSLN-associated cancers. Subject anti-CD28×anti-MSLN antibodies are capable of agonistically binding to CD28 costimulatory molecules on T cells and MSLN on tumor cells. Thus, such antibodies selectively enhance anti-tumor activity at tumor sites while minimizing peripheral toxicity. The subject antibodies provided herein are particularly useful in combination with other anti-cancer therapies, including, for example, bispecific antibodies for the treatment of MSLN-associated cancers (e.g., ovarian cancers).

In a first aspect, provided herein is an anti-CD28×anti-MSLN heterodimeric antibody that comprises a) a first monomer; b) a second monomer; and c) a light chain. The first monomer comprises: i) a single chain variable fragment (scFv); and ii) a first Fc domain, wherein the scFv is covalently attached to the N-terminus of the first Fc domain using a domain linker. The second monomer comprises, from N-terminal to C-terminal, a VH1-CH1-hinge-CH2-CH3, wherein VH1 is a first variable heavy domain and CH2-CH3 is a second Fc domain. The light chain comprises, from N-terminal to C-terminal, VL1-CL, wherein VL1 is a first variable light domain and CL is a constant light domain. The scFv comprises a second VH domain (VH2), a scFv linker, and a second variable light domain (VL2). The VH1 and the VL1 together form a first antigen binding domain (ABD) and the VH2 and the VL2 together form a second ABD. Further, one of the first ABD and second ABD is a CD28 binding domain and the other of the first ABD and second ABD is a mesothelin (MSLN) binding domain.

In some embodiments, the scFv comprises, from N-terminal to C-terminal, VH2-scFv linker-VL2. In some embodiments, the scFv comprises, from N-terminal to C-terminal, VL2-scFv linker-VH2.

In exemplary embodiments, the first ABDs is the MSLN binding domain and the second ABD is the CD28 binding domain. In some embodiments, VH1 and VL1 are selected from one of the following: 1) a VH and VL of any of the MSLN binding domains from FIGS. 15, 16, 17 and 23; or 2) (i) a VH from FIG. 18; and (ii) a VL from FIG. 19. In some embodiments, VH2 and VL2 are selected from one of the following: 1) a VH and VL of any of the CD28 binding domains from FIGS. 27, 30, and 33; or 2) (i) a VH from FIG. 28; and (ii) a VL from FIG. 29.

In some embodiments, the first Fc domain and second Fc domain are each variant Fc domains.

In some embodiments, the first and second Fc domains comprise a set of heterodimerization skew variants selected from the following heterodimerization variants: S364K/E357Q:L368D/K370S; S364K:L368D/K370S; S364K:L368E/K370S; D401K:T411E/K360E/Q362E; and T366W:T366S/L368A/Y407V, wherein numbering is according to EU numbering. In some embodiments, the first and second Fc domains comprise heterodimerization skew variants S364K/E357Q:L368D/K370S, wherein numbering is according to EU numbering

In certain embodiments, the first and second Fc domains each comprise one or more ablation variants. In exemplary embodiments, the one or more ablation variants comprise E233P/L234V/L235A/G236del/S267K, wherein numbering is according to EU numbering.

In some embodiments, the one of the first or second monomer further comprises one or more pI variants. In some embodiments, the CH1-hinge-CH2-CH3 of the second monomer comprises pI variants N208D/Q295E/N384D/Q418E/N421D, wherein numbering is according to EU numbering.

In some embodiments, the CH1-hinge-CH2-CH3 of the second monomer comprises amino acid variants E233P/L234V/L235A/G236del/S267K/L368D/K370S/N208D/Q295E/N384D/Q418E/N421D, the first Fc domain comprises amino acid variants E233P/L234V/L235A/G236del/S267K/S364K/E357Q, wherein numbering is according to EU numbering. In exemplary embodiments, the first and second variant Fc domains each further comprise amino acid variants 428L/434S.

In some embodiments, the scFv linker is GKPGSGKPGSGKPGSGKPGS (SEQ ID NO: 1).

In another aspect, provided herein is an anti-CD28×anti-MSLN heterodimeric antibody that comprises a) a first monomer; b) a second monomer; c) a first light chain; and d) a second light chain. The first monomer comprises, from N-terminal to C-terminal, VH1-CH1-first domain linker-scFv-second domain linker-CH2-CH3, wherein VH1 is a first variable heavy domain, and CH2-CH3 is a first Fc domain. The second monomer comprises, from N-terminal to C-terminal, a VH1-CH1-hinge-CH2-CH3, wherein CH2-CH3 is a second Fc domain. The first and second light chains each comprise, from N-terminal to C-terminal, VL1-CL, wherein VL1 is a first variable light domain and CL is a constant light domain. The scFv comprises a second VH domain (VH2), a scFv linker, and a second variable light domain (VL2). The VH1 of the first monomer and the VL1 of the first light chain and the VH1 of the second monomer and the VL1 of the second light chain each form a first antigen binding domain (ABD), and the VH2 and the VL2 form a second ABD. Further, one of the first ABDs and second ABD is a CD28 binding domain and the other of the first ABDs and second ABD is a mesothelin (MSLN) binding domain.

In some embodiments, the scFv comprises, from N-terminal to C-terminal, VH2-scFv linker-VL2. In some embodiments, the scFv comprises, from N-terminal to C-terminal, VL2-scFv linker-VH2.

In exemplary embodiments, the first ABDs are the MSLN binding domains and the second ABD is the CD28 binding domain. In some embodiments, VH1 and VL1 are selected from one of the following: 1) a VH and VL of any of the MSLN binding domains from FIGS. 15, 16, 17 and 23; or 2) (i) a VH from FIG. 18; and (ii) a VL from FIG. 19. In some embodiments, VH2 and VL2 are selected from one of the following: 1) a VH and VL of any of the CD28 binding domains from FIGS. 27, 30, and 33; or 2) (i) a VH from FIG. 28; and (ii) a VL from FIG. 29.

In some embodiments, the first Fc domain and second Fc domain are each variant Fc domains.

In some embodiments, the first and second Fc domains comprise a set of heterodimerization skew variants selected from the following heterodimerization variants: S364K/E357Q:L368D/K370S; S364K:L368D/K370S; S364K:L368E/K370S; D401K:T411E/K360E/Q362E; and T366W:T366S/L368A/Y407V, wherein numbering is according to EU numbering. In some embodiments, the first and second Fc domains comprise heterodimerization skew variants S364K/E357Q:L368D/K370S, wherein numbering is according to EU numbering

In certain embodiments, the first and second Fc domains each comprise one or more ablation variants. In exemplary embodiments, the one or more ablation variants comprise E233P/L234V/L235A/G236del/S267K, wherein numbering is according to EU numbering.

In some embodiments, the one of the first or second monomer further comprises one or more pI variants. In some embodiments, the CH1-hinge-CH2-CH3 of the second monomer comprises pI variants N208D/Q295E/N384D/Q418E/N421D, wherein numbering is according to EU numbering.

In some embodiments, the CH1-hinge-CH2-CH3 of the second monomer comprises amino acid variants E233P/L234V/L235A/G236del/S267K/L368D/K370S/N208D/Q295E/N384D/Q418E/N421D, the first Fc domain comprises amino acid variants E233P/L234V/L235A/G236del/S267K/S364K/E357Q, wherein numbering is according to EU numbering. In exemplary embodiments, the first and second variant Fc domains each further comprise amino acid variants 428L/434S.

In some embodiments, the scFv linker is GKPGSGKPGSGKPGSGKPGS (SEQ ID NO: 1).

In another aspect, provided herein is an anti-CD28×anti-MSLN heterodimeric antibody that comprises a) a first monomer; b) a second monomer; c) a first light chain; and d) a second light chain. The first monomer comprises, from N-terminal to C-terminal, VH1-CH1-hinge-CH2-CH3-domain linker-scFv, wherein VH1 is a first variable heavy domain, and CH2-CH3 is a first Fc domain. The second monomer comprises, from N-terminal to C-terminal, a VH1-CH1-hinge-CH2-CH3, wherein VH1 is a first variable heavy domain and CH2-CH3 is a second Fc domain. The first and second light chains each comprise, from N-terminal to C-terminal, VL1-CL, wherein VL1 is a first variable light domain and CL is a constant light domain. The scFv comprises a second VH domain (VH2), a scFv linker, and a second variable light domain (VL2). The VH1 of the first monomer and the VL1 of the first light chain, and the VH1 of the second monomer and the VL1 of the second light chain each form a first antigen binding domain (ABD), and the VH2 and the VL2 form a second ABD. Further, one of the first and second ABDs bind human CD28 and the other of the first and second ABDs binds MSLN.

In some embodiments, the scFv comprises, from N-terminal to C-terminal, VH2-scFv linker-VL2. In some embodiments, the scFv comprises, from N-terminal to C-terminal, VL2-scFv linker-VH2.

In exemplary embodiments, the first ABDs are the MSLN binding domains and the second ABD is the CD28 binding domain. In some embodiments, VH1 and VL1 are selected from one of the following: 1) a VH and VL of any of the MSLN binding domains from FIGS. 15, 16, 17 and 23; or 2) (i) a VH from FIG. 18; and (ii) a VL from FIG. 19. In some embodiments, VH2 and VL2 are selected from one of the following: 1) a VH and VL of any of the CD28 binding domains from FIGS. 27, 30, and 33; or 2) (i) a VH from FIG. 28; and (ii) a VL from FIG. 29.

In some embodiments, the first Fc domain and second Fc domain are each variant Fc domains.

In some embodiments, the first and second Fc domains comprise a set of heterodimerization skew variants selected from the following heterodimerization variants: S364K/E357Q:L368D/K370S; S364K:L368D/K370S; S364K:L368E/K370S; D401K:T411E/K360E/Q362E; and T366W:T366S/L368A/Y407V, wherein numbering is according to EU numbering. In some embodiments, the first and second Fc domains comprise heterodimerization skew variants S364K/E357Q:L368D/K370S, wherein numbering is according to EU numbering.

In certain embodiments, the first and second Fc domains each comprise one or more ablation variants. In exemplary embodiments, the one or more ablation variants comprise E233P/L234V/L235A/G236del/S267K, wherein numbering is according to EU numbering.

In some embodiments, the one of the first or second monomer further comprises one or more pI variants. In some embodiments, the CH1-hinge-CH2-CH3 of the second monomer comprises pI variants N208D/Q295E/N384D/Q418E/N421D, wherein numbering is according to EU numbering.

In some embodiments, the CH1-hinge-CH2-CH3 of the second monomer comprises amino acid variants E233P/L234V/L235A/G236del/S267K/L368D/K370S/N208D/Q295E/N384D/Q418E/N421 D, the first Fc domain comprises amino acid variants E233P/L234V/L235A/G236del/S267K/S364K/E357Q, wherein numbering is according to EU numbering. In exemplary embodiments, the first and second variant Fc domains each further comprise amino acid variants 428L/434S.

In some embodiments, the scFv linker is GKPGSGKPGSGKPGSGKPGS (SEQ ID NO: 1).

In another aspect, provided herein is an anti-CD28×anti-MSLN heterodimeric antibody that comprises a) a first monomer; b) a second monomer; c) a first light chain; and d) a second light chain. The first monomer comprises, from N-terminal to C-terminal, a VH1-CH1-domain linker-VH1-CH1-hinge-CH2-CH3, wherein each of the VH1s is a first variable heavy domain, and CH2-CH3 is a first Fc domain. The second monomer comprises, from N-terminal to C-terminal, an scFv-domain linker-CH2-CH3, wherein VH1 is a first variable heavy domain and CH2-CH3 is a second Fc domain. The first and second light chains each comprise, from N-terminal to C-terminal, VL1-CL, wherein VL1 is a first variable light domain and CL is a constant light domain. The scFv comprises a second VH domain (VH2), a scFv linker, and a second variable light domain (VL2). The VH1 of the first monomer and the VL1 of the first light chain and the VH1 of the second monomer and the VL1 of the second light chain each form a first antigen binding domain (ABD), and the VH2 and the VL2 form a second ABD. Further, one of the first and second ABDs bind human CD28 and the other of the first and second ABDs binds MSLN.

In some embodiments, the scFv comprises, from N-terminal to C-terminal, VH2-scFv linker-VL2. In some embodiments, the scFv comprises, from N-terminal to C-terminal, VL2-scFv linker-VH2.

In exemplary embodiments, the first ABDs are the MSLN binding domains and the second ABD is the CD28 binding domain. In some embodiments, VH1 and VL1 are selected from one of the following: 1) a VH and VL of any of the MSLN binding domains from FIGS. 15, 16, 17 and 23; or 2) (i) a VH from FIG. 18; and (ii) a VL from FIG. 19. In some embodiments, VH2 and VL2 are selected from one of the following: 1) a VH and VL of any of the CD28 binding domains from FIGS. 27, 30, and 33; or 2) (i) a VH from FIG. 28; and (ii) a VL from FIG. 29.

In some embodiments, the first Fc domain and second Fc domain are each variant Fc domains.

In some embodiments, the first and second Fc domains comprise a set of heterodimerization skew variants selected from the following heterodimerization variants: S364K/E357Q:L368D/K370S; S364K:L368D/K370S; S364K:L368E/K370S; D401K:T411E/K360E/Q362E; and T366W:T366S/L368A/Y407V, wherein numbering is according to EU numbering. In some embodiments, the first and second Fc domains comprise heterodimerization skew variants S364K/E357Q:L368D/K370S, wherein numbering is according to EU numbering.

In certain embodiments, the first and second Fc domains each comprise one or more ablation variants. In exemplary embodiments, the one or more ablation variants comprise E233P/L234V/L235A/G236del/S267K, wherein numbering is according to EU numbering.

In some embodiments, the one of the first or second monomer further comprises one or more pI variants. In some embodiments, the CH1-hinge-CH2-CH3 of the second monomer comprises pI variants N208D/Q295E/N384D/Q418E/N421D, wherein numbering is according to EU numbering.

In some embodiments, the CH1-hinge-CH2-CH3 of the second monomer comprises amino acid variants E233P/L234V/L235A/G236del/S267K/L368D/K370S/N208D/Q295E/N384D/Q418E/N421D, the first Fc domain comprises amino acid variants E233P/L234V/L235A/G236del/S267K/S364K/E357Q, wherein numbering is according to EU numbering. In exemplary embodiments, the first and second variant Fc domains each further comprise amino acid variants 428L/434S.

In some embodiments, the scFv linker is GKPGSGKPGSGKPGSGKPGS (SEQ ID NO: 1).

In another aspect, provided herein is a bispecific antibody that comprises: a) a MSLN binding domain comprising i) a first variable heavy domain (VH1), and ii) a first variable light domain (VL1); and b) an anti-CD28 binding domain comprising i) a second variable heavy domain (VH2), and ii) a second variable light domain (VL2). In some embodiments, VH1 and VL1 are selected from one of the following: 1) a VH and VL of any of the MSLN binding domains from FIGS. 15, 16, 17 and 23; or 2) a VH from FIG. 18; and (ii) a VL from FIG. 19. In some embodiments, VH2 and VL2 are selected from one of the following: 1) a VH and VL of any of the CD28 binding domains from FIGS. 27, 30, and 33; or 2) a VH from FIG. 28; and (ii) a VL from FIG. 29.

In some embodiments of the bispecific antibodies, the first Fc domain and second Fc domain are each variant Fc domains.

In some embodiments of the bispecific antibodies, the first and second Fc domains comprise a set of heterodimerization skew variants selected from the following heterodimerization variants: S364K/E357Q:L368D/K370S; S364K:L368D/K370S; S364K:L368E/K370S; D401K:T411E/K360E/Q362E; and T366W:T366S/L368A/Y407V, wherein numbering is according to EU numbering. In some embodiments, the first and second Fc domains comprise heterodimerization skew variants S364K/E357Q: L368D/K370S, wherein numbering is according to EU numbering.

In certain embodiments, the first and second Fc domains each comprise one or more ablation variants. In exemplary embodiments, the one or more ablation variants comprise E233P/L234V/L235A/G236del/S267K, wherein numbering is according to EU numbering.

In some embodiments, the one of the first or second monomer further comprises one or more pI variants. In some embodiments, the CH1-hinge-CH2-CH3 of the second monomer comprises pI variants N208D/Q295E/N384D/Q418E/N421D, wherein numbering is according to EU numbering.

In another aspect, provided herein is a heterodimeric antibody that comprises: a) a first monomer having the amino acid sequence of SEQ ID NO:620; b) a second monomer having the amino acid sequence of SEQ ID NO:621; and c) a light chain having the amino acid sequence of SEQ ID NO:622.

Also provided herein are nucleic acid compositions comprising nucleic acids encoding the antibodies described herein, expression vector compositions that include such nucleic acids, host cells for making the antibodies that comprise the expression vector compositions, and methods of making the antibodies.

In another aspect, provided herein is a method of treating a MSLN-associated cancer in a patient in need thereof, comprising administering to the patient according an anti-CD28×anti-MSLN bispecific antibody as described herein.

In another aspect, provided herein is a method of treating a MSLN-associated cancer in a patient in need thereof, comprising administering to the patient an anti-CD28×anti-MSLN bispecific antibody as described herein; and an anti-CD3×anti-MSLN bispecific antibody.

### BRIEF DESCRIPTION OF THE DRAWINGS

FIG. 1A-1E depict useful pairs of Fe heterodimerization variant sets (including skew and pI variants). There are variants for which there are no corresponding “monomer 2” variants; these are pI variants which can be used alone on either monomer.

FIG. 2 depicts a list of isosteric variant antibody constant regions and their respective substitutions. pI_(−) indicates lower pI variants, while pI_(+) indicates higher pI variants. These can be optionally and independently combined with other heterodimerization variants of the inventions (and other variant types as well, as outlined herein.)

FIG. 3 depicts useful ablation variants that ablate FcγR binding (sometimes referred to as “knock outs” or “KO” variants). Generally, ablation variants are found on both monomers, although in some cases they may be on only one monomer.

FIG. 4 depicts particularly useful embodiments of “non-Fv” components of the antibodies described herein.

FIG. 5 depicts a number of charged scFv linkers that find use in increasing or decreasing the pI of the subject heterodimeric bsAbs that utilize one or more scFv as a component, as described herein. The (+H) positive linker finds particular use herein, particularly with anti-CD3 V_{L} and V_{H} sequences shown herein. A single prior art scFv linker with a single charge is referenced as “Whitlow”, from Whitlow et al., Protein Engineering 6(8):989-995 (1993). It should be noted that this linker was used for reducing aggregation and enhancing proteolytic stability in scFvs. Such charged scFv linkers can be used in any of the subject antibody formats disclosed herein that include scFvs (e.g., 1+1 Fab-scFv-Fc and 2+1 Fab2-scFv-Fc formats).

FIG. 6 depicts a number of exemplary domain linkers. In some embodiments, these linkers find use linking a single-chain Fv to an Fc chain. In some embodiments, these linkers may be combined. For example, a GGGGS linker (SEQ ID NO: 2) may be combined with a “half hinge” linker.

FIGS. 7A-7D shows the sequences of several useful heterodimeric anti-CD28×anti-MSLN bsAb backbones based on human IgG1, without the cytokine sequences. Heterodimeric Fc backbone 1 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants on both chains. Heterodimeric Fc backbone 2 is based on human IgG1 (356E/358M allotype), and includes the L368D/K370S skew variants and the Q295E/N384D/Q418E/N421D pI variants on a first heterodimeric Fc chain, the S364K skew variant on a second heterodimeric Fc chain, and the E233P/L234V/L235A/G236del/S267K ablation variants on both chains. Heterodimeric Fc backbone 3 is based on human IgG1 (356E/358M allotype), and includes the L368E/K370S skew variants and the Q295E/N384D/Q418E/N421D pI variants on a first heterodimeric Fc chain, the S364K skew variant on a second heterodimeric Fc chain, and the E233P/L234V/L235A/G236del/S267K ablation variants on both chains. Heterodimeric Fc backbone 4 is based on human IgG1 (356E/358M allotype), and includes the K360E/Q362E/T411E skew variants and the Q295E/N384D/Q418E/N421D pI variants on a first heterodimeric Fc chain, the D401K skew variant on a second heterodimeric Fc chain, and the E233P/L234V/L235A/G236del/S267K ablation variants on both chains. Heterodimeric Fc backbone 5 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants on both chains. Heterodimeric Fc backbone 6 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and N297A variant that removes glycosylation on both chains. Heterodimeric Fc backbone 7 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and N297S variant that removes glycosylation on both chains. Heterodimeric Fc backbone 8 is based on human IgG4, and includes the L368D/K370S skew variants and the Q295E/N384D/Q418E/N421D pI variants on a first heterodimeric Fc chain, the S364K/E357Q skew variants on a second heterodimeric Fc chain, and the S228P (according to EU numbering, S241P in Kabat) variant that ablates Fab arm exchange (as is known in the art) on both chains. Heterodimeric Fc backbone 9 is based on human IgG2, and includes the L368D/K370S skew variants and the Q295E/N384D/Q418E/N421D pI variants on a first heterodimeric Fc chain, the S364K/E357Q skew variants on a second heterodimeric Fc chain. Heterodimeric Fc backbone 10 is based on human IgG2, and includes the L368D/K370S skew variants and the Q295E/N384D/Q418E/N421D pI variants on a first heterodimeric Fc chain, the S364K/E357Q skew variants on a second heterodimeric Fc chain, and the S267K ablation variant on both chains. Heterodimeric Fc backbone 11 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434S Xtend variants on both chains. Heterodimeric Fc backbone 12 is based on human IgG1 (356E/358M allotype), and includes the L368D/K370S skew variants on a first heterodimeric Fc chain, the S364K/E357Q skew variants and P217R/P229R/N276K pI variants on a second heterodimeric Fc chain, and the E233P/L234V/L235A/G236del/S267K ablation variants on both chains. Heterodimeric Fc backbone 13 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434S Xtend variants on both chains. Heterodimeric Fc backbone 14 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434A Xtend variants on both chains. Heterodimeric Fc backbone 15 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434A Xtend variants on both chains.

Included within each of these backbones are sequences that are 90, 95, 98 and 99% identical (as defined herein) to the recited sequences, and/or contain from 1, 2, 3, 4, 5, 6, 7, 8, 9 or 10 additional amino acid substitutions (as compared to the “parent” of the Figure, which, as will be appreciated by those in the art, already contain a number of amino acid modifications as compared to the parental human IgG1 (or IgG2 or IgG4, depending on the backbone). That is, the recited backbones may contain additional amino acid modifications (generally amino acid substitutions) in addition or as an alternative to the skew, pI and ablation variants contained within the backbones of this Figure. Additionally, the backbones depicted herein may include deletion of the C-terminal glycine (K446_) and/or lysine (K447_). The C-terminal glycine and/or lysine deletion may be intentionally engineered to reduce heterogeneity or in the context of certain bispecific formats, such as the mAb-scFv format. Additionally, C-terminal glycine and/or lysine deletion may occur naturally for example during production and storage.

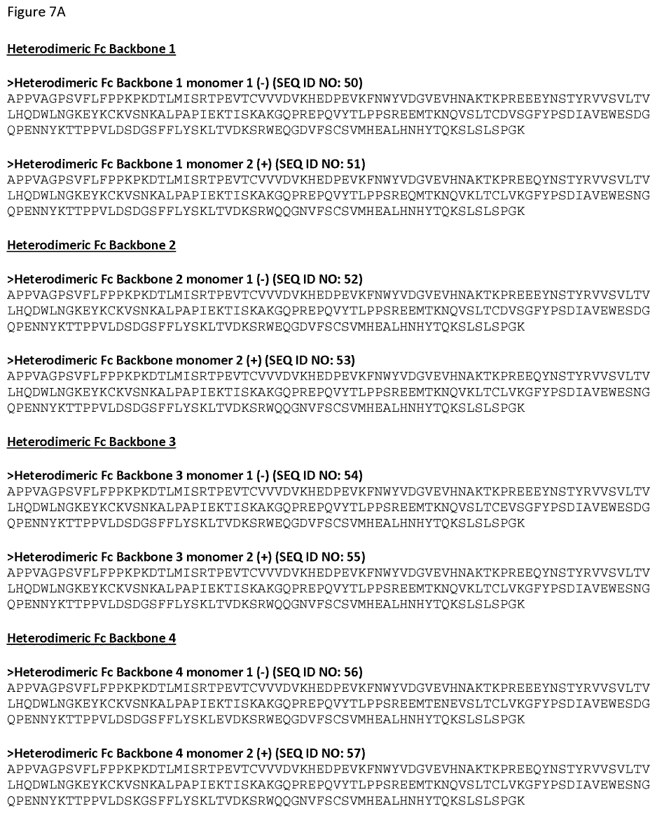

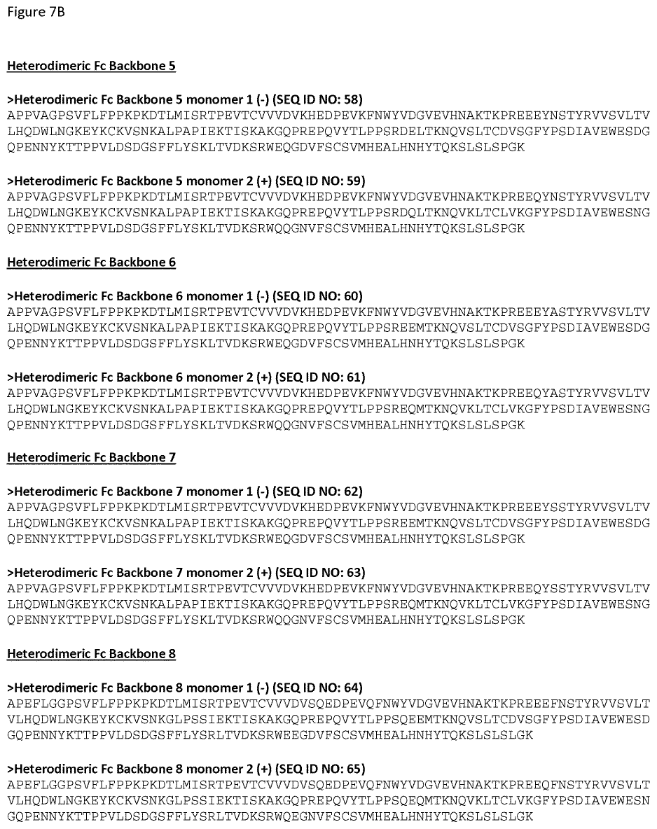

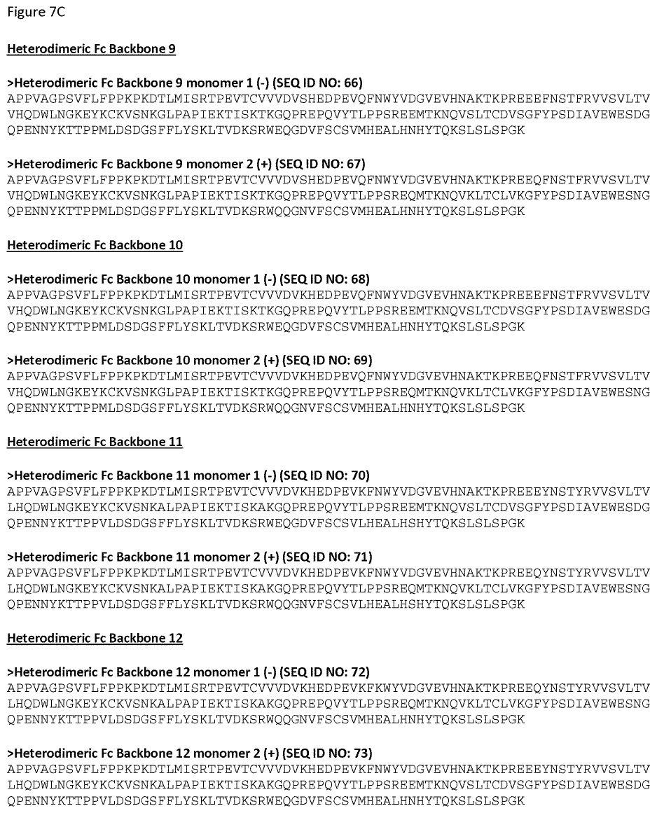

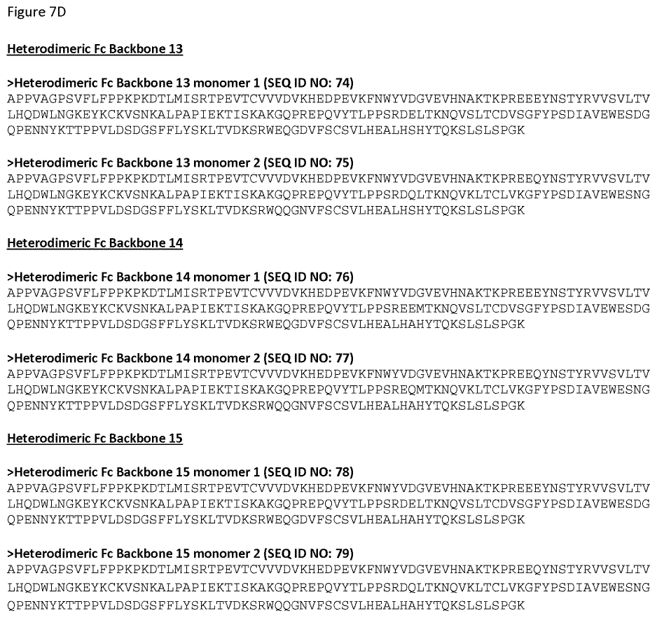

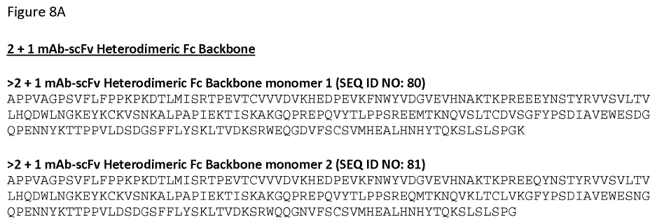

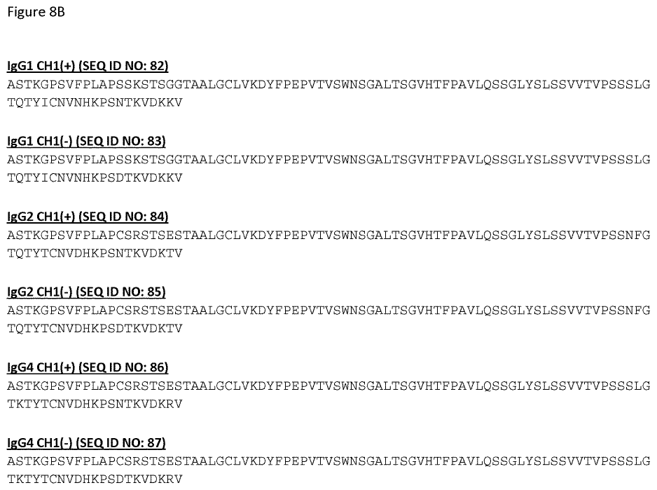

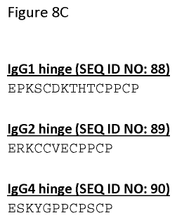

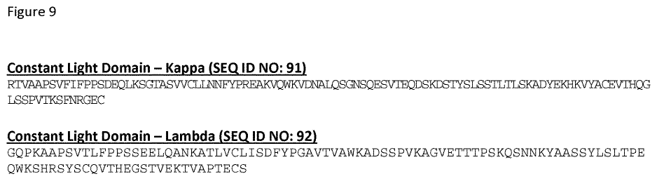

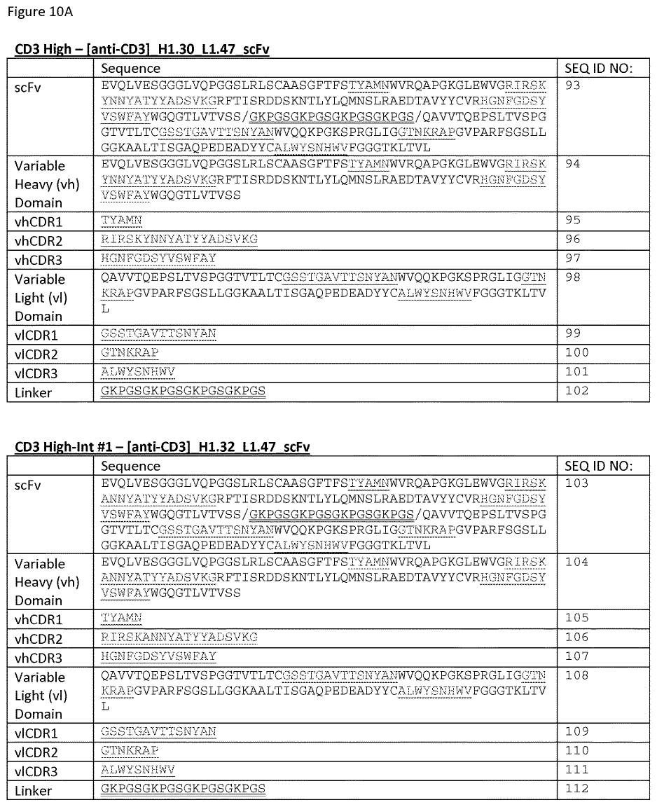

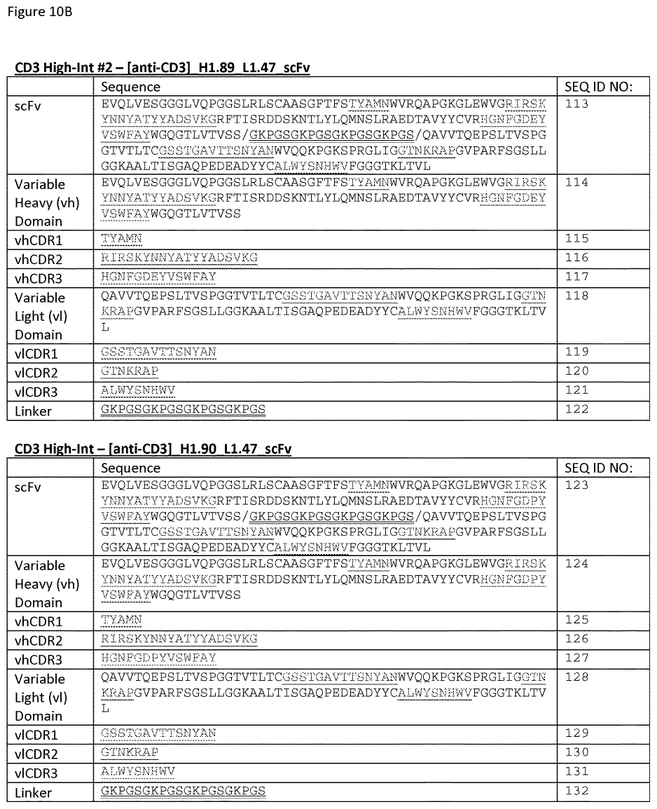

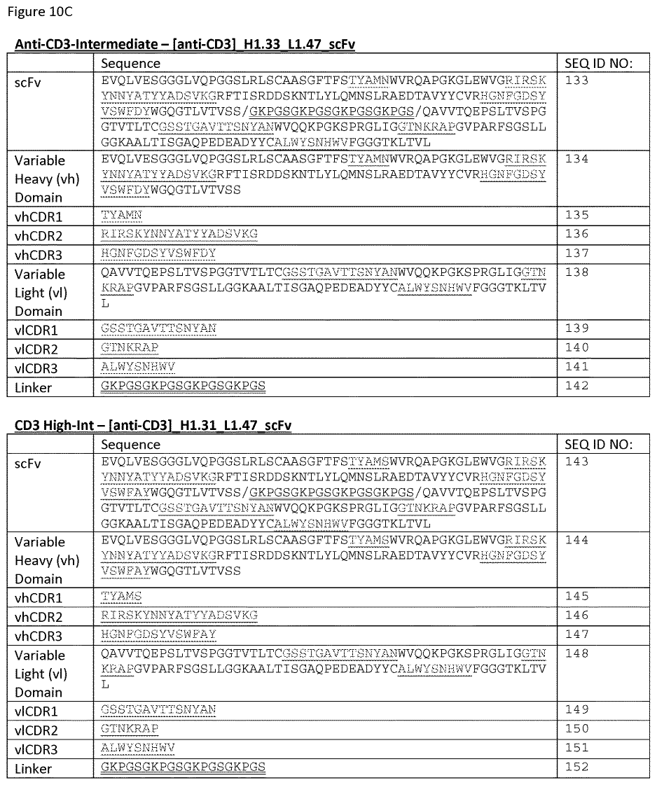

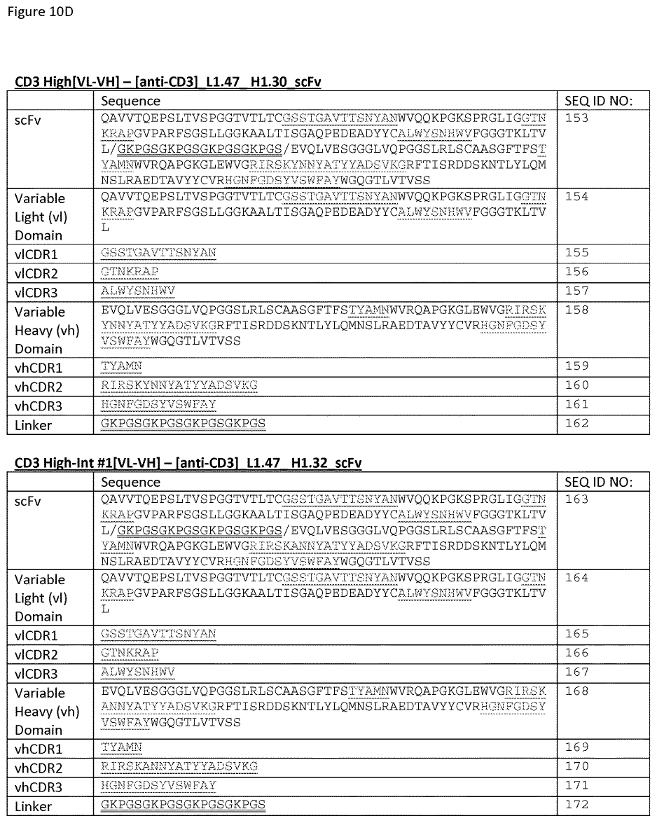

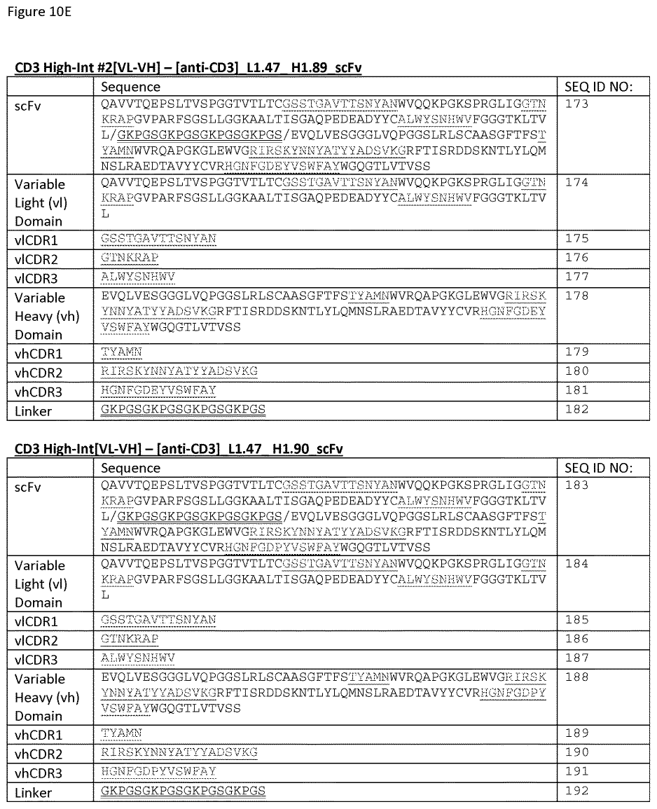

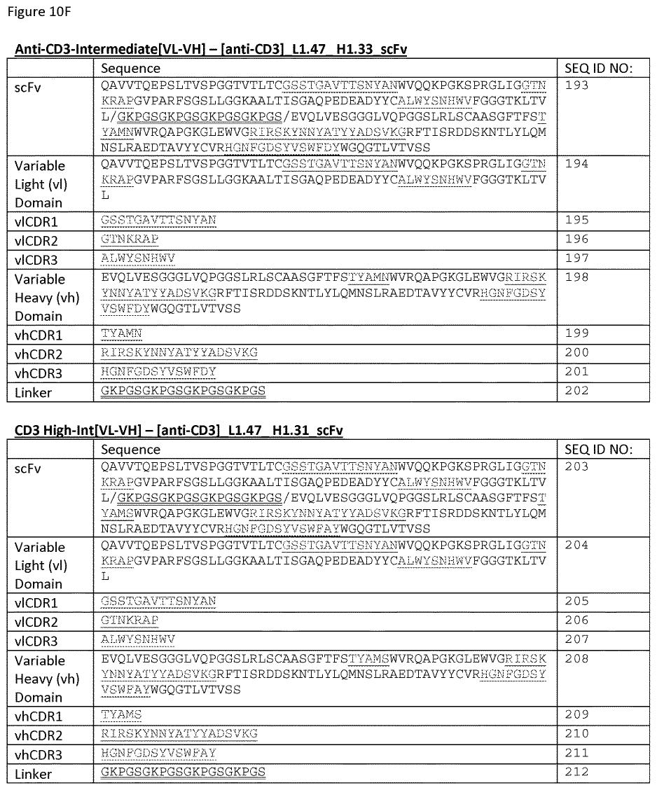

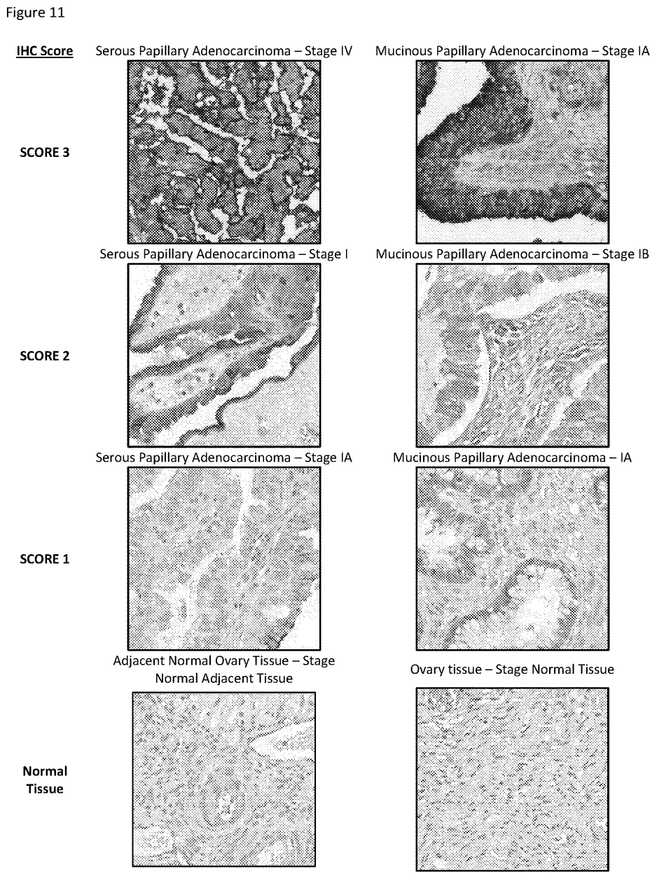

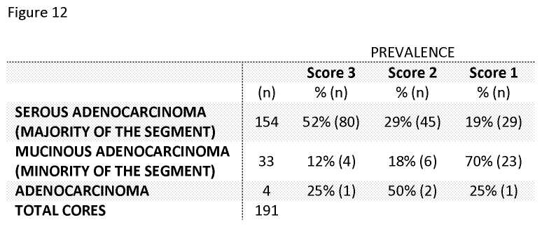

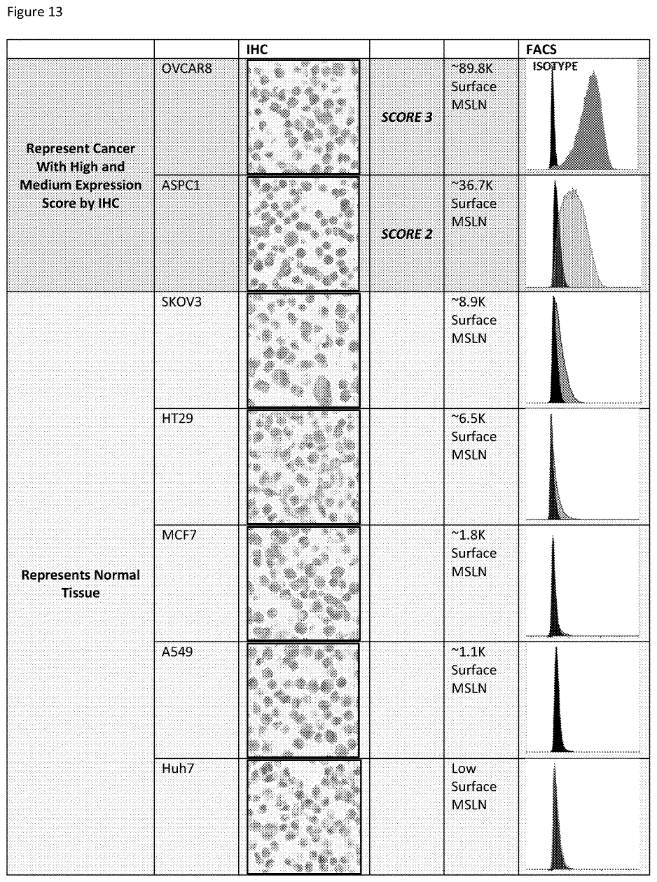

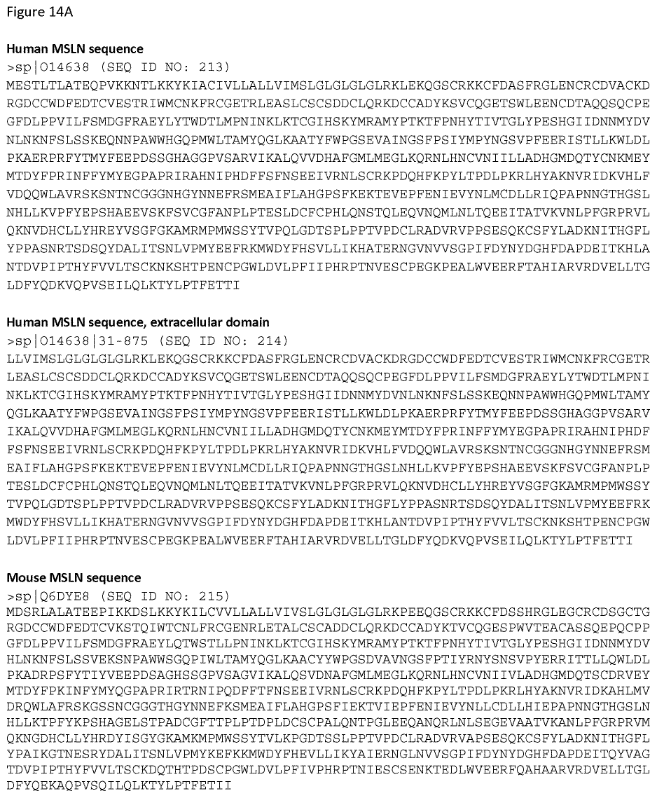

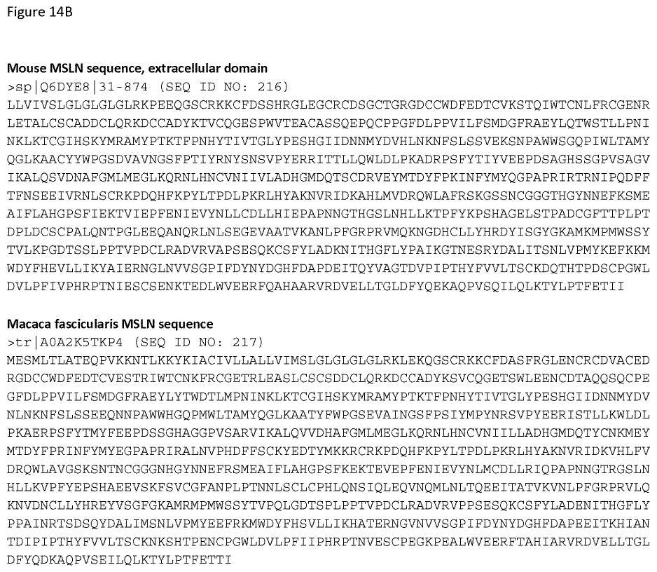

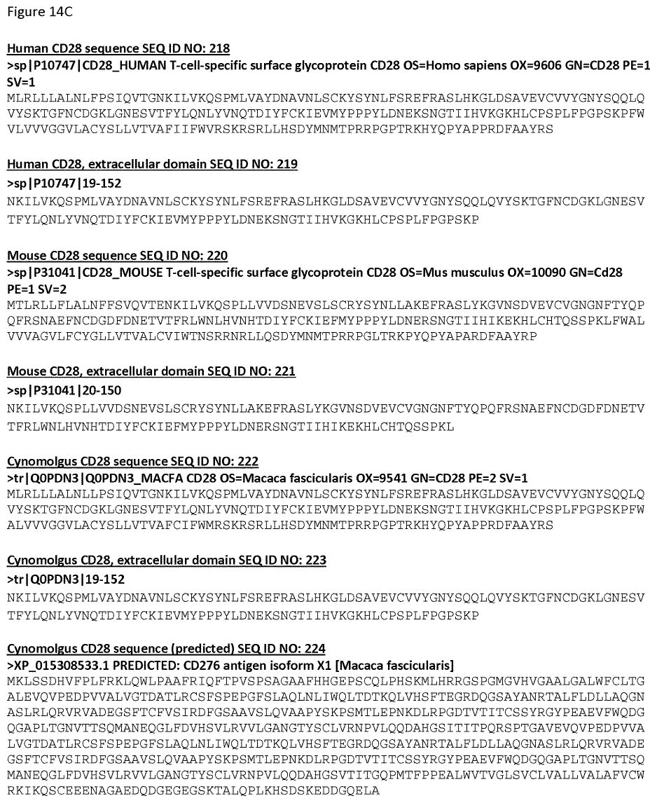

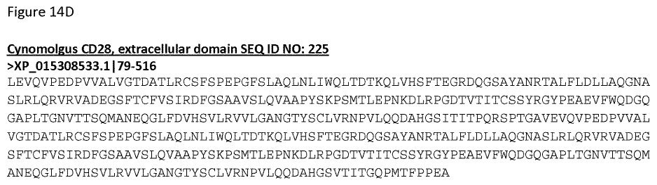

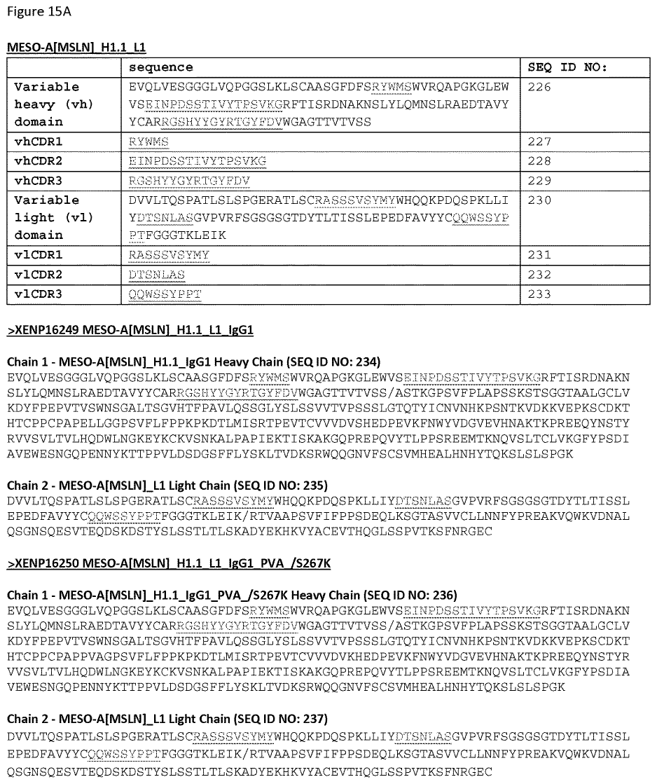

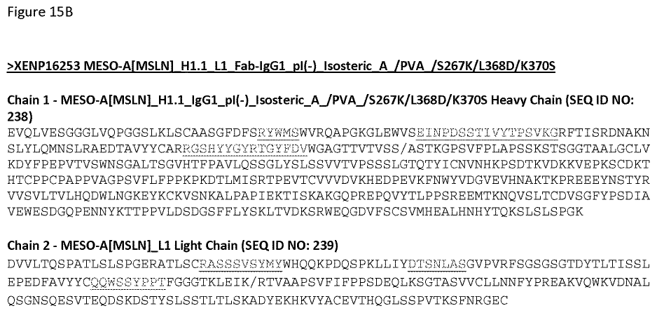

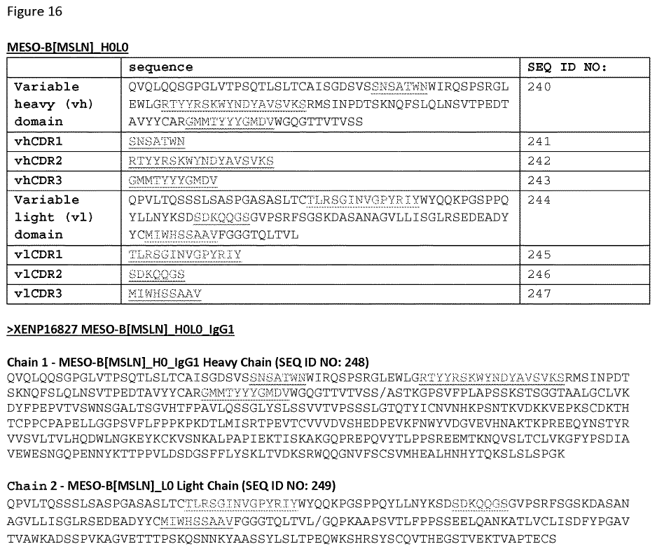

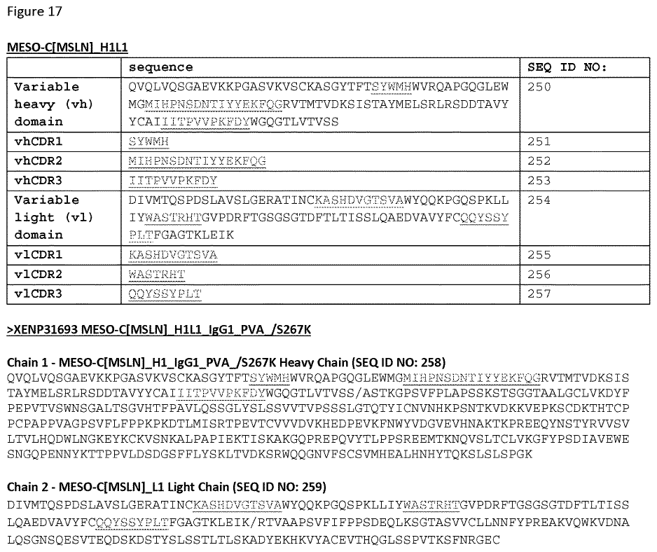

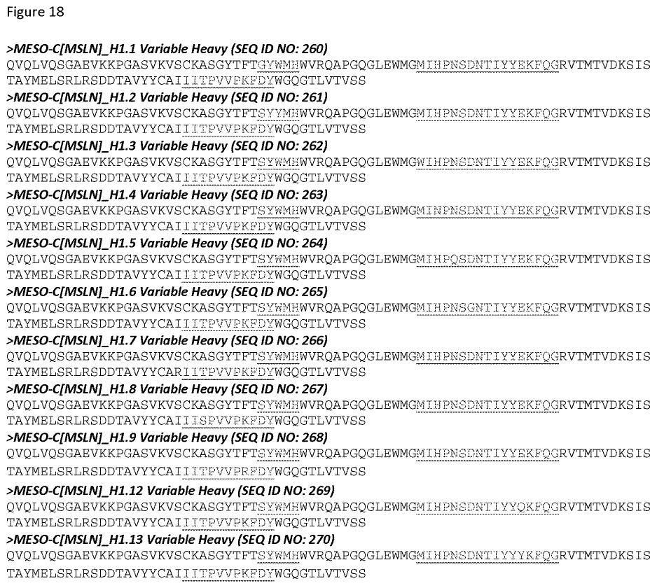

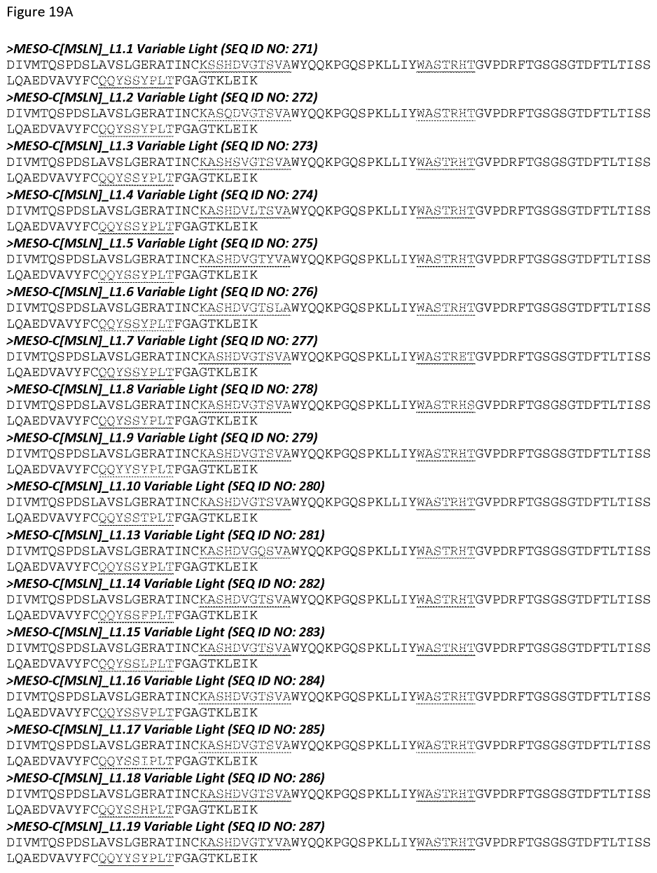

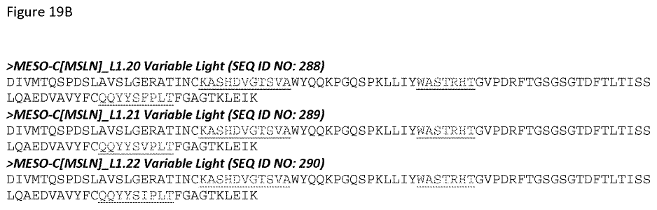

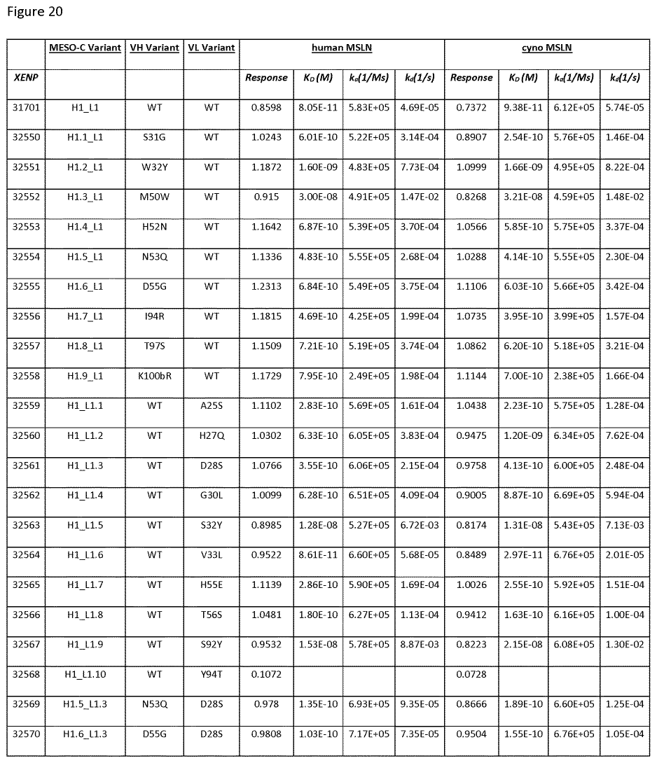

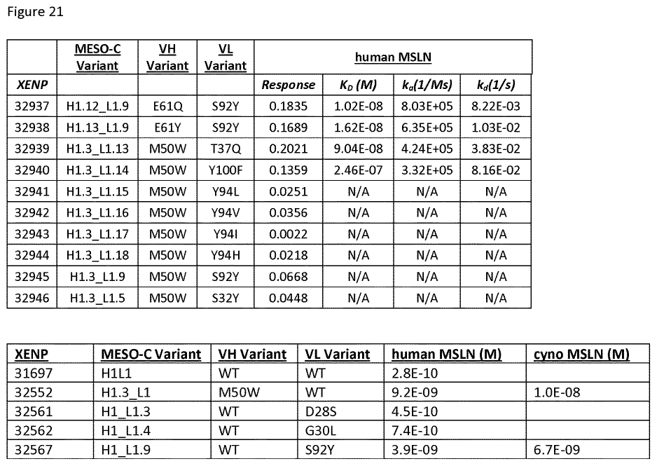

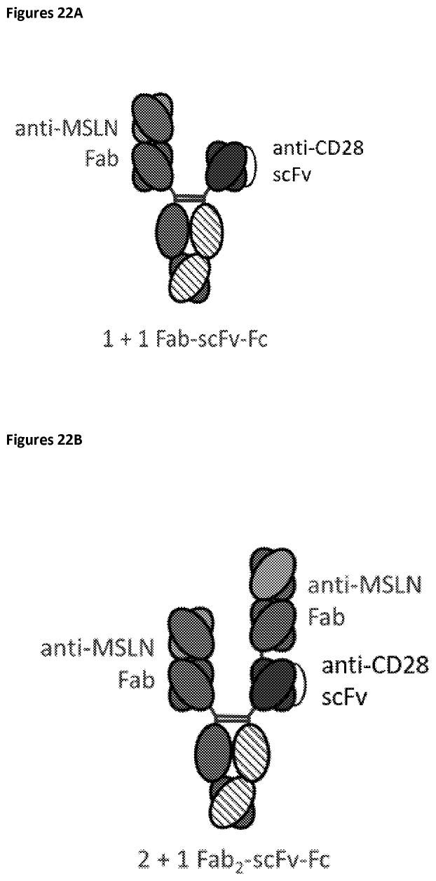

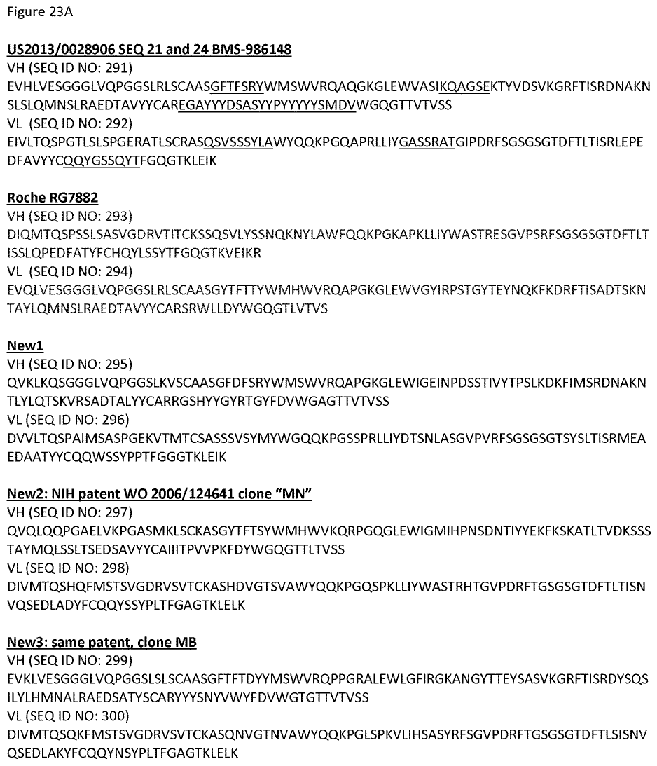

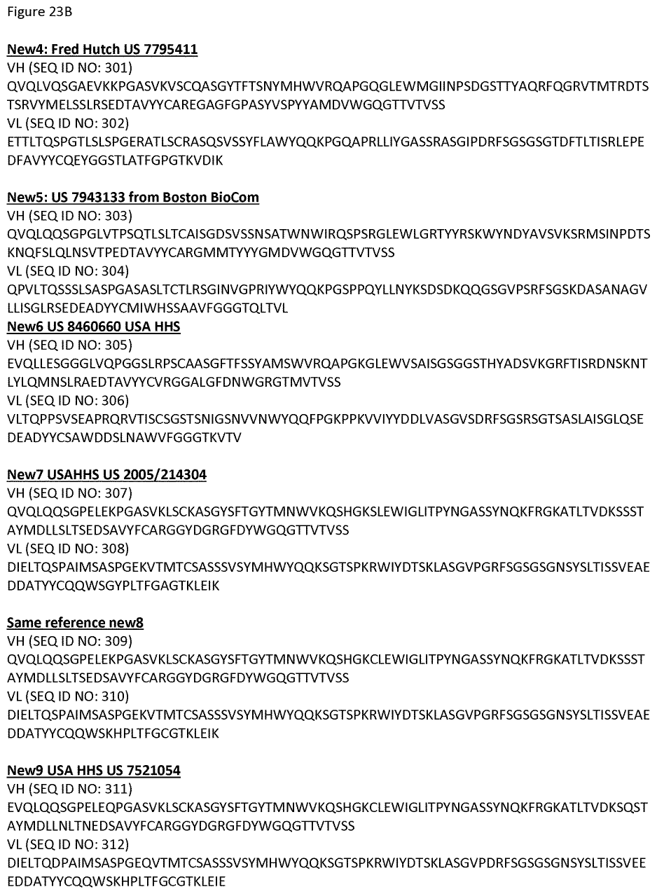

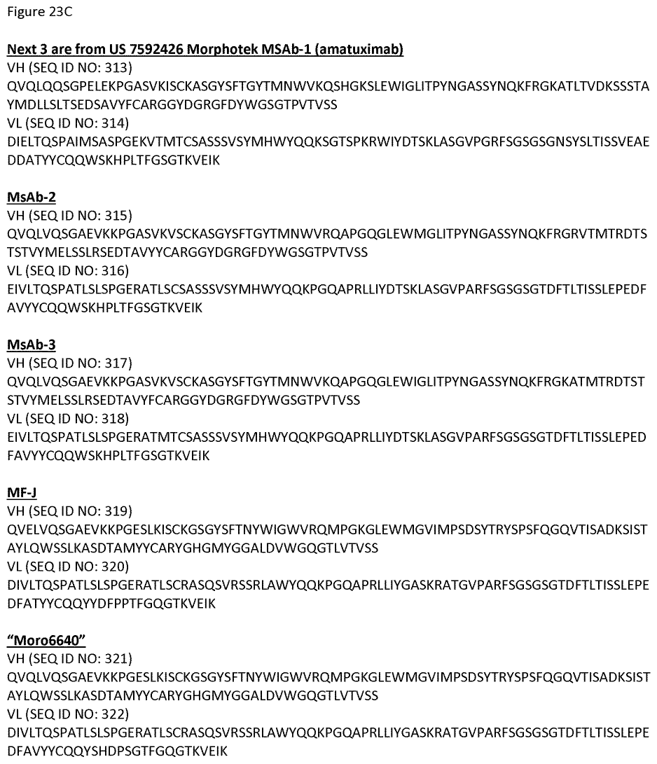

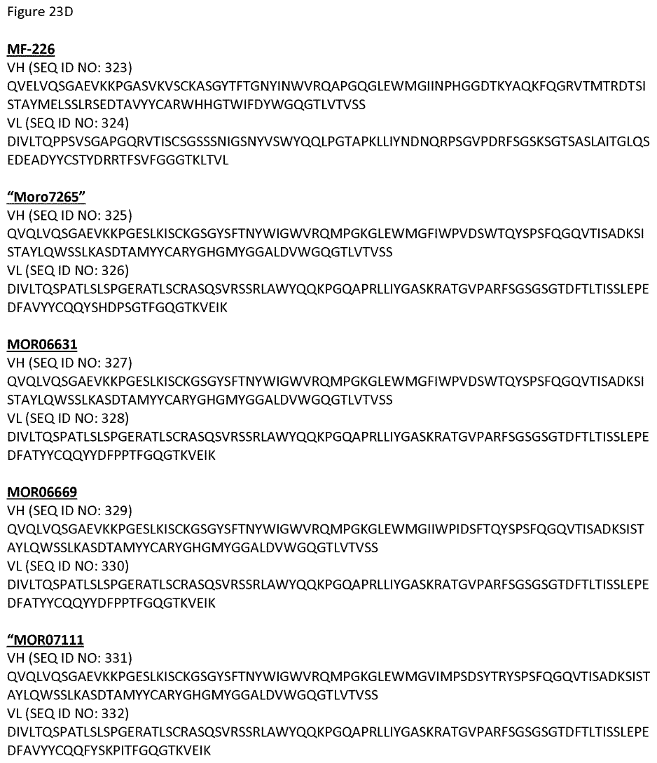

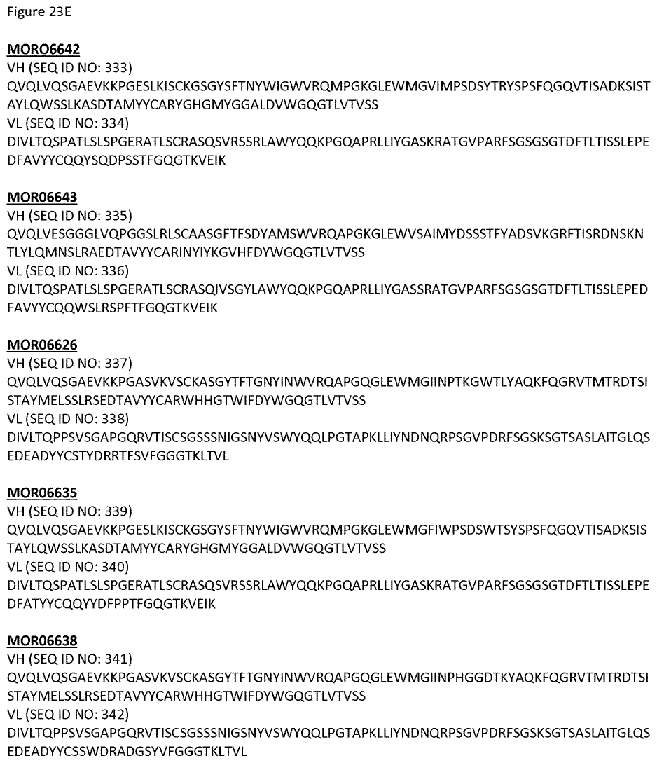

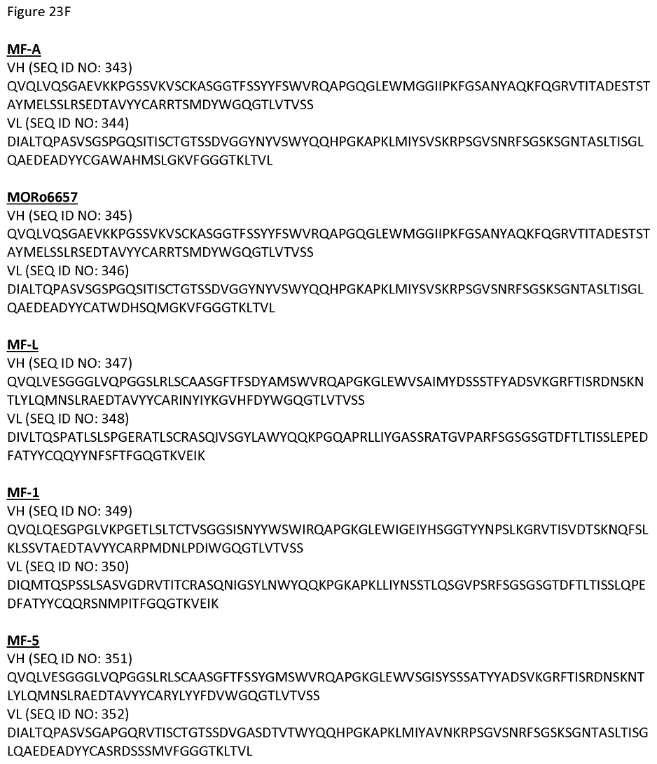

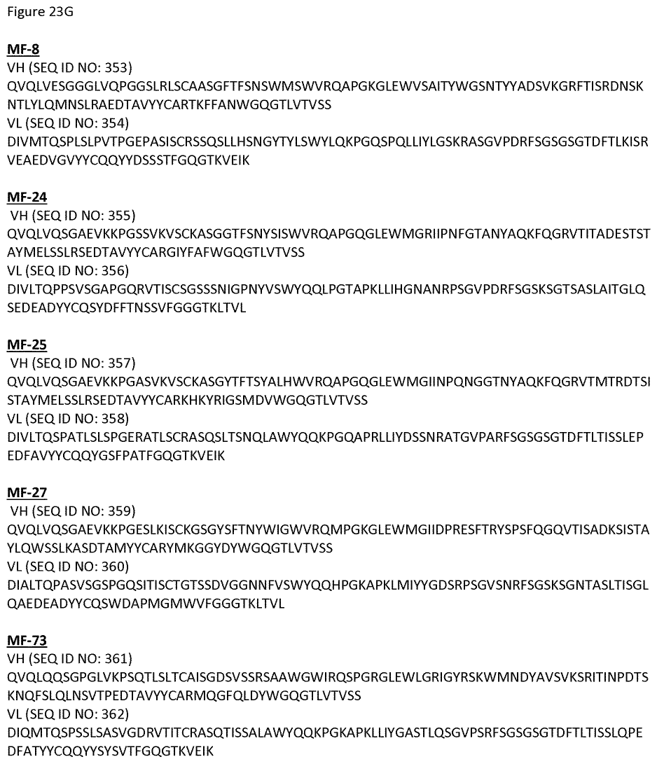

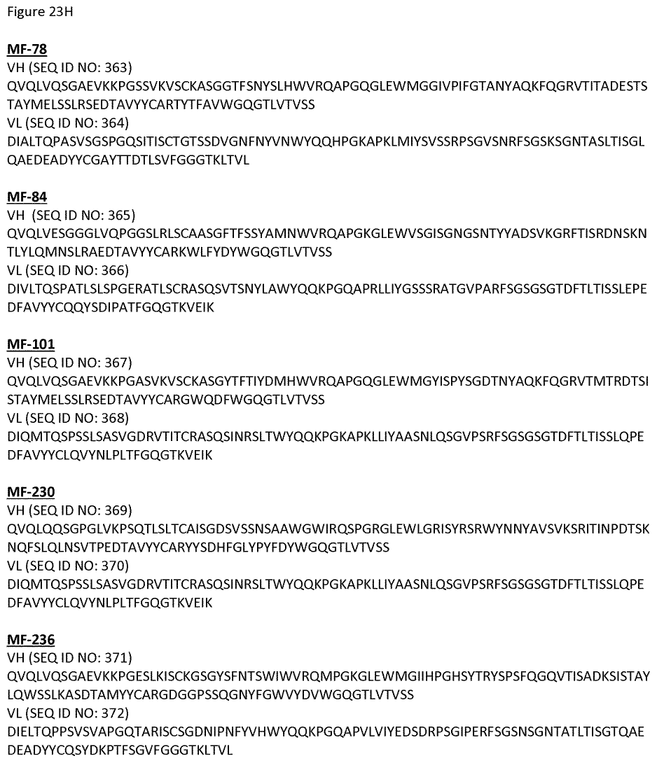

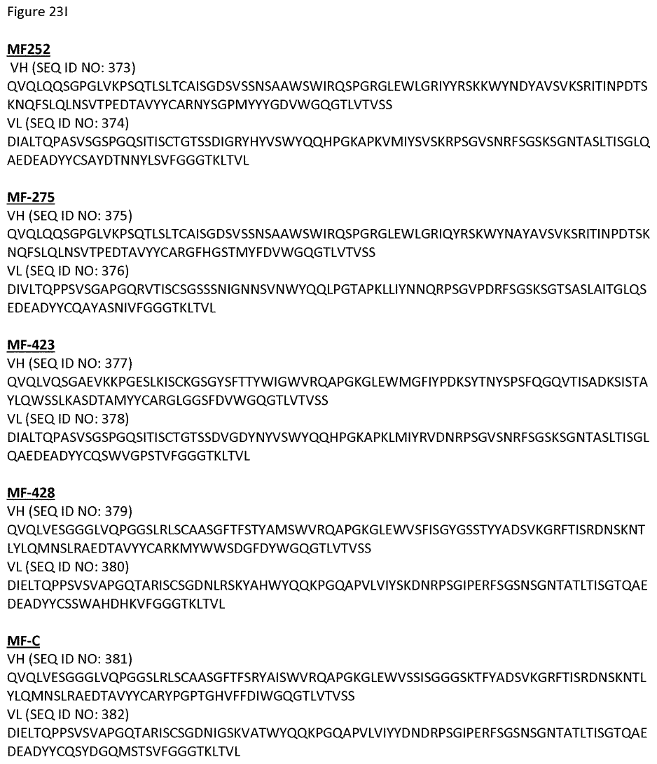

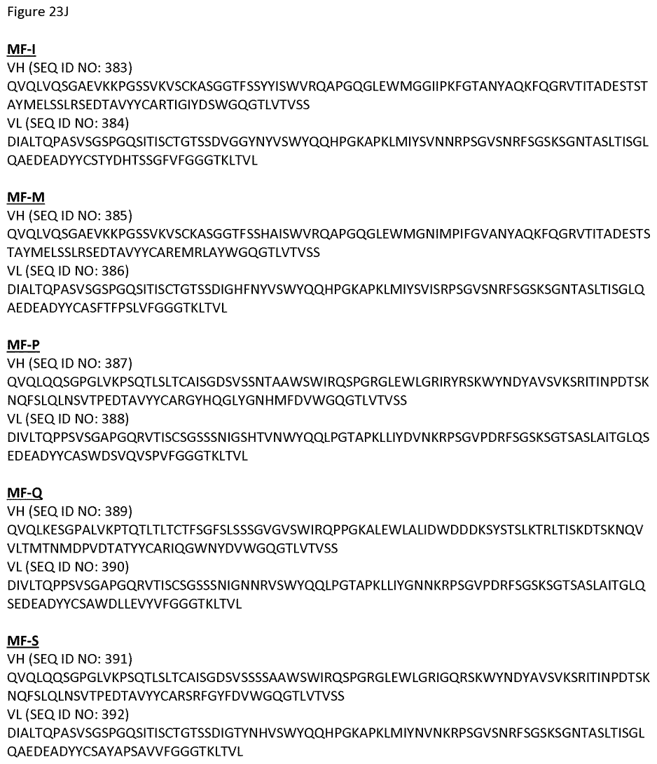

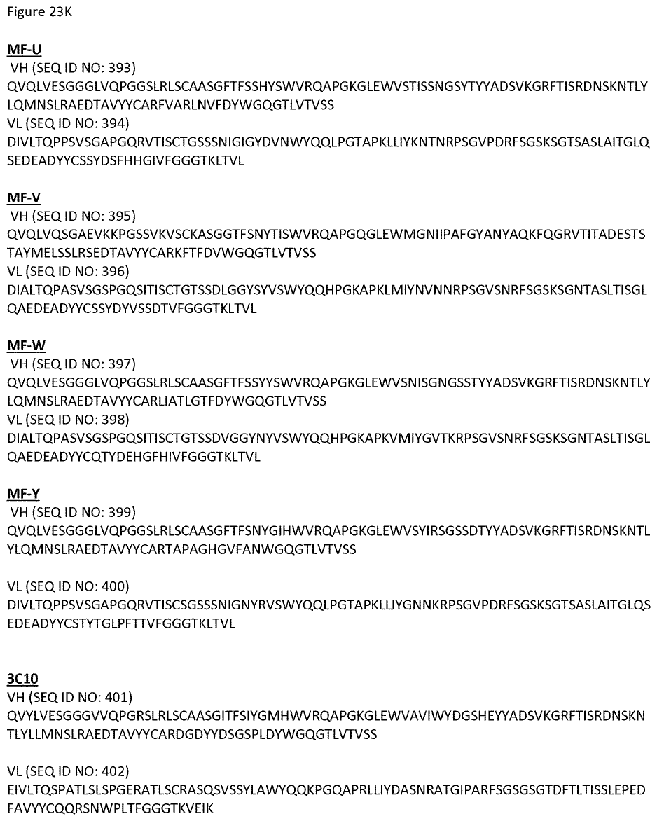

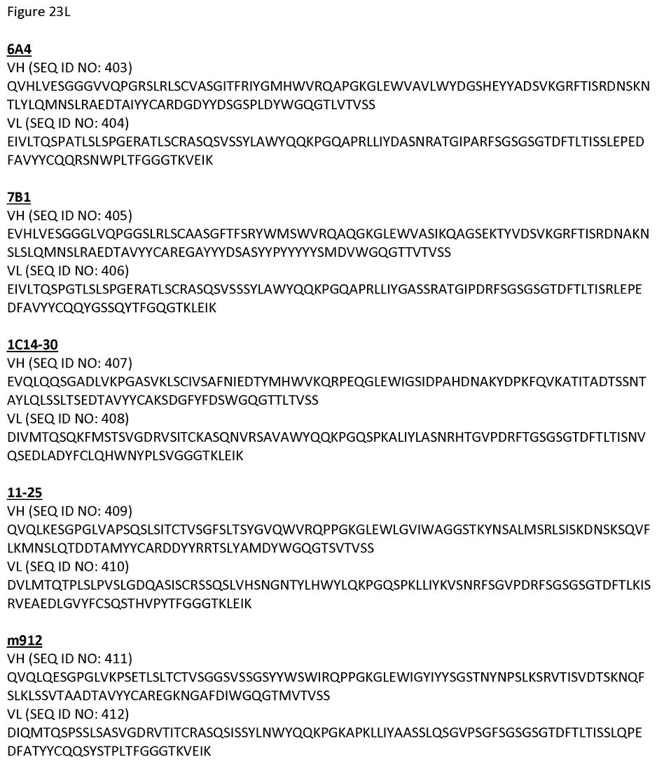

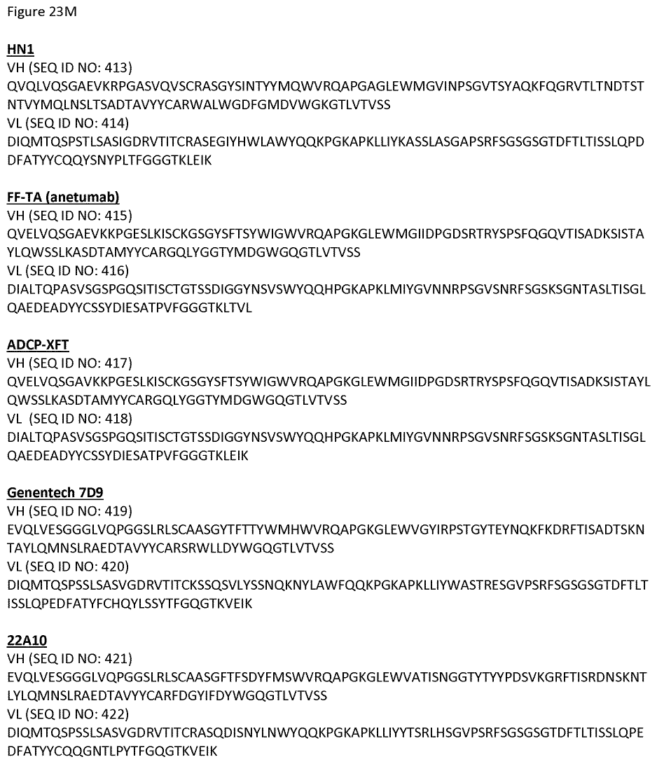

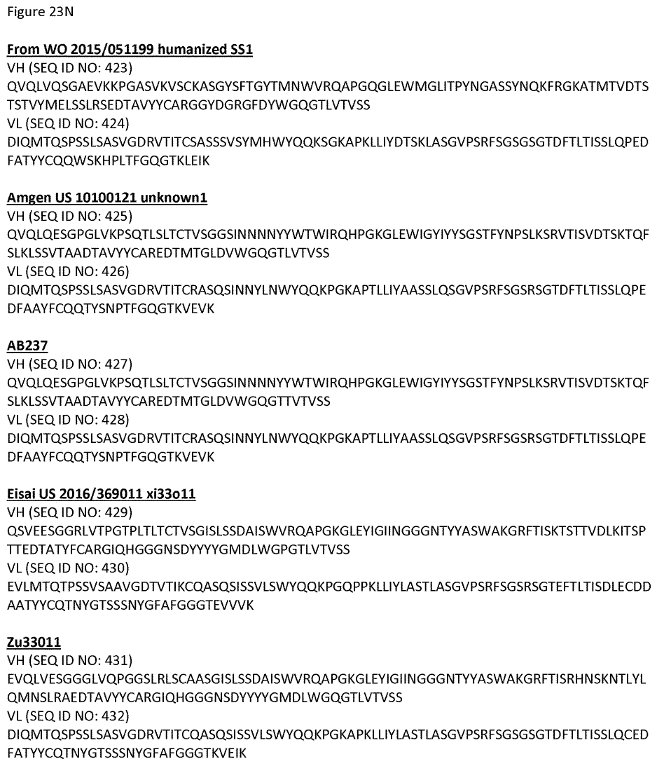

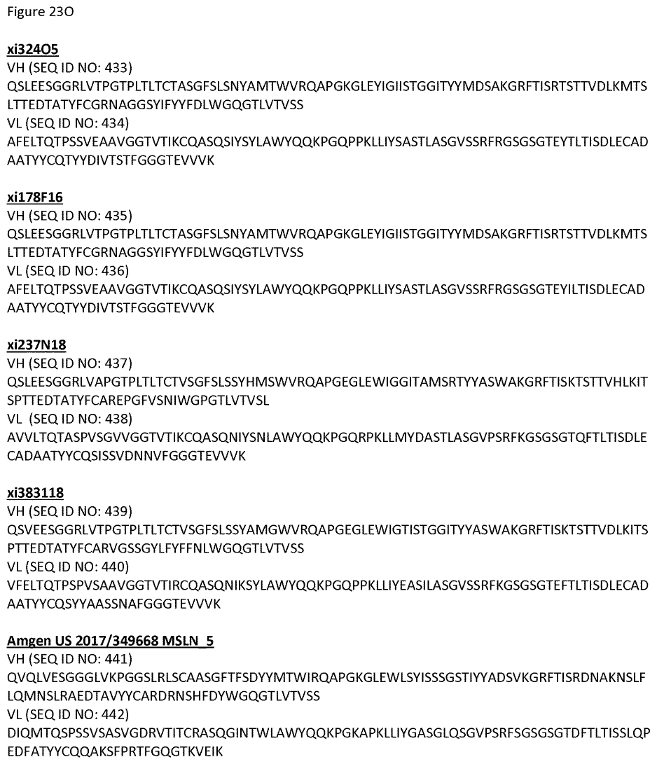

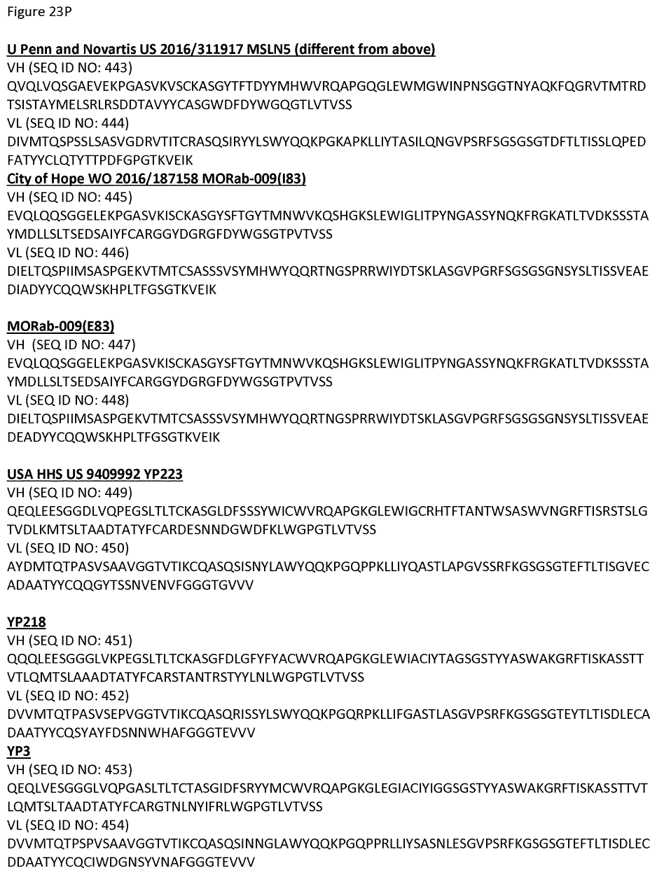

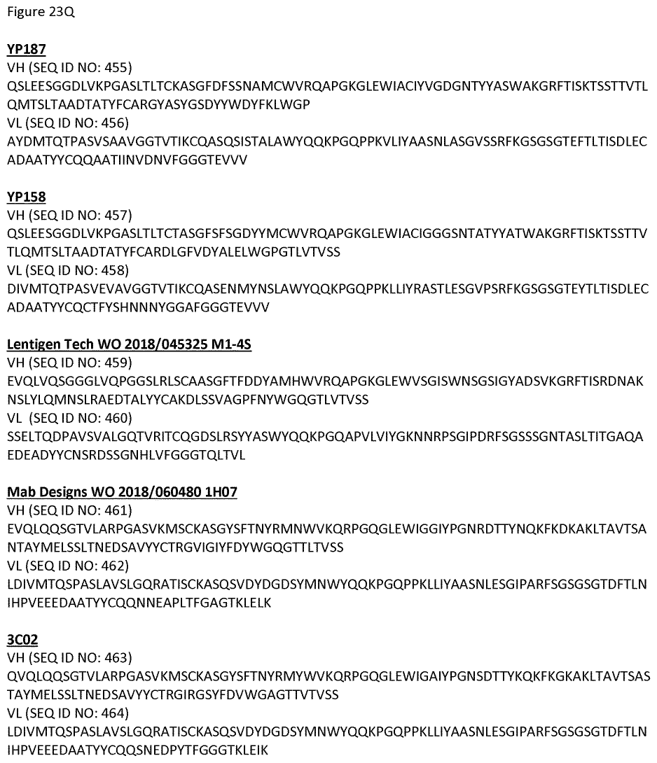

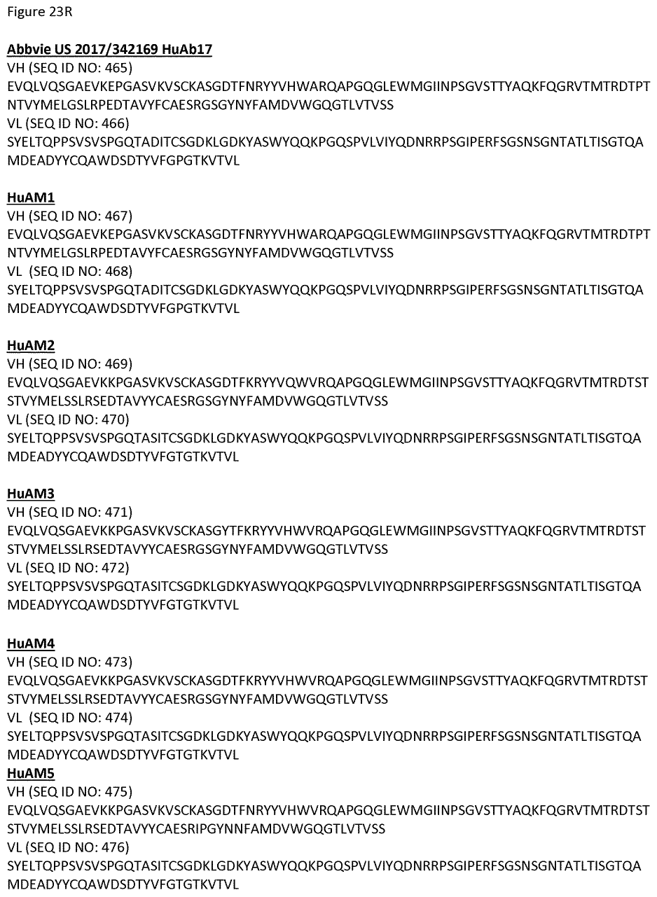

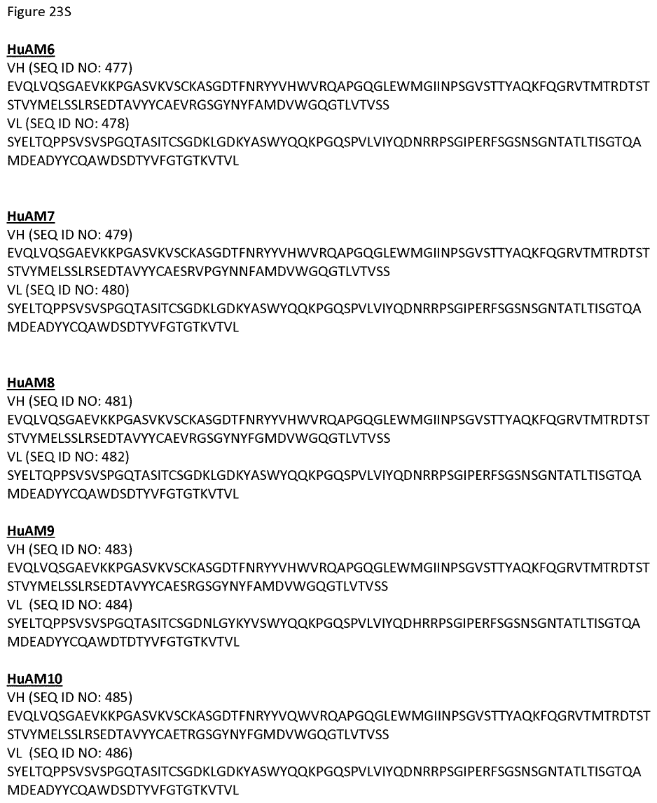

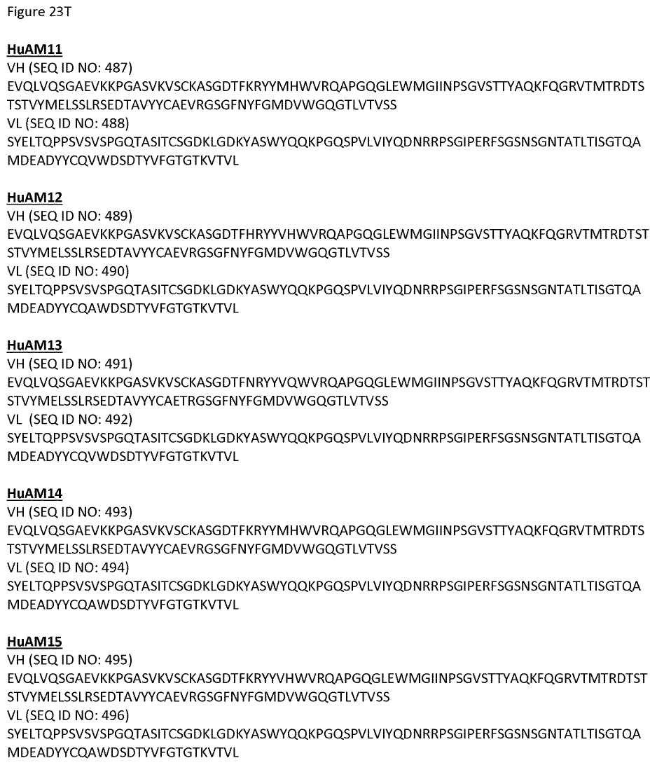

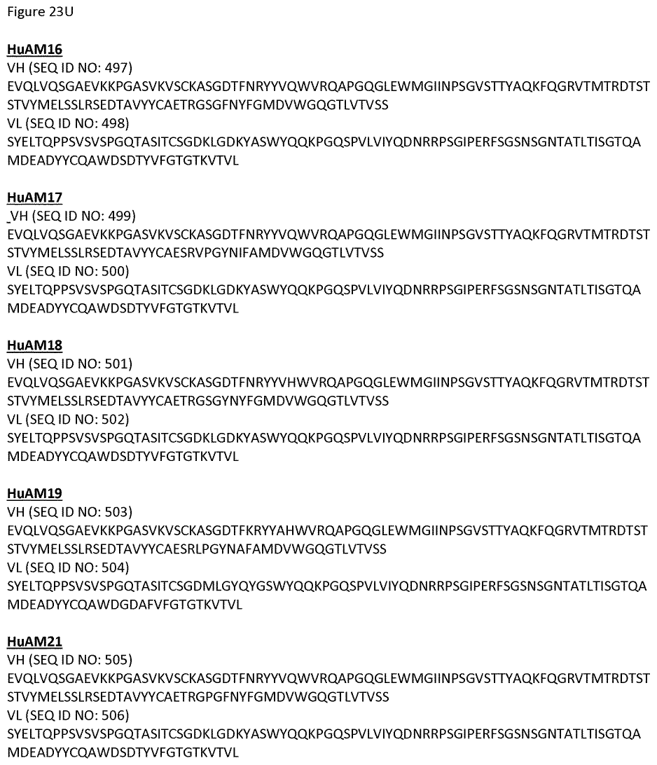

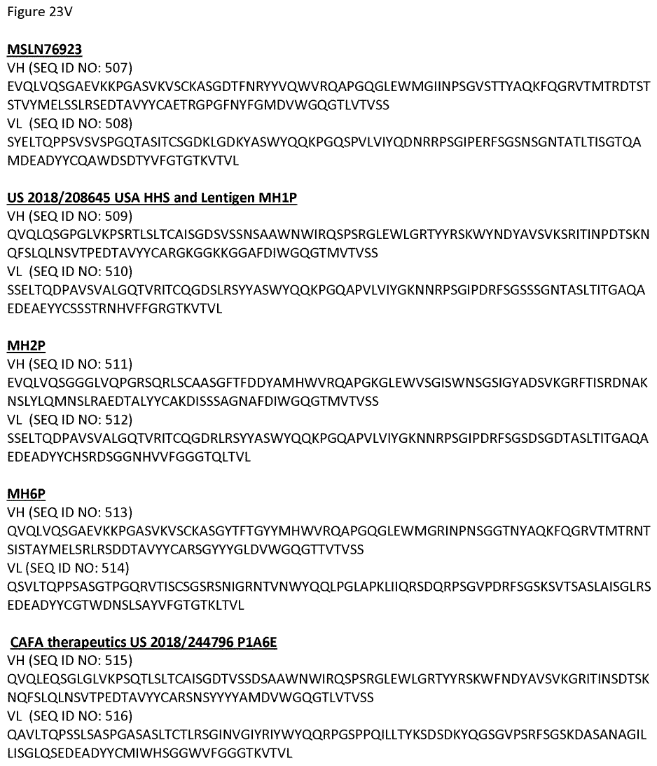

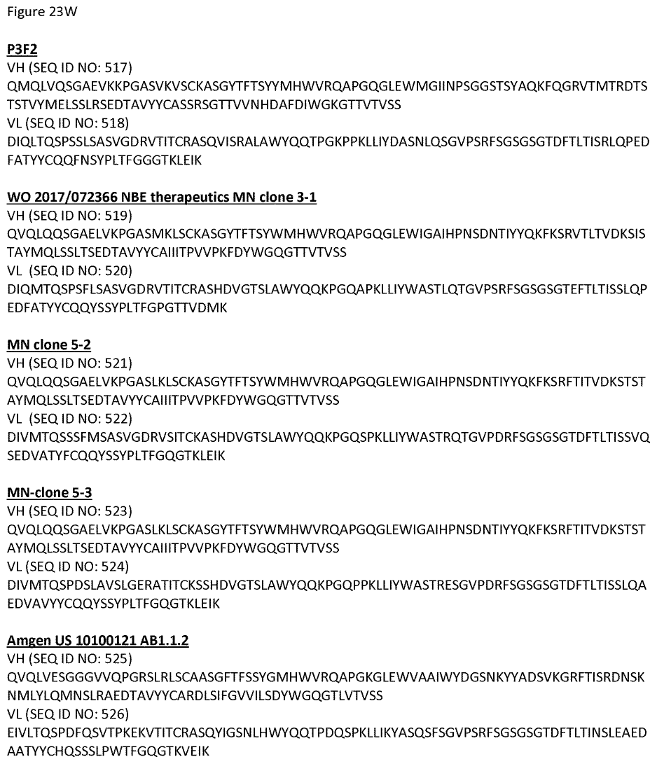

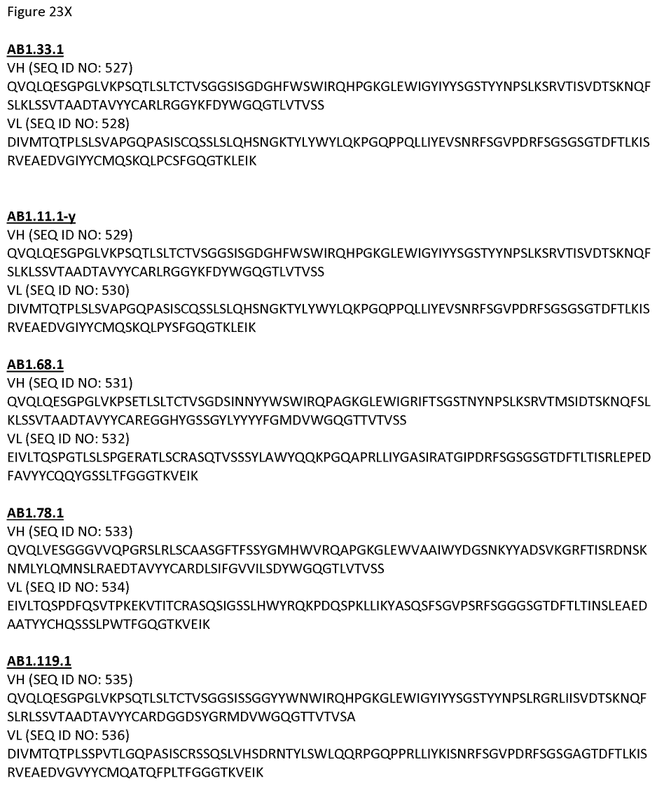

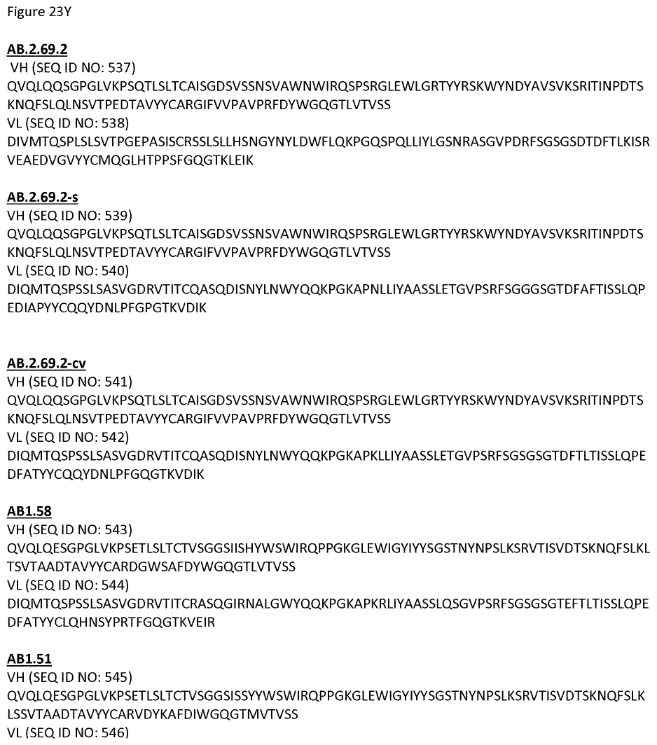

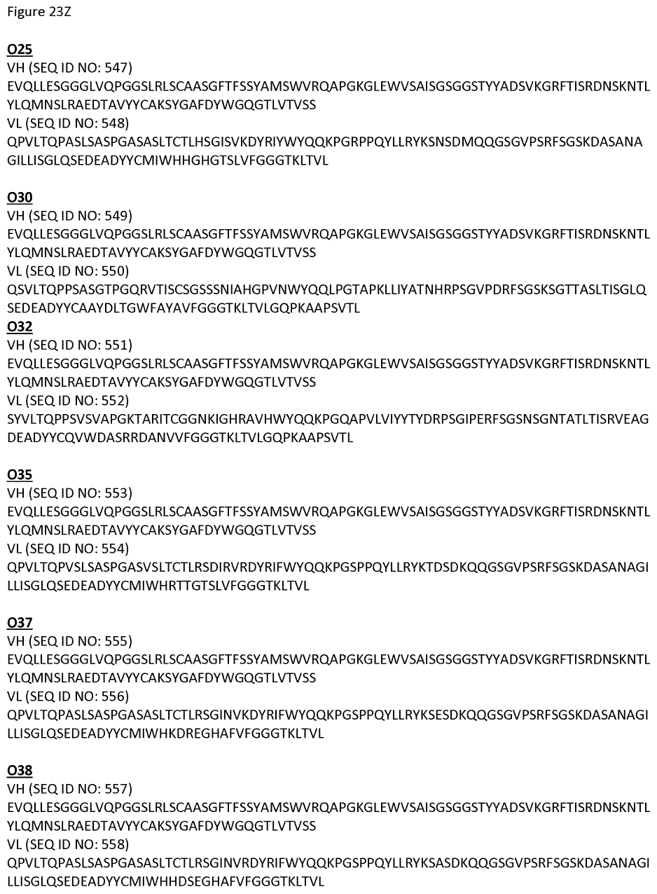

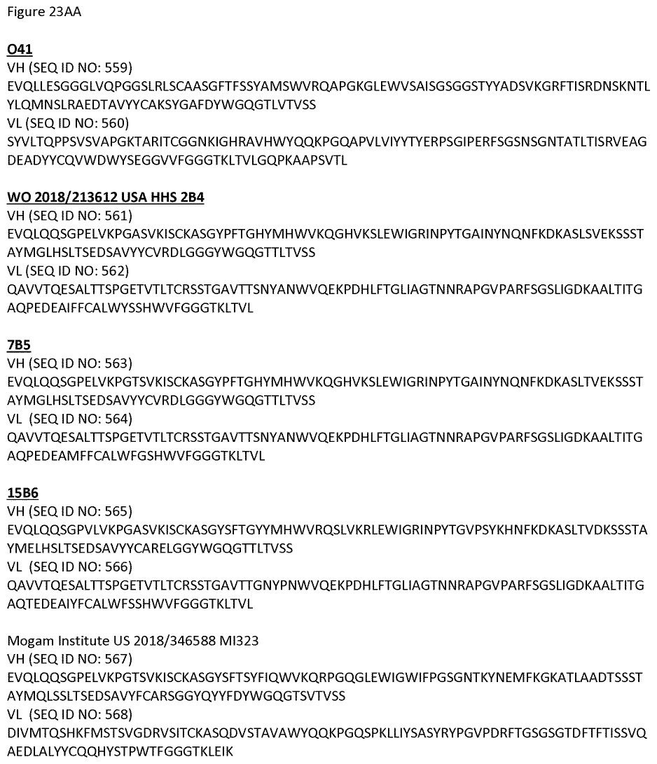

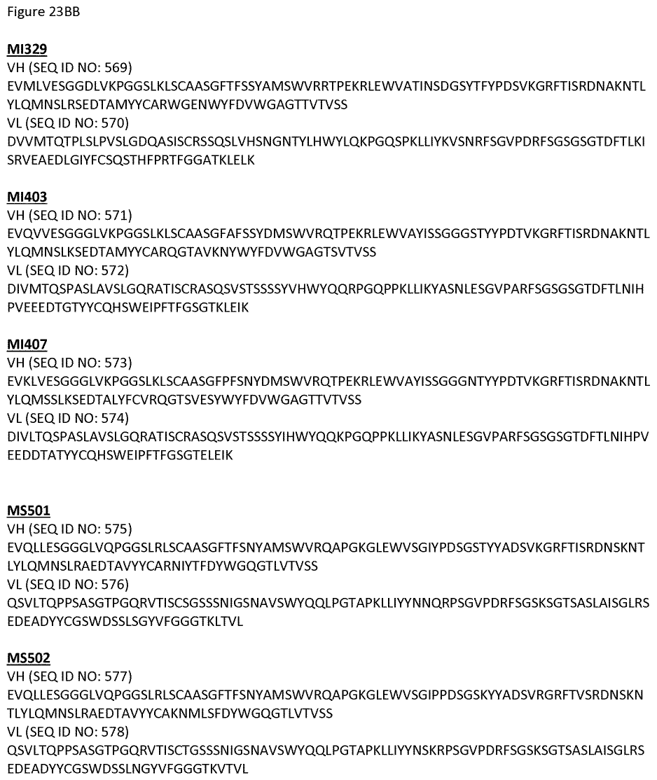

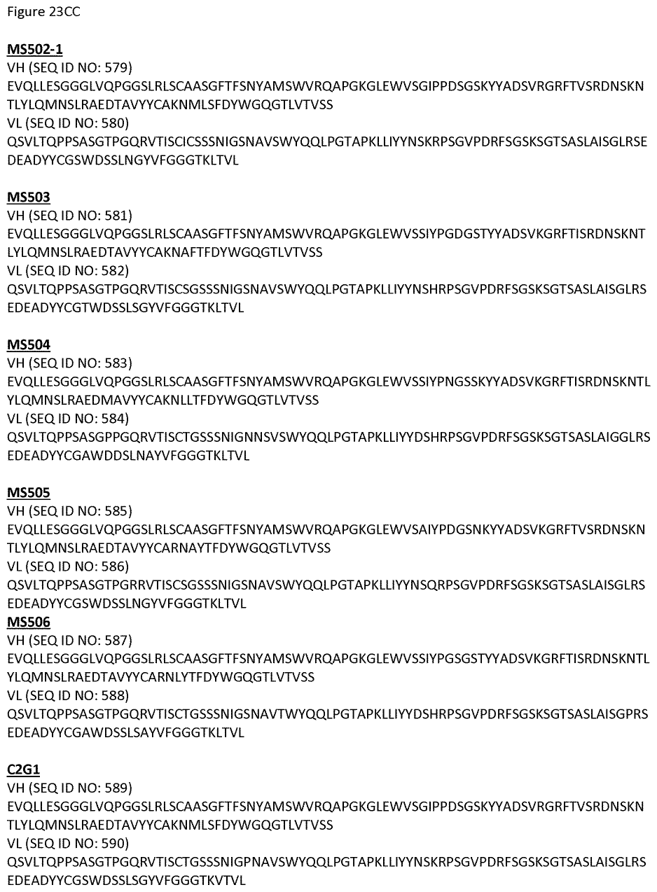

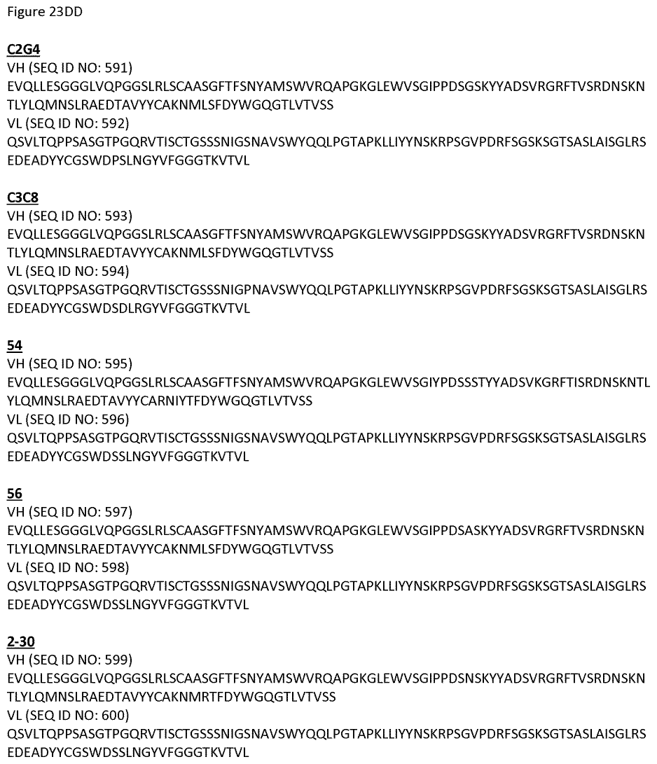

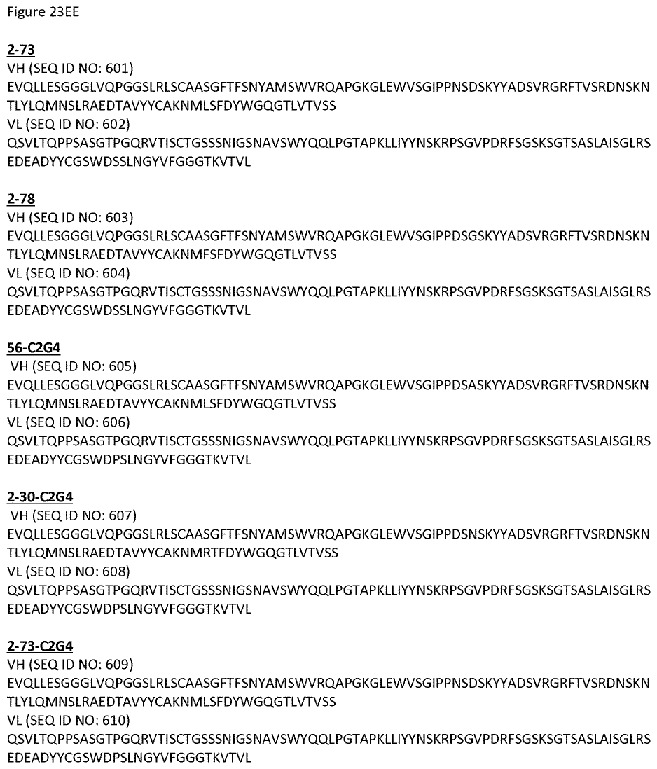

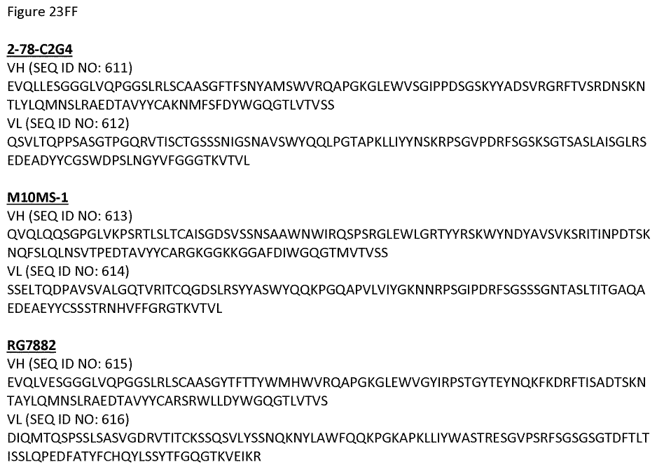

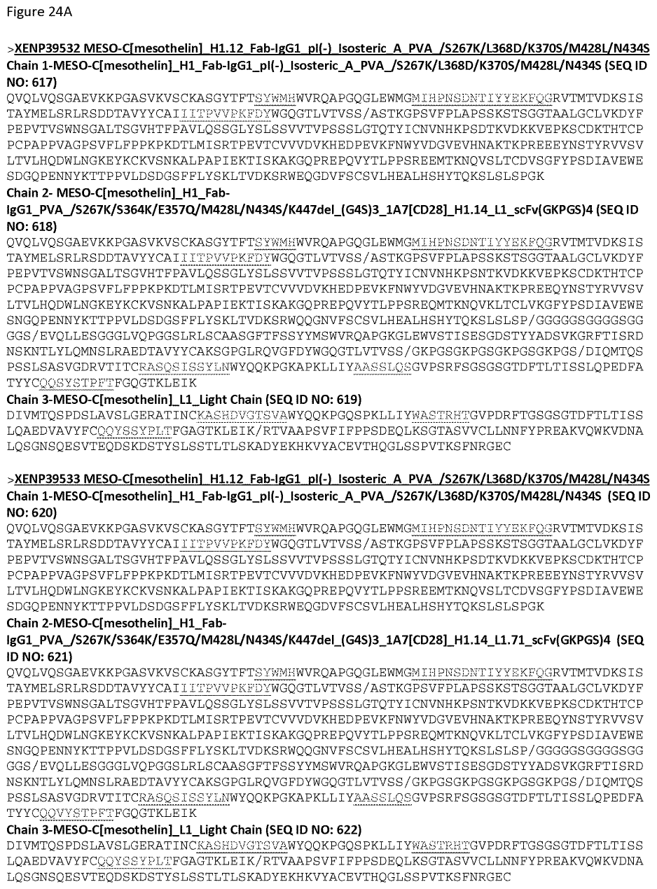

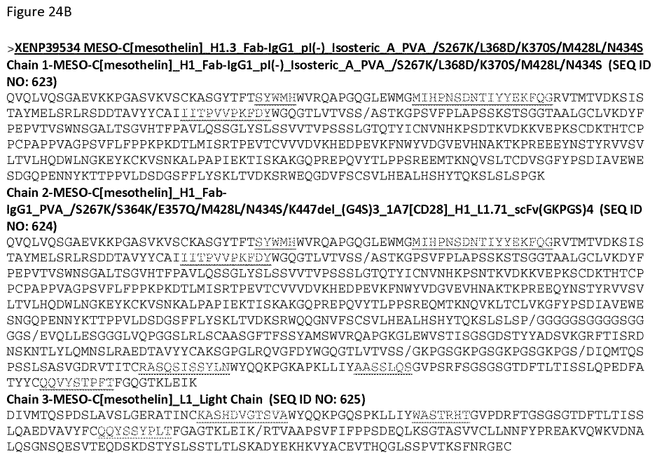

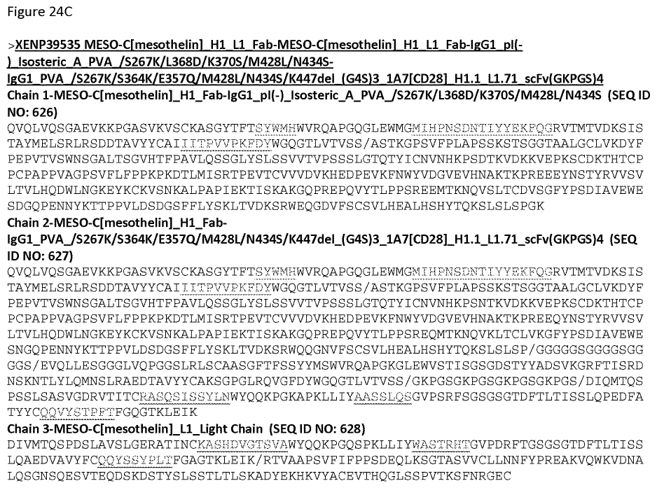

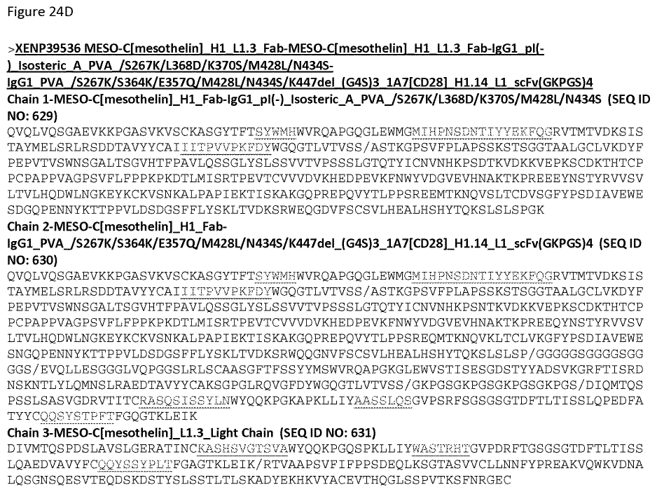

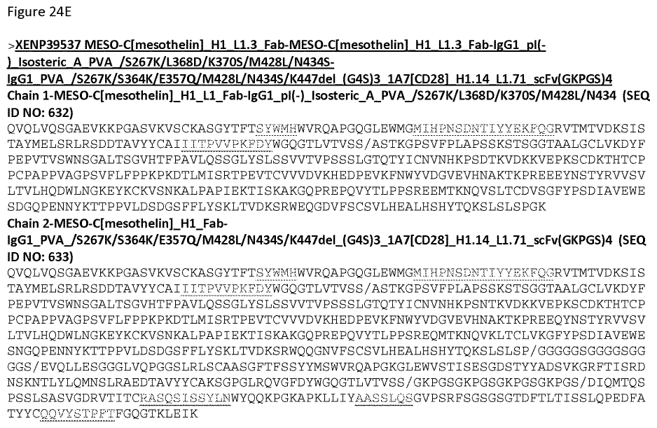

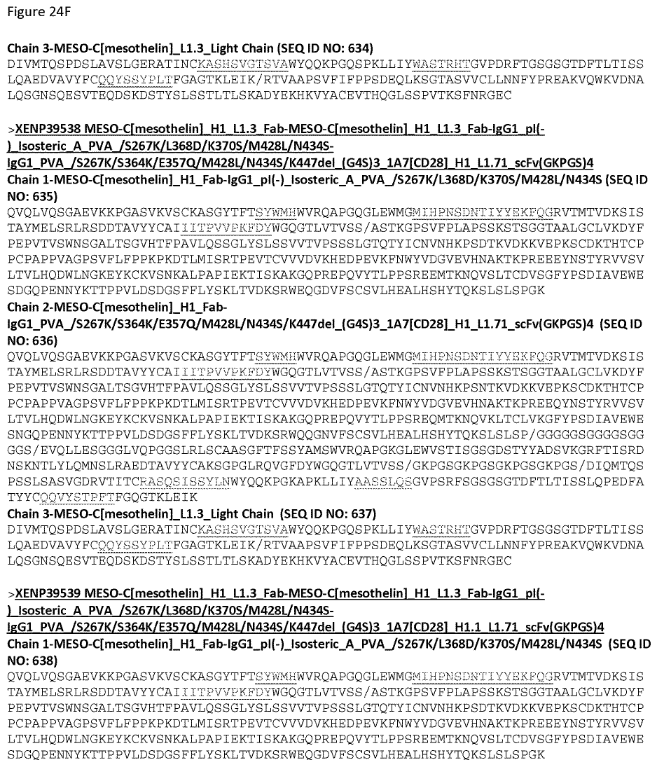

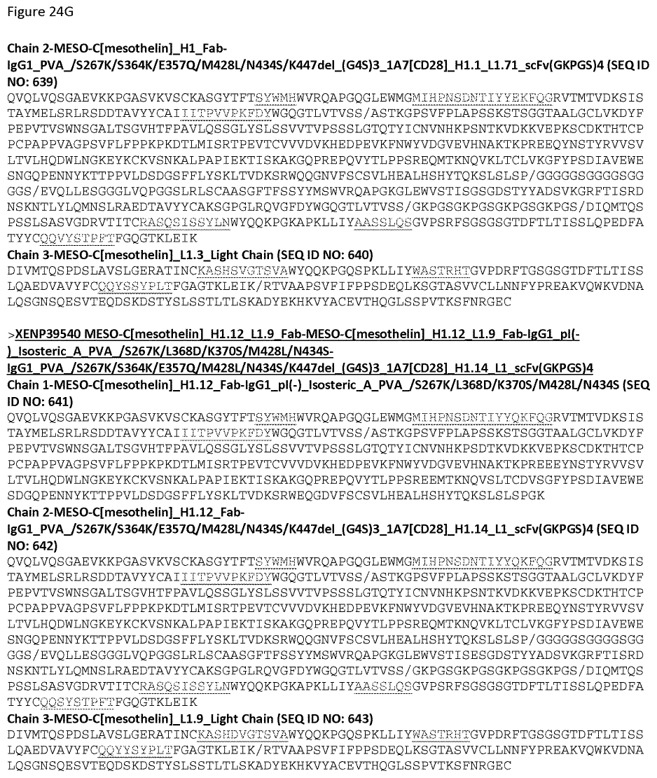

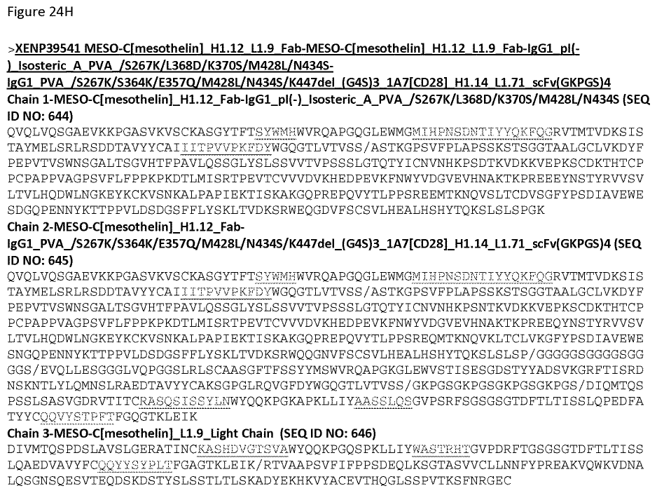

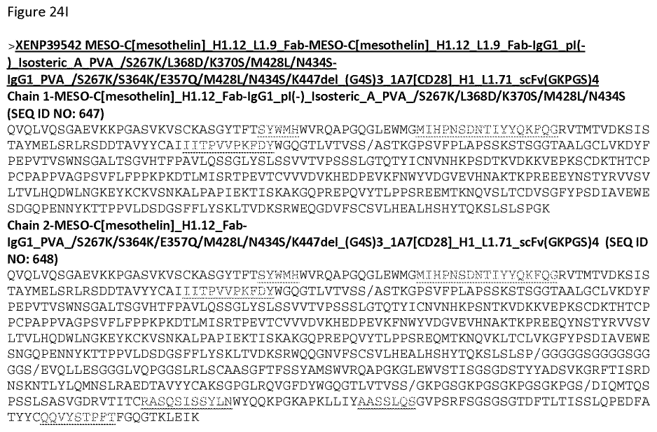

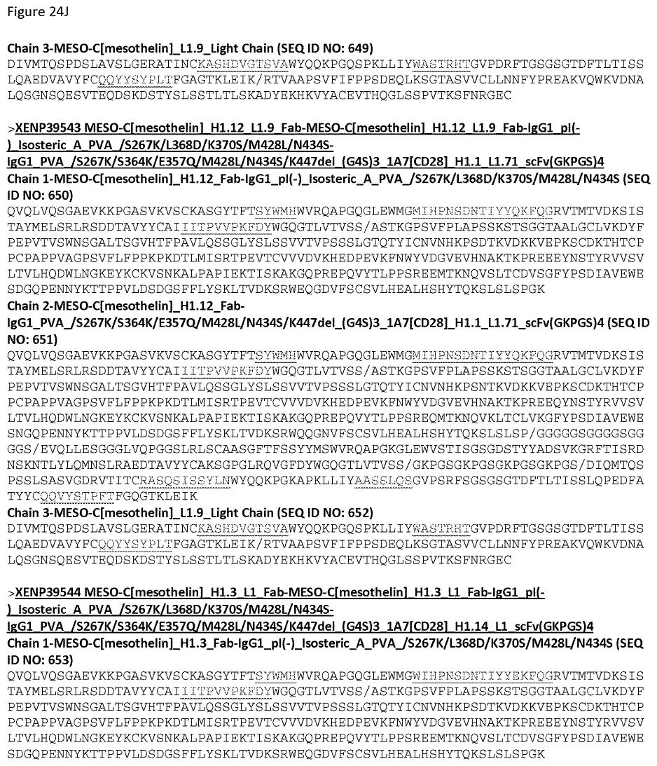

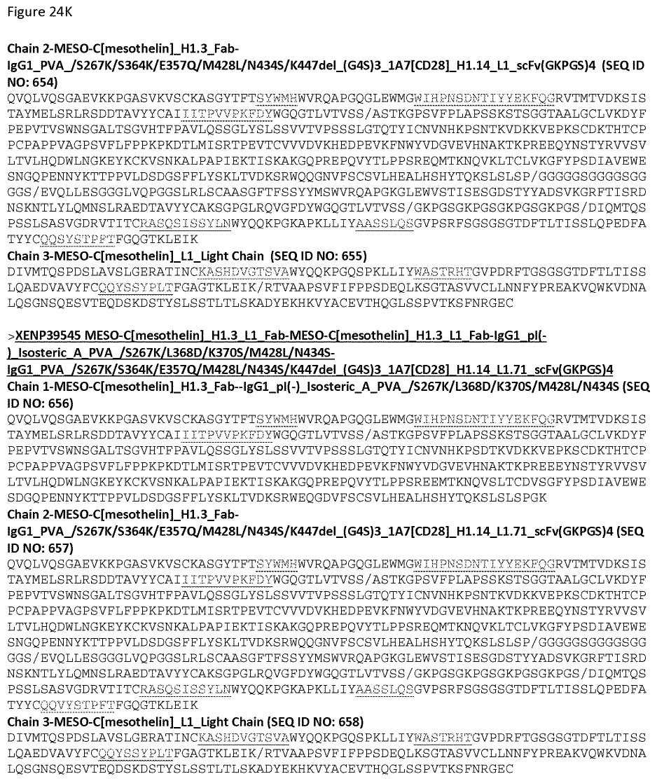

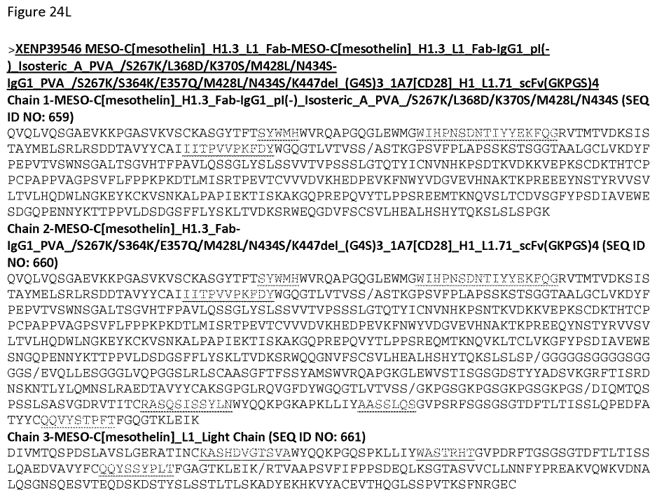

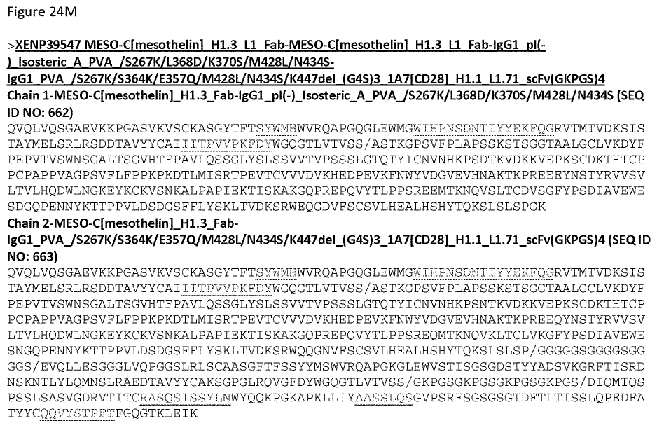

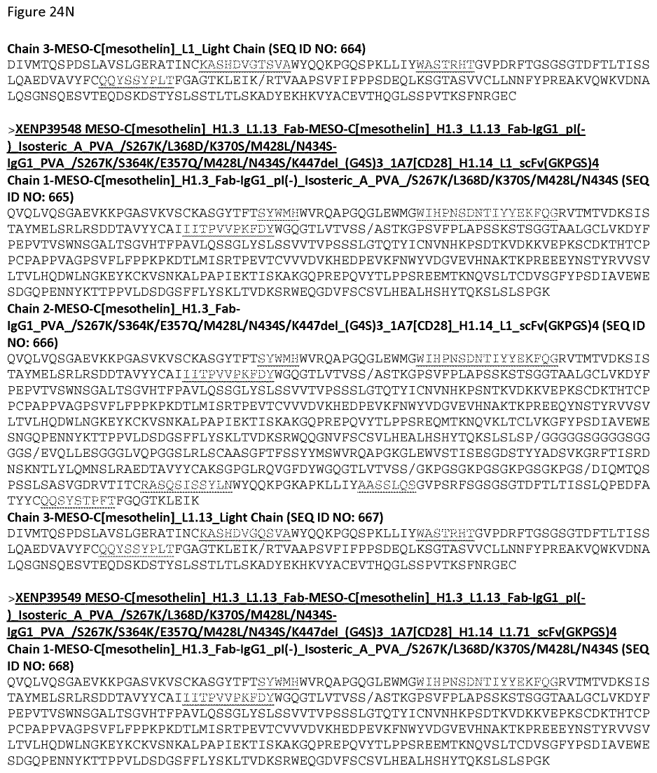

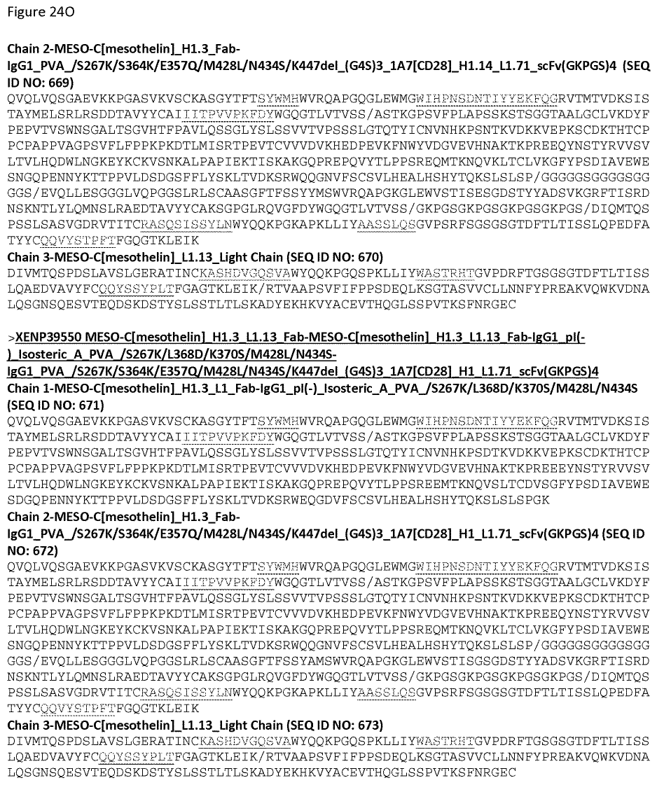

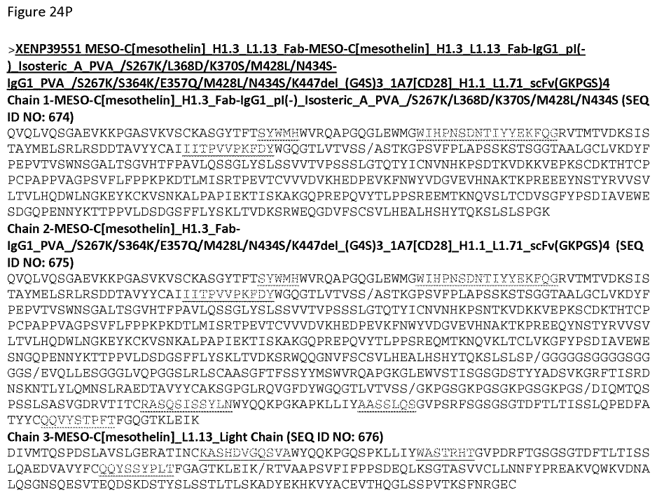

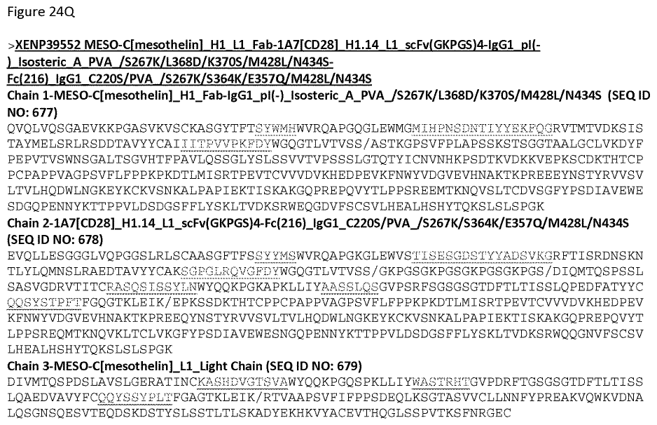

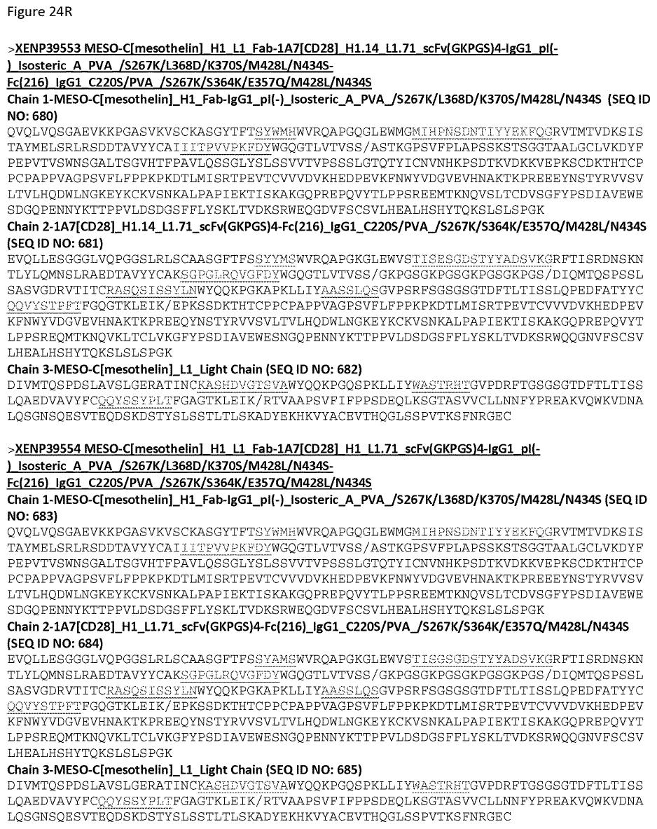

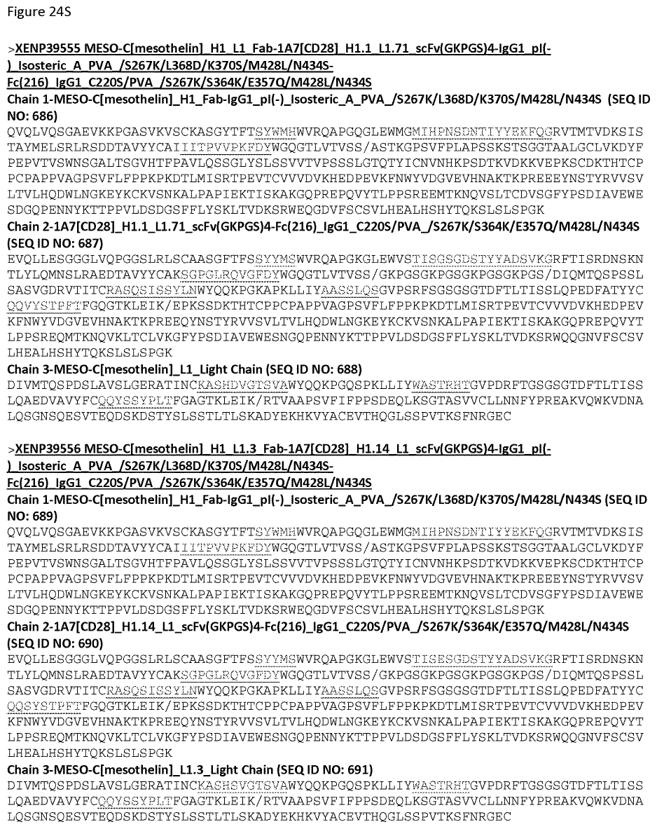

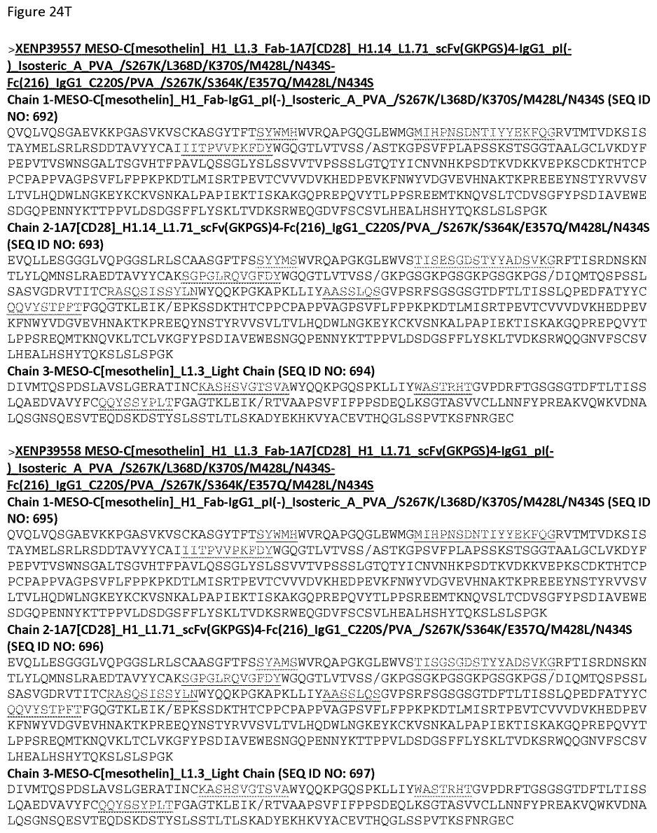

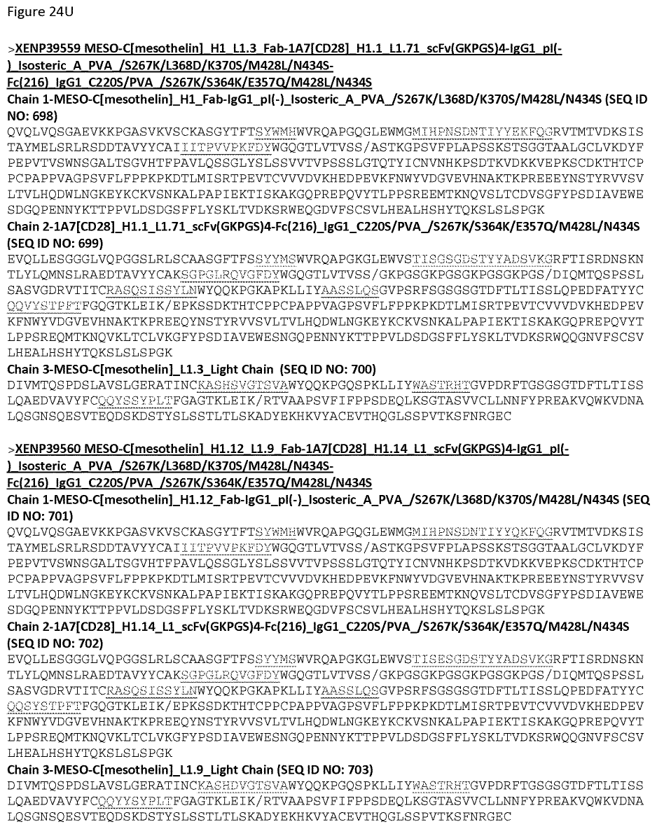

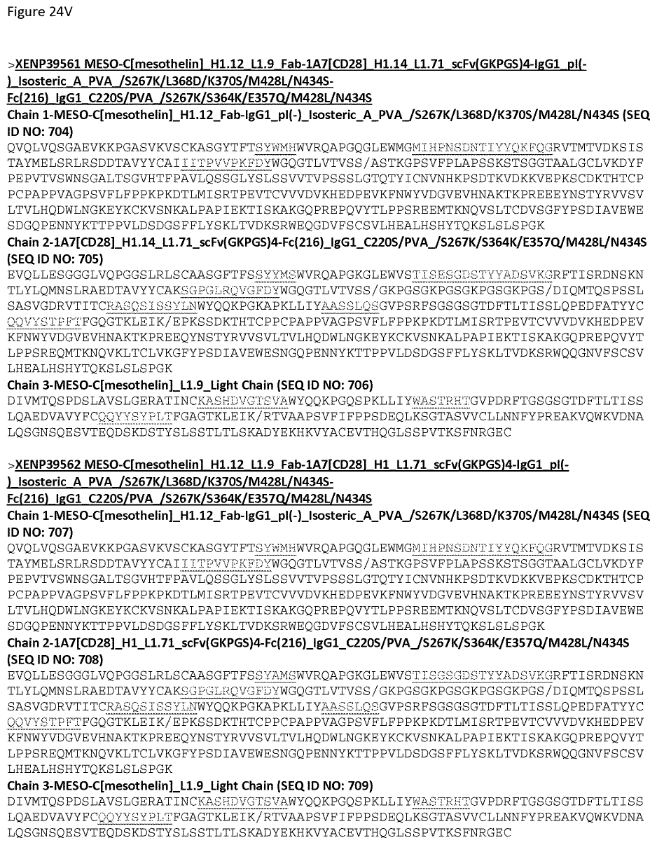

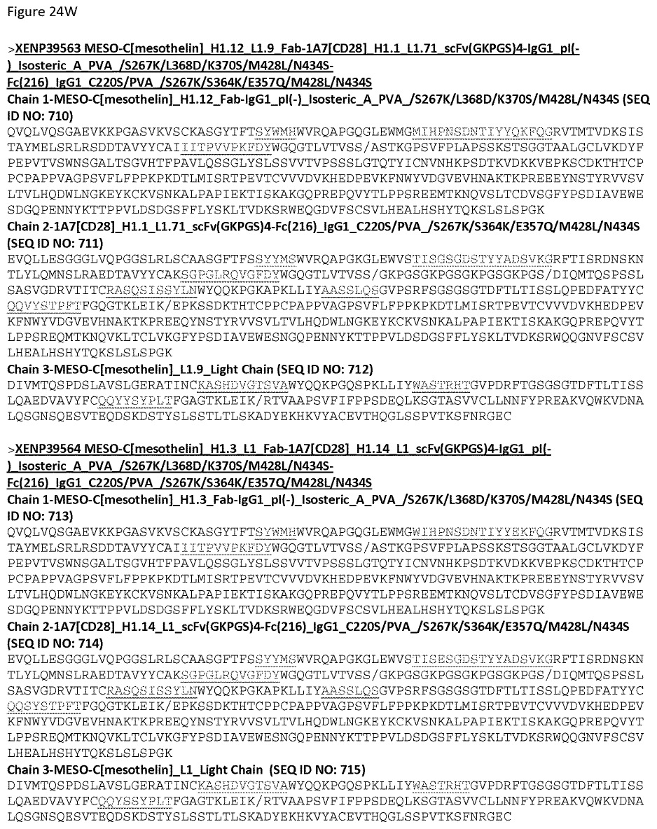

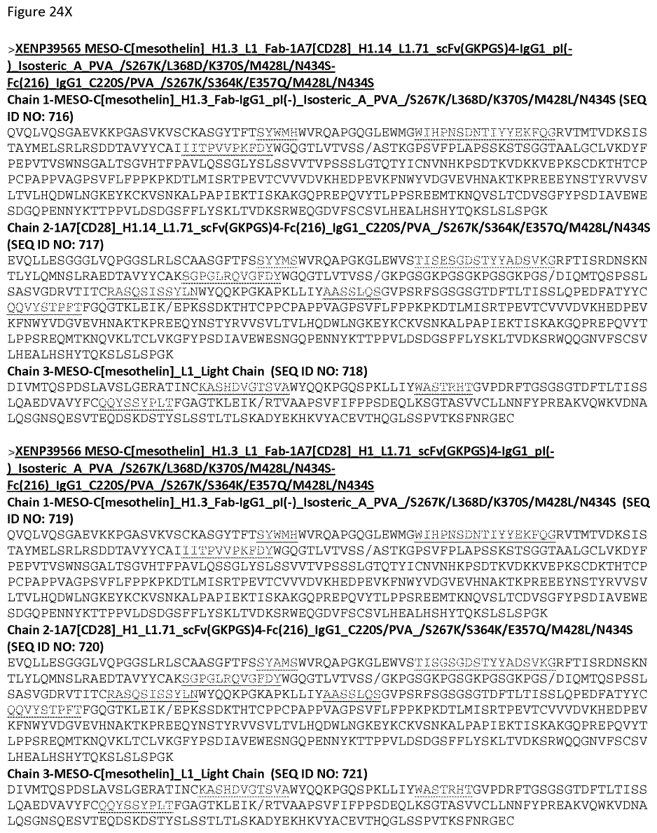

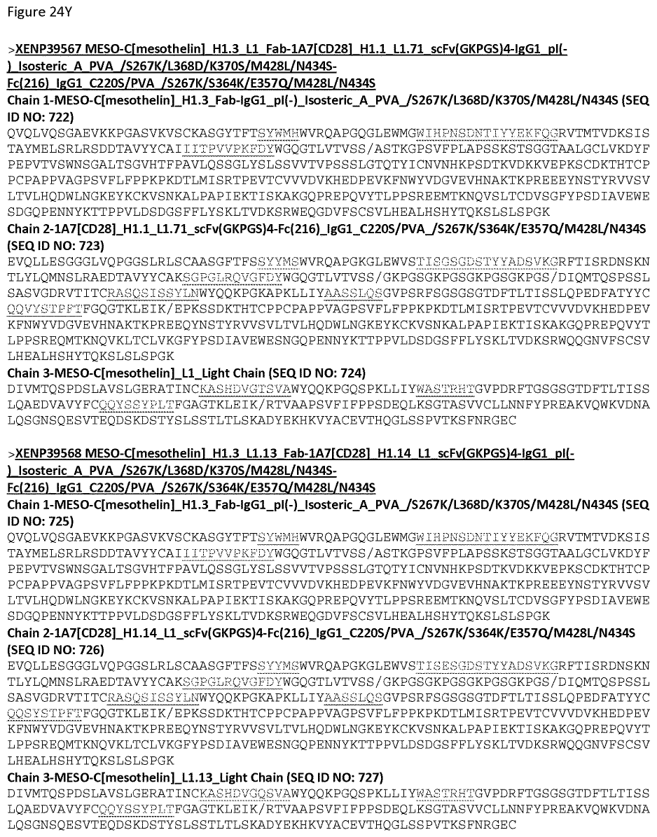

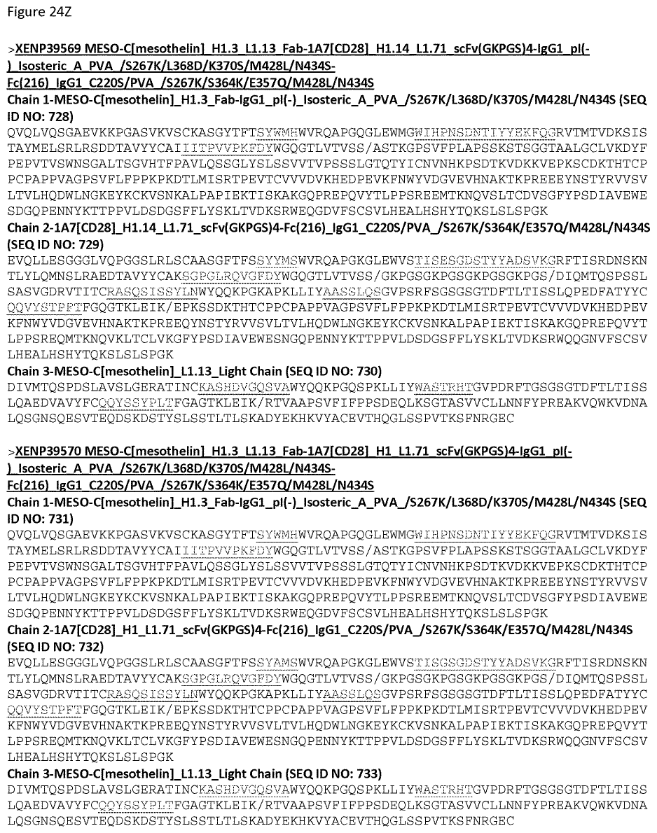

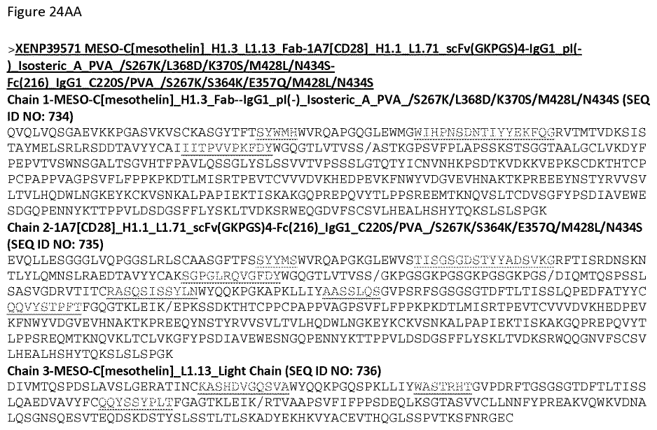

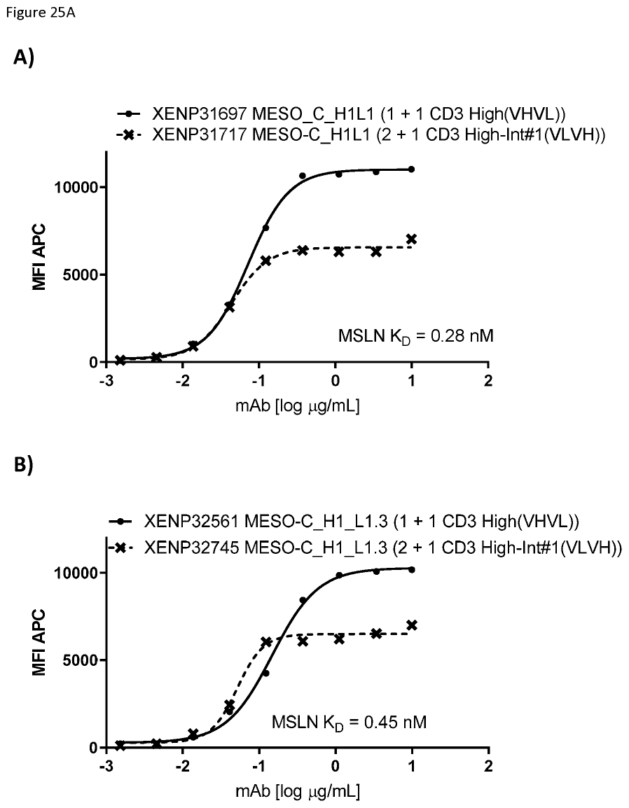

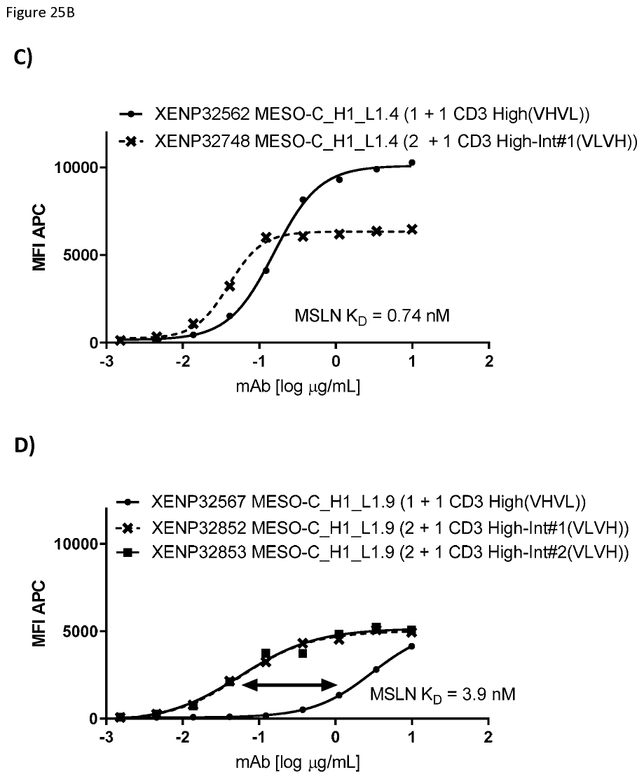

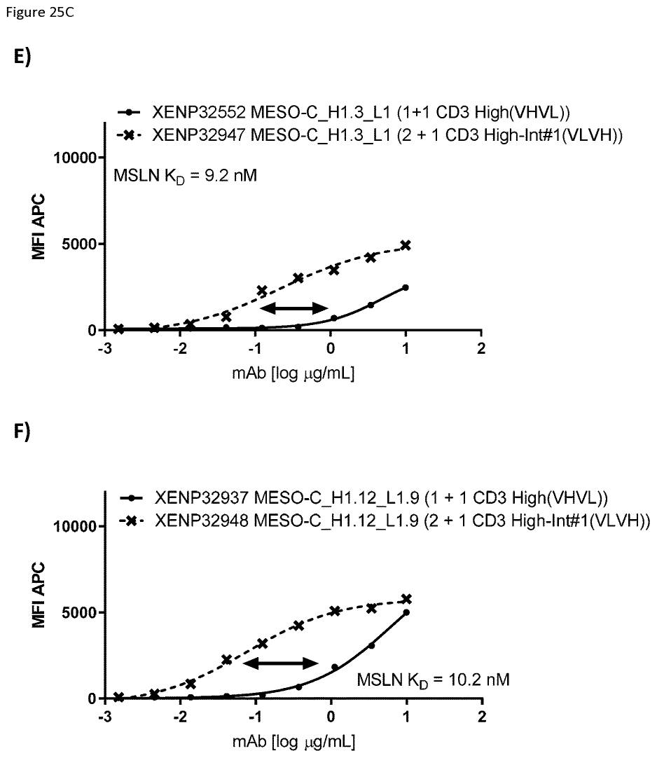

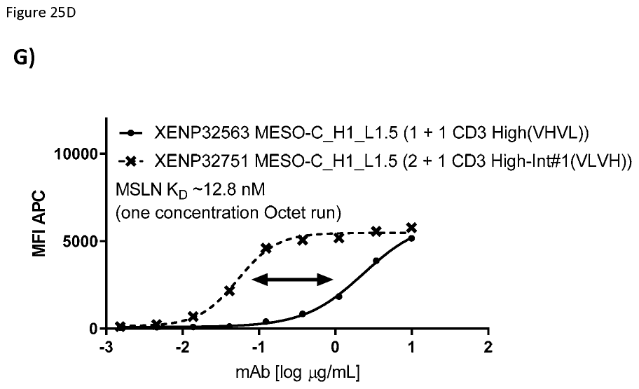

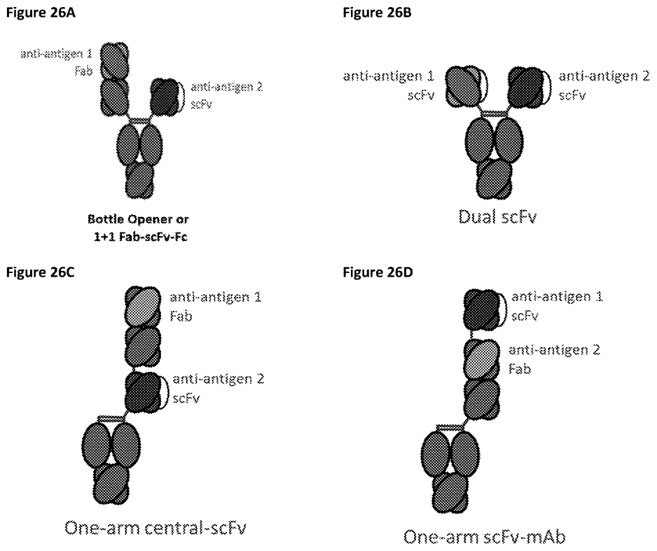

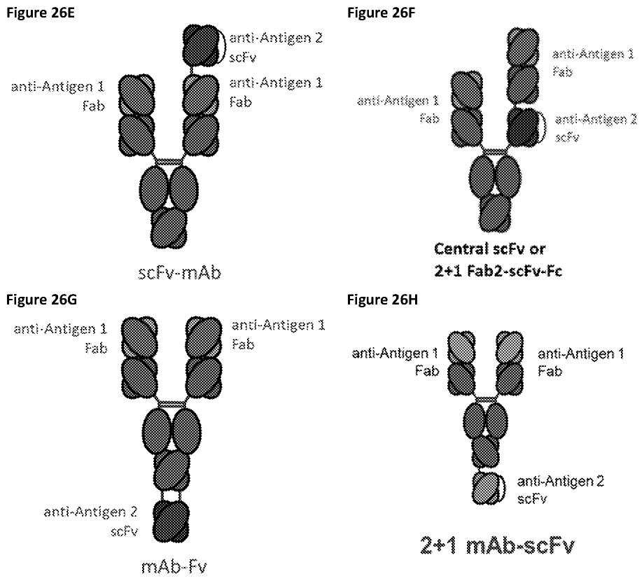

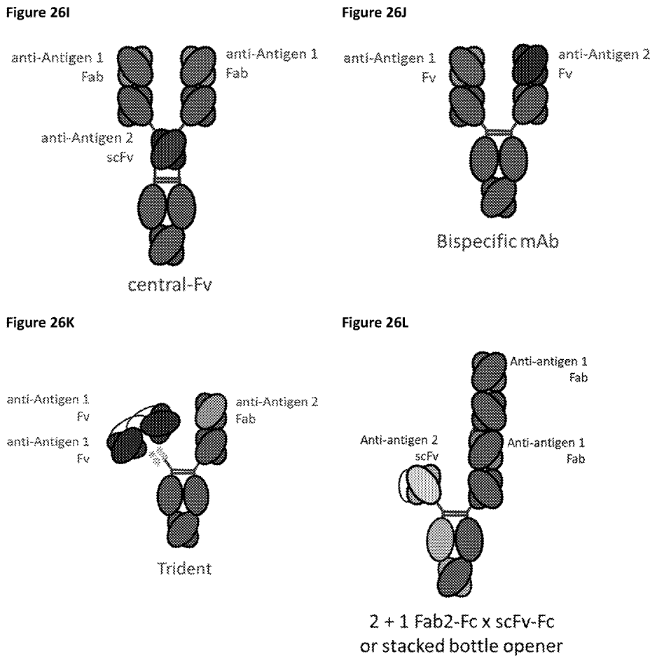

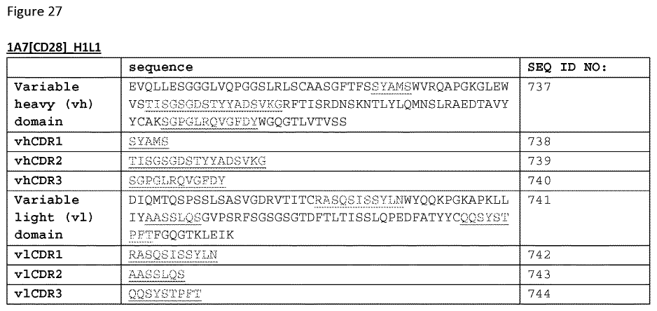

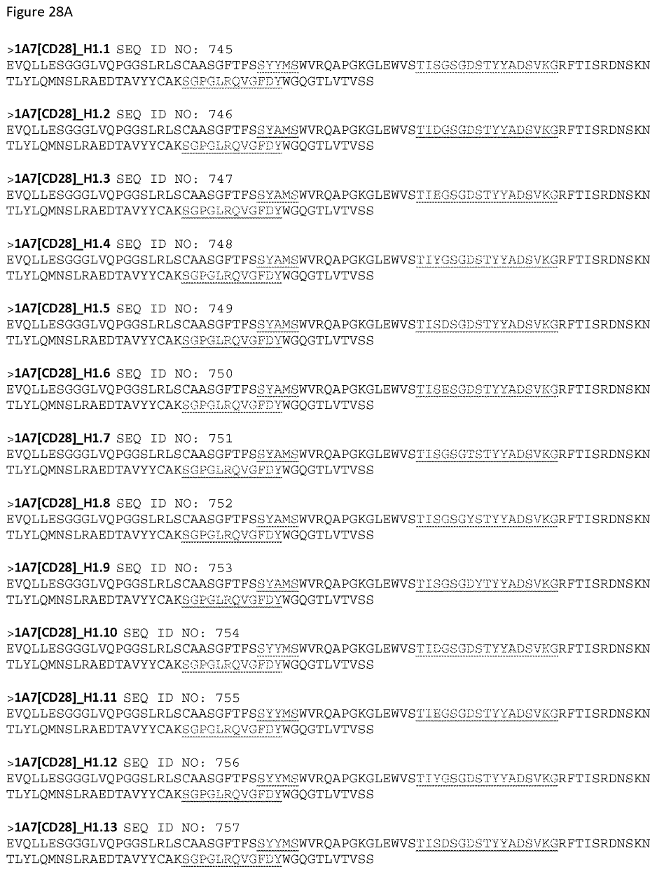

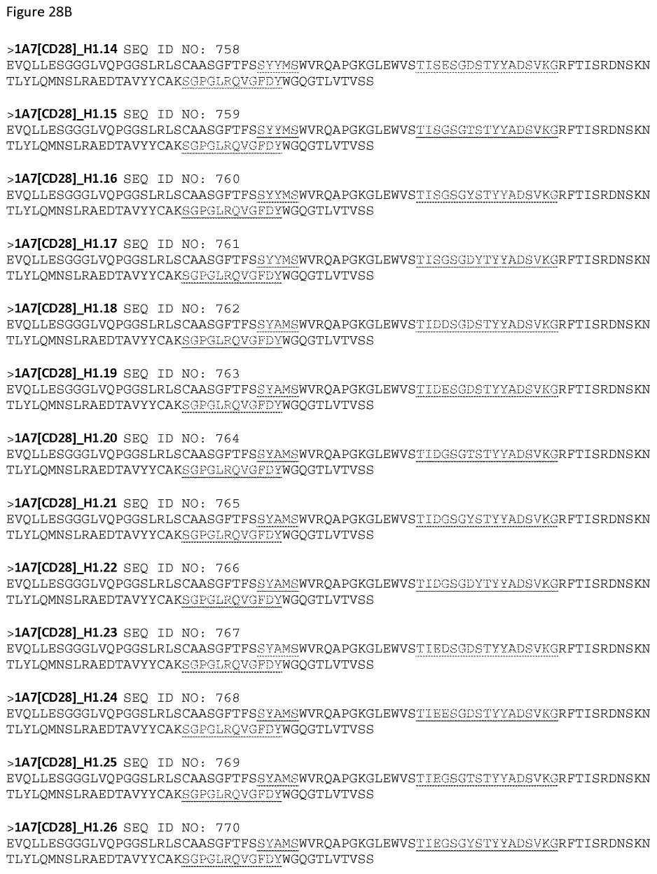

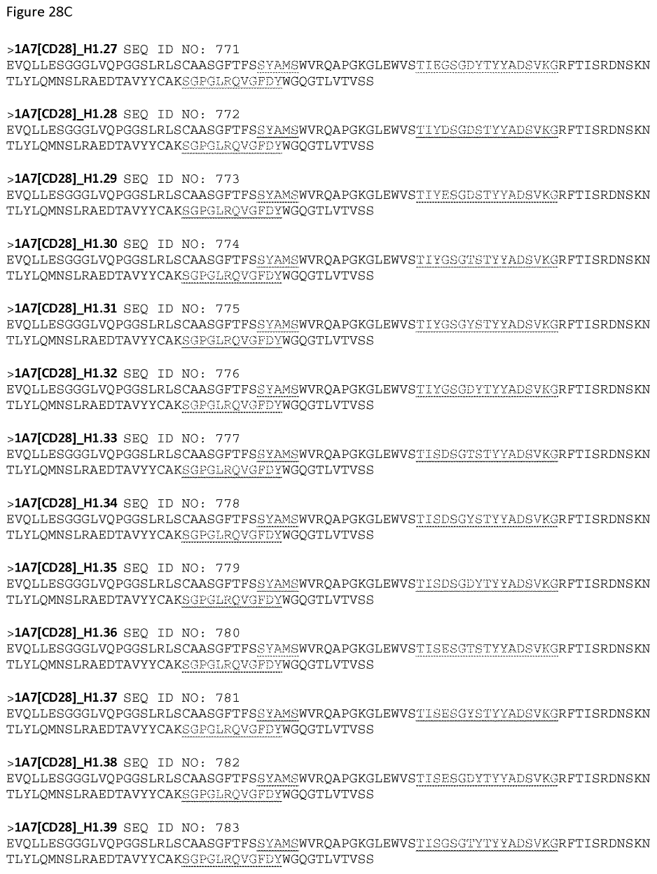

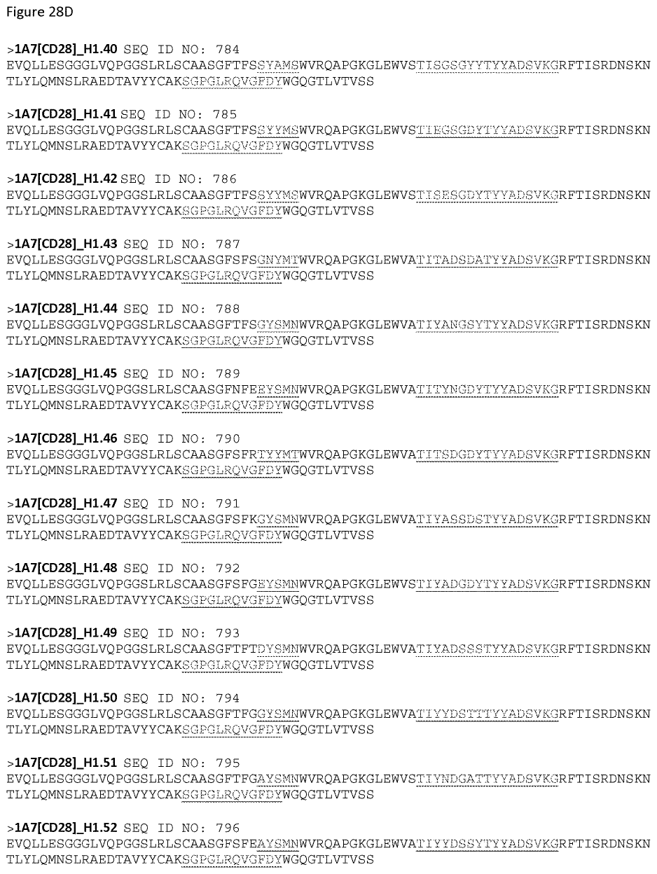

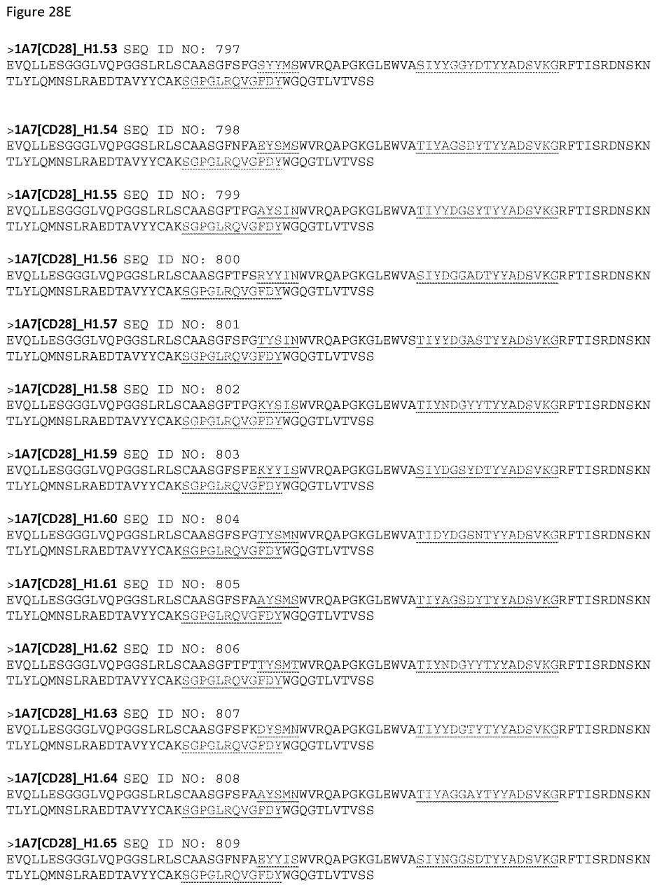

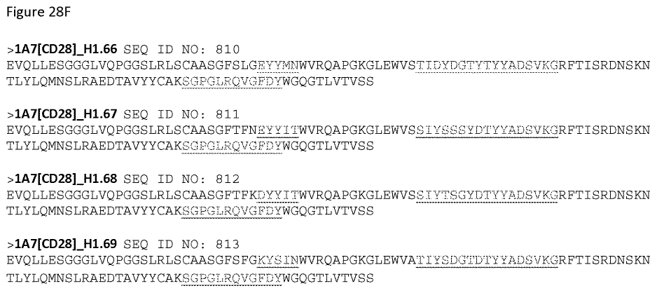

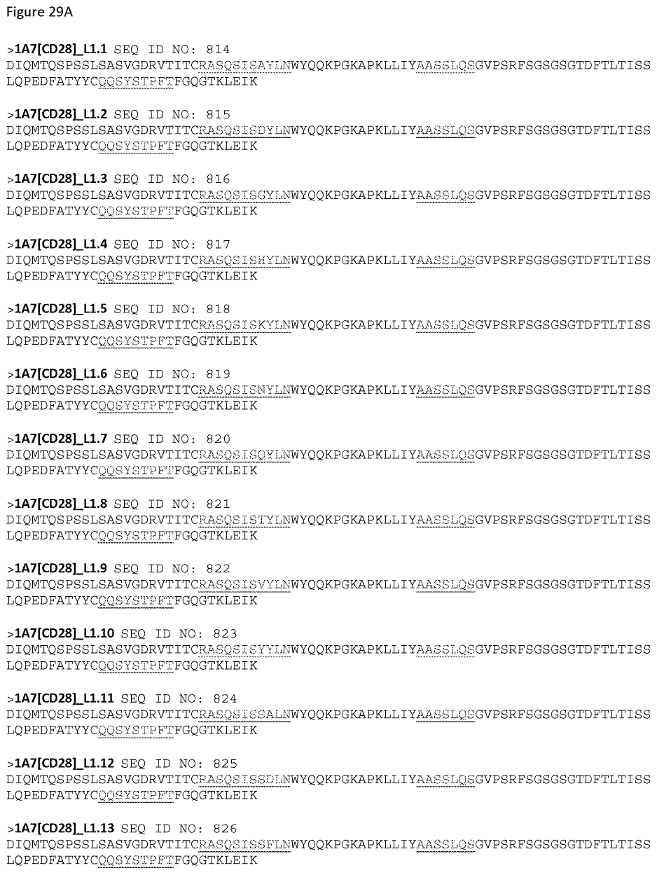

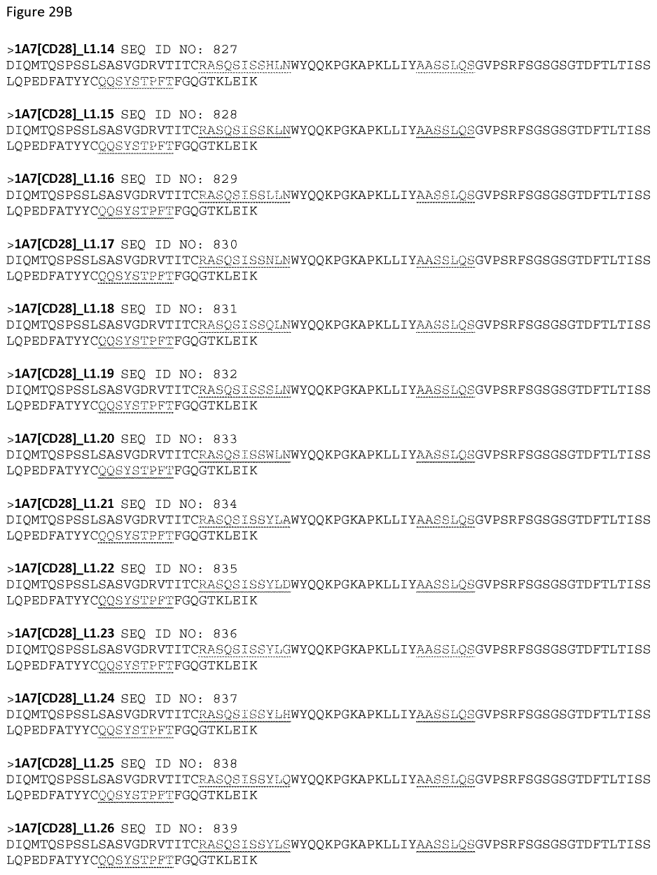

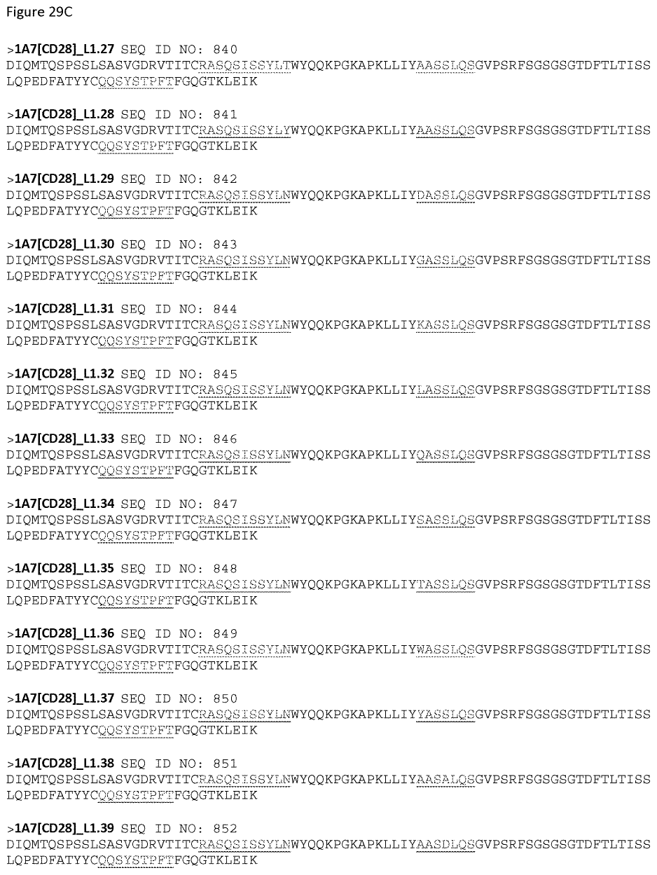

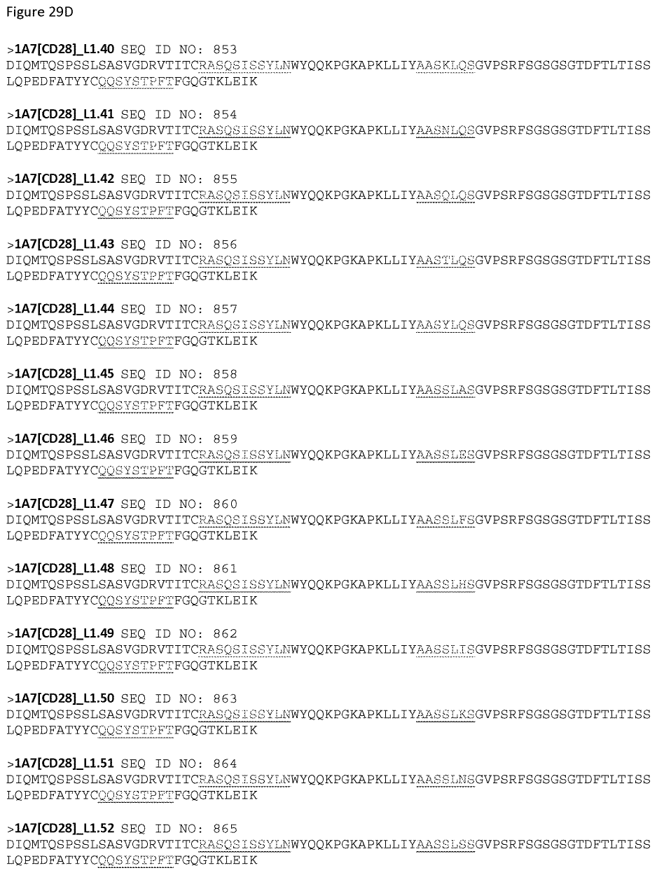

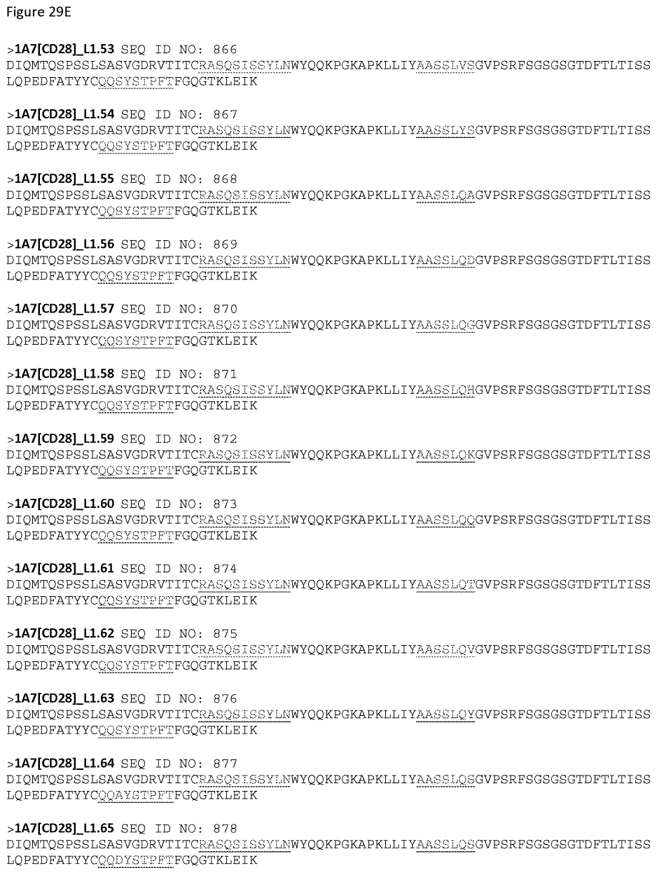

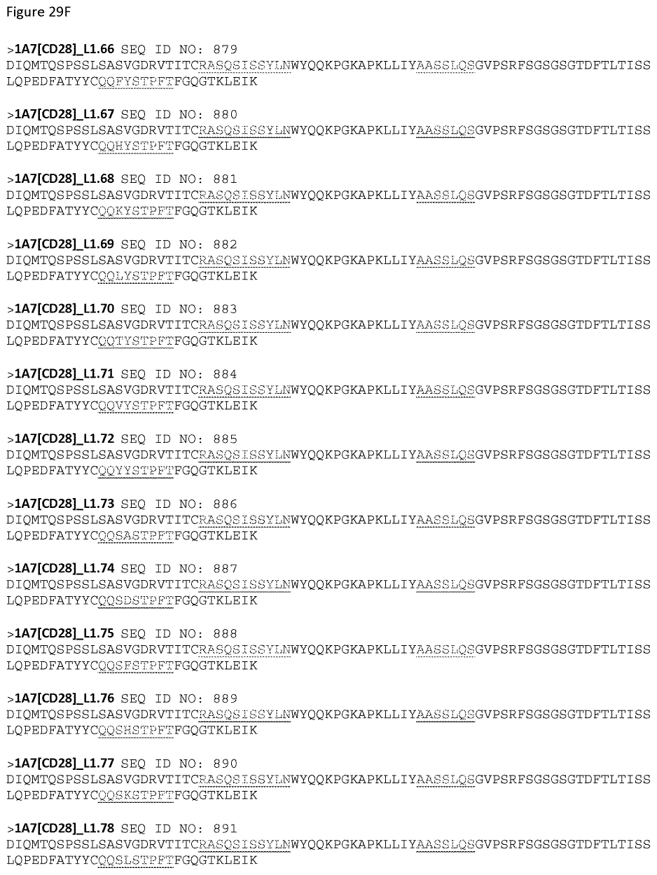

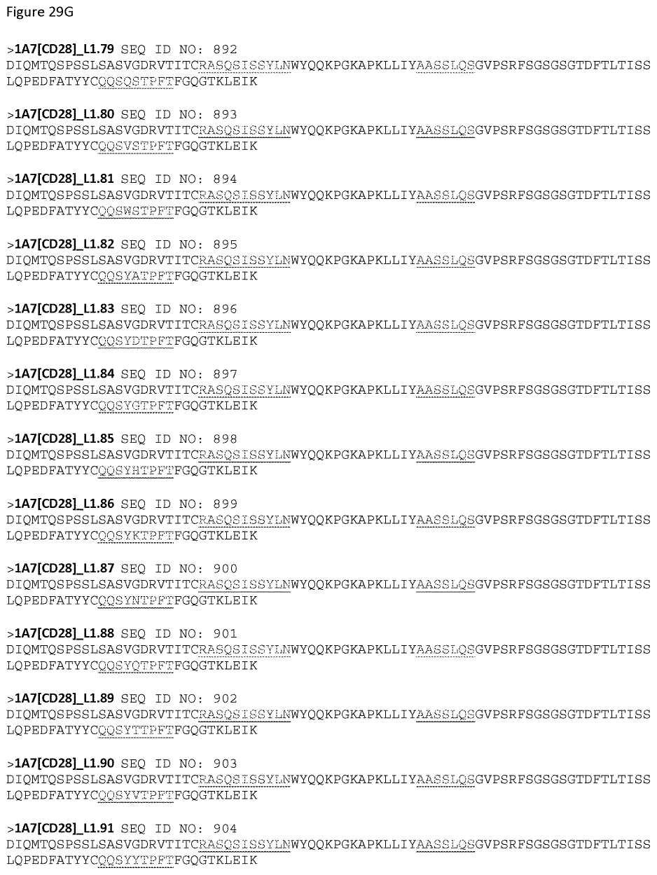

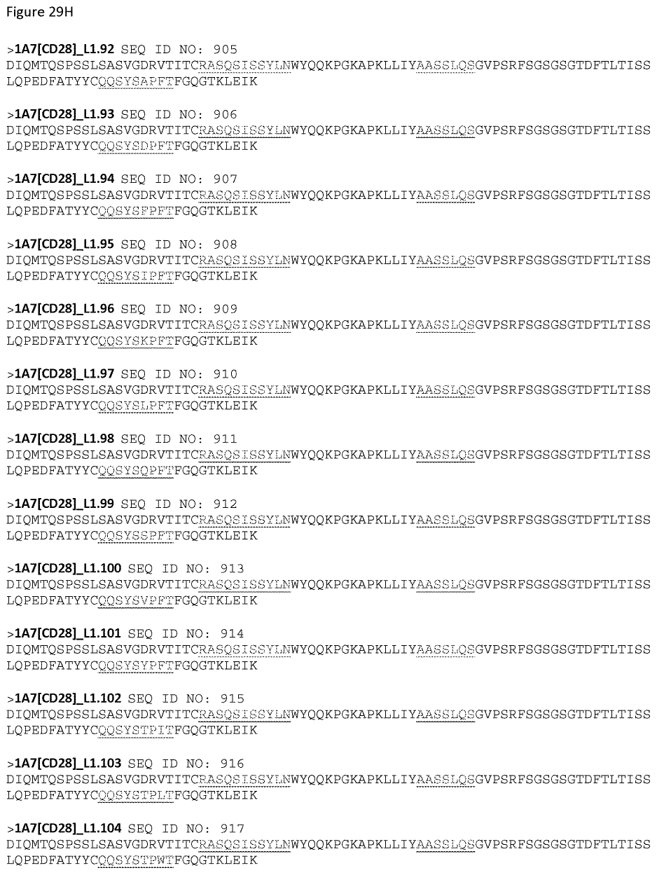

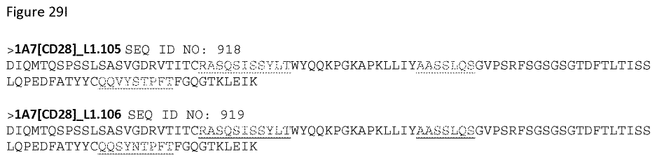

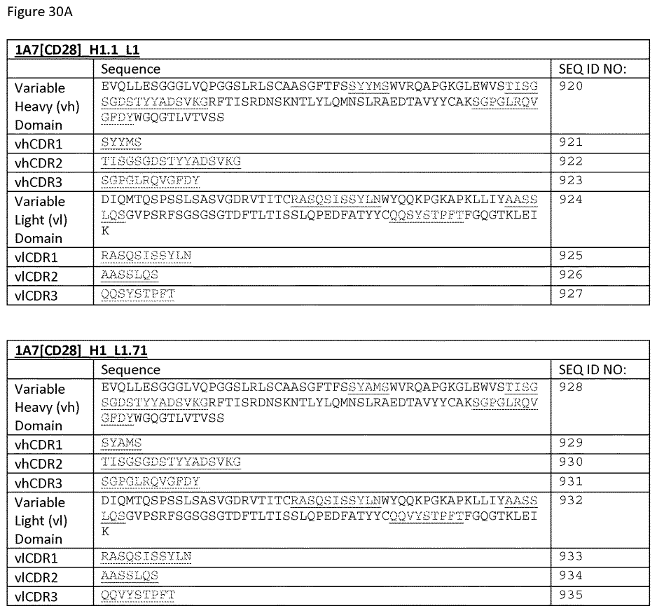

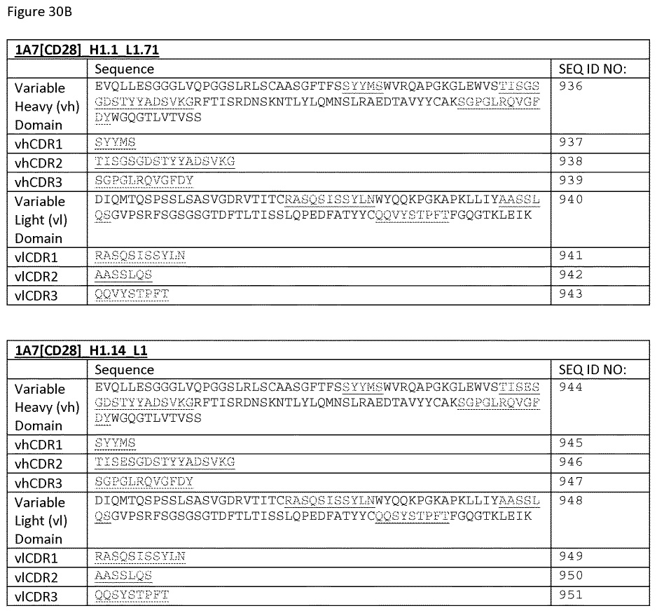

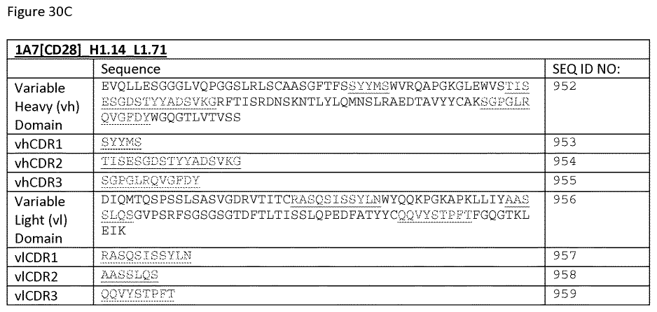

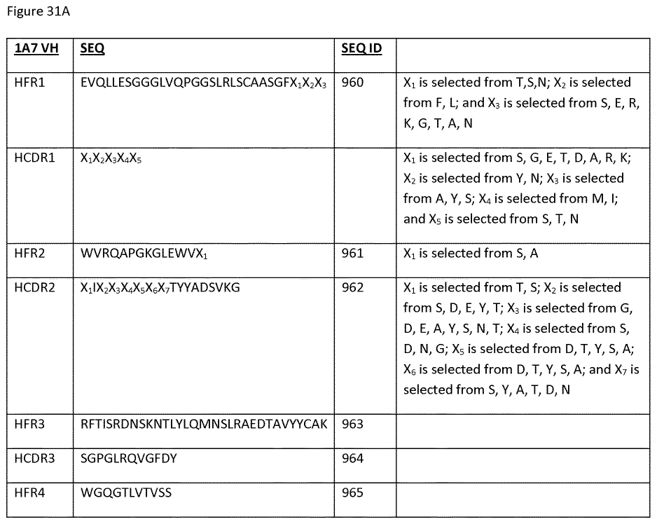

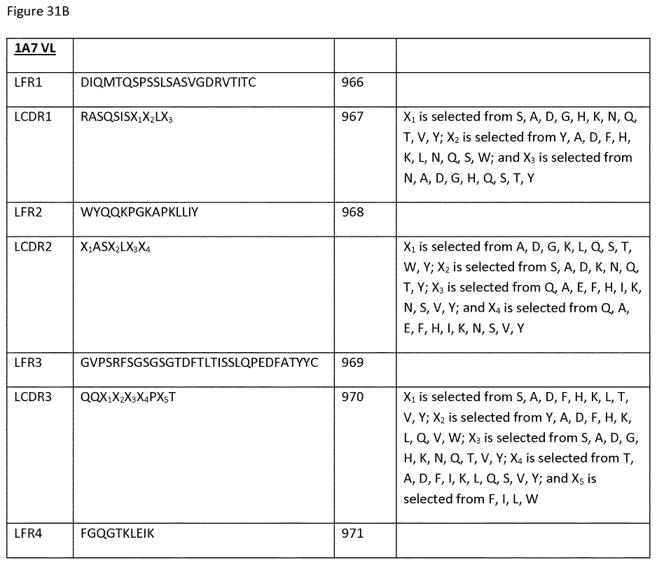

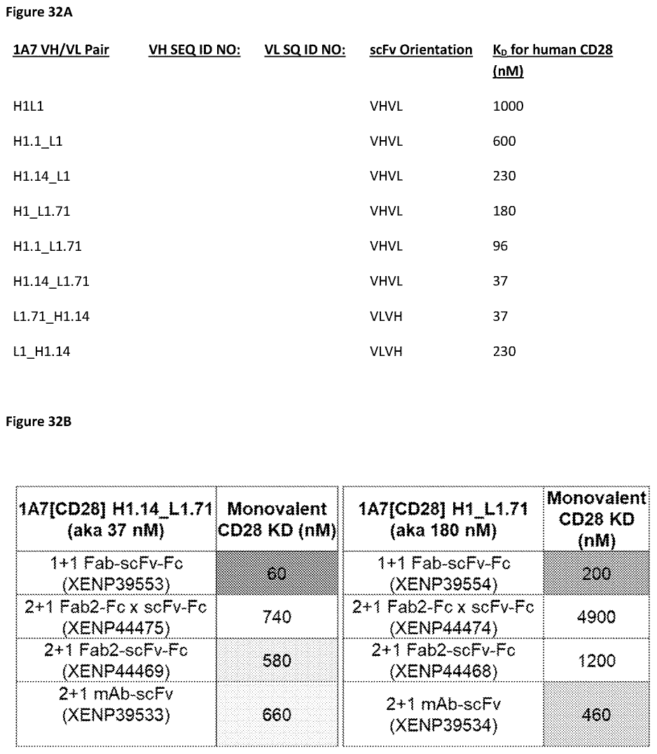

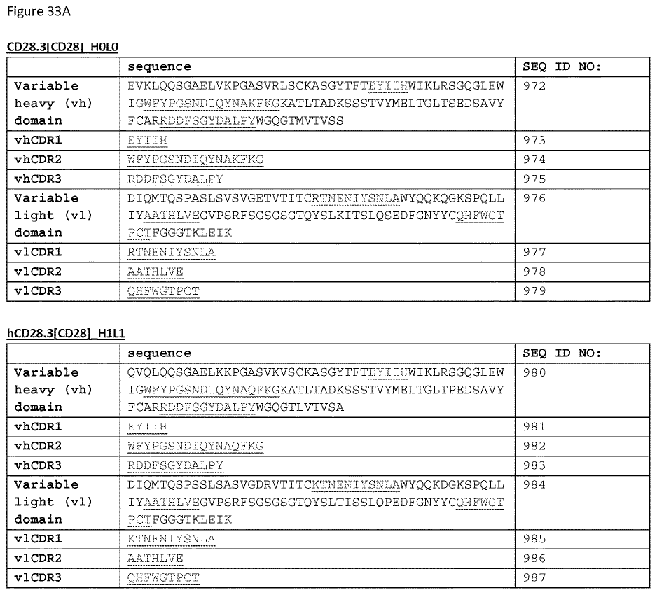

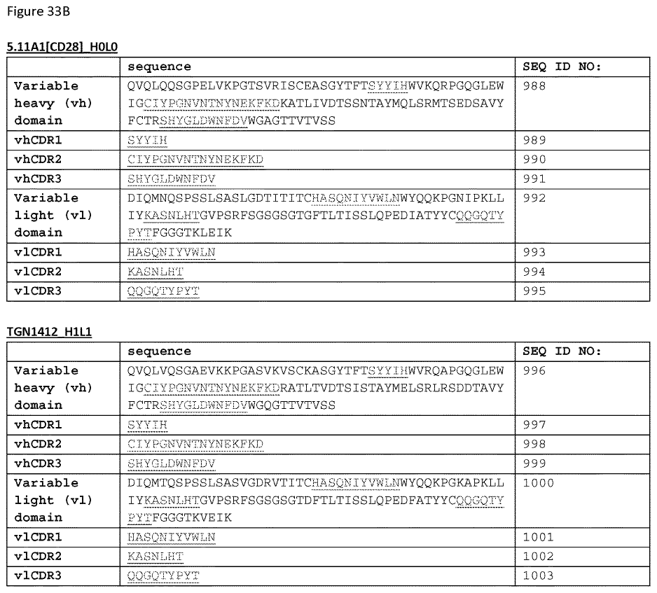

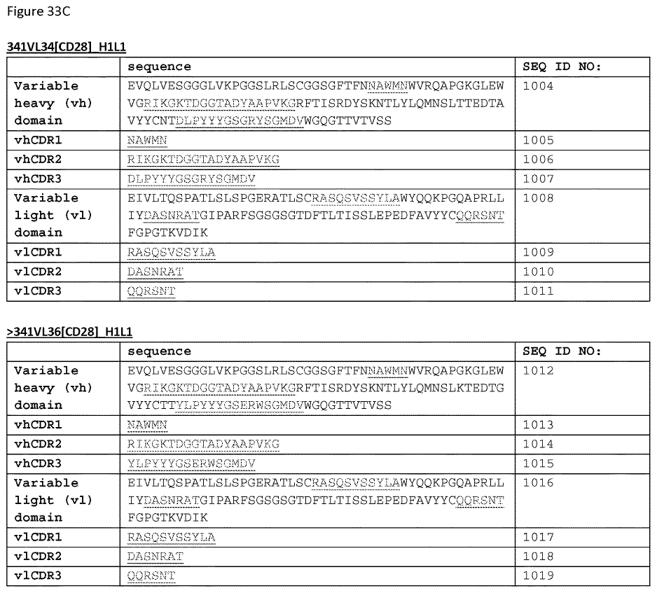

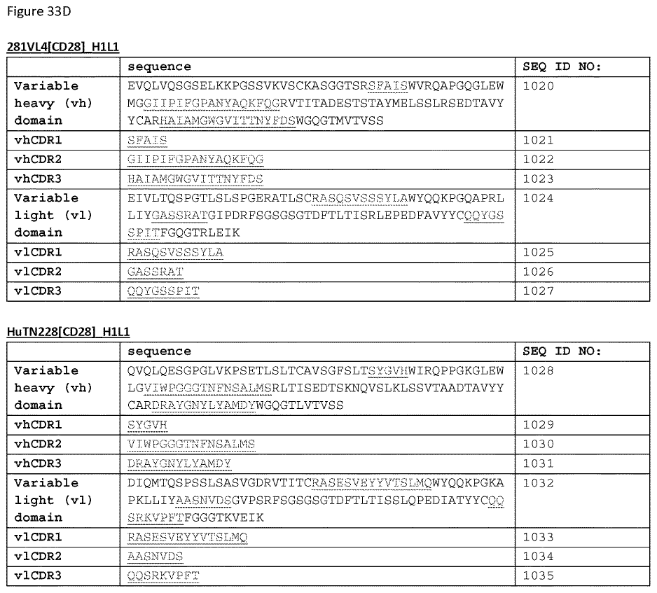

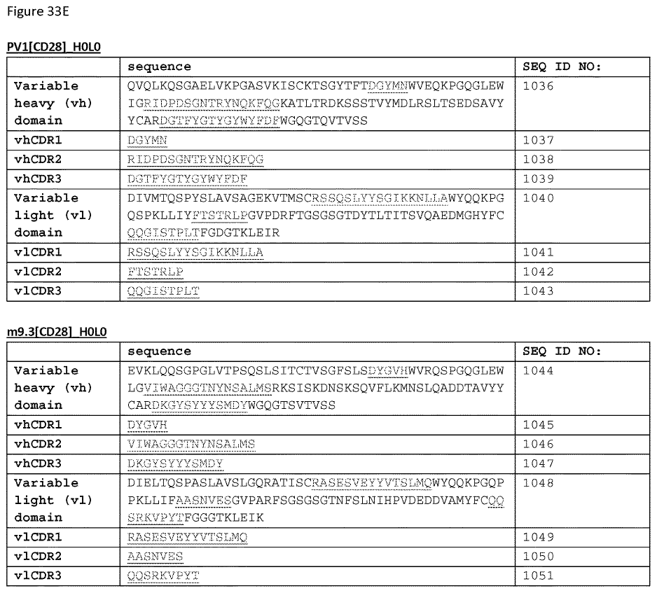

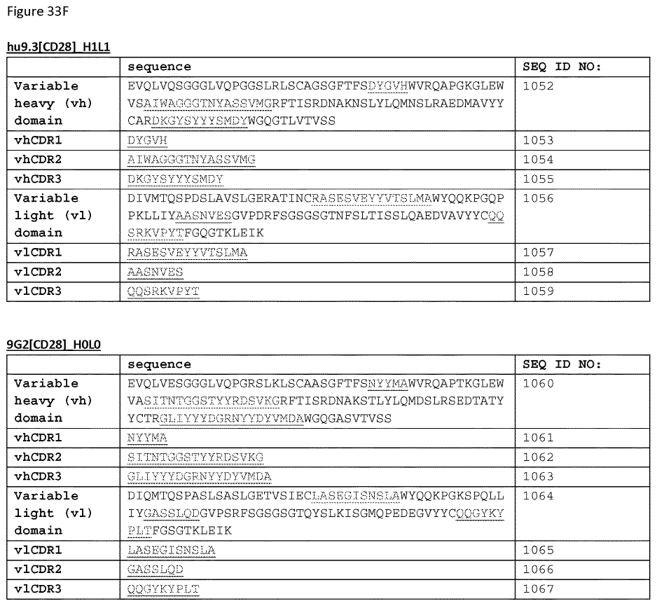

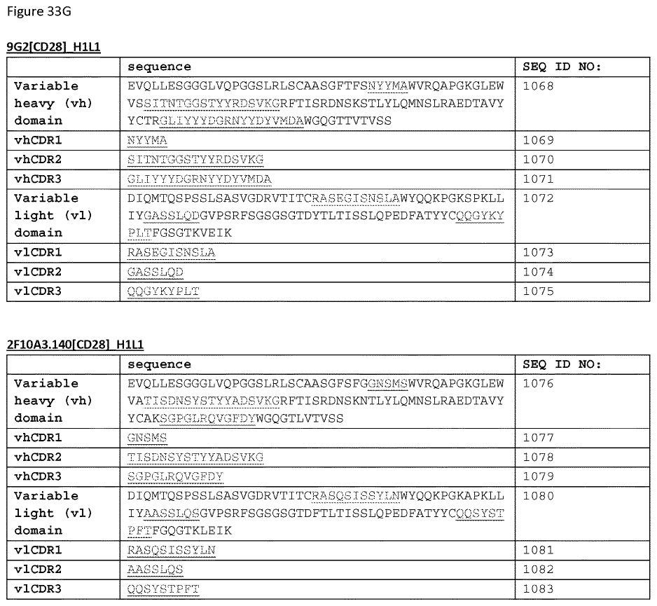

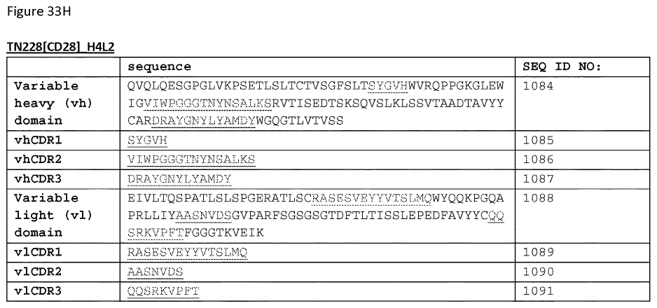

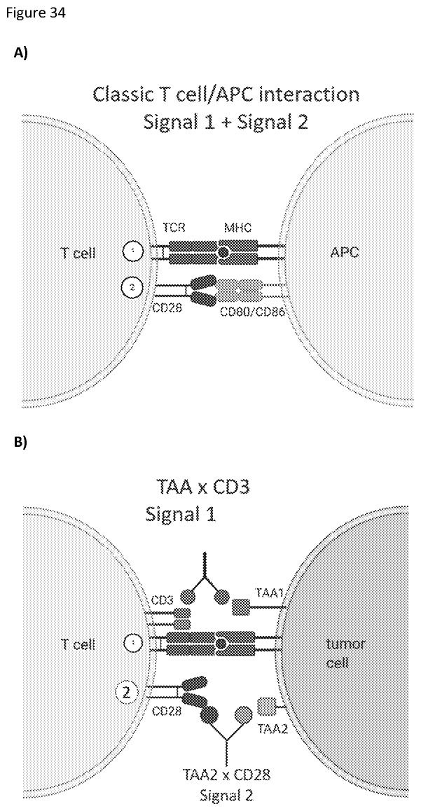

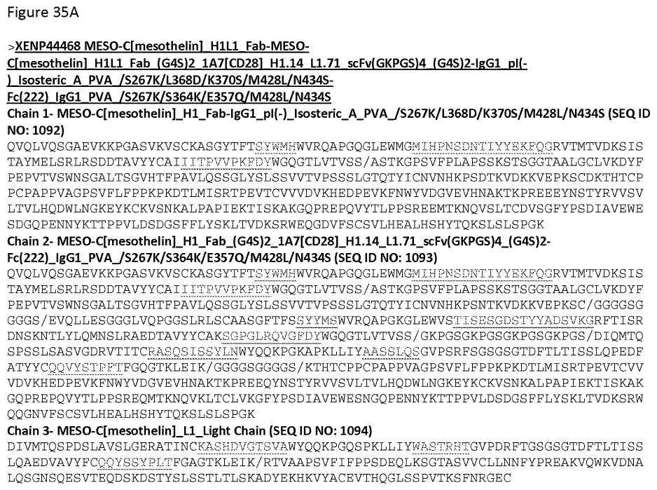

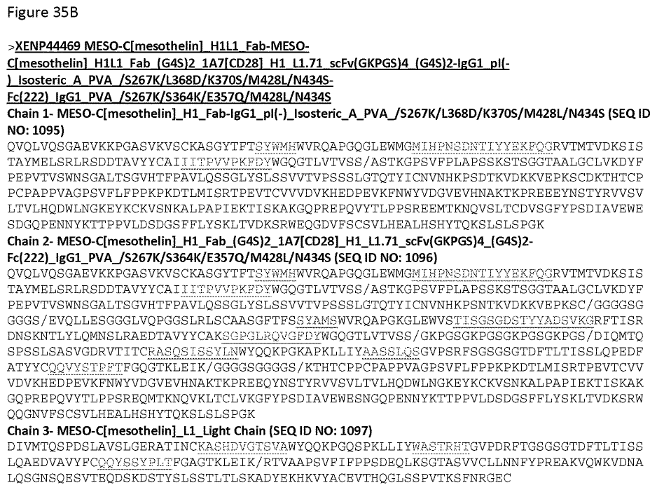

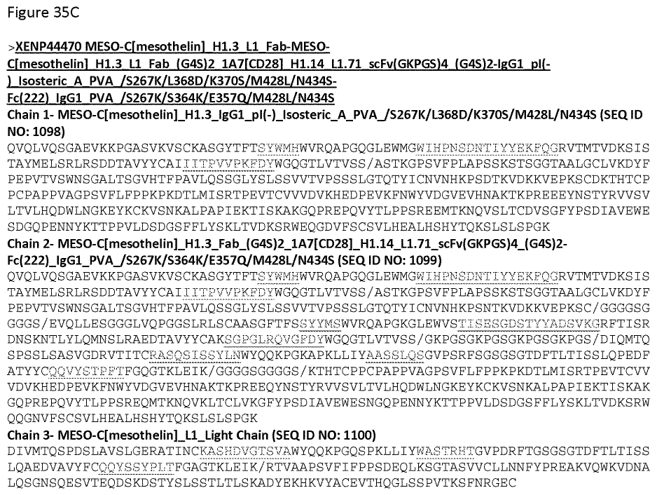

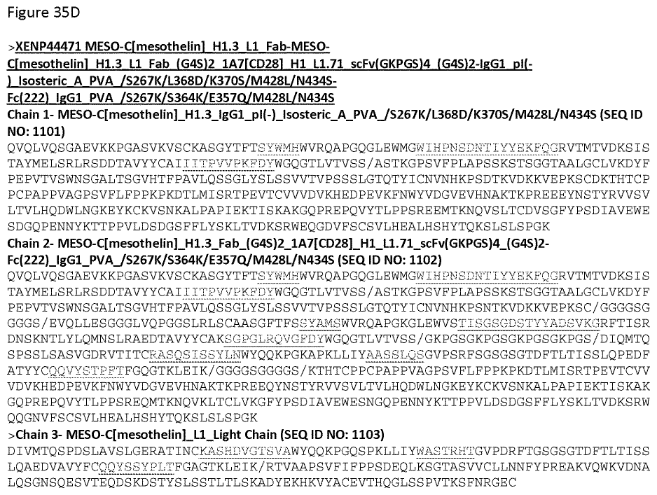

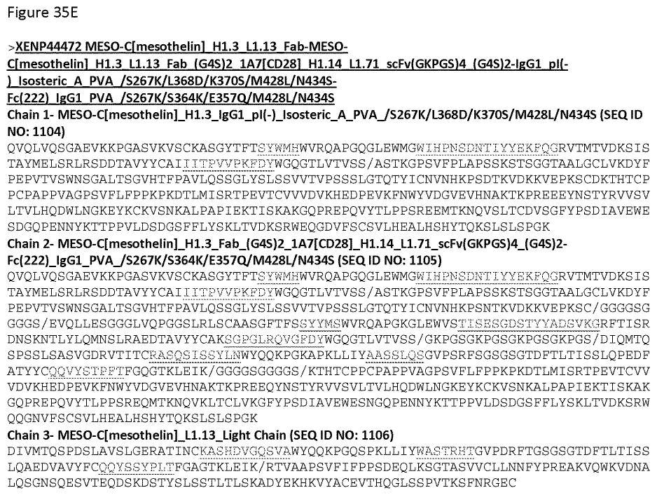

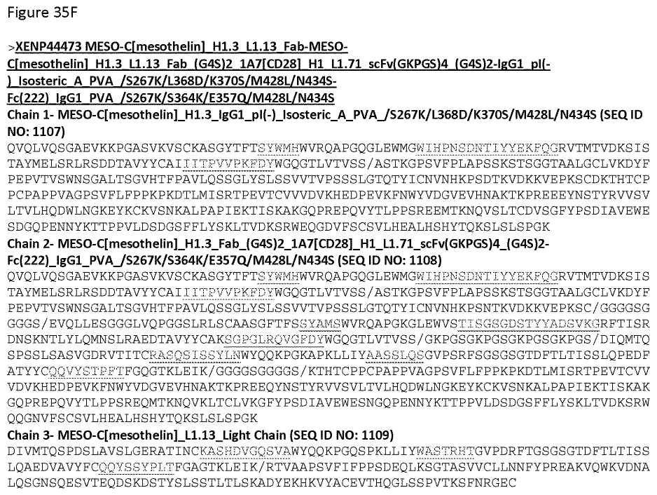

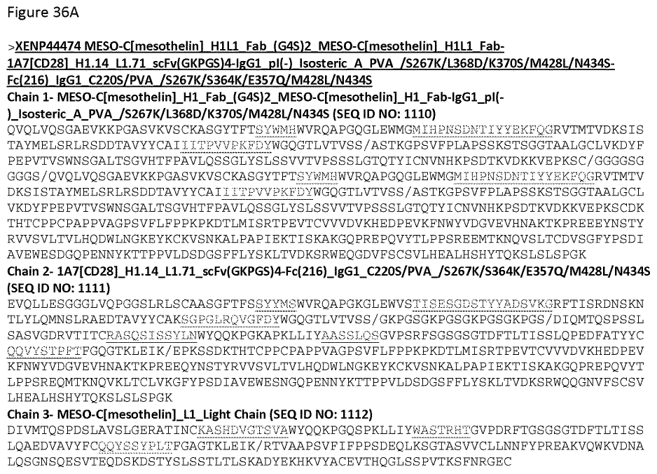

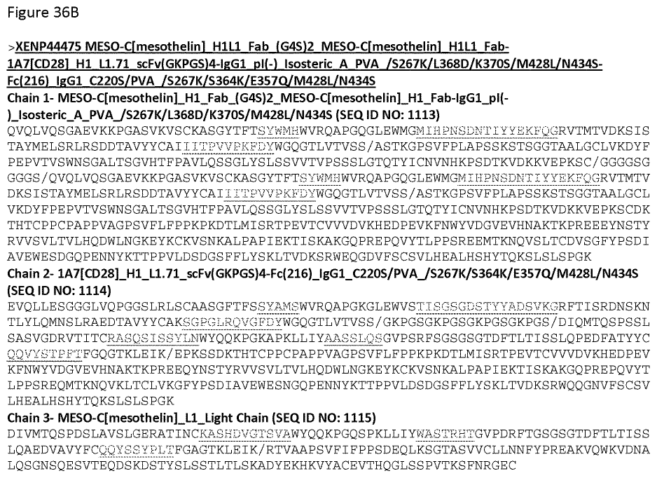

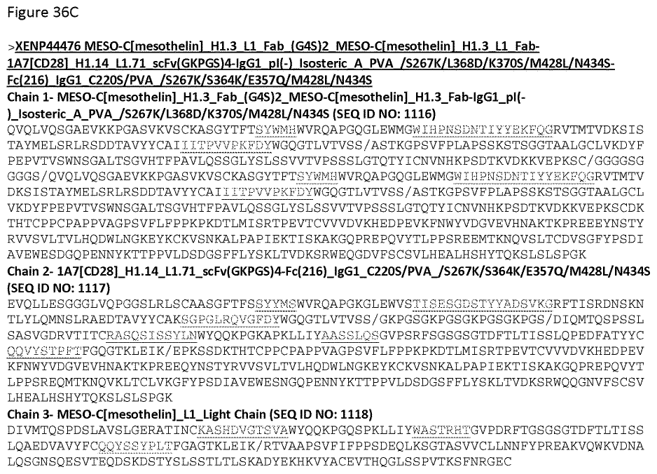

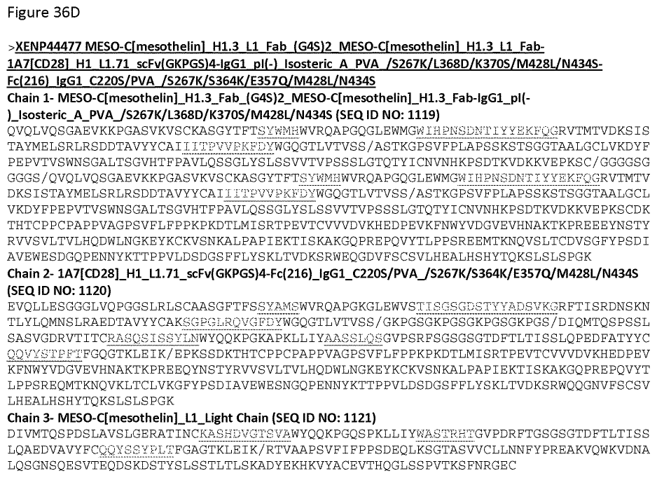

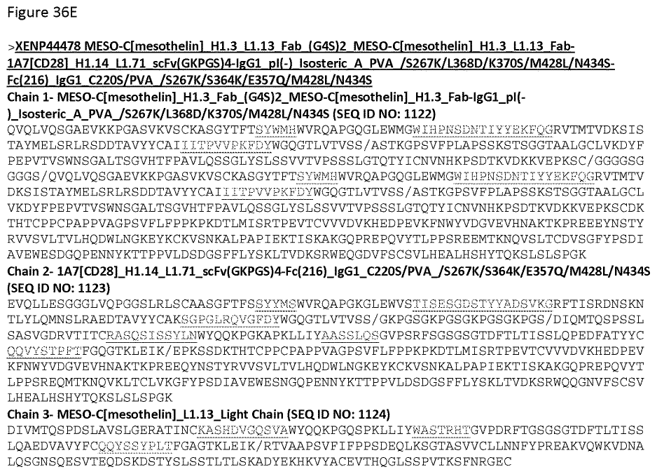

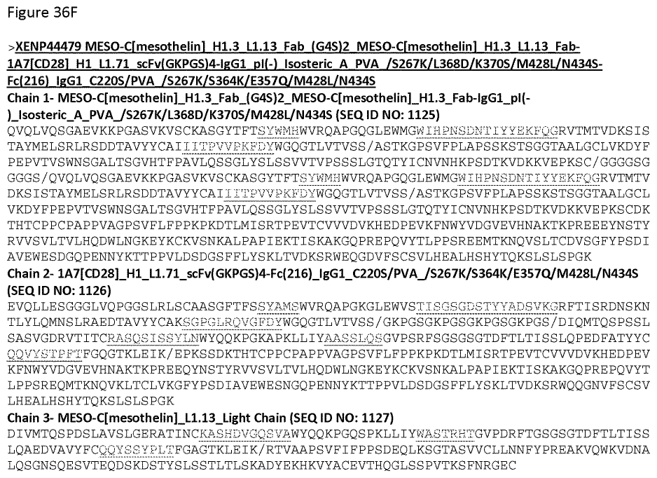

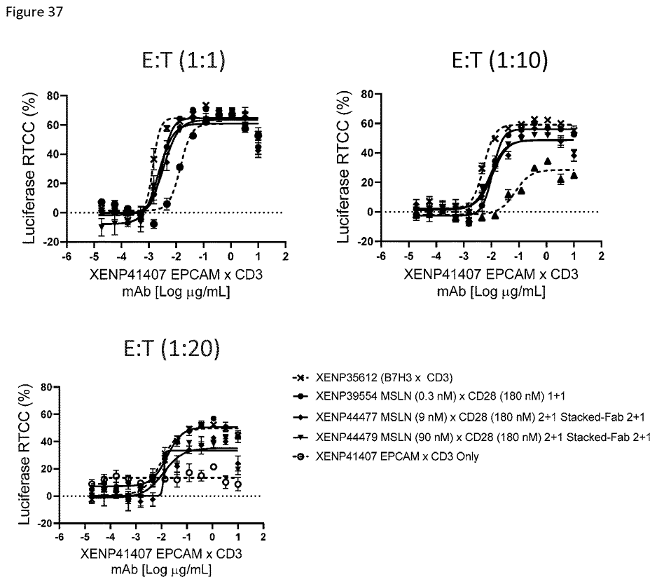

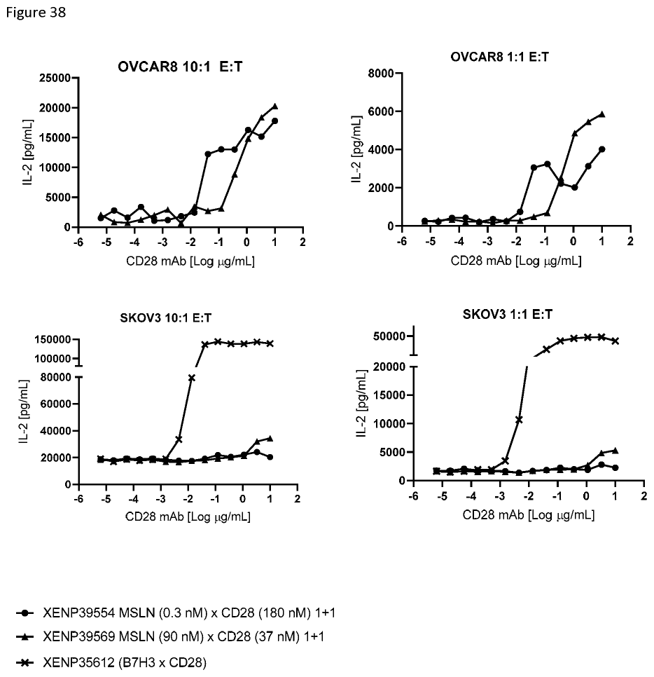

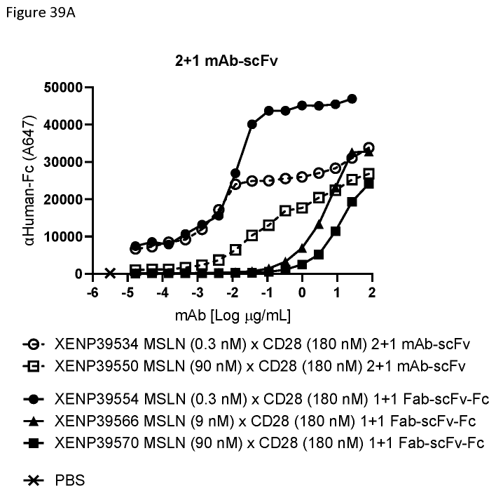

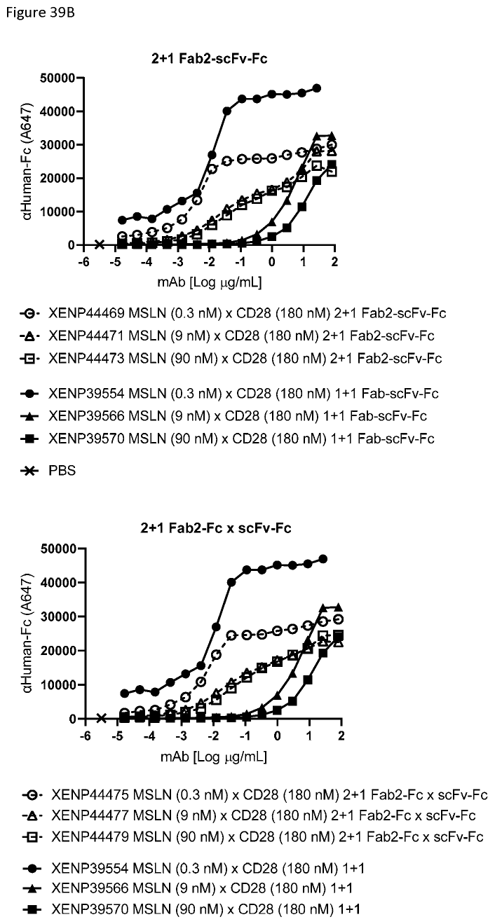

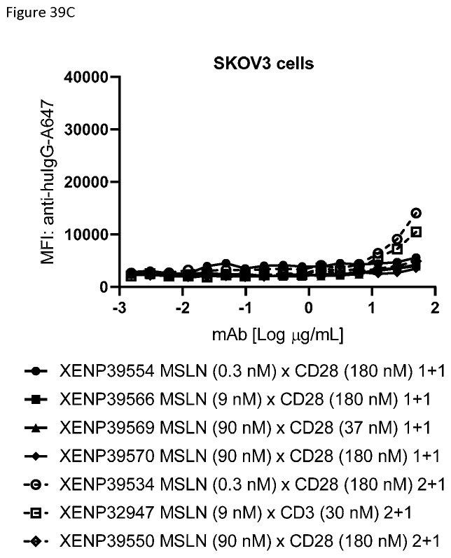

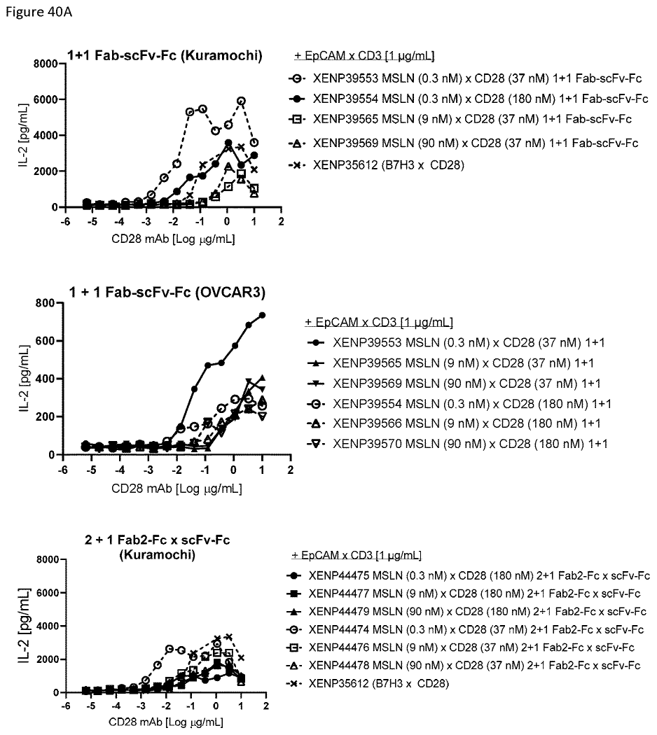

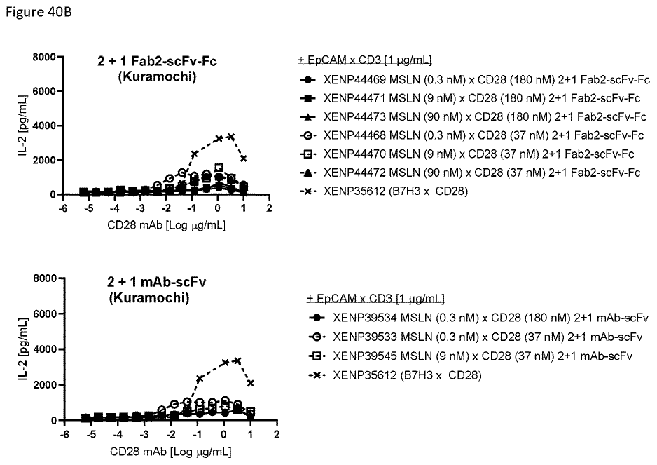

FIGS. 8A-8C depicts exemplary sequences that can be used in the subject anti-CD28×anti-MSLN antibodies provided herein. FIG. 8A depicts illustrative sequences of heterodimeric anti-CD28×anti-MSLN bsAb backbone for use in the 2+1 mAb-scFv format. The format depicted here is based on heterodimeric Fc backbone 1 as depicted in FIG. 7, except further including G446_on monomer 1 (−) and G446_/K447_on monomer 2 (+). It should be noted that any of the additional backbones depicted in Figure X may be adapted for use in the 2+1 mAb-scFv format with or without including K447_on one or both chains. It should be noted that these sequences may further include the M428L/N434S variants. FIG. 8B depicts sequences for “CH1” that find use in embodiments of anti-CD28×anti-MSLN bsAbs. FIG. 8C depicts sequences for “CH1” that find use in embodiments of anti-CD28×anti-MSLN bsAbs.

FIG. 9 depicts the sequences of several useful constant light domain backbones based on human IgG1, without the Fv sequences (e.g. the scFv or the Fab). Included herein are constant light backbone sequences that are 90, 95, 98 and 99% identical (as defined herein) to the recited sequences, and/or contain from 1, 2, 3, 4, 5, 6, 7, 8, 9 or 10 additional amino acid modifications.

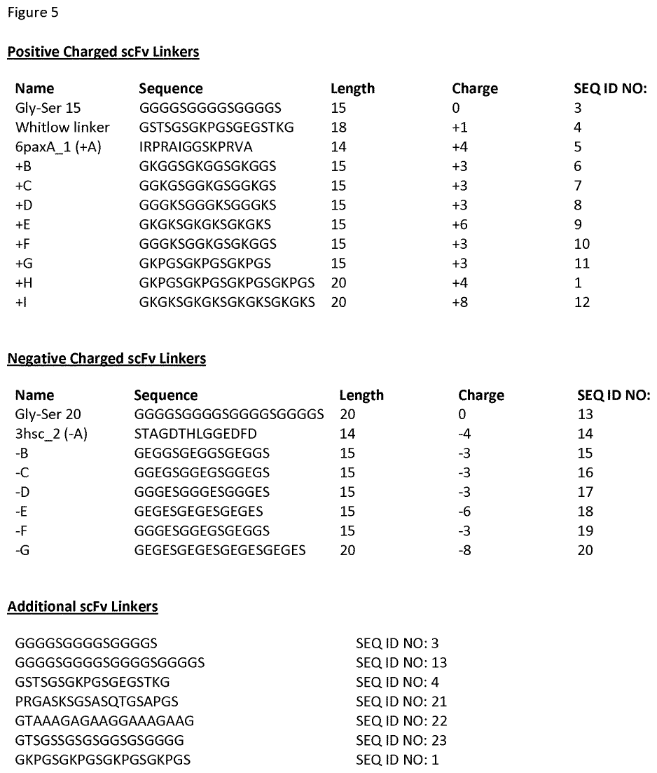

FIGS. 10A-10F depicts sequences for exemplary anti-CD3 scFvs suitable for use in the bispecific antibodies of the invention. The CDRs are underlined, the scFv linker is double underlined (in the sequences, the scFv linker is a positively charged scFv (GKPGS)₄ linker (SEQ ID NO: 1), although as will be appreciated by those in the art, this linker can be replaced by other linkers, including uncharged or negatively charged linkers, some of which are depicted in FIG. 5), and the slashes indicate the border(s) of the variable domains. In addition, the naming convention illustrates the orientation of the scFv from N- to C-terminus. As noted herein and is true for every sequence herein containing CDRs, the exact identification of the CDR locations may be slightly different depending on the numbering used as is shown in Table 2, and thus included herein are not only the CDRs that are underlined but also CDRs included within the V_{H} and V_{L} domains using other numbering systems. Furthermore, as for all the sequences in the Figures, these V_{H} and V_{L} sequences can be used either in a scFv format or in a Fab format.

FIG. 11 depicts illustrative IHC of ovarian cancer biopsy cores and adjacent normal tissue showing mesothelin expression.

FIG. 12 depicts breakdown of IHC scores of 191 ovarian cancer biopsy cores showing mesothelin expression.

FIG. 13 depicts endogenous mesothelin expression on cancer cell lines as determined by IHC and flow.

FIGS. 14A-14D depict the antigen sequences for a number of antigens of use in the antibodies described herein, including both human and cyno, to facilitate the development of antigen binding domains that bind to both for ease of clinical development.

FIGS. 15A-15B depict the variable heavy and variable light chain sequences for an exemplary MSLN binding domain referred to herein as MESO-A, as well as the sequences for XENP16249, XENP16250, and XENP16253, anti-MSLN mAbs based on MESO-A and IgG1 backbone. CDRs are underlined and slashes indicate the border(s) between the variable regions and constant domain. As noted herein and is true for every sequence herein containing CDRs, the exact identification of the CDR locations may be slightly different depending on the numbering used as is shown in Table 2, and thus included herein are not only the CDRs that are underlined but also CDRs included within the V_{H} and V_{L} domains using other numbering systems. Furthermore, as for all the sequences in the Figures, these V_{H} and VL sequences can be used either in a scFv format or in a Fab format.

FIG. 16 depicts the variable heavy and variable light chain sequences for an exemplary MSLN binding domain referred to herein as MESO-B, as well as the sequences for XENP16827, an anti-MSLN mAb based on MESO-B and IgG1 backbone. CDRs are underlined and slashes indicate the border(s) between the variable regions and constant domain. As noted herein and is true for every sequence herein containing CDRs, the exact identification of the CDR locations may be slightly different depending on the numbering used as is shown in Table 2, and thus included herein are not only the CDRs that are underlined but also CDRs included within the V_{H} and V_{L} domains using other numbering systems. Furthermore, as for all the sequences in the Figures, these V_{H} and V_{L} sequences can be used either in a scFv format or in a Fab format.

FIG. 17 depicts the variable heavy and variable light chain sequences for an exemplary MSLN binding domain referred to herein as MESO-C, as well as the sequences for XENP31693, an anti-MSLN mAb based on MESO-C and IgG1 backbone. CDRs are underlined and slashes indicate the border(s) between the variable regions and constant domain. As noted herein and is true for every sequence herein containing CDRs, the exact identification of the CDR locations may be slightly different depending on the numbering used as is shown in Table 2, and thus included herein are not only the CDRs that are underlined but also CDRs included within the V_{H} and V_{L} domains using other numbering systems. Furthermore, as for all the sequences in the Figures, these V_{H} and V_{L} sequences can be used either in a scFv format or in a Fab format.

FIG. 18 depicts the variable heavy chain sequences for MESO-C variants engineered with the aim to tune binding affinity for human MSLN. CDRs are underlined and slashes indicate the border(s) between the variable regions and constant domain. As noted herein and is true for every sequence herein containing CDRs, the exact identification of the CDR locations may be slightly different depending on the numbering used as is shown in Table 2, and thus included herein are not only the CDRs that are underlined but also CDRs included within V_{H} domains using other numbering systems. Further, as for all the sequences in the Figures, these V_{L} sequences can be used either in a scFv format or in a Fab format. Each of the variable heavy domains depicted herein can be paired with any other αMSLN variable light domain.

FIGS. 19A-19B depict the variable light chain sequences for MESO-C variants engineered with the aim to tune binding affinity for human MSLN. CDRs are underlined and slashes indicate the border(s) between the variable regions and constant domain. As noted herein and is true for every sequence herein containing CDRs, the exact identification of the CDR locations may be slightly different depending on the numbering used as is shown in Table 2, and thus included herein are not only the CDRs that are underlined but also CDRs included within V_{L} domains using other numbering systems. Further, as for all the sequences in the Figures, these V_{L} sequences can be used either in a scFv format or in a Fab format. Each of the variable light domains depicted herein can be paired with any other αMSLN variable heavy domain.

FIG. 20 depicts BLI-response, dissociation constant (K_{D}), association rate (kₐ), and dissociation rate (k_{d}) of affinity-engineered MESO-C variants (in the context of 1+1 Fab-scFv-Fc anti-MSLN×anti-CD3 bispecific antibodies, as described in U.S. Ser. No. 17/407,135) for human and cynomolgus MSLN as determined by Octet. Substitutions in variable regions are based on Kabat numbering. It should be noted that for Kabat numbering, some positions are numbered using letters as well; for example, in Kabat numbering, there is one amino acid at position 100b (K100b) but a different amino acid at position 100a (P100a); that is, the inclusion of the small letters denotes a position, not a particular amino acid in that position.

FIG. 21 depicts BLI-response, dissociation constant (K_{D}), association rate (kₐ), and dissociation rate (k_{d}) of affinity-engineered MESO-C variants (in the context of 1+1 Fab-scFv-Fc anti-MSLN×anti-CD3 bispecific antibodies, as described in U.S. Ser. No. 17/407,135) for human MSLN as determined by Octet (multiple concentration Octet run). Substitutions in variable regions are based on Kabat numbering.

FIGS. 22A-22B depict a couple of formats of the present invention. FIG. 22A depicts the “1+1 Fab-scFv-Fc” format, with a first Fab arm binding MSLN and a second scFv arm binding CD28. FIG. 22B depicts the “2+1 Fab2-scFv-Fc” format, with a first Fab arm binding MSLN and a second Fab-scFv arm, wherein the Fab binds MSLN and the scFv binds CD28.

FIGS. 23A-23FF depicts a number of additional human mesothelin antigen binding domains for use in the bispecific formats of the invention. **

FIGS. 24A-24AA depicts a number of sequences of exemplary anti-MSLN×anti-CD28 antibodies in the 1+1 Fab-scFv-Fc and 2+1 mab-scFv formats described herein.

FIGS. 25A-25D the binding to OVCAR8 cells by A) XENP31697 vs. XENP31717 (both having 0.28 nM MSLN K_{D}), B) XENP32561 vs. XENP32745 (both having 0.45 nM MSLN K_{D}), C) XENP32562 vs. XENP32748 (both having 0.74 nM MSLN K_{D}), D) XENP32567 vs. XENP32852 vs. XENP32853 (each having 3.9 nM MSLN K_{D}), E) XENP32552 vs. XENP32947 (both having 9.2 nM MSLN K_{D}), F) XENP32937 vs. XENP32948 (both having 10.2 nM MSLN K_{D}), and G) XENP32751 vs. XENP32563 (both having ~12.8 nM MSLN K_{D}). The data show that as the MSLN binding affinity is decreased, the 2+1 Fab2-scFv-Fc format provides more potent binding to OVCAR8 cells than the 1+1 Fab-scFv-Fc format. Further, XENP32852 and XENP32853 which differ only in CD3 binding affinity have identical binding to OVCAR8 cells confirming that avid binding is due to the MSLN binding domain, which can be combined with a CD28 antigen binding domain.

FIGS. 26A-26L depict several formats for use in the anti-MSLN×anti-CD28 bispecific antibodies disclosed herein. The first is the “1+1 Fab-scFv-Fc” format (also referred to as the “bottle opener” or “Triple F” format), with a first antigen binding domain that is a Fab domain and a second anti-antigen binding domain that is an scFv domain. Additionally, “mAb-Fv,” “mAb-scFv,” “2+1 Fab2-scFv-Fc” (also referred to as the “central scFv” or “central-scFv” format”), “central-Fv,” “one-armed central-scFv,” “one scFv-mAb,” “scFv-mAb,” “dual scFv,” “trident,” “2+1 Fab2-Fc×scFv-Fc,” and non-heterodimeric bispecific formats are all shown. The scFv domains can be either, from N- to C-terminus orientation: variable heavy-(optional linker)-variable light, or variable light-(optional linker)-variable heavy. In addition, for the one-armed scFv-mAb, the scFv can be attached either to the N-terminus of a heavy chain monomer or to the N-terminus of the light chain. In certain embodiments, “Anti-antigen 1” in FIGS. 26A-26L refers to a MSLN binding domain. In certain embodiments, “Anti-antigen 1” in FIGS. 26A-26L refers to a CD28 binding domain. In certain embodiments, “Anti-antigen 2” in FIGS. 26A-26L refers to a MSLN binding domain. In certain embodiments “Anti-antigen 2” in FIGS. 26A-26L refers to a CD28 binding domain. In some embodiments, “Anti-antigen 1” in FIGS. 26A-26L refers to a MSLN binding domain and “Anti-antigen 2” in FIGS. 26A-26L refers to a CD28 binding domain. In some embodiments, “Anti-antigen 1” in FIGS. 26A-26L refers to a CD28 binding domain and “Anti-antigen 2” in FIGS. 26A-26L refers to a MSLN binding domain. Any of the MSLN binding domains and CD28 binding domains disclosed can be included in the bispecific formats of FIGS. 26A-26L.

FIG. 27 depicts the variable heavy and variable light chain sequences for 1A7, an exemplary phage-derived CD28 binding domain.

FIGS. 28A-28F depicts additional sequences for affinity-optimized variable heavy domains from anti-CD28 clone 1A7. It should be noted that the variable heavy domains can be paired with any of the other variable light domains depicted herein including those depicted in FIGS. 27, 29, 30 and 33.

FIGS. 29A-29I depicts additional sequences for affinity-optimized variable light domains from anti-CD28 clone 1A7. It should be noted that the variable light domains can be paired with any of the other variable heavy domains depicted herein including those depicted in 27, 28, 30 and 33.

FIGS. 30A-30C depicts the sequence for illustrative affinity-optimized 1A7 VH/VH pairs. It should be noted that these pairs may be formatted as Fabs or as scFvs. Additionally, in the scFv format, these pairs may be formatted in the VHVL orientation or the VLVH orientation.

FIGS. 31A-31B depicts consensus framework regions (FR) and complementarity determining regions (CDRs) (as in Kabat) for anti-CD28 clone 1A7 variable heavy and variable light domain variants.

FIGS. 32A-32B depicts illustrative affinity-engineered 1A7 VH/VL pairs and their binding affinities in the context of scFvs (in the context of 1+1 Fab-scFv-Fc bsAb format).

FIGS. 33A-33H depicts the variable heavy and variable light chain sequences for additional CD28 binding domains which find use in the MSLN×CD28 bsAbs of the invention. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 34 depicts A) classic T cell/APC interaction and B) replication of the classic T cell/APC interaction by combining CD3 bispecific antibodies with CD28 bispecific antibodies. In classic T cell/APC interaction, there is a first signal provided by TCR reactivity with peptide-MHC (Signal 1) and a second signal provided by CD28 crosslinking by CD80/CD86 being expressed on APCs (Signal 2) which together fully activate T cells. In contrast in treatment with CD3 bispecifics, only the first signal is provided. The CD28 signal may be provided by a CD28 bispecific with the idea to promote activation and proliferation through CD28 costimulation. In some embodiments, TAA1 and TAA2 may be different antigens. In some embodiments, TAA1 and TAA2 may be same antigen but different epitopes. In some embodiments, TAA1 and TAA2 may be same antigen and same epitope.

FIGS. 35A-35F depict MSLN×CD28 bsAbs in the 2+1 Fab-scFv-Fc format.

FIGS. 36A-36F depict MSLN×CD28 bsAbs in the 2+1 Fab2-Fc×scFv-Fc format.

FIG. 37 depicts the ability of MSLN×CD28 antibodies to enhance RTCC driven by a CD3 engager even at the lower E:T ratio of 1:20. In this experiment, 10,000 OVCAR8-Luc cells (90K MSLN antigens) were seeded per well with T-cells at an effector to target ratio of 1:1, 1:10, and 1:20 in the presence of a constant 1 g/mL of each MSLN×CD28 bsAb and the CD3 engager, XENP41407 (EPCAM×CD3), at the concentrations indicated. RTCC was assessed after 3 and 5 days via Luciferase.

FIG. 38 depicts the ability of MSLN×CD28 bsAbs to selectively enhance B7H3×CD3 bsAbs on cells with high MSLN expression. 10,000 OVCAR8 (~90K MSLN antigens/cell) or SKOV3 cells (~9K MSLN antigens/cell) were seeded per well in a plate. The following day, T cells were seeded at an effector to target ratio of 10:1 and 1:10 in the presence of a constant 1 g/ml B7H3×CD3 bsAb, and a MSLN×CD28 bsAb at the concentrations indicated. IL-2 release was assessed after 1 day via Meso Scale Discovery (MSD).

FIGS. 39A-39C depict that bivalent engagement by all of the 2+1 formats (2+1 Fab2-Fc×scFv-Fc, 2+1 Fab2-scFv-Fc, or 2+1 mAb-scFv) displays avid binding, while monovalent engagement by the highest affinity MSLN binding domain in the 1+1 Fab-scFv-Fc format maximizes binding efficacy. In this experiment, 50,000 OVCAR8 cells (~90K MSLN antigens/cell) were seeded per well in a plate. Next, antibodies were added at the doses indicated in FIG. 39 for 1 hour at 4° C. Anti-human-Fc secondary (A647) was added for 30 min at 4° C., and binding was assessed via FACs. FIG. 39C depicts that the CD28×MSLN bsAbs in neither 1+1-Fab-scFv format nor the 2+1 mAb-scFv format antibodies show any significant off target binding. In this experiment, 50,000 SKOV3 cells (~9K MSLN antigens/cell) were seeded per well in a plate. Next, antibodies were added at the doses indicated in FIG. 39 for 1 hour at 4° C. Anti-human-Fc secondary (A647) was added for 30 min at 4° C., and binding was assessed via FACs.

FIGS. 40A-40B depict that XENP39553 displayed enhanced IL-2 release at a significantly higher level than any of the other MSLN×CD28 bsAbs. In FIG. 40, 10,000 Kuramochi cells (having ~120K MSLN antigens/cell) or OVCAR3 cells were seeded per well in a plate. The following day, T cells were seeded at an effector to target ratio of 1:1 in the presence of 1 g/ml EPCAM×CD3 and the indicated concentrations. IL-2 release was assessed after 1 days via MSD.

### DETAILED DESCRIPTION

### I. Overview

MSLN is overexpressed in several cancers, including ovarian cancer, colon cancer, breast cancer, hepatocellular carcinoma, lung cancer, mesothelioma, pancreatic cancer, non-small cell lung cancer (such as lung adenocarcinoma) and endometrial cancer. Thus, MSLN is a potential target for treating such cancers.

The activation of T cells in the treatment of cancer is being widely investigated. T cells require multiple signals for complete activation and differentiation. As shown in FIG. 34A, Signal 1, promoted by recognition of a peptide-MHC (pMHC) complex by the T cell receptor (TCR), is absolutely required for T cell activation. Signal 2, which synergizes with, and amplifies signal 1, is typically provided by the interaction of the CD28 ligands CD80 and CD86 with CD28 itself. Although CD28 engagement alone is typically inert, when combined with signal 1 activation, it promotes additional activation, survival, and proliferative signals, including IL-2 secretion. As CD80 and CD86 are only naturally expressed by professional antigen-presenting cells (APC), the extent of CD28 costimulation in the tumor setting can be highly variable. Accordingly, the present invention is directed to a novel class of tumor-targeted anti-CD28×anti-MSLN bispecific antibodies, the CD80/CD86 engagement of CD28 that mimic the CD80/CD86 engagement of CD28, thereby providing an artificial source of signal 2. Notably, signal 1 can either be provided by the natural TCR:pMHC recognition of tumor cells, or it can be provided by combination of the CD28 bispecific with a CD3 bispecific (e.g., anti-CD3×anti-MSLN), which can mimic signal 1.

Accordingly, provided herein are novel anti-CD28×anti-MSLN (also referred to as “αCD28×αMSLN” and sometimes “CD28×MSLN”) bispecific antibodies and methods of using such antibodies for the treatment of MSLN-associated cancers. In many cases, these bispecific antibodies are heterodimeric. Subject αCD28×αMSLN antibodies are capable of agonistically binding to CD28 costimulatory molecules on T cells and targeting to MSLN on MSLN-expressing tumor cells. Thus, such antibodies selectively enhance anti-tumor activity at MSLN-expressing tumor sites while minimizing peripheral toxicity. The subject antibodies provided herein are particularly useful for enhancing anti-tumor activity either alone, as a monotherapy, or when used in combination with other anti-cancer therapies as more fully described herein.

Accordingly, in one aspect, provided herein are heterodimeric antibodies that bind to two different antigens, e.g., the antibodies are “bispecific,” in that they bind two different target antigens, generally CD28 and MSLN as described below. These heterodimeric antibodies can bind each of the target antigens either monovalently (e.g., there is a single antigen binding domain such as a variable heavy and variable light domain pair) or bivalently (there are two antigen binding domains that each independently bind the antigen). In some embodiments, the heterodimeric antibody provided herein includes one CD28 binding domain and one MSLN binding domain (e.g., heterodimeric antibodies in the “1+1 Fab-scFv-Fc” format described herein, which are thus bispecific and bivalent). In other embodiments, the heterodimeric antibody provided herein includes one CD28 binding domain and two MSLN binding domains (e.g., heterodimeric antibodies in the “2+1 Fab2-scFv-Fc” formats described herein, which are thus bispecific but trivalent, as they contain three antigen binding domains (ABDs)). The heterodimeric antibodies provided herein are based on the use of different monomers that contain amino acid substitutions (i.e., skew variants”) that “skew” formation of heterodimers over homodimers, as is more fully outlined below. In some embodiments, the heterodimer antibodies are also coupled with purification variants (e.g., “pI variants”) that allow simple purification of the heterodimers away from the homodimers, as is similarly outlined below. The heterodimeric bispecific antibodies provided generally rely on the use of engineered or variant Fc domains that can self-assemble in production cells to produce heterodimeric proteins, and methods to generate and purify such heterodimeric proteins.

### II. Nomenclature

The naming nomenclature of particular antigen binding domains (e.g., MSLN and CD28 binding domains) use a “Hx.xx_Ly.yy” type of format, with the numbers being unique identifiers to particular variable chain sequences. Thus, for example, the CD28 binding domain “1A7[CD28]_H1_L1” (FIG. 27) includes a variable heavy domain, H1, and a variable light domain L1. In the case that these sequences are used as scFvs, the designation “H1_L1”, indicates that the binding domain includes a variable heavy domain “H1” combined with a variable light domain “L1,” and is in VH-linker-VL orientation, from N- to C-terminus. This molecule with the identical sequences of the heavy and light variable domains but in the reverse order (VL-linker-VH orientation, from N- to C-terminus) would be designated “L1_1”. Similarly, different constructs may “mix and match” the heavy and light chains as will be evident from the sequence listing and the Figures.

### III. Definitions

In order that the application may be more completely understood, several definitions are set forth below. Such definitions are meant to encompass grammatical equivalents.

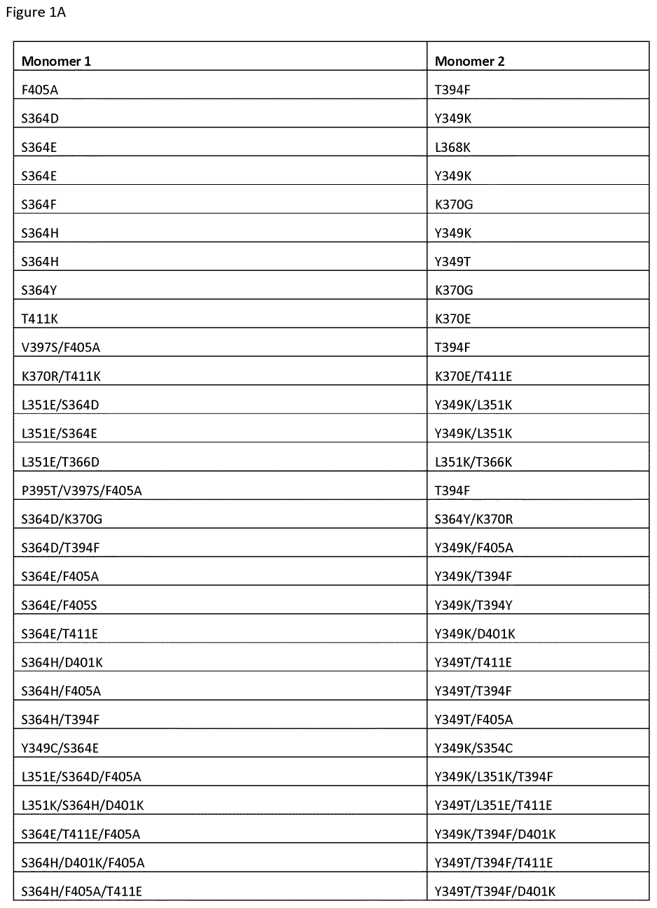

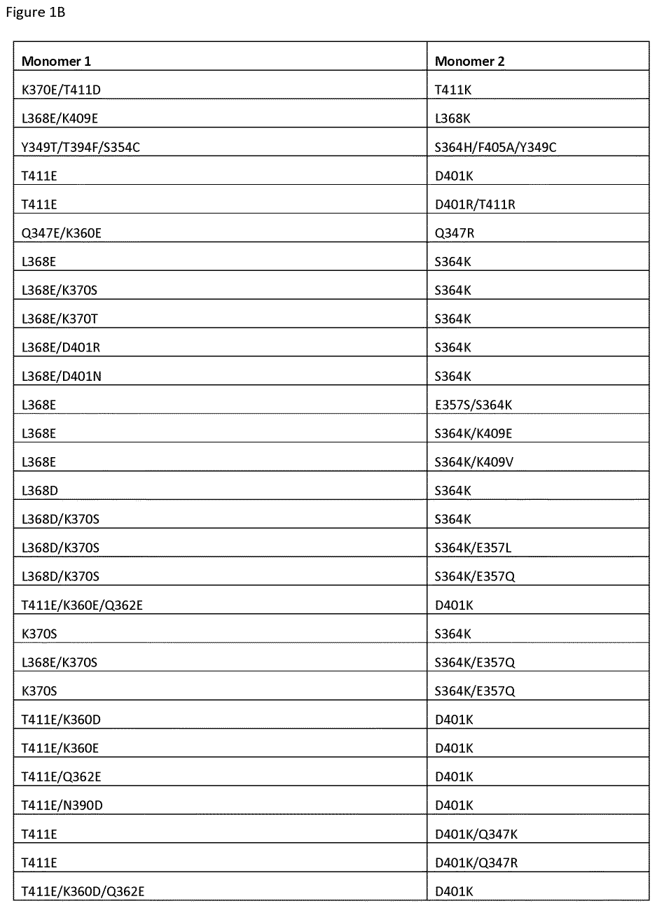

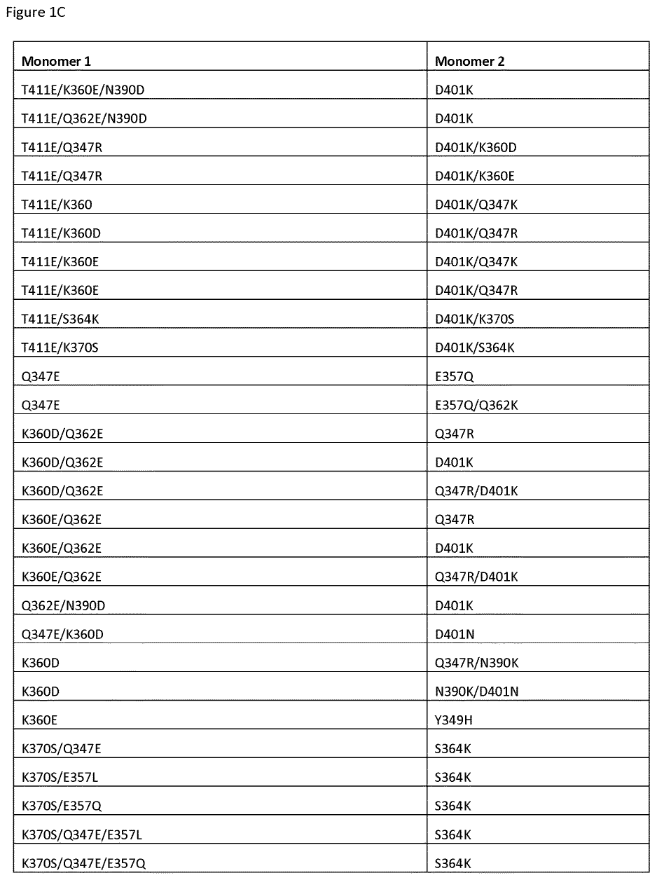

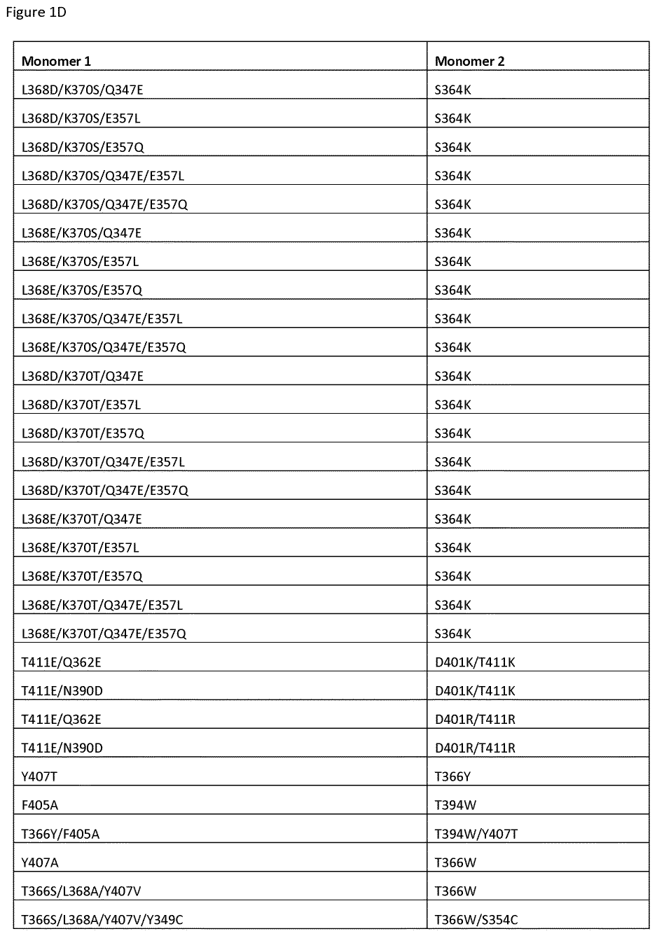

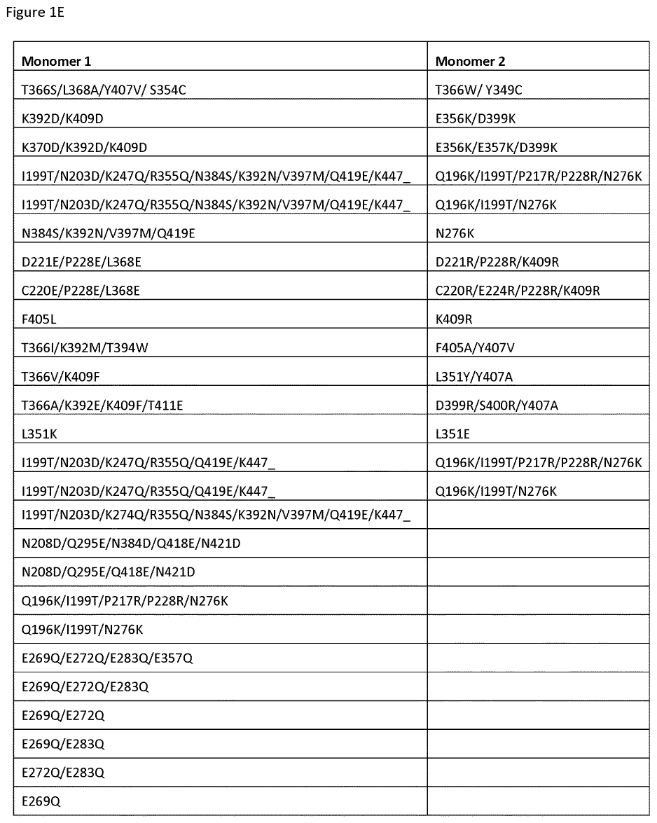

By “CD28,” “Cluster of Differentiation 28,” and “Tp44” (e.g., Genebank Accession Numbers NP_001230006 (human), NP_001230007 (human), NP_006130 (human), and NP_031668 (mouse)) herein is meant a B7 receptor expressed on T cells that provides co-stimulatory signals required for T cell activation and survival. T cell stimulation through CD28 in addition to the T cell receptor (TCR) provides a potent signal for the production of various interleukins. CD28 is the receptor for CD80 (B7.1) and CD86 (B7.2) proteins. CD28 includes an intercellular domain with a YNM motif critical for the recruitment of SH2-domain containing proteins, particularly PI3K. CD28 also includes two proline-rich motifs that are able to bind SH3-containing proteins. Exemplary CD28 sequences are depicted in FIG. 1. Unless otherwise noted, references to CD28 are to the human CD28 sequence.

By “MSLN” or “Mesothelin” or “MESO” (e.g., FIG. 14A-14B) herein is meant a protein belonging to a series of ectoenzymes that are involved in hydrolysis of extracellular nucleotides. MSLN sequences are depicted, for example, in FIGS. 14A and 14B. MSLN is expressed in particular cancers, including ovarian cancer, colon cancer, breast cancer, hepatocellular carcinoma, lung cancer, mesothelioma, pancreatic cancer, non-small cell lung cancer (such as lung adenocarcinoma) and endometrial cancer.

By “B7H3,” “B7-H3,” “B7RP-2,” “CD276,” “Cluster of Differentiation 276,” (e.g., Genebank Accession Numbers NP_001019907 (human), NP_001316557 (human), NP_001316558 (human), NP_079516 (human), and NP_598744 (mouse)) herein is meant a type-1 transmembrane protein that is a member of the B7 family possessing an ectodomain composed of a single IgV-IgC domain pair. B7H3 is an immune checkpoint molecule and is aberrantly overexpressed in many types of cancers. Unless otherwise noted, references to B7H3 are to the human B7H3 sequence.

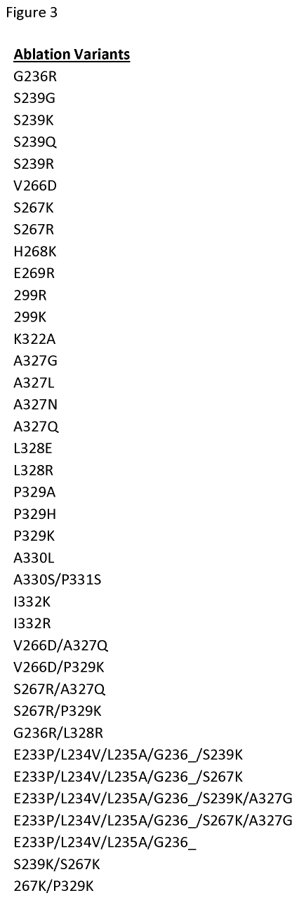

By “ablation” herein is meant a decrease or removal of activity. Thus, for example, “ablating FcγR binding” means the Fc region amino acid variant has less than 50% starting binding as compared to an Fc region not containing the specific variant, with more than 70-80-90-95-98% loss of activity being preferred, and in general, with the activity being below the level of detectable binding in a Biacore, SPR or BLI assay. Of particular use in the ablation of FcγR binding are those shown in FIG. 3, which generally are added to both monomers.

By “ADCC” or “antibody dependent cell-mediated cytotoxicity” as used herein is meant the cell-mediated reaction, wherein nonspecific cytotoxic cells that express FcγRs recognize bound antibody on a target cell and subsequently cause lysis of the target cell. ADCC is correlated with binding to FcγRIIIa; increased binding to FcγRIIIa leads to an increase in ADCC activity.

By “ADCP” or antibody dependent cell-mediated phagocytosis as used herein is meant the cell-mediated reaction wherein nonspecific phagocytic cells that express FcγRs recognize bound antibody on a target cell and subsequently cause phagocytosis of the target cell.

As used herein, the term “antibody” is used generally. Antibodies provided herein can take on a number of formats as described herein, including traditional antibodies as well as antibody derivatives, fragments and mimetics, described herein.

Traditional immunoglobulin (Ig) antibodies are “Y” shaped tetramers. Each tetramer is typically composed of two identical pairs of polypeptide chains, each pair having one “light chain” monomer (typically having a molecular weight of about 25 kDa) and one “heavy chain” monomer (typically having a molecular weight of about 50-70 kDa).

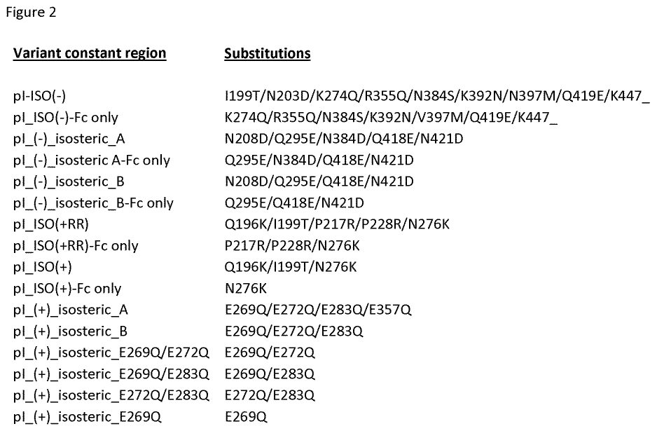

Other useful antibody formats include, but are not limited to, the “1+1 Fab-scFv-Fc,” and “2+1 Fab2-scFv-Fc” formats provided herein (see, e.g., FIG. 26). Additional useful antibody formats include, but are not limited to, “1+1 common light chain,” and “2+1 common light chain,” “mAb-Fv,” “mAb-scFv,” “central-Fv”, “one-armed scFv-mAb,” “scFv-mAb,” “dual scFv,” and “trident” format antibodies (FIG. 26). See also, US20180127501A1, which is incorporated by reference herein, particularly in pertinent part relating to antibody formats (see, e.g., FIG. 2 of US20180127501A1).

Antibody heavy chains typically include a variable heavy (VH) domain, which includes vhCDR1-3, and an Fc domain, which includes a CH2-CH3 monomer. In some embodiments, antibody heavy chains include a hinge and CH1 domain. Traditional antibody heavy chains are monomers that are organized, from N- to C-terminus: VH-CH1-hinge-CH2-CH3. The CH1-hinge-CH2-CH3 is collectively referred to as the heavy chain “constant domain” or “constant region” of the antibody, of which there are five different categories or “isotypes”: IgA, IgD, IgG, IgE and IgM.

In some embodiments, the antibodies provided herein include IgG isotype constant domains, which has several subclasses, including, but not limited to IgG1, IgG2, IgG3, and IgG4. In the IgG subclass of immunoglobulins, there are several immunoglobulin domains in the heavy chain. By “immunoglobulin (Ig) domain” herein is meant a region of an immunoglobulin having a distinct tertiary structure. Of interest in the present invention are the heavy chain domains, including, the constant heavy (CH) domains and the hinge domains. In the context of IgG antibodies, the IgG isotypes each have three CH regions. Accordingly, “CH” domains in the context of IgG are as follows: “CH1” refers to positions 118-215 according to the EU index as in Kabat. “Hinge” refers to positions 216-230 according to the EU index as in Kabat. “CH2” refers to positions 231-340 according to the EU index as in Kabat, and “CH3” refers to positions 341-447 according to the EU index as in Kabat. As shown in Table 1, the exact numbering and placement of the heavy chain domains can be different among different numbering systems. As shown herein and described below, the pI variants can be in one or more of the CH regions, as well as the hinge region, discussed below.

It should be noted that IgG1 has different allotypes with polymorphisms at 356 (D or E) and 358 (L or M). The sequences depicted herein use the 356E/358M allotype, however the other allotype is included herein. That is, any sequence inclusive of an IgG1 Fc domain included herein can have 356D/358L replacing the 356E/358M allotype. It should be understood that therapeutic antibodies can also comprise hybrids of isotypes and/or subclasses. For example, as shown in US Publication 2009/0163699, incorporated by reference, the present antibodies, in some embodiments, include human IgG1/G2 hybrids.

By “Fc” or “Fc region” or “Fc domain” as used herein is meant the polypeptide comprising the constant region of an antibody, in some instances, excluding all of the first constant region immunoglobulin domain (e.g., CH1) or a portion thereof, and in some cases, optionally including all or part of the hinge. For IgG, the Fc domain comprises immunoglobulin domains CH2 and CH3 (Cγ2 and Cγ3), and optionally all or a portion of the hinge region between CH1 (Cγ1) and CH2 (Cγ2). Thus, in some cases, the Fc domain includes, from N- to C-terminal, CH2-CH3 and hinge-CH2-CH3. In some embodiments, the Fc domain is that from IgG1, IgG2, IgG3 or IgG4, with IgG1 hinge-CH2-CH3 and IgG4 hinge-CH2-CH3 finding particular use in many embodiments. Additionally, in the case of human IgG1 Fc domains, the hinge may include a C220S amino acid substitution. Furthermore, in the case of human IgG4 Fc domains, the hinge may include a S228P amino acid substitution. Although the boundaries of the Fc region may vary, the human IgG heavy chain Fc region is usually defined to include residues E216, C226, or A231 to its carboxyl-terminal, wherein the numbering is according to the EU index as in Kabat. In some embodiments, as is more fully described below, amino acid modifications are made to the Fc region, for example to alter binding to one or more FcγR or to the FcRn.

By “heavy chain constant region” herein is meant the CH1-hinge-CH2-CH3 portion of an antibody (or fragments thereof), excluding the variable heavy domain; in EU numbering of human IgG1 this is amino acids 118-447. By “heavy chain constant region fragment” herein is meant a heavy chain constant region that contains fewer amino acids from either or both of the N- and C-termini but still retains the ability to form a dimer with another heavy chain constant region.

Another type of domain of the heavy chain is the hinge region. By “hinge” or “hinge region” or “antibody hinge region” or “hinge domain” herein is meant the flexible polypeptide comprising the amino acids between the first and second constant domains of an antibody. Structurally, the IgG CH1 domain ends at EU position 215, and the IgG CH2 domain begins at residue EU position 231. Thus for IgG the antibody hinge is herein defined to include positions 216 (E216 in IgG1) to 230 (P230 in IgG1), wherein the numbering is according to the EU index as in Kabat. In some cases, a “hinge fragment” is used, which contains fewer amino acids at either or both of the N- and C-termini of the hinge domain. As noted herein, pI variants can be made in the hinge region as well. Many of the antibodies herein have at least one the cysteines at position 220 according to EU numbering (hinge region) replaced by a serine. Generally, this modification is on the “scFv monomer” side (when 1+1 or 2+1 formats are used) for most of the sequences depicted herein, although it can also be on the “Fab monomer” side, or both, to reduce disulfide formation. Specifically included within the sequences herein are one or both of these cysteines replaced (C220S).

As will be appreciated by those in the art, the exact numbering and placement of the heavy chain constant region domains (i.e., CH1, hinge, CH2 and CH3 domains) can be different among different numbering systems. A useful comparison of heavy constant region numbering according to EU and Kabat is as below, see Edelman et al., 1969, Proc Natl Acad Sci USA 63:78-85 and Kabat et al., 1991, Sequences of Proteins of Immunological Interest, 5th Ed., United States Public Health Service, National Institutes of Health, Bethesda, entirely incorporated by reference.

TABLE 1

| | EU Numbering | Kabat Numbering |
|---|---|---|
| CH1 | 118-215 | 114-223 |
| Hinge | 216-230 | 226-243 |
| CH2 | 231-340 | 244-360 |
| CH3 | 341-447 | 361-478 |

The antibody light chain generally comprises two domains: the variable light domain (VL), which includes light chain CDRs vlCDR1-3, and a constant light chain region (often referred to as CL or Cκ). The antibody light chain is typically organized from N- to C-terminus: VL-CL.

By “antigen binding domain” or “ABD” herein is meant a set of six Complementary Determining Regions (CDRs) that, when present as part of a polypeptide sequence, specifically binds a target antigen (e.g., MSLN or CD28) as discussed herein. As is known in the art, these CDRs are generally present as a first set of variable heavy CDRs (vhCDRs or VHCDRs) and a second set of variable light CDRs (vlCDRs or VLCDRs), each comprising three CDRs: vhCDR1, vhCDR2, vhCDR3 variable heavy CDRs and vlCDR1, vlCDR2 and vlCDR3 vhCDR3 variable light CDRs. The CDRs are present in the variable heavy domain (vhCDR1-3) and variable light domain (vlCDR1-3). The variable heavy domain and variable light domain from an Fv region.

The present invention provides a large number of different CDR sets. In this case, a “full CDR set” comprises the three variable light and three variable heavy CDRs, e.g., a vlCDR1, vlCDR2, vlCDR3, vhCDR1, vhCDR2 and vhCDR3. These can be part of a larger variable light or variable heavy domain, respectfully. In addition, as more fully outlined herein, the variable heavy and variable light domains can be on separate polypeptide chains, when a heavy and light chain is used (for example when Fabs are used), or on a single polypeptide chain in the case of scFv sequences.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the vhCDRs (e.g., vhCDR1, vhCDR2 and vhCDR3) and the disclosure of each variable light region is a disclosure of the vlCDRs (e.g., vlCDR1, vlCDR2 and vlCDR3). A useful comparison of CDR numbering is as below, see Lafranc et al., Dev. Comp. Immunol. 27(1):55-77 (2003):

TABLE 2

| | Kabat + | | | | | | |
|---|---|---|---|---|---|---|---|
| | Chothia | IMGT | Kabat | AbM | Chothia | Contact | Xencor |
| vhCDR1 | 26-35 | 27-38 | 31-35 | 26-35 | 26-32 | 30-35 | 27-35 |
| vhCDR2 | 50-65 | 56-65 | 50-65 | 50-58 | 52-56 | 47-58 | 54-61 |
| vhCDR3 | 95-102 | 105-117 | 95-102 | 95-102 | 95-102 | 93-101 | 103-116 |
| vlCDR1 | 24-34 | 27-38 | 24-34 | 24-34 | 24-34 | 30-36 | 27-38 |
| vlCDR2 | 50-56 | 56-65 | 50-56 | 50-56 | 50-56 | 46-55 | 56-62 |
| vlCDR3 | 89-97 | 105-117 | 89-97 | 89-97 | 89-97 | 89-96 | 97-105 |

Throughout the present specification, the Kabat numbering system is generally used when referring to a residue in the variable domain (approximately, residues 1-107 of the light chain variable region and residues 1-113 of the heavy chain variable region) and the EU numbering system for Fc regions (e.g., Kabat et al., supra (1991)).

The CDRs contribute to the formation of the antigen-binding, or more specifically, epitope binding site of the antigen binding domains and antibodies. “Epitope” refers to a determinant that interacts with a specific antigen binding site in the variable region of an antibody molecule known as a paratope. Epitopes are groupings of molecules such as amino acids or sugar side chains and usually have specific structural characteristics, as well as specific charge characteristics. A single antigen may have more than one epitope.

The epitope may comprise amino acid residues directly involved in the binding (also called immunodominant component of the epitope) and other amino acid residues, which are not directly involved in the binding, such as amino acid residues which are effectively blocked by the specifically antigen binding peptide; in other words, the amino acid residue is within the footprint of the specifically antigen binding peptide.

Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and nonconformational epitopes may be distinguished in that the binding to the former but not the latter is lost in the presence of denaturing solvents.

An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen, for example “binning.” As outlined below, the invention not only includes the enumerated antigen binding domains and antibodies herein, but those that compete for binding with the epitopes bound by the enumerated antigen binding domains.

In some embodiments, the six CDRs of the antigen binding domain are contributed by a variable heavy and a variable light domain. In a “Fab” format, the set of 6 CDRs are contributed by two different polypeptide sequences, the variable heavy domain (vh or VH; containing the vhCDR1, vhCDR2 and vhCDR3) and the variable light domain (vl or VL; containing the vlCDR1, vlCDR2 and vlCDR3), with the C-terminus of the vh domain being attached to the N-terminus of the CH1 domain of the heavy chain and the C-terminus of the vl domain being attached to the N-terminus of the constant light domain (and thus forming the light chain). In a scFv format, the vh and vl domains are covalently attached, generally through the use of a linker (a “scFv linker”) as outlined herein, into a single polypeptide sequence, which can be either (starting from the N-terminus) vh-linker-vl or vl-linker-vh, with the former being generally preferred (including optional domain linkers on each side, depending on the format used. In general, the C-terminus of the scFv domain is attached to the N-terminus of all or part of the hinge in the second monomer.

By “variable region” or “variable domain” as used herein is meant the region of an immunoglobulin that comprises one or more Ig domains substantially encoded by any of the Vx, VX, and/or VH genes that make up the kappa, lambda, and heavy chain immunoglobulin genetic loci respectively, and contains the CDRs that confer antigen specificity. Thus, a “variable heavy domain” pairs with a “variable light domain” to form an antigen binding domain (“ABD”). In addition, each variable domain comprises three hypervariable regions (“complementary determining regions,” “CDRs”) (vhCDR1, vhCDR2 and vhCDR3 for the variable heavy domain and vlCDR1, vlCDR2 and vlCDR3 for the variable light domain) and four framework (FR) regions, arranged from amino-terminus to carboxy-terminus in the following order: FR1-CDR1-FR2-CDR2-FR3-CDR3-FR4.

By “Fab” or “Fab region” as used herein is meant the antibody region that comprises the VH, CH1, VL, and CL immunoglobulin domains, generally on two different polypeptide chains (e.g., VH-CH1 on one chain and VL-CL on the other). Fab may refer to this region in isolation, or this region in the context of a bispecific antibody of the invention. In the context of a Fab, the Fab comprises an Fv region in addition to the CH1 and CL domains.

By “Fv” or “Fv fragment” or “Fv region” as used herein is meant the antibody region that comprises the VL and VH domains. Fv regions can be formatted as both Fabs (as discussed above, generally two different polypeptides that also include the constant regions as outlined above) and single chain Fvs (scFvs), where the vl and vh domains are included in a single peptide, attached generally with a linker as discussed herein.

By “single chain Fv” or “scFv” herein is meant a variable heavy domain covalently attached to a variable light domain, generally using a scFv linker as discussed herein, to form a scFv or scFv domain. A scFv domain can be in either orientation from N- to C-terminus (vh-linker-vl or vl-linker-vh). In the sequences depicted in the sequence listing and in the figures, the order of the vh and vl domain is indicated in the name, e.g., H.X_L.Y means N- to C-terminal is vh-linker-vl, and L.Y_H.X is vl-linker-vh.

Some embodiments of the subject antibodies provided herein comprise at least one scFv domain, which, while not naturally occurring, generally includes a variable heavy domain and a variable light domain, linked together by a scFv linker. As outlined herein, while the scFv domain is generally from N- to C-terminus oriented as VH-scFv linker-VL, this can be reversed for any of the scFv domains (or those constructed using vh and vl sequences from Fabs), to VL-scFv linker-VH, with optional linkers at one or both ends depending on the format.

By “modification” or “variant” herein is meant an amino acid substitution, insertion, and/or deletion in a polypeptide sequence or an alteration to a moiety chemically linked to a protein. For example, a modification may be an altered carbohydrate or PEG structure attached to a protein. By “amino acid modification” herein is meant an amino acid substitution, insertion, and/or deletion in a polypeptide sequence. For clarity, unless otherwise noted, the amino acid modification is always to an amino acid coded for by DNA, e.g., the 20 amino acids that have codons in DNA and RNA.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution E272Y refers to a variant polypeptide, in this case an Fc variant, in which the glutamic acid at position 272 is replaced with tyrosine. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution;” that is, despite the creation of a new gene encoding the same protein, if the protein has the same amino acid at the particular position that it started with, it is not an amino acid substitution.

By “amino acid insertion” or “insertion” as used herein is meant the addition of an amino acid sequence at a particular position in a parent polypeptide sequence. For example, −233E or 233E designates an insertion of glutamic acid after position 233 and before position 234. Additionally, −233ADE or A233ADE designates an insertion of AlaAspGlu after position 233 and before position 234.

By “amino acid deletion” or “deletion” as used herein is meant the removal of an amino acid sequence at a particular position in a parent polypeptide sequence. For example, E233- or E233 #, E233( ) or E233del designates a deletion of glutamic acid at position 233. Additionally, EDA233- or EDA233 #designates a deletion of the sequence GluAspAla that begins at position 233.

By “variant protein” or “protein variant”, or “variant” as used herein is meant a protein that differs from that of a parent protein by virtue of at least one amino acid modification. The protein variant has at least one amino acid modification compared to the parent protein, yet not so many that the variant protein will not align with the parental protein using an alignment program such as that described below. In general, variant proteins (such as variant Fc domains, etc., outlined herein, are generally at least 75, 80, 85, 90, 91, 92, 93, 94, 95, 96, 97, 98 or 99% identical to the parent protein, using the alignment programs described below, such as BLAST.

“Variant” as used herein also refers to particular amino acid modifications that confer particular function (e.g., a “heterodimerization variant,” “pI variant,” “ablation variant,” etc.).

As described below, in some embodiments the parent polypeptide, for example an Fc parent polypeptide, is a human wild type sequence, such as the heavy constant domain or Fc region from IgG1, IgG2, IgG3 or IgG4, although human sequences with variants can also serve as “parent polypeptides”, for example the IgG1/2 hybrid of US Publication 2006/0134105 can be included. The protein variant sequence herein will preferably possess at least about 80% identity with a parent protein sequence, and most preferably at least about 90% identity, more preferably at least about 95-98-99% identity. Accordingly, by “antibody variant” or “variant antibody” as used herein is meant an antibody that differs from a parent antibody by virtue of at least one amino acid modification, “IgG variant” or “variant IgG” as used herein is meant an antibody that differs from a parent IgG (again, in many cases, from a human IgG sequence) by virtue of at least one amino acid modification, and “immunoglobulin variant” or “variant immunoglobulin” as used herein is meant an immunoglobulin sequence that differs from that of a parent immunoglobulin sequence by virtue of at least one amino acid modification. “Fc variant” or “variant Fc” as used herein is meant a protein comprising an amino acid modification in an Fc domain as compared to an Fc domain of human IgG1, IgG2 or IgG4.

“Fc variant” or “variant Fc” as used herein is meant a protein comprising an amino acid modification in an Fc domain. The modification can be an addition, deletion, or substitution. The Fc variants are defined according to the amino acid modifications that compose them. Thus, for example, N434S or 434S is an Fc variant with the substitution for serine at position 434 relative to the parent Fc polypeptide, wherein the numbering is according to the EU index. Likewise, M428L/N434S defines an Fc variant with the substitutions M428L and N434S relative to the parent Fc polypeptide. The identity of the WT amino acid may be unspecified, in which case the aforementioned variant is referred to as 428L/434S. It is noted that the order in which substitutions are provided is arbitrary, that is to say that, for example, 428L/434S is the same Fc variant as 434S/428L, and so on. For all positions discussed herein that relate to antibodies or derivatives and fragments thereof (e.g., Fc domains), unless otherwise noted, amino acid position numbering is according to the EU index. The “EU index” or “EU index as in Kabat” or “EU numbering” scheme refers to the numbering of the EU antibody (Edelman et al., 1969, Proc Natl Acad Sci USA 63:78-85, hereby entirely incorporated by reference). The modification can be an addition, deletion, or substitution.

In general, variant Fc domains have at least about 80, 85, 90, 95, 97, 98 or 99 percent identity to the corresponding parental human IgG Fc domain (using the identity algorithms discussed below, with one embodiment utilizing the BLAST algorithm as is known in the art, using default parameters). Alternatively, the variant Fc domains can have from 1, 2, 3, 4, 5, 6, 7, 8, 9, 10, 11, 12, 13, 14, 15, 16, 11, 12, 13, 14, 15, 16, 17, 18, 19 or 20 amino acid modifications as compared to the parental Fc domain. Alternatively, the variant Fc domains can have up to 1, 2, 3, 4, 5, 6, 7, 8, 9, 10, 11, 12, 13, 14, 15, 16, 11, 12, 13, 14, 15, 16, 17, 18, 19 or 20 amino acid modifications as compared to the parental Fc domain. Additionally, as discussed herein, the variant Fc domains described herein still retain the ability to form a dimer with another Fc domain as measured using known techniques as described herein, such as non-denaturing gel electrophoresis.

By “protein” as used herein is meant at least two covalently attached amino acids, which includes proteins, polypeptides, oligopeptides and peptides. In addition, polypeptides that make up the antibodies of the invention may include synthetic derivatization of one or more side chains or termini, glycosylation, PEGylation, circular permutation, cyclization, linkers to other molecules, fusion to proteins or protein domains, and addition of peptide tags or labels.

By “residue” as used herein is meant a position in a protein and its associated amino acid identity. For example, Asparagine 297 (also referred to as Asn297 or N297) is a residue at position 297 in the human antibody IgG1.

By “IgG subclass modification” or “isotype modification” as used herein is meant an amino acid modification that converts one amino acid of one IgG isotype to the corresponding amino acid in a different, aligned IgG isotype. For example, because IgG1 comprises a tyrosine and IgG2 a phenylalanine at EU position 296, a F296Y substitution in IgG2 is considered an IgG subclass modification.

By “non-naturally occurring modification” as used herein is meant an amino acid modification that is not isotypic. For example, because none of the human IgGs comprise a serine at position 434, the substitution 434S in IgG1, IgG2, IgG3, or IgG4 (or hybrids thereof) is considered a non-naturally occurring modification.

By “amino acid” and “amino acid identity” as used herein is meant one of the 20 naturally occurring amino acids that are coded for by DNA and RNA.

By “effector function” as used herein is meant a biochemical event that results from the interaction of an antibody Fc region with an Fc receptor or ligand. Effector functions include but are not limited to ADCC, ADCP, and CDC.

By “IgG Fc ligand” as used herein is meant a molecule, preferably a polypeptide, from any organism that binds to the Fc region of an IgG antibody to form an Fc/Fc ligand complex. Fc ligands include but are not limited to FcγRIs, FcγRIIs, FcγRIIIs, FcRn, C1q, C3, mannan binding lectin, mannose receptor, staphylococcal protein A, streptococcal protein G, and viral FcγR. Fc ligands also include Fc receptor homologs (FcRH), which are a family of Fc receptors that are homologous to the FcγRs (Davis et al., 2002, Immunological Reviews 190:123-136, entirely incorporated by reference). Fc ligands may include undiscovered molecules that bind Fc. Particular IgG Fc ligands are FcRn and Fc gamma receptors. By “Fc ligand” as used herein is meant a molecule, preferably a polypeptide, from any organism that binds to the Fc region of an antibody to form an Fc/Fc ligand complex.

By “Fc gamma receptor”, “FcγR” or “FcgammaR” as used herein is meant any member of the family of proteins that bind the IgG antibody Fc region and is encoded by an FcγR gene. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIb-NA1 and FcγRIIb-NA2) (Jefferis et al., 2002, Immunol Lett 82:57-65, entirely incorporated by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII (CD16), and FcγRIII-2 (CD16-2), as well as any undiscovered mouse FcγRs or FcγR isoforms or allotypes.

By “FcRn” or “neonatal Fc Receptor” as used herein is meant a protein that binds the IgG antibody Fc region and is encoded at least in part by an FcRn gene. The FcRn may be from any organism, including but not limited to humans, mice, rats, rabbits, and monkeys. As is known in the art, the functional FcRn protein comprises two polypeptides, often referred to as the heavy chain and light chain. The light chain is beta-2-microglobulin and the heavy chain is encoded by the FcRn gene. Unless otherwise noted herein, FcRn or an FcRn protein refers to the complex of FcRn heavy chain with beta-2-microglobulin. A variety of FcRn variants used to increase binding to the FcRn receptor, and in some cases, to increase serum half-life. An “FcRn variant” is an amino acid modification that contributes to increased binding to the FcRn receptor, and suitable FcRn variants are shown below.

By “parent polypeptide” as used herein is meant a starting polypeptide that is subsequently modified to generate a variant. The parent polypeptide may be a naturally occurring polypeptide, or a variant or engineered version of a naturally occurring polypeptide. Accordingly, by “parent immunoglobulin” as used herein is meant an unmodified immunoglobulin polypeptide that is modified to generate a variant, and by “parent antibody” as used herein is meant an unmodified antibody that is modified to generate a variant antibody. It should be noted that “parent antibody” includes known commercial, recombinantly produced antibodies as outlined below. In this context, a “parent Fc domain” will be relative to the recited variant; thus, a “variant human IgG1 Fc domain” is compared to the parent Fc domain of human IgG1, a “variant human IgG4 Fc domain” is compared to the parent Fc domain human IgG4, etc.

By “position” as used herein is meant a location in the sequence of a protein. Positions may be numbered sequentially, or according to an established format, for example the EU index for numbering of antibody domains (e.g., a CH1, CH2, CH3 or hinge domain).

By “target antigen” as used herein is meant the molecule that is bound specifically by the antigen binding domain comprising the variable regions of a given antibody.

By “strandedness” in the context of the monomers of the heterodimeric antibodies of the invention herein is meant that, similar to the two strands of DNA that “match”, heterodimerization variants are incorporated into each monomer so as to preserve the ability to “match” to form heterodimers. For example, if some pI variants are engineered into monomer A (e.g., making the pI higher) then steric variants that are “charge pairs” that can be utilized as well do not interfere with the pI variants, e.g., the charge variants that make a pI higher are put on the same “strand” or “monomer” to preserve both functionalities. Similarly, for “skew” variants that come in pairs of a set as more fully outlined below, the skilled artisan will consider pI in deciding into which strand or monomer one set of the pair will go, such that pI separation is maximized using the pI of the skews as well.

By “target cell” as used herein is meant a cell that expresses a target antigen.

By “host cell” in the context of producing a bispecific antibody according to the invention herein is meant a cell that contains the exogeneous nucleic acids encoding the components of the bispecific antibody and is capable of expressing the bispecific antibody under suitable conditions. Suitable host cells are discussed below.

By “wild type” or “WT” herein is meant an amino acid sequence or a nucleotide sequence that is found in nature, including allelic variations. A WT protein has an amino acid sequence or a nucleotide sequence that has not been intentionally modified.

Provided herein are a number of antibody domains (e.g., Fc domains) that have sequence identity to human antibody domains. Sequence identity between two similar sequences (e.g., antibody variable domains) can be measured by algorithms such as that of Smith, T. F. & Waterman, M. S. (1981) “Comparison Of Biosequences,” Adv. Appl. Math. 2:482 [local homology algorithm]; Needleman, S. B. & Wunsch, C D. (1970) “A General Method Applicable To The Search For Similarities In The Amino Acid Sequence Of Two Proteins,” J. Mol. Biol. 48:443 [homology alignment algorithm], Pearson, W. R. & Lipman, D. J. (1988) “Improved Tools For Biological Sequence Comparison,” Proc. Natl. Acad. Sci. (U.S.A.) 85:2444 [search for similarity method]; or Altschul, S. F. et al, (1990) “Basic Local Alignment Search Tool,” J. Mol. Biol. 215:403-10, the “BLAST” algorithm, see https://blast.ncbi.nlm.nih.gov/Blast.cgi. When using any of the aforementioned algorithms, the default parameters (for Window length, gap penalty, etc.) are used. In one embodiment, sequence identity is done using the BLAST algorithm, using default parameters

The antibodies of the present invention are generally isolated or recombinant. “Isolated,” when used to describe the various polypeptides disclosed herein, means a polypeptide that has been identified and separated and/or recovered from a cell or cell culture from which it was expressed. Ordinarily, an isolated polypeptide will be prepared by at least one purification step. An “isolated antibody,” refers to an antibody which is substantially free of other antibodies having different antigenic specificities. “Recombinant” means the antibodies are generated using recombinant nucleic acid techniques in exogeneous host cells, and they can be isolated as well.

“Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope can be exhibited, for example, by an antibody having a KD for an antigen or epitope of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the antigen or epitope.

Also, specific binding for a particular antigen or an epitope can be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction. Binding affinity is generally measured using a Biacore, SPR or BLI assay.

### IV. Anti-CD28×Anti-MSLN Antibodies

In one aspect, provided herein are novel anti-CD28×anti-MSLN antibodies In some embodiments, the anti-CD28×anti-MSLN antibodies described herein are capable of agonistically binding to CD28 costimulatory molecules on T cells and MSLN on tumor cells. Such antibodies selectively enhance anti-tumor activity at MSLN-associated tumor sites while minimizing peripheral toxicity. The subject antibodies provided herein are particularly useful in combination with other anti-cancer therapies, including, for example, bispecific antibodies for the treatment of MSLN-associated cancers (e.g., ovarian cancer).

The anti-CD28×anti-MSLN antibodies are multivalent and include at least two antigen binding domains (ABDs), wherein at least one antigen binding domain is a CD28 binding domain and at least one antigen binding domain is a MSLN binding domain. Any suitable CD28 binding domain and MSLN binding domain can be included in the subject anti-CD28×anti-MSLN antibodies, including, for example, the CD28 binding domains and MSLN binding domains provided herein.

The antigen binding domains provided herein generally include a variable heavy domain (VH) having a VH-CDR1, VH-CDR-2, and VH-CDR-3; and a variable light domain (VL), and a variable light domain (VL) having a VL-CDR1, VL-CDR-2, and VL-CDR-3.

In addition, as discussed above, the numbering used in the sequence listing and figures for the identification of the CDRs is Kabat, however, different numbering can be used, which will change the amino acid sequences of the CDRs as shown in Table 2.

For all of the variable heavy and light domains listed herein, further variants can be made. As outlined herein, in some embodiments the set of 6 CDRs can have from 0, 1, 2, 3, 4 or 5 amino acid modifications (with amino acid substitutions finding particular use), as well as changes in the framework regions of the variable heavy and light domains, as long as the frameworks (excluding the CDRs) retain at least about 80, 85, 90, 95 or 99% identity to a human germline sequence selected from those listed in FIG. 1 of U.S. Pat. No. 7,657,380, which Figure and Legend is incorporated by reference in its entirety herein. Thus, for example, the identical CDRs as described herein can be combined with different framework sequences from human germline sequences, as long as the framework regions retain at least 80, 85 or 90% identity to a human germline sequence selected from those listed in FIG. 1 of U.S. Pat. No. 7,657,380. Alternatively, the CDRs can have amino acid modifications (e.g., from 1, 2, 3, 4 or 5 amino acid modifications in the set of CDRs (that is, the CDRs can be modified as long as the total number of changes in the set of 6 CDRs is less than 6 amino acid modifications, with any combination of CDRs being changed; e.g., there may be one change in vlCDR1, two in vhCDR2, none in vhCDR3, etc.)), as well as having framework region changes, as long as the framework regions retain at least 80, 85, 95 to 99% identity to a human germline sequence selected from those listed in FIG. 1 of U.S. Pat. No. 7,657,380.

As will be appreciated by those in the art, any set of 6 CDRs or VH and VL domains can be in the scFv format or in the Fab format, which is then added to the heavy and light constant domains, where the heavy constant domains comprise variants (including within the CH1 domain as well as the Fc domain).

In addition, in embodiments wherein the subject antibody includes an scFv, the scFv can be in an orientation from N- to C-terminus of VH-scFv linker-VL or VL-scFv linker-VH. In some formats, one or more of the ABDs generally is a Fab that includes a VH domain on one protein chain (generally as a component of a heavy chain) and a VL on another protein chain (generally as a component of a light chain). Exemplary scFv linkers for use in the subject antibodies are depicted in FIG. 5.

In some embodiments, the anti-CD28×anti-MSLN antibody is a bispecific antibody. In some embodiments, the anti-CD28×anti-MSLN antibody is a bivalent antibody. In some embodiments, the anti-CD28×anti-MSLN antibody is a trivalent antibody. In some embodiments, the anti-CD28×anti-MSLN antibody is a bispecific, bivalent antibody. In some embodiments, the anti-CD28×anti-MSLN antibodies include one CD28 binding domain and one MSLN binding domain. In exemplary embodiments, the anti-CD28×anti-MSLN antibody is a bispecific, trivalent antibody. In some embodiments, the anti-CD28×anti-MSLN antibodies include one CD28 binding domain and two MSLN binding domains.

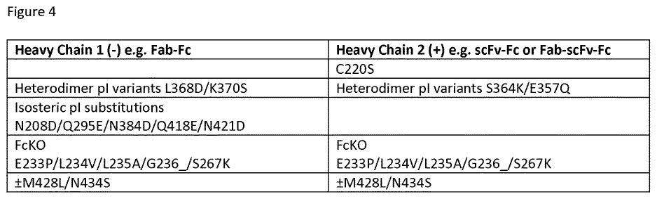

The anti-CD28×anti-MSLN antibodies provided herein can be in any useful format, including, including, for example, canonical immunoglobulin, as well as the “1+1 Fab-scFv-Fc,” “2+1 “mAb-scFv,” and “2+1 Fab2-scFv-Fc,” formats described herein (FIG. 26). Additional useful formats include, but are not limited to: “mAb-Fv,” “central-Fv”, “one-armed scFv-mAb,” “scFv-mAb,” “dual scFv,” and “trident” formats provided herein (see, e.g., FIG. 26). See also, US20180127501A1, which is incorporated by reference herein, particularly in pertinent part relating to antibody formats (see, e.g., FIG. 2). In some embodiments, the anti-CD28×anti-MSLN antibodies are heterodimeric bispecific antibodies that include variant Fc domains having any of the heterodimerization skew variants, pI variants and/or ablation variants described herein. See, e.g. FIG. 4.

Note that unless specified herein, the order of the antigen list in the name does not confer structure; that is an anti-MSLN×anti-CD28 1+1 Fab-scFv-Fc antibody can have the scFv bind to MSLN or CD28, although in some cases, the order specifies structure as indicated.

The anti-CD28×anti-MSLN antibodies provided herein further include different antibody domains. As described herein and known in the art, the antibodies described herein include different domains within the heavy and light chains, which can be overlapping as well. These domains include, but are not limited to, the Fc domain, the CH1 domain, the CH2 domain, the CH3 domain, the hinge domain, the heavy constant domain (CH1-hinge-Fc domain or CH1-hinge-CH2-CH3), the variable heavy domain, the variable light domain, the light constant domain, Fab domains and scFv domains.

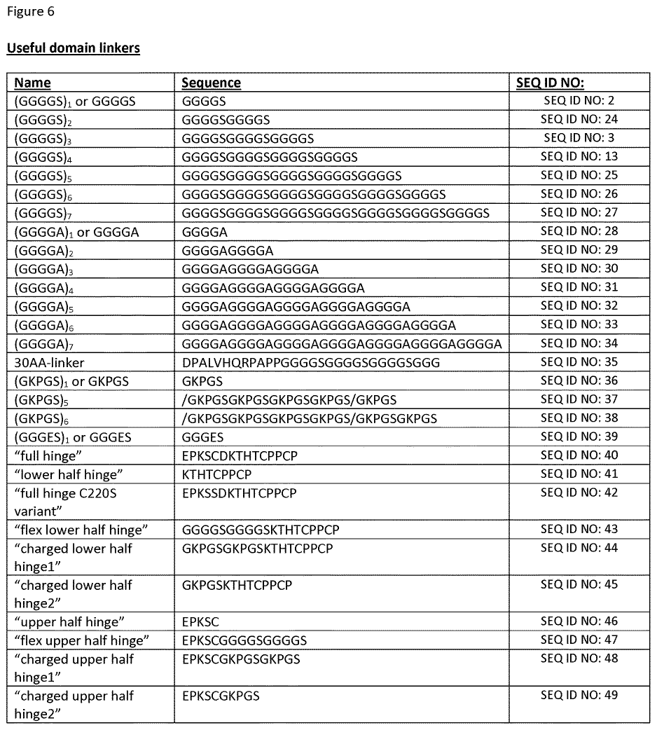

As shown herein, there are a number of suitable linkers (for use as either domain linkers or scFv linkers) that can be used to covalently attach the recited domains (e.g., scFvs, Fabs, Fc domains, VH domains, VL domains, etc.), including traditional peptide bonds, generated by recombinant techniques. Exemplary linkers to attach domains of the subject antibody to each other are depicted in FIG. 6. In some embodiments, the linker peptide may predominantly include the following amino acid residues: Gly, Ser, Ala, or Thr. The linker peptide should have a length that is adequate to link two molecules in such a way that they assume the correct conformation relative to one another so that they retain the desired activity. In one embodiment, the linker is from about 1 to 50 amino acids in length, preferably about 1 to 30 amino acids in length. In one embodiment, linkers of 1 to 20 amino acids in length may be used, with from about 5 to about 10 amino acids finding use in some embodiments. Useful linkers include glycine-serine polymers, including for example (GS)n, (GSGGS)n (SEQ ID NO: 1129), (GGGGS)n (SEQ ID NO: 1130), and (GGGS)n (SEQ ID NO: 1131), where n is an integer of at least one (and generally from 3 to 4), glycine-alanine polymers, alanine-serine polymers, and other flexible linkers. Alternatively, a variety of nonproteinaceous polymers, including but not limited to polyethylene glycol (PEG), polypropylene glycol, polyoxyalkylenes, or copolymers of polyethylene glycol and polypropylene glycol, may find use as linkers.

Other linker sequences may include any sequence of any length of CL/CH1 domain but not all residues of CL/CH1 domain; for example the first 5-12 amino acid residues of the CL/CH1 domains. Linkers can be derived from immunoglobulin light chain, for example CK or Cλ. Linkers can be derived from immunoglobulin heavy chains of any isotype, including for example Cγ1, Cγ2, Cγ3, Cγ4, Cα1, Cα2, Cδ, Cε, and Cμ. Linker sequences may also be derived from other proteins such as Ig-like proteins (e.g., TCR, FcR, KIR), hinge region-derived sequences, and other natural sequences from other proteins.

In some embodiments, the linker is a “domain linker”, used to link any two domains as outlined herein together. For example, in the 2+1 Fab2-scFv-Fc format, there may be a domain linker that attaches the C-terminus of the CH1 domain of the Fab to the N-terminus of the scFv, with another optional domain linker attaching the C-terminus of the scFv to the CH2 domain (although in many embodiments the hinge is used as this domain linker). While any suitable linker can be used, many embodiments utilize a glycine-serine polymer as the domain linker, including for (GS)n, (GSGGS)n (SEQ ID NO: 1129), (GGGGS)n (SEQ ID NO: 1130), and (GGGS)n (SEQ ID NO: 1131), where n is an integer of at least one (and generally from 3 to 4 to 5) as well as any peptide sequence that allows for recombinant attachment of the two domains with sufficient length and flexibility to allow each domain to retain its biological function. In some cases, and with attention being paid to “strandedness”, as outlined below, charged domain linkers, as used in some embodiments of scFv linkers can be used. Exemplary useful domain linkers are depicted in FIG. 6.

In some embodiments, the linker is a scFv linker that is used to covalently attach the VH and VL domains as discussed herein. In many cases, the scFv linker is a charged scFv linker, a number of which are shown in FIG. 5. Accordingly, provided herein are charged scFv linkers, to facilitate the separation in pI between a first and a second monomer. That is, by incorporating a charged scFv linker, either positive or negative (or both, in the case of scaffolds that use scFvs on different monomers), this allows the monomer comprising the charged linker to alter the pI without making further changes in the Fc domains. These charged linkers can be substituted into any scFv containing standard linkers. Again, as will be appreciated by those in the art, charged scFv linkers are used on the correct “strand” or monomer, according to the desired changes in pI. For example, as discussed herein, to make 1+1 Fab-scFv-Fc format heterodimeric antibody, the original pI of the Fv region for each of the desired antigen binding domains are calculated, and one is chosen to make an scFv, and depending on the pI, either positive or negative linkers are chosen. Charged domain linkers can also be used to increase the pI separation of the monomers of the invention as well, and thus those included in FIG. 6 can be used in any embodiment herein where a linker is utilized.

Exemplary subject anti-CD28×anti-MSLN antibodies are depicted, for example, in FIG. 24. During the cell culture production of the anti-CD28×anti-MSLN antibodies provided herein, the C-terminal lysine residue or C-terminal lysine and glycine residues may be cleaved from the heavy chain monomers, thereby leading to variants with C-terminal “clipping.” See, e.g., Jiang et al., Journal of Pharmaceutical Sciences 105:2066-2072 (2016). Thus, in some embodiments provided herein, the anti-CD28×anti-MSLN antibody is a variant of one of the anti-CD28×anti-MSLN antibodies includes a deletion of a C-terminal lysine (-K) terminal or C-terminal lysine and glycine (-GK) residues in one or both Fe domains of an anti-CD28×anti-MSLN antibody described herein. In some embodiments, the deletion is G446del and/or K447del (EU numbering).

Aspects of the anti-CD28×anti-MSLN antibodies are further described in detail below.

### A. CD28 Binding Domains

The anti-CD28×anti-MSLN antibodies provided herein include at least one CD28 binding domain. Any suitable CD28 binding domain can be included in the anti-CD28×anti-MSLN antibodies provided herein. In exemplary embodiments, the CD28 binding domain is an agonistic CD28 ABDs that advantageously provide T cell costimulatory activity.

As will be appreciated by those in the art, suitable CD28 binding domains can comprise a set of 6 CDRs as depicted in the figures, either as they are underlined or, in the case where a different numbering scheme is used, as described herein and as shown in Table 2, as the CDRs that are identified using other alignments within the variable heavy (VH) domain and variable light domain (VL) sequences of those CD28 binding domains depicted in FIGS. 27, 30 and 33. Additional VH and VL sequences of exemplary CD28 binding domains that can be used in the subject antibodies are depicted in FIGS. 28 and 29. Suitable CD28 ABDs can also include the entire VH and VL sequences as depicted in these sequences and figures, used as scFvs or as Fabs.

In one embodiment, the CD28 antigen binding domain includes the 6 CDRs (i.e., vhCDR1-3 and vlCDR1-3) of any of the CD28 binding domains described herein, including, but not limited to those depicted in FIGS. 27, 30 and 33. In some embodiments, the CD28 ABD that binds human CD28 is one of the following CD28 ABDs: 1A7[CD28]_H1L1, 1A7[CD28]_H1.1_L1, 1A7[CD28]_H1_L1.71, 1A7[CD28]_H1.1_L1.71, 1A7[CD28]_H1.14_L1, 1A7[CD28]_H1.14_L1.71, CD28.3[CD28]_H0L0, hCD28.3[CD28]_H1L1, 5.11A1[CD28]_H0L0, TGN1412_H1L1, 341VL34[CD28]_H1L1, 341VL36[CD28]_H1L1, 281VL4[CD28]_H1L1, HuTN228[CD28]_H1L1, PV1[CD28]_H0L0, m9.3[CD28]_H0L0, hu9.3[CD28]_H1L1, 9G2[CD28]_H0L0, 9G2[CD28]_H1L1, 2F10A3.140[CD28]_H1L1, and TN228[CD28]_H4L2 (FIGS. 27, 30 and 33). In some embodiments, the CD28 ABD includes a VH/VL pair selected from the VHs and VLs depicted in FIGS. 28 and 29, respectively.

In addition to the parental CDR sets disclosed in the figures and sequence listing that form an ABD to CD28, provided herein are variant CD28 ABDS having CDRs that include at least one modification of the CD28 ABD CDRs disclosed herein (e.g., (FIGS. 27-30 and 33 and the sequence listing). In one embodiment, the CD28 ABD of the subject anti-CD28×anti-MSLN antibody includes a set of 6 CDRs with 1, 2, 3, 4, 5, 6, 7, 8, 9, 10 amino acid modifications as compared to the 6 CDRs of a CD28 ABD as described herein, including the figures and sequence listing. In exemplary embodiments, the CD28 ABD of the subject anti-CD28×anti-MSLN antibody includes a set of 6 CDRs with 1, 2, 3, 4, 5, 6, 7, 8, 9, 10 amino acid modifications as compared to the 6 CDRs of one of the following CD28 ABDs: 1A7[CD28]_H1L1, 1A7[CD28]_H1.1_L1, 1A7[CD28]_H1_L1.71, 1A7[CD28]_H1.1_L1.71, 1A7[CD28]_H1.14_L1, 1A7[CD28]_H1.14_L1.71, CD28.3[CD28]_H0L0, hCD28.3[CD28]_H1L1, 5.11A1[CD28]_H0L0, TGN1412_H1L1, 341VL34[CD28]_H1L1, 341VL36[CD28]_H1L1, 281VL4[CD28]_H1L1, HuTN228[CD28]_H1L1, PV1[CD28]_H0L0, m9.3[CD28]_H0L0, hu9.3[CD28]_H1L1, 9G2[CD28]_H0L0, 9G2[CD28]_H1L1, 2F10A3.140[CD28]_H1L1, and TN228[CD28]_H4L2 (FIGS. 27, 30 and 33). In certain embodiments, the CD28 ABD of the subject anti-CD28×anti-MSLN antibody is capable of binding CD28 antigen, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CD28 ABD is capable of binding human CD28 antigen (see FIG. 14C).

In some embodiments, the CD28 ABD of the subject anti-CD28×anti-MSLN antibody includes 6 CDRs that are at least 90, 95, 97, 98 or 99% identical to the 6 CDRs of a CD28 ABD as described herein, including the figures and sequence listing. In exemplary embodiments, the CD28 ABD of the subject anti-CD28×anti-MSLN antibody includes 6 CDRs that are at least 90, 95, 97, 98 or 99% identical to the 6 CDRs of one of the following CD28 ABDs: 1A7[CD28]_H1L1, 1A7[CD28]_H1.1_L1, 1A7[CD28]_H1_L1.71, 1A7[CD28]_H1.1_L1.71, 1A7[CD28]_H1.14_L1, 1A7[CD28]_H1.14_L1.71, CD28.3[CD28]_H0L0, hCD28.3[CD28]_H1L1, 5.11A1[CD28]_H0L0, TGN1412_H1L1, 341VL34[CD28]_H1L1, 341VL36[CD28]_H1L1, 281VL4[CD28]_H1L1, HuTN228[CD28]_H1L1, PV1[CD28]_H0L0, m9.3[CD28]_H0L0, hu9.3[CD28]_H1L1, 9G2[CD28]_H0L0, 9G2[CD28]_H1L1, 2F10A3.140[CD28]_H1L1, and TN228[CD28]_H4L2 (FIGS. 27, 30 and 33). In certain embodiments, the CD28 ABD is capable of binding to the CD28, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CD28 ABD is capable of binding human CD28 antigen (see FIG. 14C).

In another exemplary embodiment, the CD28 ABD of the subject anti-CD28×anti-MSLN antibody includes the variable heavy (VH) domain and variable light (VL) domain of any one of the CD28 ABDs described herein, including the figures and sequence listing. In exemplary embodiments, the CD28 ABD is one of the following CD28 ABDs: 1A7[CD28]_H1L1, 1A7[CD28]_H1.1_L1, 1A7[CD28]_H1_L1.71, 1A7[CD28]_H1.1_L1.71, 1A7[CD28]_H1.14_L1, 1A7[CD28]_H1.14_L1.71, CD28.3[CD28]_H0L0, hCD28.3[CD28]_H1L1, 5.11A1[CD28]_H0L0, TGN1412_H1L1, 341VL34[CD28]_H1L1, 341VL36[CD28]_H1L1, 281VL4[CD28]_H1L1, HuTN228[CD28]_H1L1, PV1[CD28]_H0L0, m9.3[CD28]_H0L0, hu9.3[CD28]_H1L1, 9G2[CD28]_H0L0, 9G2[CD28]_H1L1, 2F10A3.140[CD28]_H1L1, and TN228[CD28]_H4L2 (FIGS. 27, 30 and 33). In some embodiments, the CD28 ABD includes a VH and VL pair selected from those depicted in FIGS. 28 and 29.

In some embodiments, the anti-CD28×anti-MSLN antibody includes a CD28 ABD that includes a variable heavy domain and/or a variable light domain that are variants of a CD28 ABD VH and VL domain disclosed herein. In one embodiment, the variant VH domain and/or VL domain has from 1, 2, 3, 4, 5, 6, 7, 8, 9 or 10 amino acid changes from a VH and/or VL domain of a CD28 ABD described herein, including the figures and sequence listing. In exemplary embodiments, the variant VH domain and/or VL domain has from 1, 2, 3, 4, 5, 6, 7, 8, 9 or 10 amino acid changes from a VH and/or VL domain of one of the following CD28 ABDs: 1A7[CD28]_H1L1, 1A7[CD28]_H1.1_L1, 1A7[CD28]_H1_L1.71, 1A7[CD28]_H1.1_L1.71, 1A7[CD28]_H1.14_L1, 1A7[CD28]_H1.14_L1.71, CD28.3[CD28]_H0L0, hCD28.3[CD28]_H1L1, 5.11A1[CD28]_H0L0, TGN1412_H1L1, 341VL34[CD28]_H1L1, 341VL36[CD28]_H1L1, 281VL4[CD28]_H1L1, HuTN228[CD28]_H1L1, PV1[CD28]_H0L0, m9.3[CD28]_H0L0, hu9.3[CD28]_H1L1, 9G2[CD28]_H0L0, 9G2[CD28]_H1L1, 2F10A3.140[CD28]_H1L1, and TN228[CD28]_H4L2 (FIGS. 27, 30 and 33). In some embodiments, the changes are in a VH domain depicted in FIGS. 27-30 and 33. In some embodiments, the changes are in a VL domain are depicted in FIGS. 27-30 and 33. In some embodiments, the changes are in a VH and VL domain are depicted in FIGS. 27-30 and 33. In some embodiments, the one or more amino acid changes are in the VH and/or VL framework regions (FR1, FR2, FR3, and/or FR4). In some embodiments, the one or more amino acid change(s) are in one or more CDRs. In certain embodiments, the CD28 ABD is capable of binding to CD28, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CD28 ABD is capable of binding human CD28 antigen (see FIG. 14C).

In one embodiment, the variant VH and/or VL domain is at least 90, 95, 97, 98 or 99% identical to the VH and/or VL of a CD28 ABD as described herein, including the figures and sequence listing. In exemplary embodiments, the variant VH and/or VL domain is at least 90, 95, 97, 98 or 99% identical to the VH and/or VL of one of the following CD28 ABDs: 1A7[CD28]_H1L1, 1A7[CD28]_H1.1_L1, 1A7[CD28]_H1_L1.71, 1A7[CD28]_H1.1_L1.71, 1A7[CD28]_H1.14_L1, 1A7[CD28]_H1.14_L1.71, CD28.3[CD28]_H0L0, hCD28.3[CD28]_H1L1, 5.11A1[CD28]_H0L0, TGN1412_H1L1, 341VL34[CD28]_H1L1, 341VL36[CD28]_H1L1, 281VL4[CD28]_H1L1, HuTN228[CD28]_H1L1, PV1[CD28]_H0L0, m9.3[CD28]_H0L0, hu9.3[CD28]_H1L1, 9G2[CD28]_H0L0, 9G2[CD28]_H1L1, 2F10A3.140[CD28]_H1L1, and TN228[CD28]_H4L2 (FIGS. 27, 30 and 33). In some embodiments, the CD28 ABD includes a VH that is at least 90, 95, 97, 98 or 99% identical to VH domain depicted in FIGS. 27-30 and 33. In some embodiments, the CD28 ABD includes a VL that is at least 90, 95, 97, 98 or 99% identical to VL domain depicted in FIGS. 27-30 and 33. In some embodiments, the CD28 ABD includes a VH and a VL that is at least 90, 95, 97, 98 or 99% identical to a VH domain and a VL domain depicted in FIGS. 27-30 and 33. In certain embodiments, the CD28 ABD is capable of binding to CD28, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CD28 ABD is capable of binding human CD28 antigen (see FIG. 14C).

In some embodiments, the CD28 binding domain of the subject anti-CD28×anti-MSLN antibody includes a VH that includes any one of the VHCDR1-3 or HFR1-4 sequences depicted in FIG. 31A. In some embodiments, the CD28 binding domain includes a VL that includes any one of the VLCDR1-3 or LFR1-4 sequences depicted in FIG. 31B.

In some embodiments, the anti-CD28×anti-MSLN antibody includes a CD28 binding domain that includes a VH and VL selected from the following:
(i) a VH comprising a vhCDR1, a vhCDR2, and a vhCDR3 having an amino acid sequence of a vhCDR1, a vhCDR2, and a vhCDR3, respectively, of a VH of any of the CD28 binding domains depicted in FIGS. 27, 30 and 33 or a variant thereof, and (ii) a VL comprising a vlCDR1, a vlCDR2, and a vlCDR3 having an amino acid sequence of a vlCDR1, a vlCDR2, and a vlCDR3, respectively, of the VL of the CD28 binding domain depicted in FIGS. 27, 30 and 33 or a variant thereof, or
(i) a VH comprising a vhCDR1, a vhCDR2, and a vhCDR3 having an amino acid sequence of a vhCDR1, a vhCDR2, and a vhCDR3, respectively, of a VH depicted in FIG. 28 or a variant thereof, and (ii) a VL comprising a vlCDR1, a vlCDR2, and a vlCDR3 having an amino acid sequence of a vlCDR1, a vlCDR2, and a vlCDR3, respectively, of a VL depicted in FIG. 29 or a variant thereof.

In some embodiments, the anti-CD28×anti-MSLN antibody includes a CD28 binding domain that includes a VH and VL selected from the following:
(i) a VH having an amino acid sequence of a VH of any of the CD28 binding domains depicted in FIGS. 27, 30 and 33 or a variant thereof, and (ii) a VL having an amino acid sequence of the VL of the CD28 binding domain depicted in FIGS. 27, 30 and 33 or a variant thereof; or
(i) a VH having an amino acid sequence of a VH depicted in FIG. 28 or a variant thereof; and (ii) a VL having an amino acid sequence of the VL of a VL depicted in FIG. 29 or a variant thereof.

### B. MSLN Binding Domains

The anti-CD28×anti-MSLN antibodies provided herein include at least one MSLN binding domain. Subject antibodies that include such MSLN antigen binding domains (e.g., anti-MSLN×anti-CD3 bispecific antibodies) advantageously target cells that express high levels of MSLN over those that express levels of MSLN (e.g., normal cells).

As will be appreciated by those in the art, suitable MSLN binding domains can comprise a set of 6 CDRs as depicted in the sequence listing and FIGS. 15-17, either as the CDRs are underlined or, in the case where a different numbering scheme is used as described herein and as shown in Table 2, as the CDRs that are identified using other alignments within the variable heavy (VH) domain and variable light domain (VL) sequences of those depicted in FIGS. 15-17 and the sequence listing (see Table 2). Suitable MSLN ABDs can also include the entire VH and VL sequences as depicted in these sequences and figures, used as scFvs or as Fab domains. Additional used MSLN binding domains and MSLN binding domain VH/VL sequences are depicted in FIGS. 18, 19 and 23.

In one embodiment, the MSLN antigen binding domain of the anti-CD28×anti-MSLN antibody includes the 6 CDRs (i.e., vhCDR1-3 and vlCDR1-3) of a MSLN ABD described herein, including the Figures and sequence listing. In some embodiments, the anti-CD28×anti-MSLN antibody includes the 6 CDRs (i.e., vhCDR1-3 and vlCDR1-3) of a MSLN ABD depicted in FIGS. 15-17 and 23. In some embodiments, the anti-CD28×anti-MSLN antibody includes the 6 CDRs (i.e., vhCDR1-3 and vlCDR1-3) of a MSLN ABD VH and VL pair selected from those depicted in FIGS. 18 and 19, respectively.

In addition to the parental CDR sets disclosed in the figures and sequence listing that form an ABD to MSLN, provided herein are variant MSLN ABDS having CDRs that include at least one modification of the MSLN ABD CDRs disclosed herein. In one embodiment, the MSLN ABD includes a set of 6 CDRs with 1, 2, 3, 4, 5, 6, 7, 8, 9, 10 amino acid modifications as compared to the 6 CDRs of a MSLN ABD described herein, including the figures and sequence listing. In exemplary embodiments, the MSLN ABD of the anti-CD28×anti-MSLN antibody includes a set of 6 CDRs with 1, 2, 3, 4, 5, 6, 7, 8, 9, 10 amino acid modifications as compared to the 6 CDRs of a MSLN ABD depicted in FIGS. 15-17 and 23. In exemplary embodiments, the MSLN ABD of the anti-CD28×anti-MSLN antibody includes a set of 6 CDRs with 1, 2, 3, 4, 5, 6, 7, 8, 9, 10 amino acid modifications as compared to the 6 CDRs of a MSLN ABD VH and VL pair selected from those depicted in FIGS. 18 and 19, respectively. In certain embodiments, the variant MSLN ABD is capable of binding MSLN antigen, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the MSLN ABD is capable of binding human MSLN antigen (see FIG. 14).

In one embodiment, the MSLN ABD of the anti-CD28×anti-MSLN antibody includes 6 CDRs that are at least 90, 95, 97, 98 or 99% identical to the 6 CDRs of a MSLN ABD as described herein, including the figures and sequence listing. In exemplary embodiments, the MSLN ABD includes 6 CDRs that are at least 90, 95, 97, 98 or 99% identical to the 6 CDRs of a MSLN ABD depicted in FIGS. 15-17 and 23. In exemplary embodiments, the MSLN ABD includes 6 CDRs that are at least 90, 95, 97, 98 or 99% identical to the 6 CDRs of a MSLN ABD VH and VL pair selected from those depicted in FIGS. 18 and 19, respectively. In certain embodiments, the MSLN ABD is capable of binding to MSLN antigen, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the MSLN ABD is capable of binding human MSLN antigen (see FIG. 14).

In another exemplary embodiment, the MSLN ABD of the anti-CD28×anti-MSLN antibody include the variable heavy (VH) domain and variable light (VL) domain of any one of the MSLN ABDs described herein, including the figures and sequence listing. In exemplary embodiments, the MSLN ABD is a MSLN ABD depicted in FIGS. 15-17 and 23. In exemplary embodiments, the MSLN ABD comprises a VH and VL pair selected from those depicted in FIGS. 18 and 19, respectively.

In addition to the parental MSLN variable heavy and variable light domains disclosed herein, provided herein are MSLN ABDs that include a variable heavy domain and/or a variable light domain that are variants of a MSLN ABD VH and VL domain disclosed herein. In one embodiment, the variant VH domain and/or VL domain has from 1, 2, 3, 4, 5, 6, 7, 8, 9 or 10 amino acid changes from a VH and/or VL domain of a MSLN ABD described herein, including the figures and sequence listing. In exemplary embodiments, the variant VH domain and/or VL domain has from 1, 2, 3, 4, 5, 6, 7, 8, 9 or 10 amino acid changes from a VH and/or VL domain of a MSLN ABD depicted in FIGS. 15-17 and 23. In exemplary embodiments, the variant VH domain and/or VL domain has from 1, 2, 3, 4, 5, 6, 7, 8, 9 or 10 amino acid changes from a VH and/or VL domain depicted in FIGS. 18 and 19. In some embodiments, the changes are in a VH domain depicted in FIGS. 15-19 and 23. In some embodiments, the changes are in a VL domain are depicted in FIGS. 15-19 and 23. In some embodiments, the changes are in a VH and VL domain are depicted in FIGS. 15-19 and 23. In some embodiments, the one or more amino acid changes are in the VH and/or VL framework regions (FR1, FR2, FR3, and/or FR4). In some embodiments, the one or more amino acid change(s) are in one or more CDRs. In certain embodiments, the MSLN ABD of the anti-CD28×anti-MSLN antibody is capable of binding to MSLN, as measured at least one of a Biacore, surface plasmon resonance (SPR), flow cytometry, and/or BLI (biolayer interferometry, e.g., Octet assay) assay, with the latter finding particular use in many embodiments. In particular embodiments, the MSLN ABD is capable of binding human MSLN antigen.

In one embodiment, the variant VH and/or VL domain is at least 90, 95, 97, 98 or 99% identical to the VH and/or VL of a MSLN ABD as described herein, including the figures and sequence listing. In exemplary embodiments, the variant VH and/or VL domain is at least 90, 95, 97, 98 or 99% identical to the VH and/or VL of a MSLN ABD depicted in FIGS. 15-17 and 23. In exemplary embodiments, the variant VH and/or VL domain is at least 90, 95, 97, 98 or 99% identical to the VH and/or VL depicted in FIGS. 18 and 19. In some embodiments, the MSLN ABD includes a VH that is at least 90, 95, 97, 98 or 99% identical to VH domain depicted in FIGS. 15-19 and 23. In some embodiments, the MSLN ABD includes a VL that is at least 90, 95, 97, 98 or 99% identical to VL domain depicted in FIGS. 15-17 and 23. In some embodiments, the MSLN ABD includes a VH and a VL that is at least 90, 95, 97, 98 or 99% identical to a VH domain and a VL domain depicted in FIGS. 15-17 and 23. In certain embodiments, the MSLN ABD of the anti-CD28×anti-MSLN antibody is capable of binding to the MSLN, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the MSLN ABD is capable of binding human MSLN antigen.

In some embodiments, the anti-CD28×anti-MSLN antibody is a bivalent antibody (e.g., 1+1 Fab-scFv-Fc format antibody) that includes one MSLN binding domain. In other embodiments, the anti-CD28×anti-MSLN antibody is a trivalent antibody (e.g., 2+1 mAb-scFv and 2+1 Fab2.scFv-Fc format antibodies) that includes two MSLN binding domains.

### C. Chimeric and Humanized Antibodies

In certain embodiments, the subject antibodies provided herein include a heavy chain variable region from a particular germline heavy chain immunoglobulin gene and/or a light chain variable region from a particular germline light chain immunoglobulin gene. For example, such antibodies may comprise or consist of a human antibody comprising heavy or light chain variable regions that are “the product of” or “derived from” a particular germline sequence. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody (using the methods outlined herein). A human antibody that is “the product of” or “derived from” a particular human germline immunoglobulin sequence may contain amino acid differences as compared to the germline sequence, due to, for example, naturally-occurring somatic mutations or intentional introduction of site-directed mutation. However, a humanized antibody typically is at least 90% identical in amino acids sequence to an amino acid sequence encoded by a human germline immunoglobulin gene and contains amino acid residues that identify the antibody as being derived from human sequences when compared to the germline immunoglobulin amino acid sequences of other species (e.g., murine germline sequences). In certain cases, a humanized antibody may be at least 95, 96, 97, 98 or 99%, or even at least 96%, 97%, 98%, or 99% identical in amino acid sequence to the amino acid sequence encoded by the germline immunoglobulin gene. Typically, a humanized antibody derived from a particular human germline sequence will display no more than 10-20 amino acid differences from the amino acid sequence encoded by the human germline immunoglobulin gene (prior to the introduction of any skew, pI and ablation variants herein; that is, the number of variants is generally low, prior to the introduction of the variants of the invention). In certain cases, the humanized antibody may display no more than 5, or even no more than 4, 3, 2, or 1 amino acid difference from the amino acid sequence encoded by the germline immunoglobulin gene (again, prior to the introduction of any skew, pI and ablation variants herein; that is, the number of variants is generally low, prior to the introduction of the variants of the invention).

In one embodiment, the parent antibody has been affinity matured, as is known in the art. Structure-based methods may be employed for humanization and affinity maturation, for example as described in U.S. Ser. No. 11/004,590. Selection based methods may be employed to humanize and/or affinity mature antibody variable regions, including but not limited to methods described in Wu et al., 1999, J. Mol. Biol. 294:151-162; Baca et al., 1997, J. Biol. Chem. 272(16):10678-10684; Rosok et al., 1996, J. Biol. Chem. 271(37): 22611-22618; Rader et al., 1998, Proc. Natl. Acad. Sci. USA 95: 8910-8915; Krauss et al., 2003, Protein Engineering 16(10):753-759, all entirely incorporated by reference. Other humanization methods may involve the grafting of only parts of the CDRs, including but not limited to methods described in U.S. Ser. No. 09/810,510; Tan et al., 2002, J. Immunol. 169:1119-1125; De Pascalis et al., 2002, J. Immunol. 169:3076-3084, all entirely incorporated by reference.

### D. Heterodimeric Antibodies

In exemplary embodiments, the anti-CD28×anti-MSLN antibodies provided herein are heterodimeric bispecific antibodies that include two variant Fc domain sequences. Such variant Fc domains include amino acid modifications to facilitate the self-assembly and/or purification of the heterodimeric antibodies.

An ongoing problem in antibody technologies is the desire for “bispecific” antibodies that bind to two different antigens simultaneously, in general thus allowing the different antigens to be brought into proximity and resulting in new functionalities and new therapies. In general, these antibodies are made by including genes for each heavy and light chain into the host cells. This generally results in the formation of the desired heterodimer (A-B), as well as the two homodimers (A-A and B-B (not including the light chain heterodimeric issues)). However, a major obstacle in the formation of bispecific antibodies is the difficulty in biasing the formation of the desired heterodimeric antibody over the formation of the homodimers and/or purifying the heterodimeric antibody away from the homodimers.

There are a number of mechanisms that can be used to generate the subject heterodimeric antibodies. In addition, as will be appreciated by those in the art, these different mechanisms can be combined to ensure high heterodimerization. Amino acid modifications that facilitate the production and purification of heterodimers are collectively referred to generally as “heterodimerization variants.” As discussed below, heterodimerization variants include “skew” variants (e.g., the “knobs and holes” and the “charge pairs” variants described below) as well as “pI variants,” which allow purification of heterodimers from homodimers. As is generally described in U.S. Pat. No. 9,605,084, hereby incorporated by reference in its entirety and specifically as below for the discussion of heterodimerization variants, useful mechanisms for heterodimerization include “knobs and holes” (“KIH”) as described in U.S. Pat. No. 9,605,084, “electrostatic steering” or “charge pairs” as described in U.S. Pat. No. 9,605,084, pI variants as described in U.S. Pat. No. 9,605,084, and general additional Fc variants as outlined in U.S. Pat. No. 9,605,084 and below.

Heterodimerization variants that are useful for the formation and purification of the subject heterodimeric antibody (e.g., bispecific antibodies) are further discussed in detailed below.

### 1. Skew Variants

In some embodiments, the heterodimeric antibody includes skew variants which are one or more amino acid modifications in a first Fc domain (A) and/or a second Fc domain (B) that favor the formation of Fc heterodimers (Fc dimers that include the first and the second Fc domain; (A-B) over Fc homodimers (Fc dimers that include two of the first Fc domain or two of the second Fc domain; A-A or B-B). Suitable skew variants are included in the FIG. 29 of US Publ. App. No. 2016/0355608, hereby incorporated by reference in its entirety and specifically for its disclosure of skew variants, as well as in FIGS. 1 and 4.

One particular type of skew variants is generally referred to in the art as “knobs and holes,” referring to amino acid engineering that creates steric influences to favor heterodimeric formation and disfavor homodimeric formation, as described in U.S. Ser. No. 61/596,846, Ridgway et al., Protein Engineering 9(7):617 (1996); Atwell et al., J. Mol. Biol. 1997 270:26; U.S. Pat. No. 8,216,805, all of which are hereby incorporated by reference in their entirety and specifically for the disclosure of “knobs and holes” mutations. This is sometime referred to herein as “steric variants.” The figures identify a number of “monomer A-monomer B” pairs that rely on “knobs and holes”. In addition, as described in Merchant et al., Nature Biotech. 16:677 (1998), these “knobs and holes” mutations can be combined with disulfide bonds to further favor formation of Fc heterodimers.

Another method that finds use in the generation of heterodimers is sometimes referred to as “electrostatic steering” as described in Gunasekaran et al., J. Biol. Chem. 285(25):19637 (2010), hereby incorporated by reference in its entirety. This is sometimes referred to herein as “charge pairs”. In this embodiment, electrostatics are used to skew the formation towards heterodimerization. As those in the art will appreciate, these may also have an effect on pI, and thus on purification, and thus could in some cases also be considered pI variants. However, as these were generated to force heterodimerization and were not used as purification tools, they are classified as “skew variants”. These include, but are not limited to, D221E/P228E/L368E paired with D221R/P228R/K409R (e.g., these are “monomer corresponding sets) and C220E/P228E/368E paired with C220R/E224R/P228R/K409R.

In some embodiments, the skew variants advantageously and simultaneously favor heterodimerization based on both the “knobs and holes” mechanism as well as the “electrostatic steering” mechanism. In some embodiments, the heterodimeric antibody includes one or more sets of such heterodimerization skew variants. These variants come in “pairs” of “sets”. That is, one set of the pair is incorporated into the first monomer and the other set of the pair is incorporated into the second monomer. It should be noted that these sets do not necessarily behave as “knobs in holes” variants, with a one-to-one correspondence between a residue on one monomer and a residue on the other. That is, these pairs of sets may instead form an interface between the two monomers that encourages heterodimer formation and discourages homodimer formation, allowing the percentage of heterodimers that spontaneously form under biological conditions to be over 90%, rather than the expected 50% (25% homodimer A/A:50% heterodimer A/B:25% homodimer B/B). Exemplary heterodimerization “skew” variants are depicted in FIG. 1. Such “skew” variants include, but are not limited to: S364K/E357Q:L368D/K370S; L368D/K370S:S364K; L368E/K370S:S364K; T411T/E360E/Q362E:D401K; L368D/K370S:S364K/E357L; K370S:S364K/E357Q (EU numbering).

In exemplary embodiments, the heterodimeric antibody includes a S364K/E357Q:L368D/K370S; L368D/K370S:S364K; L368E/K370S:S364K; T411T/E360E/Q362E:D401K; L368D/K370S:S364K/E357L; K370S:S364K/E357Q; or a T366S/L368A/Y407V:T366W (optionally including a bridging disulfide, T366S/L368A/Y407V/Y349C:T366W/S354C) “skew” variant amino acid substitution set (EU numbering). In an exemplary embodiment, the heterodimeric antibody includes a “S364K/E357Q:L368D/K370S” amino acid substitution set. In terms of nomenclature, the pair “S364K/E357Q:L368D/K370S” means that one of the monomers includes an Fc domain that includes the amino acid substitutions S364K and E357Q and the other monomer includes an Fc domain that includes the amino acid substitutions L368D and K370S; as above, the “strandedness” of these pairs depends on the starting pI.

In some embodiments, the skew variants provided herein can be optionally and independently incorporated with any other modifications, including, but not limited to, other skew variants (see, e.g., in FIG. 37 of US Publ. App. No. 2012/0149876, herein incorporated by reference, particularly for its disclosure of skew variants), pI variants, isotpypic variants, FcRn variants, ablation variants, etc. into one or both of the first and second Fc domains of the heterodimeric antibody. Further, individual modifications can also independently and optionally be included or excluded from the subject the heterodimeric antibody.

In some embodiments, the skew variants outlined herein can be optionally and independently incorporated with any pI variant (or other variants such as Fc variants, FcRn variants, etc.) into one or both heavy chain monomers, and can be independently and optionally included or excluded from the subject heterodimeric antibodies.

### 2. Purification Variants

In some embodiments, the heterodimeric antibody includes purification variants that advantageously allow for the separation of heterodimeric proteins (e.g., anti-CD28×anti-MSLN bispecific antibody) from homodimeric proteins.

There are several basic mechanisms that can lead to ease of purifying heterodimeric antibodies. For example, modifications to one or both of the antibody heavy chain monomers A and B such that each monomer has a different pI allows for the isoelectric purification of heterodimeric A-B antibody from monomeric A-A and B-B proteins. Alternatively, some scaffold formats, such as the “1+1 Fab-scFv-Fc” format, and the “2+1 Fab2-scFv-Fc” format, allows separation on the basis of size. As described above, it is also possible to “skew” the formation of heterodimers over homodimers using skew variants. Thus, a combination of heterodimerization skew variants and purification variants find particular use in the heterodimeric antibodies provided herein.

Additionally, as more fully outlined below, depending on the format of the heterodimeric antibody, purification variants either contained within the constant region and/or Fc domains of a monomer, and/or domain linkers can be used. In some embodiments, the heterodimeric antibody includes additional modifications for alternative functionalities that can also create pI changes, such as Fc, FcRn and KO variants.

In some embodiments, the subject heterodimeric antibodies provided herein include at least one monomer with one or more modifications that alter the pI of the monomer (i.e., a “pI variant”). In general, as will be appreciated by those in the art, there are two general categories of pI variants: those that increase the pI of the protein (basic changes) and those that decrease the pI of the protein (acidic changes). As described herein, all combinations of these variants can be done: one monomer may be wild type, or a variant that does not display a significantly different pI from wild-type, and the other can be either more basic or more acidic. Alternatively, each monomer is changed, one to more basic and one to more acidic.

Depending on the format of the heterodimer antibody, pI variants can be either contained within the constant and/or Fc domains of a monomer, or charged linkers, either domain linkers or scFv linkers, can be used. That is, antibody formats that utilize scFv(s) such as “1+1 Fab-scFv-Fc”, format can include charged scFv linkers (either positive or negative), that give a further pI boost for purification purposes. As will be appreciated by those in the art, some 1+1 Fab-scFv-Fc and 2+1 Fab2-scFv-Fc formats are useful with just charged scFv linkers and no additional pI adjustments, although the invention does provide pI variants that are on one or both of the monomers, and/or charged domain linkers as well. In addition, additional amino acid engineering for alternative functionalities may also confer pI changes, such as Fc, FcRn and KO variants.

In subject heterodimeric antibodies that utilizes pI as a separation mechanism to allow the purification of heterodimeric proteins, amino acid variants are introduced into one or both of the monomer polypeptides. That is, the pI of one of the monomers (referred to herein for simplicity as “monomer A”) can be engineered away from monomer B, or both monomer A and B change be changed, with the pI of monomer A increasing and the pI of monomer B decreasing. As is outlined more fully below, the pI changes of either or both monomers can be done by removing or adding a charged residue (e.g., a neutral amino acid is replaced by a positively or negatively charged amino acid residue, e.g., glycine to glutamic acid), changing a charged residue from positive or negative to the opposite charge (aspartic acid to lysine) or changing a charged residue to a neutral residue (e.g., loss of a charge; lysine to serine.). A number of these variants are shown in the FIG. 2.

Thus, in some embodiments, the subject heterodimeric antibody includes amino acid modifications in the constant regions that alter the isoelectric point (pI) of at least one, if not both, of the monomers of a dimeric protein to form “pI antibodies”) by incorporating amino acid substitutions (“pI variants” or “pI substitutions”) into one or both of the monomers. As shown herein, the separation of the heterodimers from the two homodimers can be accomplished if the pIs of the two monomers differ by as little as 0.1 pH unit, with 0.2, 0.3, 0.4 and 0.5 or greater all finding use in the present invention.

As will be appreciated by those in the art, the number of pI variants to be included on each or both monomer(s) to get good separation will depend in part on the starting pI of the components, for example in the 1+1 Fab-scFv-Fc, 2+1 Fab2-scFv-Fc, 1+1 CLC and 2+1 CLC formats, the starting pI of the scFv (1+1 Fab-scFv-Fc, 2+1 Fab2-scFv-Fc) and Fab(s) of interest. That is, to determine which monomer to engineer or in which “direction” (e.g., more positive or more negative), the Fv sequences of the two target antigens are calculated and a decision is made from there. As is known in the art, different Fvs will have different starting pIs which are exploited in the present invention. In general, as outlined herein, the pIs are engineered to result in a total pI difference of each monomer of at least about 0.1 logs, with 0.2 to 0.5 being preferred as outlined herein.

In the case where pI variants are used to achieve heterodimerization, by using the constant region(s) of the heavy chain(s), a more modular approach to designing and purifying bispecific proteins, including antibodies, is provided. Thus, in some embodiments, heterodimerization variants (including skew and pI heterodimerization variants) are not included in the variable regions, such that each individual antibody must be engineered. In addition, in some embodiments, the possibility of immunogenicity resulting from the pI variants is significantly reduced by importing pI variants from different IgG isotypes such that pI is changed without introducing significant immunogenicity. Thus, an additional problem to be solved is the elucidation of low pI constant domains with high human sequence content, e.g., the minimization or avoidance of non-human residues at any particular position. Alternatively or in addition to isotypic substitutions, the possibility of immunogenicity resulting from the pI variants is significantly reduced by utilizing isosteric substitutions (e.g. Asn to Asp; and Gln to Glu).

As discussed below, a side benefit that can occur with this pI engineering is also the extension of serum half-life and increased FcRn binding. That is, as described in US Publ. App. No. US 2012/0028304 (incorporated by reference in its entirety), lowering the pI of antibody constant domains (including those found in antibodies and Fc fusions) can lead to longer serum retention in vivo. These pI variants for increased serum half-life also facilitate pI changes for purification.

In addition, it should be noted that the pI variants give an additional benefit for the analytics and quality control process of bispecific antibodies, as the ability to either eliminate, minimize and distinguish when homodimers are present is significant. Similarly, the ability to reliably test the reproducibility of the heterodimeric antibody production is important.

In general, embodiments of particular use rely on sets of variants that include skew variants, which encourage heterodimerization formation over homodimerization formation, coupled with pI variants, which increase the pI difference between the two monomers to facilitate purification of heterodimers away from homodimers.

Exemplary combinations of pI variants are shown in FIGS. 1 and 2, and FIG. 30 of US Publ. App. No. 2016/0355608, all of which are herein incorporated by reference in its entirety and specifically for the disclosure of pI variants. Preferred combinations of pI variants are shown in FIGS. 1 and 2. As outlined herein and shown in the figures, these changes are shown relative to IgG1, but all isotypes can be altered this way, as well as isotype hybrids. In the case where the heavy chain constant domain is from IgG2-4, R133E and R133Q can also be used.

In one embodiment, a preferred combination of pI variants has one monomer (the negative Fab side) comprising 208D/295E/384D/418E/421D variants (N208D/Q295E/N384D/Q418E/N421D when relative to human IgG1) and a second monomer (the positive scFv side) comprising a positively charged scFv linker, including (GKPGS)₄ (SEQ ID NO:1). However, as will be appreciated by those in the art, the first monomer includes a CH1 domain, including position 208. Accordingly, in constructs that do not include a CH1 domain (for example for antibodies that do not utilize a CH1 domain on one of the domains), a preferred negative pI variant Fc set includes 295E/384D/418E/421D variants (Q295E/N384D/Q418E/N421D when relative to human IgG1).

Accordingly, in some embodiments, one monomer has a set of substitutions from FIG. 4 and the other monomer has a charged linker (either in the form of a charged scFv linker because that monomer comprises an scFv or a charged domain linker, as the format dictates, which can be selected from those depicted in FIG. 5).

In some embodiments, modifications are made in the hinge of the Fc domain, including positions 216, 217, 218, 219, 220, 221, 222, 223, 224, 225, 226, 227, 228, 229, and 230 based on EU numbering. Thus, pI mutations and particularly substitutions can be made in one or more of positions 216-230, with 1, 2, 3, 4 or 5 mutations finding use. Again, all possible combinations are contemplated, alone or with other pI variants in other domains.

Specific substitutions that find use in lowering the pI of hinge domains include, but are not limited to, a deletion at position 221, a non-native valine or threonine at position 222, a deletion at position 223, a non-native glutamic acid at position 224, a deletion at position 225, a deletion at position 235 and a deletion or a non-native alanine at position 236. In some cases, only pI substitutions are done in the hinge domain, and in others, these substitution(s) are added to other pI variants in other domains in any combination.

In some embodiments, mutations can be made in the CH2 region, including positions 233, 234, 235, 236, 274, 296, 300, 309, 320, 322, 326, 327, 334 and 339, based on EU numbering. It should be noted that changes in 233-236 can be made to increase effector function (along with 327A) in the IgG2 backbone. Again, all possible combinations of these 14 positions can be made; e.g., an antibody provided herein may include a variant Fc domain with 1, 2, 3, 4, 5, 6, 7, 8, 9 or 10 CH2 pI substitutions.

Specific substitutions that find use in lowering the pI of CH2 domains include, but are not limited to, a non-native glutamine or glutamic acid at position 274, a non-native phenylalanine at position 296, a non-native phenylalanine at position 300, a non-native valine at position 309, a non-native glutamic acid at position 320, a non-native glutamic acid at position 322, a non-native glutamic acid at position 326, a non-native glycine at position 327, a non-native glutamic acid at position 334, a non-native threonine at position 339, and all possible combinations within CH2 and with other domains.

In this embodiment, the modifications can be independently and optionally selected from position 355, 359, 362, 384, 389,392, 397, 418, 419, 444 and 447 (EU numbering) of the CH3 region. Specific substitutions that find use in lowering the pI of CH3 domains include, but are not limited to, a non-native glutamine or glutamic acid at position 355, a non-native serine at position 384, a non-native asparagine or glutamic acid at position 392, a non-native methionine at position 397, a non-native glutamic acid at position 419, a non-native glutamic acid at position 359, a non-native glutamic acid at position 362, a non-native glutamic acid at position 389, a non-native glutamic acid at position 418, a non-native glutamic acid at position 444, and a deletion or non-native aspartic acid at position 447.

In some embodiments, the anti-CD28×anti-MSLN antibody includes amino acid substitutions in one of its Fc domains that reduces binding to Protein A. Such purification variants produces heterodimers with asymmetric binding to Protein A, which can in turn be used for separation of heterodimeric from homodimeric populations by a pH gradient. Exemplary purification amino acid substitutions that reduce binding to Protein A include, but are not limited to H435R and Y436F (IgG1 CH3 domain, EU numbering). See, e.g., US2010331527, which is incorporated by reference in its entirety, and specifically for pertinent disclosures relating to Fc domain modifications to reduce Protein A binding.

### 3. Isotypic Variants

In addition, many embodiments of the subject heterodimeric antibodies rely on the “importation” of pI amino acids at particular positions from one IgG isotype into another, thus reducing or eliminating the possibility of unwanted immunogenicity being introduced into the variants. A number of these are shown in FIG. 21 of US Publ. 2014/0370013, hereby incorporated by reference. That is, IgG1 is a common isotype for therapeutic antibodies for a variety of reasons, including high effector function. However, the heavy constant region of IgG1 has a higher pI than that of IgG2 (8.10 versus 7.31). By introducing IgG2 residues at particular positions into the IgG1 backbone, the pI of the resulting monomer is lowered (or increased) and additionally exhibits longer serum half-life. For example, IgG1 has a glycine (pI 5.97) at position 137, and IgG2 has a glutamic acid (pI 3.22); importing the glutamic acid will affect the pI of the resulting protein. As is described below, a number of amino acid substitutions are generally required to significant affect the pI of the variant antibody. However, it should be noted as discussed below that even changes in IgG2 molecules allow for increased serum half-life.

In other embodiments, non-isotypic amino acid changes are made, either to reduce the overall charge state of the resulting protein (e.g., by changing a higher pI amino acid to a lower pI amino acid), or to allow accommodations in structure for stability, etc. as is further described below.

In addition, by pI engineering both the heavy and light constant domains, significant changes in each monomer of the heterodimer can be seen. As discussed herein, having the pIs of the two monomers differ by at least 0.5 can allow separation by ion exchange chromatography or isoelectric focusing, or other methods sensitive to isoelectric point.

### 4. Calculating pI

The pI of each monomer of the antibodies provided herein can depend on the pI of the variant heavy chain constant domain and the pI of the total monomer, including the variant heavy chain constant domain and the fusion partner. Thus, in some embodiments, the change in pI is calculated on the basis of the variant heavy chain constant domain, using the chart in the FIG. 19 of US Pub. 2014/0370013. As discussed herein, which monomer to engineer is generally decided by the inherent pI of the Fv and scaffold regions. Alternatively, the pI of each monomer can be compared.

5. pI Variants that Also Confer Better FcRn In Vivo Binding

In the case where the pI variant decreases the pI of the monomer, the pI variant can have the added benefit of improving serum retention in vivo.

Although still under examination, Fc regions are believed to have longer half-lives in vivo, because binding to FcRn at pH 6 in an endosome sequesters the Fc (Ghetie and Ward, 1997 Immunol Today. 18(12): 592-598, entirely incorporated by reference). The endosomal compartment then recycles the Fc to the cell surface. Once the compartment opens to the extracellular space, the higher pH, ~7.4, induces the release of Fc back into the blood. In mice, Dall' Acqua et al. showed that Fc mutants with increased FcRn binding at pH 6 and pH 7.4 actually had reduced serum concentrations and the same half-life as wild-type Fc (Dall' Acqua et al. 2002, J. Immunol. 169:5171-5180, entirely incorporated by reference). The increased affinity of Fc for FcRn at pH 7.4 is thought to forbid the release of the Fc back into the blood. Therefore, the Fc mutations that will increase Fc's half-life in vivo will ideally increase FcRn binding at the lower pH while still allowing release of Fc at higher pH. The amino acid histidine changes its charge state in the pH range of 6.0 to 7.4. Therefore, it is not surprising to find His residues at important positions in the Fc/FcRn complex.

Recently it has been suggested that antibodies with variable regions that have lower isoelectric points may also have longer serum half-lives (Igawa et al., 2010 PEDS. 23(5): 385-392, entirely incorporated by reference). However, the mechanism of this is still poorly understood. Moreover, variable regions differ from antibody to antibody. Constant region variants with reduced pI and extended half-life would provide a more modular approach to improving the pharmacokinetic properties of antibodies, as described herein.

### E. Additional Fc Variants for Additional Functionality

In addition to the heterodimerization variants discussed above, there are a number of useful Fc amino acid modification that can be made for a variety of reasons, including, but not limited to, altering binding to one or more FcγR receptors, altered binding to FcRn receptors, etc., as discussed below.

Accordingly, the antibodies provided herein (heterodimeric, as well as homodimeric) can include such amino acid modifications with or without the heterodimerization variants outlined herein (e.g., the pI variants and steric variants). Each set of variants can be independently and optionally included or excluded from any particular heterodimeric protein.

### 1. FcγR and FcRn Variants

Accordingly, there are a number of useful Fc substitutions that can be made to alter binding to one or more of the FcγR receptors. In certain embodiments, the subject antibody includes modifications that alter the binding to one or more FcγR receptors (i.e., “FcγR variants”). Substitutions that result in increased binding as well as decreased binding can be useful. For example, it is known that increased binding to FcγRIIIa generally results in increased ADCC (antibody dependent cell-mediated cytotoxicity; the cell-mediated reaction wherein nonspecific cytotoxic cells that express FcγRs recognize bound antibody on a target cell and subsequently cause lysis of the target cell). Similarly, decreased binding to FcγRIIb (an inhibitory receptor) can be beneficial as well in some circumstances. Amino acid substitutions that find use in the subject antibodies include those listed in U.S. Pat. No. 8,188,321 (particularly FIG. 41) and U.S. Pat. No. 8,084,582, and US Publ. App. Nos. 20060235208 and 20070148170, all of which are expressly incorporated herein by reference in their entirety and specifically for the variants disclosed therein that affect Fc7 receptor binding. Particular variants that find use include, but are not limited to, 236A, 239D, 239E, 332E, 332D, 239D/332E, 267D, 267E, 328F, 267E/328F, 236A/332E, 239D/332E/330Y, 239D, 332E/330L, 243A, 243L, 264A, 264V and 299T. Such modification may be included in one or both Fc domains of the subject antibody.

In some embodiments, the subject antibody includes one or more Fc modifications that increase serum half-life. Fc substitutions that find use in increased binding to the FcRn receptor and increased serum half-life, as specifically disclosed in U.S. Ser. No. 12/341,769, hereby incorporated by reference in its entirety, including, but not limited to, 434S, 434A, 428L, 308F, 259I, 428L/434S, 259I/308F, 436I/428L, 436I or V/434S, 436V/428L, 259I/308F/428L, and M252Y/S254T/T256E. Such modification may be included in one or both Fc domains of the subject antibody.

### 2. Ablation Variants

In some embodiments, the heterodimeric antibody includes one or more modifications that reduce or remove the normal binding of the Fc domain to one or more or all of the Fcγ receptors (e.g., FcγR1, FcγRIIa, FcγRIIb, FcγRIIIa, etc.) to avoid additional mechanisms of action. Such modifications are referred to as “FcγR ablation variants” or “Fc knock out (FcKO or KO)” variants. In these embodiments, for some therapeutic applications, it is desirable to reduce or remove the normal binding of the Fc domain to one or more or all of the Fcγ receptors (e.g., FcγR1, FcγRIIa, FcγRIIb, FcγRIIIa, etc.) to avoid additional mechanisms of action. That is, for example, in many embodiments, particularly in the use of bispecific antibodies that bind CD28 monovalently, it is generally desirable to ablate FcγRIIIa binding to eliminate or significantly reduce ADCC activity. In some embodiments, of the subject antibodies described herein, at least one of the Fc domains comprises one or more Fcγ receptor ablation variants. In some embodiments, of the subject antibodies described herein, both of the Fc domains comprises one or more Fcγ receptor ablation variants. These ablation variants are depicted in FIG. 3, and each can be independently and optionally included or excluded, with preferred aspects utilizing ablation variants selected from the group consisting of: L234A/L235A/D265S, G236R/L328R, E233P/L234V/L235A/G236del/S239K, E233P/L234V/L235A/G236del/S267K, E233P/L234V/L235A/G236del/S239K/A327G, E233P/L234V/L235A/G236del/S267K/A327G and E233P/L234V/L235A/G236del. It should be noted that the ablation variants referenced herein ablate FcγR binding but generally not FcRn binding.

As is known in the art, the Fc domain of human IgG1 has the highest binding to the Fcγ receptors, and thus ablation variants can be used when the constant domain (or Fc domain) in the backbone of the heterodimeric antibody is IgG1. Alternatively, or in addition to ablation variants in an IgG1 background, mutations at the glycosylation position 297 (generally to A or S) can significantly ablate binding to FcγRIIIa, for example. Human IgG2 and IgG4 have naturally reduced binding to the Fcγ receptors, and thus those backbones can be used with or without the ablation variants.

### F. Combination of Heterodimeric and Fc Variants

As will be appreciated by those in the art, all of the recited heterodimerization variants (including skew and/or purification variants) can be optionally and independently combined in any way, as long as they retain their “strandedness” or “monomer partition”. In addition, all of these variants can be combined into any of the heterodimerization formats.

In the case of pI variants, while embodiments finding particular use are shown in the figures, other combinations can be generated, following the basic rule of altering the pI difference between two monomers to facilitate purification.

In addition, any of the heterodimerization variants (skew and purification variants), are also independently and optionally combined with Fc ablation variants, Fc variants, FcRn variants, as generally outlined herein.

Exemplary combination of variants that are included in some embodiments of the heterodimeric 1+1 Fab-scFv-Fc, 2+1 mAb-Fc, and 2+1 Fab2-scFv-Fc format antibodies are included in FIG. 4. In some embodiments, the heterodimeric antibody includes a combination of variants as depicted in FIG. 4.

### G. Useful Antibody Formats

As will be appreciated by those in the art and discussed more fully below, the heterodimeric bispecific antibodies provided herein can take on several different configurations as generally depicted in FIG. 26.

As will be appreciated by those in the art, the heterodimeric formats of the invention can have different valencies as well as be bispecific. That is, heterodimeric antibodies of the invention can be bivalent and bispecific, or trivalent and bispecific, wherein the first antigen is bound by two binding domains and the second antigen by a second binding domain. As is outlined herein, when CD28 is one of the target antigens, it is preferable that the CD28 is bound only monovalently.

The present invention utilizes CD28 binding domains in combination with MSLN binding domains. As will be appreciated by those in the art, any collection of anti-CD28 CDRs, anti-CD28 variable light and variable heavy domains, Fabs and scFvs as depicted in any of the Figures (see particularly FIGS. 27-30 and 33) or variants thereof can be used. Similarly, any of the MSLN antigen binding domains can be used, whether CDRs, variable light and variable heavy domains, Fabs and scFvs as depicted in any of the Figures (e.g., FIGS. 15-19 and 23) or variants thereof can be used, optionally and independently combined in any combination.

1. 1+1 Fab-scFv-Fc format

One heterodimeric antibody format that finds particular use in subject anti-CD28×anti-MSLN antibodies provided herein is the “1+1 Fab-scFv-Fc” or “bottle opener” format as shown in FIGS. 22A and 26A. The 1+1 Fab-scFv-Fc format antibody includes a first monomer that is a “regular” heavy chain (VH1-CH1-hinge-CH2-CH3), wherein VH1 is a first variable heavy domain and CH2-CH3 is a first Fc domain. The 1+1 Fab-scFv-Fc also includes a light chain that includes a first variable light domain VL1 and a constant light domain CL. The light chain interacts with the VH1-CH1 of the first monomer to form a first antigen binding domain that is a Fab. The second monomer of the antibody includes a second binding domain that is a single chain Fv (“scFv”, as defined below) and a second Fc domain. The scFv includes a second variable heavy domain (VH2) and a second variable light domain (VL2), wherein the VH2 is attached to the VL2 using an scFv linker that can be charged (see, e.g., FIG. 5). The scFv is attached to the heavy chain using a domain linker (see, e.g., FIG. 6). The two monomers are brought together by the use of amino acid variants (e.g., heterodimerization variants, discussed above) in the constant regions (e.g., the Fc domain, the CH1 domain and/or the hinge region) that promote the formation of heterodimeric antibodies as is described more fully below. This structure is sometimes referred to herein as the “bottle-opener” format, due to a rough visual similarity to a bottle-opener. In some embodiments, the 1+1 Fab-scFv-Fc format antibody is a bivalent antibody.

There are several distinct advantages to the present “1+1 Fab-scFv-Fc” format. As is known in the art, antibody analogs relying on two scFv constructs often have stability and aggregation problems, which can be alleviated in the present invention by the addition of a “regular” heavy and light chain pairing. In addition, as opposed to formats that rely on two heavy chains and two light chains, there is no issue with the incorrect pairing of heavy and light chains (e.g., heavy 1 pairing with light 2, etc.).

In some embodiments of the 1+1 Fab-scFv-Fc format antibody, one of the first or second antigen binding domain is a CD28 binding domain and the other binding domain is a MSLN binding domain. In some embodiments where the 1+1 Fab-scFv-Fc, it is the scFv that binds to the CD28, and the Fab that binds MSLN. Exemplary anti-CD28×anti-MSLN bispecific antibodies in the 1+1 Fab-scFv-Fc format are depicted in FIG. 24.

In some embodiments, the first and second Fc domains of the 1+1 Fab-scFv-Fc format antibody are variant Fc domains that include heterodimerization skew variants (e.g., a set of amino acid substitutions as shown in FIGS. 1 and 4). Particularly useful heterodimerization skew variants include S364K/E357Q:L368D/K370S; L368D/K370S:S364K; L368E/K370S:S364K; T411T/E360E/Q362E:D401K; L368D/K370S:S364K/E357L; K370S:S364K/E357Q; T366S/L368A/Y407V:T366W and T366S/L368A/Y407V/Y349C:T366W/S354C (EU numbering)). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in FIG. 3). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the first monomer includes pI variants (including those shown in FIG. 2). In exemplary embodiments, the constant domain (CH1-hinge-CH2-CH3) of the first monomer includes pI variants N208D/Q295E/N384D/Q418E/N421D, wherein numbering is according to EU numbering.

In exemplary embodiments, the 1+1 Fab-scFv-Fc format antibody includes a combination of amino acid modifications as depicted in FIG. 4. In such embodiments, the CH1-hinge-CH2-CH3 of the first monomer comprises amino acid variants L368D/K370S/N208D/Q295E/N384D/Q418E/N421D/E233P/L234V/L235A/G236del/S267 K, the second Fc domain comprises amino acid variants S364K/E357Q/E233P/L234V/L235A/G236del/S267K, wherein numbering is according to EU numbering.

In some embodiments, the scFv of the 1+1 Fab-scFv-Fc format antibody provided herein includes a charged scFv linker (including those shown in FIG. 5). In some embodiments, the 1+1 Fab-scFv-Fc format antibody provided herein includes FcRn variants M428L/N434S, wherein numbering is according to EU numbering.

In exemplary embodiments 1+1 Fab-scFv-Fc format antibody, the first Fc domain includes heterodimerization skew variants L368D/K370S and the second Fc domain includes heterodimerization skew variants S364K/E357Q; each of the first and second Fc domains include ablation variants E233P/L234V/L235A/G236_/S267K; and the constant domain (CH1-hinge-CH2-CH3) of the first monomer includes pI variants N208D/Q295E/N384D/Q418E/N421D, wherein numbering is according to EU numbering. In some embodiments, the scFv of the 1+1 Fab-scFv-Fc format antibody provided herein includes a (GKPGS)₄ charged scFv linker (SEQ ID NO: 1). In some embodiments, the 1+1 Fab-scFv-Fc format antibody provided herein includes FcRn variants M428L/N434S, wherein numbering is according to EU numbering. In some embodiments, the scFv of the 1+1 Fab-scFv-Fc format antibody provided herein includes a charged scFv linker (including those shown in FIG. 5).

Any suitable CD28 binding domain can be included in subject 1+1 Fab-scFv-Fc format antibody, including any of the CD28 binding domains provided herein. In some embodiments, the CD28 binding domain is one of the following CD28 binding domains or a variant thereof: 1A7[CD28]_H1L1, 1A7[CD28]_H1.1_L1, 1A7[CD28]_H1_L1.71, 1A7[CD28]_H1.1_L1.71, 1A7[CD28]_H1.14_L1, 1A7[CD28]_H1.14_L1.71, CD28.3[CD28]_H0L0, hCD28.3[CD28]_H1L1, 5.11A1[CD28]_H0L0, TGN1412_H1L1, 341VL34[CD28]_H1L1, 341VL36[CD28]_H1L1, 281VL4[CD28]_H1L1, HuTN228[CD28]_H1L1, PV1[CD28]_H0L0, m9.3[CD28]_H0L0, hu9.3[CD28]_H1L1, 9G2[CD28]_H0L0, 9G2[CD28]_H1L1, 2F10A3.140[CD28]_H1L1, and TN228[CD28]_H4L2 (FIGS. 27, 30 and 33). Additional VH and VL sequences of exemplary CD28 binding domains that can be used in the subject 1+1 Fab-scFv-Fc format antibody are depicted in FIGS. 28 and 29.

In some embodiments of the 1+1 Fab-scFv-Fc format, the anti-CD28 ABD has a VH and VL domain selected from the following:
(i) a VH comprising a vhCDR1, a vhCDR2, and a vhCDR3 having an amino acid sequence of a vhCDR1, a vhCDR2, and a vhCDR3, respectively, of a VH of any of the CD28 binding domains depicted in FIGS. 27, 30 and 33 or a variant thereof; and (ii) a VL comprising a vlCDR1, a vlCDR2, and a vlCDR3 having an amino acid sequence of a vlCDR1, a vlCDR2, and a vlCDR3, respectively, of the VL of the CD28 binding domain depicted in FIGS. 27, 30 and 33 or a variant thereof, or
(i) a VH comprising a vhCDR1, a vhCDR2, and a vhCDR3 having an amino acid sequence of a vhCDR1, a vhCDR2, and a vhCDR3, respectively, of a VH depicted in FIG. 28 or a variant thereof, and (ii) a VL comprising a vlCDR1, a vlCDR2, and a vlCDR3 having an amino acid sequence of a vlCDR1, a vlCDR2, and a vlCDR3, respectively, of a VL depicted in FIG. 29 or a variant thereof.

In some embodiments of the 1+1 Fab-scFv-Fc format, the anti-CD28 ABD has a VH and VL domain selected from the following:
(i) a VH having an amino acid sequence of a VH of any of the CD28 binding domains depicted in FIGS. 27, 30 and 33 or a variant thereof; and (ii) a VL having an amino acid sequence of the VL of the CD28 binding domain depicted in FIGS. 27, 30 and 33 or a variant thereof; or
(i) a VH having an amino acid sequence of a VH depicted in FIG. 28 or a variant thereof; and (ii) a VL having an amino acid sequence of the VL of a VL depicted in FIG. 29 or a variant thereof.

Any suitable MSLN binding domain can be included in subject 1+1 Fab-scFv-Fc format antibody, including any of the MSLN binding domains provided herein. Exemplary MSLN binding domains that can be used in the subject 1+1 Fab-scFv-Fc format antibody are depicted in FIGS. 15-17 and 23. Additional VH and VL sequences of exemplary MSLN binding domains that can be used in the subject 1+1 Fab-scFv-Fc format antibody are depicted in FIGS. 18 and 19.

In some embodiments of the 1+1 Fab-scFv-Fc format, the anti-MSLN ABD has a VH and VL domain selected from the following:
(i) a VH comprising a vhCDR1, a vhCDR2, and a vhCDR3 having an amino acid sequence of a vhCDR1, a vhCDR2, and a vhCDR3, respectively, of a VH of any of the MSLN binding domains depicted in FIGS. 15, 16, 17 and 23 or a variant thereof, and (ii) a VL comprising a vlCDR1, a vlCDR2, and a vlCDR3 having an amino acid sequence of a vlCDR1, a vlCDR2, and a vlCDR3, respectively, of the VL of the MSLN binding domain depicted in FIGS. 15, 16, 17 and 23 or a variant thereof, or
(i) a VH comprising a vhCDR1, a vhCDR2, and a vhCDR3 having an amino acid sequence of a vhCDR1, a vhCDR2, and a vhCDR3, respectively, of a VH depicted in FIG. 18 or a variant thereof, and (ii) a VL comprising a vlCDR1, a vlCDR2, and a vlCDR3 having an amino acid sequence of a vlCDR1, a vlCDR2, and a vlCDR3, respectively, of a VL depicted in FIG. 19 or a variant thereof.

In some embodiments of the 1+1 Fab-scFv-Fc format, the anti-MSLN ABD has a VH and VL domain selected from the following:
(i) a VH having an amino acid sequence of a VH of any one of the MSLN binding domains depicted in FIGS. 15, 16, 17 and 23 or a variant thereof, and (ii) a VL having an amino acid sequence of the VL of the MSLN binding domain depicted in FIGS. 15, 16, 17 and 23 or a variant thereof, or
(i) a VH having an amino acid sequence of a VH depicted in FIG. 18 or a variant thereof; and (ii) a VL having an amino acid sequence of the VL of a VL depicted in FIG. 19 or a variant thereof.

FIG. 7 shows some exemplary Fc domain sequences that are useful in the 1+1 Fab-scFv-Fc format antibodies. The “monomer 1” sequences depicted in FIG. 7 typically refer to the Fc domain of the “Fab-Fc heavy chain” and the “monomer 2” sequences refer to the Fc domain of the “scFv-Fc heavy chain.” In addition, FIGS. 8B and 8C provide exemplary CH1-hinge domains, CH1 domains, and hinge domains that can be included in the first or second monomer of the 1+1 Fab-scFv-Fc format. Further, FIG. 9 provides useful CL sequences that can be used with this format.

2. 2+1 mAb-scFv Format

One heterodimeric antibody format that finds particular use in the subject bispecific anti-CD28×anti-MSLN antibodies is the 2+1 mAb-scFv format shown in FIG. 26H. This antibody format includes three antigen binding domains: two Fab portions and an scFv that is attached to the C-terminal of one of the heavy chains. In some embodiments of this format, the Fab portions each bind MSLN (e.g., human MSLN) and the “extra” scFv domain binds CD28. That is, this 2+1 mAb-scFv format is a trivalent antibody.

In some of these embodiments, the first chain or monomer comprises, from N- to C-terminal, VH1-CH1-hinge-CH2-CH3, the second chain or monomer comprises, from N- to C-terminal, VH1-CH1-hinge-CH2-CH3-domain linker-scFv domain, wherein the VH1s of the first and second monomers are a first variable heavy domain. The antibody also includes two light chains that each include a VL1-CL, wherein VL1 is a first variable light domain. The scFv domain comprises a second VH (VH2), a second VL (VL2) and a scFv linker. In some embodiments, the VH1 of the first monomer and the VL1 of one of the two light chains, and the VH1 of the second monomer and the VL1 of the other of the two light chains each form a first antigen binding domain (ABD). The VH2 and VL2 form a second ABD. In some embodiments, the first ABDs each bind to human MSLN, and the second ABD binds human CD28.

As for all the scFv domains herein, the scFv domain can be in either orientation, from N- to C-terminal, VH2-scFv linker-VL2 or VL2-scFv linker-VH2. Accordingly, the second monomer may comprise, from N- to C-terminal, VH1-CH1-hinge-CH2-CH3-domain linker-VH2-scFv linker-VL2 or VH1-CH1-hinge-CH2-CH3-domain linker-VL2-scFv linker-VH2.

In some embodiments, the first and second Fc domains of the 2+1 mAb-scFv format antibody are variant Fc domains that include heterodimerization skew variants (e.g., a set of amino acid substitutions as shown in FIGS. 1 and 4). Particularly useful heterodimerization skew variants include S364K/E357Q:L368D/K370S; L368D/K370S:S364K; L368E/K370S:S364K; T411T/E360E/Q362E:D401K; L368D/K370S S364K/E357L; K370S:S364K/E357Q; T366S/L368A/Y407V:T366W and T366S/L368A/Y407V/Y349C:T366W/S354C (EU numbering)). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in FIG. 3). In some embodiments, each of the first and second variant Fe domains include ablation variants E233P/L234V/L235A/G236_/S267K, wherein numbering is according to EU numbering.

In some embodiments, the constant domain (CH1-hinge-CH2-CH3) of the first monomer includes pI variants (including those shown in FIG. 2). In exemplary embodiments, the constant domain (CH1-hinge-CH2-CH3) of the first monomer includes pI variants N208D/Q295E/N384D/Q418E/N421D, wherein numbering is according to EU numbering.

In some embodiments, the scFv of the 2+1 mAb-scFv format antibody provided herein includes a charged scFv linker (including those shown in FIG. 5). In some embodiments, the 2+1 mAb-scFv format antibody provided herein includes FcRn variants M428L/N434S, wherein numbering is according to EU numbering.

In exemplary embodiments the 2+1 mAb-scFv format antibody includes a combination of amino acid modifications as depicted in FIG. 4. In such embodiments, the first variant Fc domain includes heterodimerization skew variants L368D/K370S and the second variant Fc domain includes heterodimerization skew variants S364K/E357Q; each of the first and second variant Fc domains include ablation variants E233P/L234V/L235A/G236_/S267K; and the constant domain (CH1-hinge-CH2-CH3) of the first monomer includes pI variants N208D/Q295E/N384D/Q418E/N421D, wherein numbering is according to EU numbering. In some embodiments, the scFv of the 2+1 mAb-scFv format antibody provided herein includes a (GKPGS)₄ charged scFv linker (SEQ ID NO: 1). In some embodiments, 2+1 mAb-scFv format antibody provided herein includes FcRn variants M428L/N434S, wherein numbering is according to EU numbering.

In some embodiments, the scFv of the second monomer of the 2+1 Fab2-scFv-Fc format antibody is a CD28 binding and the VH1 of the first and second monomer and the VL1 of the common light chain each form binding domains that bind MSLN. Any suitable MSLN binding domain and CD28 binding domain can be included in the 2+1 mAb-scFv format antibody, including any of the MSLN binding domains and CD28 binding domains and related VHs and VLs provided herein or a variant thereof (see, e.g., FIGS. 15-19, 23, 27-30 and 33).

In some embodiments of the 2+1 mAb-scFv format, the anti-CD28 ABD has a VH and VL domain selected from the following:
(i) a VH comprising a vhCDR1, a vhCDR2, and a vhCDR3 having an amino acid sequence of a vhCDR1, a vhCDR2, and a vhCDR3, respectively, of a VH of any of the CD28 binding domains depicted in FIGS. 27, 30 and 33 or a variant thereof, and (ii) a VL comprising a vlCDR1, a vlCDR2, and a vlCDR3 having an amino acid sequence of a vlCDR1, a vlCDR2, and a vlCDR3, respectively, of the VL of the CD28 binding domain depicted in FIGS. 27, 30 and 33 or a variant thereof, or
(i) a VH comprising a vhCDR1, a vhCDR2, and a vhCDR3 having an amino acid sequence of a vhCDR1, a vhCDR2, and a vhCDR3, respectively, of a VH depicted in FIG. 28 or a variant thereof, and (ii) a VL comprising a vlCDR1, a vlCDR2, and a vlCDR3 having an amino acid sequence of a vlCDR1, a vlCDR2, and a vlCDR3, respectively, of a VL depicted in FIG. 29 or a variant thereof.

In some embodiments of the 2+1 mAb-scFv format, the anti-CD28 ABD has a VH and VL domain selected from the following:
(i) a VH having an amino acid sequence of a VH of any of the CD28 binding domains depicted in FIGS. 27, 30 and 33 or a variant thereof, and (ii) a VL having an amino acid sequence of the VL of the CD28 binding domain depicted in FIGS. 27, 30 and 33 or a variant thereof; or
(i) a VH having an amino acid sequence of a VH depicted in FIG. 28 or a variant thereof, and (ii) a VL having an amino acid sequence of the VL of a VL depicted in FIG. 29 or a variant thereof.

In some embodiments of the 2+1 mAb-scFv format, each of the anti-MSLN ABDs has a VH and VL domain selected from the following:
(i) a VH comprising a vhCDR1, a vhCDR2, and a vhCDR3 having an amino acid sequence of a vhCDR1, a vhCDR2, and a vhCDR3, respectively, of a VH of any of the MSLN binding domains depicted in FIGS. 15, 16, 17 and 23 or a variant thereof, and (ii) a VL comprising a vlCDR1, a vlCDR2, and a vlCDR3 having an amino acid sequence of a vlCDR1, a vlCDR2, and a vlCDR3, respectively, of the VL of the MSLN binding domain depicted in FIGS. 15, 16, 17 and 23 or a variant thereof, or
(i) a VH comprising a vhCDR1, a vhCDR2, and a vhCDR3 having an amino acid sequence of a vhCDR1, a vhCDR2, and a vhCDR3, respectively, of a VH depicted in FIG. 18 or a variant thereof, and (ii) a VL comprising a vlCDR1, a vlCDR2, and a vlCDR3 having an amino acid sequence of a vlCDR1, a vlCDR2, and a vlCDR3, respectively, of a VL depicted in FIG. 19 or a variant thereof.

In some embodiments of the 2+1 mAb-scFv format, each of the anti-MSLN ABDs has a VH and VL domain selected from the following:
(i) a VH having an amino acid sequence of a VH of any one of the MSLN binding domains depicted in FIGS. 15, 16, 17 and 23 or a variant thereof, and (ii) a VL having an amino acid sequence of the VL of the MSLN binding domain depicted in FIGS. 15, 16, 17 and 23 or a variant thereof, or
(i) a VH having an amino acid sequence of a VH depicted in FIG. 18 or a variant thereof; and (ii) a VL having an amino acid sequence of the VL of a VL depicted in FIG. 19 or a variant thereof.

Exemplary anti-CD28×anti-MSLN antibodies in the 2+1 mAb-scFv format are depicted in FIG. 24.

FIG. 7 shows some exemplary Fc domain sequences that are useful in the 2+1 mAb-scFv format antibodies. In addition, FIGS. 8B and 8C provide exemplary CH1-hinge domains, CH1 domains, and hinge domains that can be included in the first or second monomer of the 2+1 mAb-scFv. Further, FIG. 9 provides useful CL sequences that can be used with this format.

3. 2+1 Fab2-scFv-Fc format

One heterodimeric antibody format that finds particular use in subject anti-CD28×anti-MSLN antibodies provided herein is the 2+1 Fab2-scFv-Fc format (also referred to as “central-scFv format”) shown in FIGS. 22B and 26F. This antibody format includes three antigen binding domains: two Fab portions and an scFv that is inserted between the VH-CH1 and CH2-CH3 regions of one of the monomers. In some embodiments of this format, the Fab portions each bind MSLN and the “extra” scFv domain binds CD28. In some embodiments, the 2+1 Fab2-scFv-Fc format antibody is a trivalent antibody.

In some embodiments of the 2+1 Fab2-scFv-Fc format, a first monomer includes a standard heavy chain (i.e., VH1-CH1-hinge-CH2-CH3), wherein VH1 is a first variable heavy domain and CH2-CH3 is a first Fc domain. A second monomer includes another first variable heavy domain (VH1), a CH1 domain (and optional hinge), a second Fc domain, and an scFv that includes an scFv variable light domain (VL2), an scFv linker and a scFv variable heavy domain (VH2). The scFv is covalently attached between the C-terminus of the CH1 domain of the second monomer and the N-terminus of the second Fc domain using optional domain linkers (VH1-CH1-[optional linker]-VH2-scFv linker-VH2-[optional linker]-CH2-CH3, or the opposite orientation for the scFv, VH1-CH1-[optional linker]-VL2-scFv linker-VH2-[optional linker]-CH2-CH3). The optional linkers can be any suitable peptide linkers, including, for example, the domain linkers included in FIG. 6. This embodiment further utilizes a first and second common light chain that each include a first variable light domain (VL1) and a constant light domain (CL). The VH1-CH1 of the first and second monomers each interacts with the one of the two common light chains to form two identical Fabs. In some embodiments, the identical Fabs are MSLN binding domains and the scFv is a CD28 binding domain. As for many of the embodiments herein, these constructs can include skew variants, pI variants, ablation variants, additional Fc variants, etc. as desired and described herein.

In some embodiments, the first and second Fc domains of the 2+1 Fab2-scFv-Fc format antibody are variant Fc domains that include heterodimerization skew variants (e.g., a set of amino acid substitutions as shown in FIGS. 1 and 4). Particularly useful heterodimerization skew variants include S364K/E357Q:L368D/K370S; L368D/K370S:S364K; L368E/K370S:S364K; T411T/E360E/Q362E:D401K; L368D/K370S:S364K/E357L; K370S:S364K/E357Q; T366S/L368A/Y407V:T366W and T366S/L368A/Y407V/Y349C:T366W/S354C (EU numbering)). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in FIG. 3). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the first monomer includes pI variants (including those shown in FIG. 2). In exemplary embodiments, the constant domain (CH1-hinge-CH2-CH3) of the first monomer includes pI variants N208D/Q295E/N384D/Q418E/N421D, wherein numbering is according to EU numbering.

In some embodiments, the scFv of the 2+1 Fab2-scFv-Fc format antibody provided herein includes a charged scFv linker (including those shown in FIG. 5). In some embodiments, the 2+1 Fab2-scFv-Fc format antibody provided herein includes FcRn variants M428L/N434S, wherein numbering is according to EU numbering.

In exemplary embodiments 2+1 Fab2-scFv-Fc format antibody includes a combination of amino acid modifications as depicted in FIG. 4. In such embodiments, the first variant Fc domain includes heterodimerization skew variants L368D/K370S and the second variant Fc domain includes heterodimerization skew variants S364K/E357Q; each of the first and second variant Fc domains include ablation variants E233P/L234V/L235A/G236_/S267K; and the constant domain (CH1-hinge-CH2-CH3) of the first monomer includes pI variants N208D/Q295E/N384D/Q418E/N421D, wherein numbering is according to EU numbering. In some embodiments, the scFv of the 2+1 Fab2-scFv-Fc format antibody provided herein includes a (GKPGS)₄ charged scFv linker (SEQ ID NO: 1). In some embodiments, the 2+1 Fab2-scFv-Fc format antibody provided herein includes FcRn variants M428L/N434S, wherein numbering is according to EU numbering.

In some embodiments, the CH1-hinge-CH2-CH3 of the first monomer comprises amino acid variants L368D/K370S/N208D/Q295E/N384D/Q418E/N421D/E233P/L234V/L235A/G236del/S267K, and the second Fc domain comprises amino acid variants S364K/E357Q/E233P/L234V/L235A/G236del/S267K, wherein numbering is according to EU numbering.

In some embodiments, the scFv of the second monomer of the 2+1 Fab2-scFv-Fc format antibody is a CD28 binding and the VH1 of the first and second monomer and the VL1 of the common light chain each form binding domains that bind MSLN. Any suitable CD28 binding domain can be included in subject 2+1 Fab2-scFv-Fc format antibody, including any of the CD28 binding domains provided herein. In some embodiments, the CD28 binding domain is one of the following CD28 binding domains or a variant thereof: 1A7[CD28]_H1L1, 1A7[CD28]_H1.1_L1, 1A7[CD28]_H1_L1.71, 1A7[CD28]_H1.1_L1.71, 1A7[CD28]_H1.14_L1, 1A7[CD28]_H1.14_L1.71, CD28.3[CD28]_H0L0, hCD28.3[CD28]_H1L1, 5.11A1[CD28]_H0L0, TGN1412_H1L1, 341VL34[CD28]_H1L1, 341VL36[CD28]_H1L1, 281VL4[CD28]_H1L1, HuTN228[CD28]_H1L1, PV1[CD28]_H0L0, m9.3[CD28]_H0L0, hu9.3[CD28]_H1L1, 9G2[CD28]_H0L0, 9G2[CD28]_H1L1, 2F10A3.140[CD28]_H1L1, and TN228[CD28]_H4L2 (FIGS. 27, 30 and 33).

In some embodiments of the 2+1 Fab2-scFv-Fc format, the anti-CD28 ABD has a VH and VL domain selected from the following:
(i) a VH comprising a vhCDR1, a vhCDR2, and a vhCDR3 having an amino acid sequence of a vhCDR1, a vhCDR2, and a vhCDR3, respectively, of a VH of any of the CD28 binding domains depicted in FIGS. 27, 30 and 33 or a variant thereof, and (ii) a VL comprising a vlCDR1, a vlCDR2, and a vlCDR3 having an amino acid sequence of a vlCDR1, a vlCDR2, and a vlCDR3, respectively, of the VL of the CD28 binding domain depicted in FIGS. 27, 30 and 33 or a variant thereof, or
(i) a VH comprising a vhCDR1, a vhCDR2, and a vhCDR3 having an amino acid sequence of a vhCDR1, a vhCDR2, and a vhCDR3, respectively, of a VH depicted in FIG. 28 or a variant thereof, and (ii) a VL comprising a vlCDR1, a vlCDR2, and a vlCDR3 having an amino acid sequence of a vlCDR1, a vlCDR2, and a vlCDR3, respectively, of a VL depicted in FIG. 29 or a variant thereof.

In some embodiments of the 2+1 Fab2-scFv-Fc format, the anti-CD28 ABD has a VH and VL domain selected from the following:
(i) a VH having an amino acid sequence of a VH of any of the CD28 binding domains depicted in FIGS. 27, 30 and 33 or a variant thereof, and (ii) a VL having an amino acid sequence of the VL of the CD28 binding domain depicted in FIGS. 27, 30 and 33 or a variant thereof; or
(i) a VH having an amino acid sequence of a VH depicted in FIG. 28 or a variant thereof; and (ii) a VL having an amino acid sequence of the VL of a VL depicted in FIG. 29 or a variant thereof.

In some embodiments, the VH1 of the first and second monomer and the VL1 of the common light chain of the 2+1 Fab2-scFv-Fc format antibody each form a binding domain that binds MSLN. Any suitable MSLN binding domain can be included in subject 2+1 Fab2-scFv-Fc format antibody, including any of the MSLN binding domains provided herein. Exemplary MSLN binding domains that can be used in the subject 2+1 Fab2-scFv-Fc format antibody are depicted in FIGS. 15-17 and 23. Additional VH and VL sequences of exemplary MSLN binding domains that can be used in the subject 2+1 Fab2-scFv-Fc format antibody are depicted in FIGS. 18 and 19.

In some embodiments of 2+1 Fab2-scFv-Fc format, the anti-MSLN ABDs each include a VH and VL domain selected from the following:
(i) a VH comprising a vhCDR1, a vhCDR2, and a vhCDR3 having an amino acid sequence of a vhCDR1, a vhCDR2, and a vhCDR3, respectively, of a VH of any of the MSLN binding domains depicted in FIGS. 15, 16, 17 and 23 or a variant thereof, and (ii) a VL comprising a vlCDR1, a vlCDR2, and a vlCDR3 having an amino acid sequence of a vlCDR1, a vlCDR2, and a vlCDR3, respectively, of the VL of the MSLN binding domain depicted in FIGS. 15, 16, 17 and 23 or a variant thereof, or
(i) a VH comprising a vhCDR1, a vhCDR2, and a vhCDR3 having an amino acid sequence of a vhCDR1, a vhCDR2, and a vhCDR3, respectively, of a VH depicted in FIG. 18 or a variant thereof, and (ii) a VL comprising a vlCDR1, a vlCDR2, and a vlCDR3 having an amino acid sequence of a vlCDR1, a vlCDR2, and a vlCDR3, respectively, of a VL depicted in FIG. 19 or a variant thereof.

In some embodiments of 2+1 Fab2-scFv-Fc format, the anti-MSLN ABDs each include a VH and VL domain selected from the following:
(i) a VH having an amino acid sequence of a VH of any one of the MSLN binding domains depicted in FIGS. 15, 16, 17 and 23 or a variant thereof, and (ii) a VL having an amino acid sequence of the VL of the MSLN binding domain depicted in FIGS. 15, 16, 17 and 23 or a variant thereof, or
(i) a VH having an amino acid sequence of a VH depicted in FIG. 18 or a variant thereof; and (ii) a VL having an amino acid sequence of the VL of a VL depicted in FIG. 19 or a variant thereof.

FIG. 7 shows some exemplary Fc domain sequences that are useful in the 2+1 Fab2-scFv-Fc format antibodies. In addition, FIGS. 8B and 8C provide exemplary CH1-hinge domains, CH1 domains, and hinge domains that can be included in the first or second monomer of the 2+1 Fab2-scFv-Fc. Further, FIG. 9 provides useful CL sequences that can be used with this format.

Sequences for illustrative MSLN×CD28 bsAbs (based on binding domains as described in Examples 1 and 2) in the 2+1 Fab2-scFv-Fc format are depicted in FIG. 35.

### 4. 2+1 Fab2-Fc×scFv-Fc Format

One heterodimeric antibody format that finds particular use in the subject anti-CD28×anti-MSLN antibodies provided herein is the 2+1 Fab2-Fc×scFv-Fc format (also referred to as “stacked bottle opener”) shown in FIGS. 26L. This format includes a first monomer, a second monomer and two common light chains. The first monomer includes, from N- to C-terminus: a VH1-CH1-domain linker-VH1-CH1-hinge-CH2-CH3, wherein the VH1s are each a first variable heavy domain and the CH2-CH3 is a first variant Fc domain. The second monomer includes a single-chain Fv (“scFv”) covalently attached to a second variant Fc domain by a domain linker (scFv-domain linker-CH2-CH3). The two common light chains each include a VL1-CL wherein the VL1s are each a first variable light domain. The scFv of the second monomer includes a second variable heavy domain (VH2) attached to a second variable light domain (VL2) by an scFv linker. In this embodiment, the two VH1-CH1s of the first monomer each interact with one of the two common light chains to form two identical first antigen binding domains, and the VH2 and VL2 form a second antigen binding domain. In some embodiments, each of the first antigen binding domains are MSLN binding domains, and the second antigen binding domain is a CD28 binding domain. As for many of the embodiments herein, these constructs can include skew variants, pI variants, ablation variants, additional Fc variants, etc. as desired and described herein.

In some embodiments, the first and second Fc domains of the 2+1 Fab2-Fc×scFv-Fc format antibody are variant Fc domains that include heterodimerization skew variants (e.g., a set of amino acid substitutions as shown in FIGS. 1 and 4). Particularly useful heterodimerization skew variants include S364K/E357Q:L368D/K370S; L368D/K370S:S364K; L368E/K370S:S364K; T411T/E360E/Q362E:D401K; L368D/K370S:S364K/E357L; K370S:S364K/E357Q; T366S/L368A/Y407V:T366W and T366S/L368A/Y407V/Y349C:T366W/S354C (EU numbering)). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in FIG. 3). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the first monomer includes pI variants (including those shown in FIG. 2). In exemplary embodiments, the constant domain (CH1-hinge-CH2-CH3) of the first monomer includes pI variants N208D/Q295E/N384D/Q418E/N421D, wherein numbering is according to EU numbering.

In some embodiments, the scFv of the 2+1 Fab2-Fc×scFv-Fc format antibody provided herein includes a charged scFv linker (including those shown in FIG. 5). In some embodiments, the 2+1 Fab2-Fc×scFv-Fc format antibody provided herein includes FcRn variants M428L/N434S, wherein numbering is according to EU numbering.

In exemplary embodiments, the 2+1 Fab2-Fc×scFv-Fc format antibody includes a combination of amino acid modifications as depicted in FIG. 4. In such embodiments, the first variant Fc domain includes heterodimerization skew variants L368D/K370S and the second variant Fc domain includes heterodimerization skew variants S364K/E357Q; each of the first and second variant Fc domains include ablation variants E233P/L234V/L235A/G236_/S267K; and the constant domain (CH1-hinge-CH2-CH3) of the first monomer optionally includes pI variants N208D/Q295E/N384D/Q418E/N421D, wherein numbering is according to EU numbering. In some embodiments, the scFv of the 2+1 Fab2-Fc×scFv-Fc format antibody provided herein includes a (GKPGS)₄ charged scFv linker. In some embodiments, the 2+1 Fab2-Fc×scFv-Fc format antibody provided herein includes FcRn variants M428L/N434S, wherein numbering is according to EU numbering.

In some embodiments, the CH1-hinge-CH2-CH3 of the first monomer comprises amino acid variants L368D/K370S/N208D/Q295E/N384D/Q418E/N421D/E233P/L234V/L235A/G236del/S267K, and the second Fc domain comprises amino acid variants S364K/E357Q/E233P/L234V/L235A/G236del/S267K, wherein numbering is according to EU numbering.

In some embodiments, the CH1-hinge-CH2-CH3 of the first monomer comprises amino acid variants L368D/K370S/E233P/L234V/L235A/G236del/S267K, and the second Fc domain comprises amino acid variants S364K/E357Q/E233P/L234V/L235A/G236del/S267K, wherein numbering is according to EU numbering.

In some embodiments, the scFv of the second monomer of the 2+1 Fab2-Fc×scFv-Fc format antibody is a CD28 binding domain and the VH1s of the first monomer and the VL1s of the two common light chain each form MSLN binding domains. Any suitable CD28 binding domain can be included in subject 2+1 Fab2-Fc×scFv-Fc format antibody, including any of the CD28 binding domains provided herein. In some embodiments, the CD28 binding domain is one of the following CD28 binding domains or a variant thereof: 1A7[CD28]_H1L1, 1A7[CD28]_H1.1_L1, 1A7[CD28]_H1_L1.71, 1A7[CD28]_H1.1_L1.71, 1A7[CD28]_H1.14_L1, 1A7[CD28]_H1.14_L1.71, CD28.3[CD28]_H0L0, hCD28.3[CD28]_H1L1, 5.11A1[CD28]_H0L0, TGN1412_H1L1, 341VL34[CD28]_H1L1, 341VL36[CD28]_H1L1, 281VL4[CD28]_H1L1, HuTN228[CD28]_H1L1, PV1[CD28]_H0L0, m9.3[CD28]_H0L0, hu9.3[CD28]_H1L1, 9G2[CD28]_H0L0, 9G2[CD28]_H1L1, 2F10A3.140[CD28]_H1L1, and TN228[CD28]_H4L2 (FIGS. 27, 30 and 33).

In some embodiments of the 2+1 Fab2-Fc×scFv-Fc format, the anti-CD28 ABD has a VH and VL domain selected from the following:
(i) a VH comprising a vhCDR1, a vhCDR2, and a vhCDR3 having an amino acid sequence of a vhCDR1, a vhCDR2, and a vhCDR3, respectively, of a VH of any of the CD28 binding domains depicted in FIGS. 27, 30 and 33 or a variant thereof, and (ii) a VL comprising a vlCDR1, a vlCDR2, and a vlCDR3 having an amino acid sequence of a vlCDR1, a vlCDR2, and a vlCDR3, respectively, of the VL of the CD28 binding domain depicted in FIGS. 27, 30 and 33 or a variant thereof, or
(i) a VH comprising a vhCDR1, a vhCDR2, and a vhCDR3 having an amino acid sequence of a vhCDR1, a vhCDR2, and a vhCDR3, respectively, of a VH depicted in FIG. 28 or a variant thereof, and (ii) a VL comprising a vlCDR1, a vlCDR2, and a vlCDR3 having an amino acid sequence of a vlCDR1, a vlCDR2, and a vlCDR3, respectively, of a VL depicted in FIG. 29 or a variant thereof.

In some embodiments of the 2+1 Fab2-Fc×scFv-Fc format, the anti-CD28 ABD has a VH and VL domain selected from the following:
(i) a VH having an amino acid sequence of a VH of any of the CD28 binding domains depicted in FIGS. 27, 30 and 33 or a variant thereof, and (ii) a VL having an amino acid sequence of the VL of the CD28 binding domain depicted in FIGS. 27, 30 and 33 or a variant thereof; or
(i) a VH having an amino acid sequence of a VH depicted in FIG. 28 or a variant thereof; and (ii) a VL having an amino acid sequence of the VL of a VL depicted in FIG. 29 or a variant thereof.

In some embodiments, the VH1s of the first monomer and the VL1s of the two common light chain of the 2+1 Fab2-Fc×scFv-Fc format antibody each form a binding domain that binds MSLN. Any suitable MSLN binding domain can be included in subject 2+1 Fab2-Fc×scFv-Fc format antibody, including any of the MSLN binding domains provided herein. Exemplary MSLN binding domains that can be used in the subject 2+1 Fab2-Fc×scFv-Fc format antibody are depicted in FIGS. 15-17 and 23. Additional VH and VL sequences of exemplary MSLN binding domains that can be used in the subject 2+1 Fab2-Fc×scFv-Fc format antibody are depicted in FIGS. 18 and 19.

In some embodiments of 2+1 Fab2-Fc×scFv-Fc format, the anti-MSLN ABDs each include a VH and VL domain selected from the following:
(i) a VH comprising a vhCDR1, a vhCDR2, and a vhCDR3 having an amino acid sequence of a vhCDR1, a vhCDR2, and a vhCDR3, respectively, of a VH of any of the MSLN binding domains depicted in FIGS. 15, 16, 17 and 23 or a variant thereof, and (ii) a VL comprising a vlCDR1, a vlCDR2, and a vlCDR3 having an amino acid sequence of a vlCDR1, a vlCDR2, and a vlCDR3, respectively, of the VL of the MSLN binding domain depicted in FIGS. 15, 16, 17 and 23 or a variant thereof, or
(i) a VH comprising a vhCDR1, a vhCDR2, and a vhCDR3 having an amino acid sequence of a vhCDR1, a vhCDR2, and a vhCDR3, respectively, of a VH depicted in FIG. 18 or a variant thereof, and (ii) a VL comprising a vlCDR1, a vlCDR2, and a vlCDR3 having an amino acid sequence of a vlCDR1, a vlCDR2, and a vlCDR3, respectively, of a VL depicted in FIG. 19 or a variant thereof.

In some embodiments of 2+1 Fab2-Fc×scFv-Fc format, the anti-MSLN ABDs each include a VH and VL domain selected from the following:
(i) a VH having an amino acid sequence of a VH of any one of the MSLN binding domains depicted in FIGS. 15, 16, 17 and 23 or a variant thereof, and (ii) a VL having an amino acid sequence of the VL of the MSLN binding domain depicted in FIGS. 15, 16, 17 and 23 or a variant thereof, or
(i) a VH having an amino acid sequence of a VH depicted in FIG. 18 or a variant thereof; and (ii) a VL having an amino acid sequence of the VL of a VL depicted in FIG. 19 or a variant thereof.

FIG. 7 shows some exemplary Fc domain sequences that are useful in the 2+1 Fab2-Fc×scFv-Fc format antibodies. In addition, FIGS. 8B and 8C provide exemplary CH1-hinge domains, CH1 domains, and hinge domains that can be included in the first or second monomer of the 2+1 Fab2-Fc×scFv-Fc. Further, FIG. 9 provides useful CL sequences that can be used with this format.

Sequences for illustrative MSLN×CD28 bsAbs (based on binding domains as described in Examples 1 and 2) in the 2+1 Fab2-Fc×scFv-Fc format are depicted in FIG. 36.

5. Dual scFv Format

One heterodimeric antibody format that finds particular use in the subject bispecific anti-CD28×anti-MSLN antibodies is the dual scFv format, as are known in the art and shown in FIG. 26B. In this embodiment, the heterodimeric bispecific antibody is made up of two scFv-Fc monomers (both in either (vh-scFv linker-vl-[optional domain linker]-CH2-CH3) format or (vl-scFv linker-vh-[optional domain linker]-CH2-CH3) format, or with one monomer in one orientation and the other in the other orientation.

In this case, all ABDs are in the scFv format. Any suitable MSLN binding domain and CD28 binding domain can be included in anti-CD28×anti-MSLN antibody in the dual scFv format, including any of the MSLN binding domains and CD28 binding domains provided herein.

In addition, the Fc domains of the dual scFv format comprise skew variants (e.g. a set of amino acid substitutions as shown in FIG. 1, with particularly useful skew variants being selected from the group consisting of S364K/E357Q:L368D/K370S; L368D/K370S:S364K; L368E/K370S:S364K; T411T/E360E/Q362E:D401K; L368D/K370S:S364K/E357L, K370S:S364K/E357Q, T366S/L368A/Y407V:T366W and T366S/L368A/Y407V/Y349C:T366W/S354C), optionally ablation variants (including those shown in FIG. 3), optionally charged scFv linkers (including those shown in FIG. 5) and the heavy chain comprises pI variants (including those shown in FIG. 2).

In some embodiments, the dual scFv format includes skew variants, pI variants, and ablation variants. Accordingly, some embodiments include formats that comprise: a) a first monomer that comprises the skew variants S364K/E357Q, the ablation variants E233P/L234V/L235A/G236del/S267K, and a scFv that binds a first antigen (VH1-scFv linker-VL1-[optional domain linker]-CH2-CH3 or VL1-scFv linker-VH1-[optional domain linker]-CH2-CH3) and b) a first monomer that comprises the skew variants L368D/K370S, the ablation variants E233P/L234V/L235A/G236del/S267K, and a scFv that binds a second antigen (VH1-scFv linker-VL1-[optional domain linker]-CH2-CH3 or VL1-scFv linker-VH1-[optional domain linker]-CH2-CH3). pI variants can be as outlined herein, but most common will be charged scFv linkers of opposite charge for each monomer. FcRn variants, particularly 428L/434S, can optionally be included.

Any suitable MSLN binding domain and CD28 binding domain can be included in the dual scFv format, including any of the MSLN binding domains and CD28 binding domains and related VHs and VLs provided herein or a variant thereof (see, e.g., FIGS. 15-19, 23, 27-30 and 33).

### 6. One-Armed Central-scFv

One heterodimeric antibody format that finds particular use in subject anti-CD28×anti-MSLN antibodies provided herein is the one-armed central-scFv format shown in FIG. 26C. In this embodiment, one monomer comprises just an Fc domain, while the other monomer includes a Fab domain (a first antigen binding domain), a scFv domain (a second antigen binding domain) and an Fc domain, where the scFv domain is inserted between the Fc domain and the Fc domain.

In this embodiment, one monomer comprises a first heavy chain comprising a first variable heavy domain, a CH1 domain and Fc domain, with a scFv comprising a scFv variable light domain, an scFv linker and a scFv variable heavy domain. The scFv is covalently attached between the C-terminus of the CH1 domain of the heavy constant domain and the N-terminus of the first Fc domain using domain linkers, in either orientation, VH1-CH1-[optional domain linker]-VH2-scFv linker-VL2-[optional domain linker]-CH2-CH3 or VH1-CH1-[optional domain linker]-VL2-scFv linker-VH2-[optional domain linker]-CH2-CH3. The second monomer comprises an Fc domain (CH2-CH3). This embodiment further utilizes a light chain comprising a variable light domain and a constant light domain, that associates with the heavy chain to form a Fab. As for many of the embodiments herein, these constructs include skew variants, pI variants, ablation variants, additional Fc variants, etc. as desired and described herein.

Any suitable MSLN binding domain and CD28 binding domain can be included in the one-armed central-scFv format, including any of the MSLN binding domains and CD28 binding domains and related VHs and VLs provided herein or a variant thereof (see, e.g., FIGS. 15-19, 23, 27-30 and 33).

7. One-Armed scFv-mAb Format

One heterodimeric antibody format that finds particular use in subject anti-CD28×anti-MSLN antibodies provided herein is the one-armed mAb-scFv format shown in FIG. 26D. This format includes: 1) a first monomer that comprises an “empty” Fc domain; 2) a second monomer that includes a first variable heavy domain (VH), a scFv domain (a second antigen binding domain) and an Fc domain, where the scFv domain is attached to the N-terminus of the first variable heavy domain; and 3) a light chain that includes a first variable light domain and a constant light domain. The first variable heavy domain and the first variable light domain form a first antigen binding domain and the scFv is a second antigen binding domain. In this format, one of the first antigen binding domain and second binding domain binds CD28, and the other antigen binding domain binds MSLN. As for many of the embodiments herein, these constructs include skew variants, pI variants, ablation variants, additional Fc variants, etc. as desired and described herein.

Any suitable MSLN binding domain and CD28 binding domain can be included in the one-armed scFv-mAb format antibody, including any of the MSLN binding domains and CD28 binding domains and related VHs and VLs provided herein or a variant thereof (see, e.g., FIGS. 15-19, 23, 27-30 and 33).

8. scFv-mAb Format

One heterodimeric antibody format that finds particular use in subject anti-CD28×anti-MSLN antibodies provided herein is the scFv-mAb format shown in FIG. 26E. In this embodiment, the format relies on the use of a N-terminal attachment of a scFv to one of the monomers, thus forming a third antigen binding domain, wherein the Fab portions of the two monomers each bind one target and the “extra” scFv domain binds a different target.

In this embodiment, the first monomer comprises a first heavy chain (comprising a variable heavy domain and a constant domain), with a N-terminally covalently attached scFv comprising a scFv variable light domain, an scFv linker and a scFv variable heavy domain in either orientation ((vh1-scFv linker-vl1-[optional domain linker]-vh2-CH1-hinge-CH2-CH3) or (with the scFv in the opposite orientation) ((vl1-scFv linker-vh1-[optional domain linker]-vh2-CH1-hinge-CH2-CH3)). The second monomer comprises a heavy chain VH2-CH1-hinge-CH2-CH3. This embodiment further utilizes a common light chain comprising a variable light domain and a constant light domain, that associates with the heavy chains to form two identical Fabs. As for many of the embodiments herein, these constructs include skew variants, pI variants, ablation variants, additional Fc variants, etc. as desired and described herein.

Any suitable MSLN binding domain and CD28 binding domain can be included in the scFv-mAb antibody format, including any of the MSLN binding domains and CD28 binding domains and related VHs and VLs provided herein or a variant thereof (see, e.g., FIGS. 15-19, 23, 27-30 and 33).

9. mAb-Fv Format

One heterodimeric antibody format that finds particular use in subject anti-CD28×anti-MSLN antibodies provided herein is the mAb-Fv format (FIG. 26G). In this embodiment, the format relies on the use of a C-terminal attachment of an “extra” variable heavy domain to one monomer and the C-terminal attachment of an “extra” variable light domain to the other monomer, thus forming a third antigen binding domain (i.e. an “extra” Fv domain), wherein the Fab portions of the two monomers bind CD28 and the “extra” Fv domain binds MSLN.

In this embodiment, the first monomer comprises a first heavy chain, comprising a first variable heavy domain and a first constant heavy domain comprising a first Fc domain, with a first variable light domain covalently attached to the C-terminus of the first Fc domain using a domain linker (vh1-CH1-hinge-CH2-CH3-[optional linker]-vl2). The second monomer comprises a second variable heavy domain, a second constant heavy domain comprising a second Fc domain, and a third variable heavy domain covalently attached to the C-terminus of the second Fc domain using a domain linker (vh1-CH1-hinge-CH2-CH3-[optional linker]-vh2. This embodiment further utilizes a common light chain comprising a variable light domain and a constant light domain, which associates with the heavy chains to form two identical Fabs that include two identical Fvs. The two C-terminally attached variable domains make up the “extra” third Fv. As for many of the embodiments herein, these constructs include skew variants, pI variants, ablation variants, additional Fc variants, etc. as desired and described herein.

Any suitable MSLN binding domain and CD28 binding domain can be included in the mAb-Fv format, including any of the MSLN binding domains and CD28 binding domains and related VHs and VLs provided herein or a variant thereof (see, e.g., FIGS. 15-19, 23, 27-30 and 33).

### 10. Central-Fv Format

One heterodimeric antibody format that finds particular use in subject anti-CD28×anti-MSLN antibodies provided herein is the central-Fv format shown in FIG. 26I. In this embodiment, the format relies on the use of an inserted Fv domain thus forming an “extra” third antigen binding domain, wherein the Fab portions of the two monomers bind MSLN and the “extra” central-Fv domain binds CD28. The Fv domain is inserted between the Fc domain and the CH1-Fv region of the monomers, thus providing a third antigen binding domain, wherein each monomer contains a component of the Fv (e.g. one monomer comprises a variable heavy domain and the other a variable light domain of the “extra” central Fv domain).

In this embodiment, one monomer comprises a first heavy chain comprising a first variable heavy domain, a CH1 domain, and Fc domain and an additional variable light domain. The additional variable light domain is covalently attached between the C-terminus of the CH1 domain of the heavy constant domain and the N-terminus of the first Fc domain using domain linkers (vh1-CH1-[optional linker]-vl2-hinge-CH2-CH3). The other monomer comprises a first heavy chain comprising a first variable heavy domain, a CH1 domain and Fc domain and an additional variable heavy domain (vh1-CH1-[optional linker]-vh2-hinge-CH2-CH3). The additional variable heavy domain is covalently attached between the C-terminus of the CH1 domain of the heavy constant domain and the N-terminus of the first Fc domain using domain linkers. This embodiment utilizes a common light chain comprising a variable light domain and a constant light domain, that associates with the heavy chains to form two identical Fabs that each bind MSLN. The additional variable heavy domain and additional variable light domain form an “extra” central Fv that binds CD28. As for many of the embodiments herein, these constructs include skew variants, pI variants, ablation variants, additional Fc variants, etc. as desired and described herein.

Any suitable MSLN binding domain and CD28 binding domain can be included in the central-Fv format, including any of the MSLN binding domains and CD28 binding domains and related VHs and VLs provided herein or a variant thereof (see, e.g., FIGS. 15-19, 23, 27-30 and 33).

### 11. Non-Heterodimeric Bispecific Antibodies

As will be appreciated by those in the art, the anti-CD28×anti-MSLN antibodies provided herein can also be included in non-heterodimeric bispecific formats (see FIG. 26J). In this format, the anti-CD28×anti-MSLN includes: 1) a first monomer comprising a VH1-CH1-hinge-CH2-CH3; 2) a second monomer comprising a VH2-CH1-hinge-CH2-CH3; 3) a first light chain comprising a VL1-CL; and 4) a second light chain comprising a VL2-CL. In such embodiments, the VH1 and VL1 form a first antigen binding domain and VH2 and VL2 form a second antigen binding domain. One of the first or second antigen binding domains binds CD28 and the other antigen binding domain binds MSLN.

Any suitable MSLN binding domain and CD28 binding domain can be included in anti-CD28×anti-MSLN antibody in the non-heterodimeric bispecific antibody format, including any of the MSLN binding domains and CD28 binding domains and related VHs and VLs provided herein or a variant thereof (see, e.g., FIGS. 15-19, 23, 27-30 and 33).

### 12. Trident Format

In some embodiments, the anti-CD28×anti-MSLN antibodies provided herein are in the “Trident” format as generally described in WO2015/184203, hereby expressly incorporated by reference in its entirety and in particular for the Figures, Legends, definitions and sequences of “Heterodimer-Promoting Domains” or “HPDs”, including “K-coil” and “E-coil” sequences. Tridents rely on using two different HPDs that associate to form a heterodimeric structure as a component of the structure, see FIG. 26K. In this embodiment, the Trident format include a “traditional” heavy and light chain (e.g. VH1-CH1-hinge-CH2-CH3 and VL1-CL), a third chain comprising a first “diabody-type binding domain” or “DART®”, VH2-(linker)-VL3-HPD1 and a fourth chain comprising a second DART®, VH3-(linker)-(linker)-VL2-HPD2. The VH1 and VL1 form a first ABD, the VH2 and VL2 form a second ABD, and the VH3 and VL3 form a third ABD. In some cases, as is shown in FIG. 26K, the second and third ABDs bind the same antigen.

Any suitable MSLN binding domain and CD28 binding domain can be included in the trident format, including any of the MSLN binding domains and CD28 binding domains and related VHs and VLs provided herein or a variant thereof (see, e.g., FIGS. 15-19, 23, 27-30 and 33).

### V. Nucleic Acids

In another aspect, provided herein are nucleic acid compositions encoding the anti-CD28×anti-MSLN antibodies provided herein. A nucleic acid composition may refer to one or multiple polynucleotides.

As will be appreciated by those in the art, the nucleic acid compositions will depend on the format and scaffold of the heterodimeric protein. Thus, for example, when the format requires three amino acid sequences, such as for the 1+1 Fab-scFv-Fc or 2+1 Fab2-scFv-Fc formats, three polynucleotides can be incorporated into one or more expression vectors for expression. In exemplary embodiments, each polynucleotide is incorporated into a different expression vector.

As is known in the art, the nucleic acids encoding the components of the binding domains and antibodies disclosed herein can be incorporated into expression vectors as is known in the art, and depending on the host cells used to produce the heterodimeric antibodies of the invention. Generally the nucleic acids are operably linked to any number of regulatory elements (promoters, origin of replication, selectable markers, ribosomal binding sites, inducers, etc.). The expression vectors can be extra-chromosomal or integrating vectors.

The polynucleotides and/or expression vectors of the invention are then transformed into any number of different types of host cells as is well known in the art, including mammalian, bacterial, yeast, insect and/or fungal cells, with mammalian cells (e.g., CHO cells), finding use in many embodiments.

In some embodiments, polynucleotides encoding each monomer are each contained within a single expression vector, generally under different or the same promoter controls. In embodiments of particular use in the present invention, each of these polynucleotides are contained on different expression vectors. As shown herein and in U.S. 62/025,931, hereby incorporated by reference, different vector ratios can be used to drive heterodimer formation. That is, surprisingly, while the proteins comprise first monomer: second monomer:light chains (in the case of many of the embodiments herein that have three polypeptides comprising the heterodimeric antibody) in a 1:1:2 ratio, these are not the ratios that give the best results.

The antibodies provided herein are made by culturing host cells comprising the expression vector(s) as is well known in the art. Once produced, traditional antibody purification steps are done, including an ion exchange chromatography step. As discussed herein, having the pIs of the two monomers differ by at least 0.5 can allow separation by ion exchange chromatography or isoelectric focusing, or other methods sensitive to isoelectric point. That is, the inclusion of pI substitutions that alter the isoelectric point (pI) of each monomer so that such that each monomer has a different pI and the heterodimer also has a distinct pI, thus facilitating isoelectric purification of the “1+1 Fab-scFv-Fc” heterodimer (e.g., anionic exchange columns, cationic exchange columns). These substitutions also aid in the determination and monitoring of any contaminating dual scFv-Fc and mAb homodimers post-purification (e.g., IEF gels, cIEF, and analytical IEX columns).

### VI. Biological and Biochemical Functionality of the Anti-CD28×Anti-MSLN Antibodies

Generally the anti-CD28×anti-MSLN antibodies described herein are administered to patients with a MSLN-associated cancer, and efficacy is assessed, in a number of ways as described herein. Thus, while standard assays of efficacy can be run, such as cancer load, size of tumor, evaluation of presence or extent of metastasis, etc., immuno-oncology treatments can be assessed on the basis of immune status evaluations as well. This can be done in a number of ways, including both in vitro and in vivo assays.

### A. Antibody Compositions for In Vivo Administration

Embodiments of the invention relate to a pharmaceutical composition comprising any one of the anti-CD28×anti-MSLN antibodies described herein and a pharmaceutically acceptable carrier. Formulations of the anti-CD28×anti-MSLN antibodies described herein are prepared for storage by mixing an antibody having the desired degree of purity with optional pharmaceutically acceptable carriers, excipients or stabilizers (Remington's Pharmaceutical Sciences 16th edition, Osol, A. Ed. [1980]), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™ PLURONICS™ or polyethylene glycol (PEG).

### VII. Treatments

Once made, the compositions of the invention find use in a number of oncology applications, by treating cancer, generally by enhancing immune responses (e.g., T cell activation and proliferation), particularly when used with anti-cancer therapies such as anti-tumor bispecific antibodies. In some embodiments, the antibodies provided herein enhance immune responses (e.g., T cell activation and proliferation) by providing agonistic co-stimulation of T cells in the microenvironment of tumors expressing MSLN.

In some embodiments, the anti-CD28×anti-MSLN bispecific antibodies provided herein are administered with an anti-tumor therapy including, for example, anti-tumor-associated antigen (TAA) bispecific antibodies.

### A. Anti-CD28×Anti-MSLN/Anti-MSLN Bispecific Antibody

In some embodiments, the anti-CD28×anti-MSLN antibodies provided herein are administered with an anti-MSLN bispecific antibody that is a T-cell engaging bispecific antibody, such as those that bind to human CD3.

In classic T cell/APC interaction, there is a first signal provided by TCR reactivity with peptide-MHC (Signal 1) and a second signal provided by CD28 crosslinking by CD80/CD86 being expressed on APCs (Signal 2) which together fully activate T cells (see FIG. 34A). In contrast, only the first signal is provided in treatment with CD3 bispecific antibodies that target a tumor-associated antigen (TAA)(i.e., anti-CD3×anti-MSLN bispecific antibodies).

Without being bound by any particular theory of operation, it is believed that the anti-CD28×anti-MSLN bispecific antibodies provided herein can enhance the anti-tumor response of an anti-CD3×anti-MSLN bispecific antibody by CD28 costimulation (see FIG. 34B). Thus, in one aspect, provided herein are methods of methods of treating a MSLN-associated cancer in a patient by administering the patient an anti-CD3×anti-MSLN bispecific antibody and an anti-CD28×anti-MSLN bispecific antibody provided herein.

Anti-CD3×anti-MSLN antibodies that are useful for providing “signal 1” in combination with the subject anti-CD28×anti-MSLN antibodies include those with any of the CD3 binding domains and MSLN binding domains provided herein (see, e.g., FIGS. 10, 15-19, and 23). Suitable antibody formats for such anti-CD3×anti-MSLN antibodies, include, but are not limited to the antibody formats described herein (see, e.g., FIG. 26). In some embodiments, the anti-CD3×anti-MSLN antibody and the anti-CD28×anti-MSLN antibody used in combination bind to the same MSLN epitope. In some embodiments, the anti-CD3×anti-MSLN antibody and the anti-CD28×anti-MSLN antibody used in combination bind to different MSLN epitopes.

### B. Administrative Modalities

The antibodies provided herein administered to a subject, in accord with known methods, such as intravenous administration as a bolus or by continuous infusion over a period of time.

### C. Treatment Modalities

In the methods of the invention, therapy is used to provide a positive therapeutic response with respect to a disease or condition.

By “positive therapeutic response” is intended an improvement in the disease or condition, and/or an improvement in the symptoms associated with the disease or condition. For example, a positive therapeutic response would refer to one or more of the following improvements in the disease: (1) a reduction in the number of neoplastic cells; (2) an increase in neoplastic cell death; (3) inhibition of neoplastic cell survival; (5) inhibition (i.e., slowing to some extent, preferably halting) of tumor growth; (6) an increased patient survival rate; and (7) some relief from one or more symptoms associated with the disease or condition.

Positive therapeutic responses in any given disease or condition can be determined by standardized response criteria specific to that disease or condition. Tumor response can be assessed for changes in tumor morphology (i.e., overall tumor burden, tumor size, and the like) using screening techniques such as magnetic resonance imaging (MRI) scan, x-radiographic imaging, computed tomographic (CT) scan, bone scan imaging, endoscopy, and tumor biopsy sampling including bone marrow aspiration (BMA) and counting of tumor cells in the circulation.

In addition to these positive therapeutic responses, the subject undergoing therapy may experience the beneficial effect of an improvement in the symptoms associated with the disease.

Treatment according to the present invention includes a “therapeutically effective amount” of the medicaments used. A “therapeutically effective amount” refers to an amount effective, at dosages and for periods of time necessary, to achieve a desired therapeutic result.

A therapeutically effective amount may vary according to factors such as the disease state, age, sex, and weight of the individual, and the ability of the medicaments to elicit a desired response in the individual. A therapeutically effective amount is also one in which any toxic or detrimental effects of the antibody or antibody portion are outweighed by the therapeutically beneficial effects.

A “therapeutically effective amount” for tumor therapy may also be measured by its ability to stabilize the progression of disease. The ability of a compound to inhibit cancer may be evaluated in an animal model system predictive of efficacy in human tumors.

Alternatively, this property of a composition may be evaluated by examining the ability of the compound to inhibit cell growth or to induce apoptosis by in vitro assays known to the skilled practitioner. A therapeutically effective amount of a therapeutic compound may decrease tumor size, or otherwise ameliorate symptoms in a subject. One of ordinary skill in the art would be able to determine such amounts based on such factors as the subject's size, the severity of the subject's symptoms, and the particular composition or route of administration selected.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. Parenteral compositions may be formulated in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

The specification for the dosage unit forms of the present invention are dictated by and directly dependent on (a) the unique characteristics of the active compound and the particular therapeutic effect to be achieved, and (b) the limitations inherent in the art of compounding such an active compound for the treatment of sensitivity in individuals.

The efficient dosages and the dosage regimens for the bispecific antibodies used in the present invention depend on the disease or condition to be treated and may be determined by the persons skilled in the art.

### EXAMPLES

### Example 1: CD28 Binding Domains

Sequences for human, mouse, and cynomolgus CD28 are depicted in FIGS. 14C-14D and are useful for the development of cross-reactive CD28 antigen binding domains for ease of clinical development.

### 1A: Novel CD28 Binding Domains

An approach considered to avoid the superagonism associated with TGN1412 was to generate novel CD28 binding domains having lower affinity binding to CD28 and/or binding to a different CD28 epitope than TGN1412. In one campaign to generate such novel CD28 binding domains, in-house de novo phage libraries were panned against CD28.

### 1A(a): Phage-Derived Clone 1A7

It should be noted that this phage library utilized a human germline VL with diversity introduced into the LCDR3. The amino acid sequences for exemplary phage-derived clone 1A7 are depicted in FIG. 27.

Towards optimization of CD28 bsAbs, numerous 1A7 affinity variants were developed by engineering VH variants (illustrative sequences as depicted in FIGS. 28A-28F and as SEQ ID NOs: 745-813), VL variants (illustrative sequences as depicted in FIGS. 29A-29I and as SEQ ID NOs: 814-919), consensus sequences for which are depicted in FIGS. 31A-31B, and combinations thereof (illustrative sequences for which are depicted in FIG. 30A-30C). Monovalent affinity of illustrative variants, in the context of scFvs, are depicted in FIG. 32.

### 1B: Additional CD28 Binding Domains

Sequences for additional CD28 binding domains which may find use in the MSLN×CD28 bsAbs of the invention are depicted in FIGS. 33A-33H.

### Example 2: MSLN Binding Domains

Expression of MSLN on cancer cell lines was correlated to 191 biopsy cores of serous and mucinous ovarian cancer tissues by IHC. Illustrative IHC of biopsy cores are depicted in FIG. 11, and samples were qualitatively scored in-house on a scale of 0-3 with 0 representing little to no MSLN expression and 3 representing high MSLN expression (herein referred to as IHC score; breakdown of score for each core is depicted in FIG. 12). Based on the results, it was determined that MSLN is a suitable tumor target for the novel CD28 bispecific antibodies of the invention and that they should target score 3 and score 2 cell lines as they represent 80% of the patient segment and should not target score 1 cell lines as they resemble normal tissue. Next, endogenous MSLN expression on cancer cell lines were investigated by IHC and flow cytometry, data for which are depicted in FIG. 13. MSLN-transfected A549 cells were found to stain as intensely as ovarian cancer tumors expressing high amounts of MSLN (data not shown). OVCAR-8 cancer cells and ASPC-1 cells represent high and medium MSLN-expressing tumors (score 3 or 2 by IHC). Finally, SKOV3, HT29, MCF7, Huh7, and A549 (parental) cancer cells were found to have MSLN-expression level correlating to non-cancerous adjacent tissues and therefore identified as appropriate surrogates of normal tissues.

Sequences for human, mouse, and cynomolgus MSLN are depicted in FIGS. 14A-14B and are useful for the development of cross-reactive MSLN antigen binding domains for ease of clinical development. Sequences for MSLN binding domains which may find use in the MSLN×CD28 bsAbs of the invention are depicted in FIGS. 15-19 and 23A-23FF.

Binding affinity for various MESO-C variants (in the context of CD3 bispecific antibodies) are depicted in FIGS. 20-21 and 25.

### Example 3: Engineering MSLN×CD28 Bsabs

T cells require multiple signals for complete activation and differentiation. Signal 1, promoted by recognition of a peptide-MHC (pMIC) complex by the T cell receptor (TCR), is absolutely required for T cell activation. Signal 2, which synergizes with, and amplifies Signal 1, is typically provided by the interaction of the CD28 ligands CD80 and CD86 with CD28 itself. Although CD28 engagement alone is typically inert, when combined with Signal 1 activation, it promotes additional activation, survival, and proliferative signals, including IL-2 secretion (see FIG. 34). As CD80 and CD86 are only naturally expressed by professional antigen-presenting cells (APC)s, the extent of CD28 costimulation in the tumor setting can be highly variable. By creating this novel class of tumor-targeted CD28 bispecific antibodies, CD80/CD86 engagement of CD28 can be mimicked, providing an artificial source of Signal 2. Notably, Signal 1 can either be provided by the natural TCR:pMHC recognition of tumor cells, or it can be provided by combination of the CD28 bispecific with a CD3 bispecific (which can mimic Signal 1). With these concepts in mind, MSLN×CD28 bsAbs were conceived, and a number of formats are contemplated for use, schematics for which are outlined in FIG. 22.

### 3A: 1+1 Fab-Fv-Fc Format

One exemplary format utilizing Fab domains and scFv is the 1+1 Fab-scFv-Fc format (depicted schematically in FIG. 22A) which comprises a first monomer comprising a single-chain Fv (“scFv”) with a first antigen binding specificity covalently attached to a first heterodimeric Fc domain i.e., scFv-domain linker-CH2-CH3, a second monomer comprising a heavy chain i.e., VH-CH1-hinge-CH2-CH3, wherein the CH2-CH3 is a second heterodimeric Fc domain complementary to the first heterodimeric Fc domain, and a light chain (LC) transfected separately so that a Fab domain having a second antigen binding specificity is formed with the variable heavy domain. Any number of heterodimerization approaches as is known in the art could find use in this (and other) bispecific formats, in combination with any number of approaches for purifying heterodimers from contaminating homodimers, including those depicted in FIGS. 1-2. Any of the number of linkers as is known in the art may find use in linking the VH and VL domains of the scFv. Finally, it may be useful to maximize serum half-life of the bsAbs, and any of the number of half-life extending variants as is known in the art may find use in these bsAbs.

In particular, the 1+1 Fab-scFv-Fc bsAbs may utilize Backbones 1 or 11 in FIG. 7. The backbones utilize the L368D/K370S (on the HC):S364K/E357Q (on the scFv-Fc) heterodimeric Fc variants. The HC side further includes pI variants N208D/Q295E/N384D/Q418E/N421D to increase negative charge of the heavy chain. The scFv utilizes a positively charged (GKPGS)4 linker (SEQ ID NO: 1) between the VH and VL domains to increase positive charge of the scFv-Fc chain. Collectively, these two approaches enable easy purification of heterodimers from contaminating homodimers. The FcγR ablation variants utilized in this platform are the E233P/L234V/L235A/G236_/S267K substitutions on both the HC and the scFv-Fc monomers. In some cases, bsAbs includes the M428L/N434S half-life extension variants. Sequences for illustrative MSLN×CD28 bsAbs in the 1+1 Fab-scFv-Fc format are depicted in FIG. 24 (for example, XENP39553). 3B: 2+1 mAb-scFv Format

Another exemplary format utilizing Fab domains and scFv is the 2+1 mAb-scFv format (depicted schematically in FIG. 26H which comprises a first monomer comprising a first heavy chain covalently attached to a single-chain Fv (“scFv”) with a first antigen binding specificity i.e., VH-CH1-hinge-CH2-CH3-domain linker-scFv, wherein CH2-CH3 is a first heterodimeric Fc domain; a second monomer comprising a heavy chain i.e., VH-CH1-hinge-CH2-CH3, wherein the CH2-CH3 is a second heterodimeric Fc domain complementary to the first heterodimeric Fc domain, and a light chain (LC) transfected separately so that a Fab domain having a second antigen binding specificity is formed with the two VH domains. Sequences for illustrative MSLN×CD28 bsAbs in the 2+1 mAb-scFv format are depicted in FIG. 24 (for example, XENP39551).

### 3C: 2+1 Fab2-scFv-Fc

Another format is the 2+1 Fab2-scFv-Fc format (depicted schematically in FIG. 26F) which comprises a first monomer comprising a VH domain covalently attached to a CH1 domain covalently attached to an scFv (having a first antigen binding specificity) covalently attached to a first heterodimeric Fc domain (VH-CH1-scFv-Fc), a second monomer which is a heavy chain comprising a VH-CH1 covalently attached to a complementary second heterodimeric Fc domain (VH-CH1-Fc), and a light chain transfected separately so that Fab domains having a second antigen binding specificity are formed with the VH domains. Sequences for illustrative MSLN×CD28 bsAbs (based on binding domains as described in Examples 1 and 2) in the 2+1 Fab2-scFv-Fc format are depicted in FIG. 35.

### 3D: 2+1 Fab2-Fc×scFv-Fc Format

An additional format is the 2+1 Fab2-Fc×scFv-Fc format (depicted schematically in FIG. 26L) which includes a first monomer, a second monomer, and a light chain. The first monomer comprises a VH domain covalently attached to a first CH1 domain covalently attached (e.g., by a domain linker) to a second VH domain covalently attached to a second CH1 domain covalently attached to a first heterodimeric Fc domain (e.g., VH-CH1-VH-CH1-hinge-CH2-CH3). The VHs of the first monomer and the VL of the light chain form two antigen binding domains with the same antigen binding specificity. The second monomer comprises a single-chain Fv (“scFv”) with a second antigen binding specificity covalently attached to a second heterodimeric Fc domain complementary to the first heterodimeric Fc domain (scFv-domain linker-CH2-CH3). Sequences for illustrative MSLN×CD28 bsAbs (based on binding domains as described in Examples 1 and 2) in the 2+1 Fab2-Fc×scFv-Fc format are depicted in FIG. 36.

Although the above provide particularly useful backbones, any number of heterodimerization approaches as is known in the art could find use in the bispecific formats, in combination with any number of approaches for purifying heterodimers from contaminating homodimers, including those depicted in FIGS. 1-2. Any of the number of linkers as is known in the art may find use in linking the VH and VL domains of the scFv. Finally, it may be useful to maximize serum half-life of the bsAbs, and any of the number of half-life extending variants as is known in the art may find use in these bsAbs. Regardless of bsAb format, the CD28 bispecific antibodies are monovalent for CD28 and incorporate Fc variants engineered to ablate FcγR binding to avoid potential superagonism. Such Fc variants include those depicted in FIG. 3.

### Example 4: Combination with CD3 Bsabs

As described in Example 3, the MSLN×CD28 bsAbs are contemplated for combination with CD3 bsAbs. Such CD3 bsAbs may utilize the CD3 binding domains as depicted in FIG. 10. Additionally, the MSLN×CD28 bsAbs may be combined with MSLN×CD3 bsAbs (see e.g., US Patent Application Publication No. 20170029502, US Patent Application Publication No. 20170096485, U.S. patent Ser. No. 10/730,954, WO2021239987, and Park Y, Lim Y, Kim K, et al. MSLN-targeting bispecific t-cell engaging antibody, MG1122, for treatment of solid tumors. J Clin Oncol 36, 2018 (suppl; abstract e14500) hereby incorporated by reference).

4A: MSLN×CD28 bsAbs Increase the Ability of T-Cell Engagers to Induce RTCC

10,000 OVCAR8-Luc cells (90K MSLN antigens) were seeded per well with T-cells at an effector to target ratio of 1:1, 1:10, and 1:20 in the presence of a constant 1 g/mL of each MSLN×CD28 bsAb and the CD3 engager, XENP41407 (EPCAM×CD3), at the concentrations indicated in FIG. 37. RTCC was assessed after 3 and 5 days via Luciferase. The results depicted in FIG. 37 illustrate the ability of MSLN×CD28 antibodies to enhance RTCC driven by a CD3 engager even at the lower E:T ratio of 1:20.

4B: MSLN×CD28 bsAbs Selectively Enhance B7H3×CD3 bsAbs on Cells with High MSLN Expression

10,000 OVCAR8 (~90K MSLN antigens/cell) or SKOV3 cells (~9K MSLN antigens/cell) were seeded per well in a plate. The following day, T cells were seeded at an effector to target ratio of 10:1 and 1:10 in the presence of a constant 1 g/ml B7H3×CD3 bsAb, and a MSLN×CD28 bsAb at the concentrations indicated in FIG. 38. IL-2 release was assessed after 1 day via MSD (Meso Scale Discovery, Rockville, Md.). As depicted in FIG. 38, IL-2 release was enhanced on OVCAR8 cells but not on SKOV3 cells. This is ideal since as discussed in Example 2, OVCAR8 cells are representative of high and medium MSLN-expressing tumors, while SKOV3 cells are representative of normal healthy tissues.

### Example 5: Further Characterization of MSLN×CD28 Bsabs

5A: Monovalent Engagement with High Affinity 1+1 Fab-scFv Formats Maximize Binding Efficacy while Bivalent MSLN×CD28 Formats Display Avid Binding

In order to assess the binding of MSLN×CD28 bsAbs in different formats, an experiment was conducted in which 50,000 OVCAR8 cells (~90K MSLN antigens/cell) were seeded per well in a plate. Next, antibodies were added at the doses indicated in FIG. 39 for 1 hour at 4° C. Anti-human-Fc secondary (A647) was added for 30 min at 4° C., and binding was assessed via FACs. As can be seen in FIG. 39A, bivalent engagement by any of the 2+1 formats (2+1 Fab2-Fc×scFv-Fc, 2+1 Fab2-scFv-Fc, or 2+1 mAb-scFv) displays avid binding, while monovalent engagement by the highest affinity MSLN binding domain in the 1+1 Fab-scFv-Fc format maximizes binding efficacy. Additionally, using the same experimental setup except with low-MSLN expressing SKOV3 cells (~9K MSLN antigens/cell) instead of OVCAR8 cells, neither the 1+1-Fab-scFv format nor the 2+1 mAb-scFv format antibodies showed any significant binding, as depicted in FIG. 39B, indicating that the MSLN×CD28 bsAbs will avoid off-target binding.

5B: MSLN (0.3 nM)×CD28 (37 nM) 1+1 Fab-scFv-Fc is Optimal for Enhancing IL-2 Release Driven by a CD3 Engager

Several different MSLN×CD28 bsAb formats of various affinities were tested for their ability to enhance IL-2 release. Either 10,000 Kuramochi cells (having ~120K MSLN antigens/cell) or OVCAR3 cells were seeded per well in a plate. OVCAR3 is still considered a MSLN-high cell line, but is estimated to have fewer MSLN antigens/cell than Kuramochi cells. The following day, T cells were seeded at an effector to target ratio of 1:1 in the presence of 1 μg/ml EPCAM×CD3 and the indicated concentrations of MSLN×CD28 bsAbs as depicted in FIG. 40. IL-2 release was assessed after 1 day via MSD (Meso Scale Discovery, Rockville, Md.). As depicted in FIG. 40, the bsAbs in the 1+1 Fab-scFv-Fc format produced the highest amplitude IL-2 release by a substantial margin. Unexpectedly, the 2+1 Fab2-Fc×scFv-Fc format resulted in higher levels of IL-2 release compared with the other 2+1 formats such as the 2+1 Fab2-scFv-Fc despite having similar cell binding efficiencies and potencies. The loss of activity may be due to the loss of affinity that occurs when CD28 scFvs are used in the various 2+1 formats as demonstrated in FIG. 32B. The results depicted in FIG. 40 also indicate that the higher affinity CD28 binding domains (1A7_H1.14_L1.71, KD value of 37 nM) correspond with higher levels of IL-2 release for MSLN×CD28 bsAbs in the 1+1 Fab-scFv-Fc format. Likewise, the higher affinity MSLN binding domains (particularly MESO-C H1_L1, KD value of 0.3 nM) corresponds with higher IL-2 release for MSLN×CD28 bsAbs in the 1+1 Fab-scFv-Fc format. XENP39553 in particular, having the 0.3 nM mesothelin binding domain the and the 37 nM CD28 binding domain, displayed enhanced IL-2 release at a significantly higher level than any of the other MSLN×CD28 bsAbs.

All cited references are herein expressly incorporated by reference in their entirety.

Whereas particular embodiments of the invention have been described above for purposes of illustration, it will be appreciated by those skilled in the art that numerous variations of the details may be made without departing from the invention as described in the appended claims.

1-83. (canceled)

84. An anti-CD28×anti-mesothelin (MSLN) antibody comprising:
a) a means for binding CD28;
b) a means for binding MSLN; and
c) a dimeric Fc domain.

85. A nucleic acid composition encoding the anti-CD28×anti-MSLN antibody of claim 84 comprising:
a) one or more nucleic acids encoding the means for binding CD28;
b) one or more nucleic acids encoding the means for binding MSLN; and
c) a nucleic acid encoding the dimeric Fc domain.

86. A host cell comprising the nucleic acid composition of claim 85.

87. A method of treating a MSLN-associated cancer in a patient in need thereof, comprising administering to the patient the anti-CD28×anti-MSLN antibody of claim 84.

88. A method of activating a T cell comprising contact the T cell with the anti-CD28×anti-MSLN antibody of claim 84.
